(19) United States
(12) Patent Application Publication
(10) Pub. No.: US 2022/0387411 A1
(43) Pub. Date: Dec. 8, 2022

(54) TARGETED AUTOPHAGY CONJUGATES AND METHODS

(71) Applicant: Frontier Medicines Corporation, South San Francisco, CA (US)

(72) Inventors: Michael B. SHAGHAFI, San Diego, CA (US); Johannes HERMANN, San Francisco, CA (US)

(21) Appl. No.: 17/763,156

(22) PCT Filed: Sep. 23, 2020
(86) PCT No.: PCT/US2020/052322

§ 371 Date: Mar. 23, 2022

Related U.S. Application Data
(60) Provisional application No. 62/905,795, filed on Sep. 25, 2019.

(51) Int. Cl. A61K 31/4468; A61K 31/426; A61K 31/453; A61K 31/454; A61K 31/55

(52) U.S. Cl. CPC A61K 31/4468; A61K 31/426; A61K 31/453; A61K 31/454; A61K 31/55

(57) ABSTRACT

Provided herein are methods and compounds for targeted autophagy.

### CROSS-REFERENCE TO RELATED APPLICATIONS

This application claims priority benefit of U.S. Provisional Patent Application No. 62/905,795 filed Sep. 25, 2019. The contents of that patent application are hereby incorporated by reference herein in their entirety.

### FIELD OF THE INVENTION

Provided herein are compounds and compositions for targeted autophagy protein binding. Also provided herein are the use of such compounds and compositions in degrading a cellular component associated with a disease, such as a neurodegenerative disease, and the treatment of the disease.

### BACKGROUND

Although numerous protein targets have been identified as potential human therapy targets, many of these proteins are difficult to target with small molecules. For example, proteins that do not possess obvious small molecule binding pockets or “druggable hotspots” are often considered “undruggable”. This represents a major challenge in developing next-generation drugs.

Autophagy is central to the maintenance of homeostasis in both physiological and pathological situations. It is an essential, conserved lysosomal degradation pathway that controls the quality of the cytoplasm by eliminating aggregated proteins and damaged organelles. Accordingly, alterations in autophagy have been linked to a wide range of diseases and conditions including aging, cancer, metabolic disorders, and neurodegenerative diseases. Autophagy begins with double-membraned autophagosomes which engulf portions of the cytoplasm, followed by fusion of these vesicles with lysososomes and degradation of the autophagic contents. This pathway is dysregulated across many human disorders, including metabolic conditions, neurodegenerative diseases, cancers, and infectious diseases. Autophagosome formation is a multi-step process that includes the biogenesis of the phagophore, followed by its elongation and closure. More than 15 autophagy-related ATG proteins, as well as class III PI3 kinases, are required to construct the autophagosome, including the only transmembrane ATG protein ATG9, along with membranes from multiple cellular sources. The proteins ATG8 and microtubule-associated protein 1 light-chain 3 (LC3) are involved in expansion and fusion of phagophore edges, and recruit adaptor proteins such as ubiquitin-binding protein p62 and NBR1 to autophagosomes via their LC3-interacting region (LIR) domains. In turn, autophagic adaptors enable the selective degradation of aged or damaged cellular structures, protein aggregates, and microorganisms.

Most neurodegenerative disease are associated with intracytoplasmic deposition of aggregate-prone proteins in neurons and with mitochondrial dysfunction. Autophagy is a powerful process for removing such proteins and for maintaining mitochondrial homeostasis.

Over recent years, evidence has accumulated to demonstrate that upregulation of autophagy is protective against neurodegeneration. Numerous studies have demonstrated that aggregate-prone proteins at the heart of neurodegenerative disease toxicity are autophagy substrates, and that pharmacological upregulators of autophagy can be beneficial in both cell and animal models of these diseases by reducing intracytoplasmic aggregates and associated cell death. Specifically targeting aggregated proteins to autophagy and lysosomal degradation could enable clearing of toxic protein aggregates and prevent neurodegeneration.

A frequently overlooked parameter that defines functional “hotspots” in the proteome is amino acid side-chain reactivity, which can vary by orders of magnitude for a given residue depending on the local protein microenvironment. Such hotspot amino acids, including cysteine, lysine, and serine, are highly enriched in functional residues that are involved in catalysis, protein-protein interactions, metal binding, post-translational modification, or allosteric regulation. These hotspot amino acids can be targeted and covalently modified using appropriate reagents.

Targeted Protein Autophagy (TPA) uses bifunctional small molecule degraders to engulf the protein cargo into an autophagosome for lysosomal degradation. The bifunctional small molecules include a first moiety that targets a protein of interest linked to a second moiety that recruits an autophagy adaptor protein (e.g., p62). TPA can be applied for therapeutically degrading any specific protein target, protein complex, or aggregated or misfolded proteins within the cell.

Described herein are compounds comprising a targeting moiety, such as a p62/SQSTM1-targeting ligand, linked to a protein-targeting moiety. These compounds may degrade a protein target in a proteasome-independent and autophagy-dependent manner.

### SUMMARY

In one aspect, provided herein is a compound comprising a monovalent cellular component binder covalently bound to a monovalent targeted autophagy protein binder, wherein the monovalent targeted autophagy protein binder is a monovalent form of the formula:

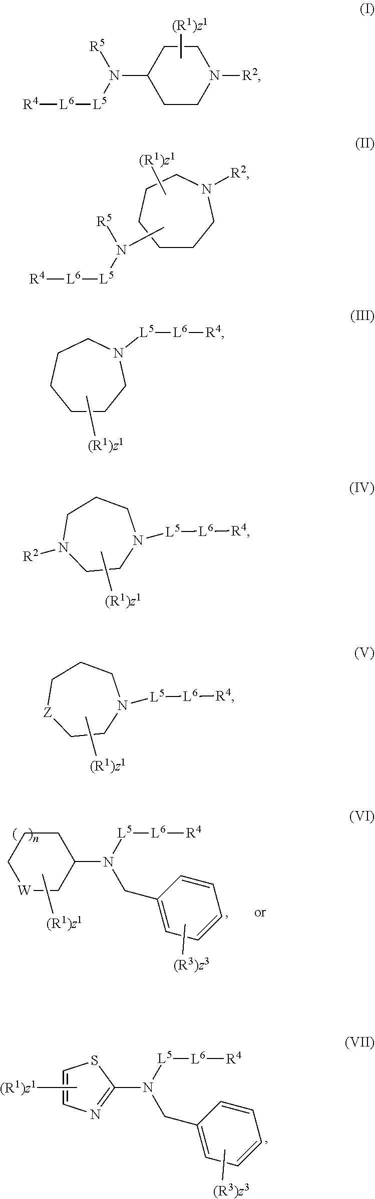

wherein R¹, R², R³, R⁴, R⁵, L⁵, L⁶, z¹, z³, Z, W, and n are as described herein.

In another aspect, provided herein is an autophagy adapter protein (e.g., p⁶²) covalently bonded to a compound described herein.

In another aspect, provided herein is a method for treating a disease associated with a cellular component (e.g., aberrant level of a cellular component), the method comprising contacting the cellular component with a targeted autophagy degrader as described herein. In a further aspect, provided herein is a method for treating a disease associated with a cellular component (e.g., aberrant level of a cellular component), the method comprising administering to a subject in need thereof a therapeutically effective amount of a compound described herein.

In another aspect, provided herein is a pharmaceutical composition comprising a compound described herein (e.g., a targeted autophagy degrader) and a pharmaceutically acceptable excipient.

### BRIEF DESCRIPTION OF THE DRAWINGS

FIG. 1 shows the dose-response of Compound 1 (A) and Compound 19 (B) against autophagy adapter protein SQSTM1 (p62) using gel-based densitometry measurements as described in Example B2. Protein SQSTM1 (p62) was pre-incubated with Compound 1 or Compound 19 at concentrations of 2, 0.5, 0.125, and 0.03 μM, followed by addition of the reactive probe 5-carboxytetramethylrhodamine (TAMRA). Measurements were made using TAMRA fluorescence (a), and protein loading was measured by silver staining (b). Decreasing fluorescence of TAMRA indicates binding of the test compound.

### DETAILED DESCRIPTION

Disclosed herein are compounds which are bifunctional small-molecule degraders comprising a protein-targeting ligand, a linker, and a recruiter for autophagy adapter proteins, such as p62, to target specific substances, for example proteins, misfolded proteins, protein aggregates, organelles, or microorganisms, to autophagasomes for lysosomal degradation.

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds. In some embodiments, alkyl refers to an aliphatic hydrocarbyl.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring.

The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkyl arylene” as an arylene moiety covalently bonded to an alkylene moiety (also referred to herein as an alkylene linker). In some embodiments, the alkyl arylene group has the formula:

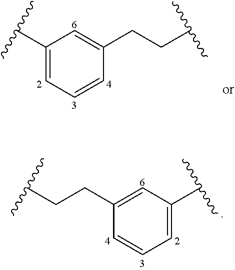 [SMILES: *CCc1cccc(*)c1 ; *CCc1cccc(*)c1]

An alkyl arylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃ —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In some embodiments, the alkyl arylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R″′)═NR″ ″, —NR—C(NR′R″)═NR″′, —S(O)R′, —S(O)₂R′, —S(O)₂NR′R″, —NRSO₂R′, —NR′NR″R″′, —ONR′R″, —NR′C(O)NR″NR″′R″ ″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R″′)═NR″ ″, —NR—C(NR′R″)═NR″′, —S(O)R′, —S(O)₂R′, —S(O)₂NR′R″, —NRSO₂R′, —NR′NR″R″′, —ONR′R″, —NR′C(O)NR″NR″′R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R″′, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3.

Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R″′)_{d}—, where s and d are independently integers of from 0 to 3, and X is —O—, —NR′—, —S—, —S(O)—, —S(O)₂—, or —S(O)₂NR′—. The substituents R, R′, R″, and R″′ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl, heteroalkyl, cycloalkyl, heterocycloalkyl, aryl, heteroaryl, substituted with at least one substituent selected from:
   (i) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C6-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl, heteroalkyl, cycloalkyl, heterocycloalkyl, aryl, heteroaryl, substituted with at least one substituent selected from:
      (a) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl, heteroalkyl, cycloalkyl, heterocycloalkyl, aryl, heteroaryl, substituted with at least one substituent selected from: oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In some embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In some embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In some embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In some embodiments, if the substituted moiety is substituted with a plurality of substituent groups, each substituent group is different.

In some embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In some embodiments, if the substituted moiety is substituted with a plurality of size-limited substituent groups, each size-limited substituent group is different.

In some embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In some embodiments, if the substituted moiety is substituted with a plurality of lower substituent groups, each lower substituent group is different.

In some embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In some embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)-, or as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the term “bioconjugate reactive moiety” and “bioconjugate linker” refers to the resulting association between atoms or molecules of bioconjugate reactive groups. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g. ionic bond, hydrogen bond, halogen bond), van der Waals interactions (e.g., dipole-dipole, dipole-induced dipole, London dispersion), ring stacking (pi effects), hydrophobic interactions and the like). In some embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In some embodiments, the first bioconjugate reactive group (e.g., maleimide moiety) is covalently attached to the second bioconjugate reactive group (e.g. a sulfhydryl). In some embodiments, the first bioconjugate reactive group (e.g., haloacetyl moiety) is covalently attached to the second bioconjugate reactive group (e.g., a sulfhydryl). In some embodiments, the first bioconjugate reactive group (e.g., pyridyl moiety) is covalently attached to the second bioconjugate reactive group (e.g., a sulfhydryl). In some embodiments, the first bioconjugate reactive group (e.g., —N-hydroxysuccinimide moiety) is covalently attached to the second bioconjugate reactive group (e.g., an amine). In some embodiments, the first bioconjugate reactive group (e.g., maleimide moiety) is covalently attached to the second bioconjugate reactive group (e.g., a sulfhydryl). In some embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example:

(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;

(b) hydroxyl groups which can be converted to esters, ethers, aldehydes, etc.;

(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;

(d) dienophile groups which are capable of participating in Diels-Alder reactions such as, for example, maleimido or maleimide groups;

(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;

(f) sulfonyl halide groups for subsequent reaction with amines, for example, to form sulfonamides;

(g) thiol groups, which can be converted to disulfides, reacted with acyl halides, or bonded to metals such as gold, or react with maleimides;

(h) amine or sulfhydryl groups (e.g., present in cysteine), which can be, for example, acylated, alkylated or oxidized;

(i) alkenes, which can undergo, for example, cycloadditions, acylation, Michael addition, etc;

(j) epoxides, which can react with, for example, amines and hydroxyl compounds;

(k) phosphoramidites and other standard functional groups useful in nucleic acid synthesis;

(l) metal silicon oxide bonding;

(m) metal bonding to reactive phosphorus groups (e.g., phosphines) to form, for example, phosphate diester bonds;

(n) azides coupled to alkynes using copper catalyzed cycloaddition click chemistry; and

(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In some embodiments, the bioconjugate comprises a molecular entity derived from the reaction of an unsaturated bond, such as a maleimide, and a sulfhydryl group.

“Analog” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound. A “derivative” is a compound derived from a chemical compound via a chemical reaction. A derivative of a compound described herein may refer to the compound described herein with the addition or removal of a substituent.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, propionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

The terms “polypeptide,” “peptide,” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer.

A polypeptide, or a cell is “recombinant” when it is artificial or engineered, or derived from or contains an artificial or engineered protein or nucleic acid (e.g., non-natural or not wild-type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Co-administer” it is meant that a composition described herein is administered at the same time, just prior to, or just after the administration of one or more additional therapies. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g., to reduce metabolic degradation). The compositions of the present invention can be delivered transdermally, by a topical route, or formulated as applicator sticks, solutions, suspensions, emulsions, gels, creams, ointments, pastes, jellies, paints, powders, and aerosols.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., Spodoptera) and human cells. Cells may be useful when they are naturally nonadherent or have been treated not to adhere to surfaces, for example by trypsinization.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat cancer by decreasing the incidence of cancer and or causing remission of cancer. In some embodiments of the compositions or methods described herein, treating cancer includes slowing the rate of growth or spread of cancer cells, reducing metastasis, or reducing the growth of metastatic tumors. The term “treating” and conjugations thereof, include prevention of an injury, pathology, condition, or disease. In some embodiments, treating is preventing. In some embodiments, treating does not include preventing. In some embodiments, the treating or treatment is no prophylactic treatment.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition (e.g., reduce signaling pathway stimulated by an autophagy adapter protein, reduce the signaling pathway activity of an autophagy protein). An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In some embodiments inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

“Disease” or “condition” refer to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule).

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head & neck, liver, kidney, lung, non-small cell lung, melanoma, mesothelioma, ovary, sarcoma, stomach, uterus, Medulloblastoma, colorectal cancer, pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cunateous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatinifori carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

As used herein, the term “neurodegenerative disease” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff's disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, or Tabes dorsalis.

Neurodegenerative diseases may be caused by (i.e., associated with) the accumulation of (e.g., insoluble) protein aggregates in and around neurons. In Huntington's disease, the huntingtin protein may form protein aggregates, also known as “huntingtin aggregates”.

The term “polyglutamine diseases” or “polyQ diseases” refers to a group of neurodegenerative diseases caused by expanded cytosine-adenine-guanine (CAG) repeats encoding a long polyQ tract in the respective proteins. The protein including the polyQ tract may form a protein aggregate (“polyQ protein aggregate”). In Huntington's disease, the huntingtin protein may include a polyQ tract and may form a protein aggregate or “polyQ huntingtin aggregate”.

As used herein, the term “metabolic disease” or “metabolic disorder” refers to a disease or condition in which a subject's metabolism or metabolic system (e.g., function of storing or utilizing energy) becomes impaired. Examples of metabolic diseases that may be treated with a compound, pharmaceutical composition, or method described herein include diabetes (e.g., type I or type II), obesity, metabolic syndrome, or a mitochondrial disease (e.g., dysfunction of mitochondria or aberrant mitochondrial function).

The term “cellular component associated disease” (e.g., the cellular component may be a protein, ion, lipid, nucleic acid, nucleotide, amino acid, protein, particle, organelle, cellular compartment, microorganism, virus, vesicle, small molecule, protein complex, protein aggregate, or macromolecule; the disease may be a neurodegenerative disease, cancer, a metabolic disease, authoimmune disease, inflammatory disease, or infectious disease) (also referred to herein as “cellular component related disease”) refers to a disease caused by the celllular component. Other diseases that are associated with aberrant activity or level of the cellular component are well known in the art and determining such diseases are within the skill of a person of skill in the art.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The term “administer (or administering) a targeted autophagy degrader” means administering a compound that inhibits the activity or level (e.g., amount) or level of a signaling pathway of a cellular component targeted by the targeted autophagy degrader to a subject. Administration may include, without being limited by mechanism, allowing sufficient time for the targeted autophagy degrader to reduce the level or activity of the cellular component or for the targeted autophagy degrader to reduce one or more symptoms of a disease.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

As a non-limiting example, the compounds described herein can be co-administered with conventional chemotherapeutic agents including alkylating agents (e.g., cyclophosphamide, ifosfamide, chlorambucil, busulfan, melphalan, mechlorethamine, uramustine, thiotepa, nitrosoureas, etc.), anti-metabolites (e.g., 5-fluorouracil, azathioprine, methotrexate, leucovorin, capecitabine, cytarabine, floxuridine, fludarabine, gemcitabine, pemetrexed, raltitrexed, etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g., cisplatin, oxaloplatin, carboplatin, etc.), and the like.

The compounds described herein can also be co-administered with conventional hormonal therapeutic agents including, but not limited to, steroids (e.g., dexamethasone), finasteride, aromatase inhibitors, tamoxifen, and gonadotropin-releasing hormone agonists (GnRH) such as goserelin.

Additionally, the compounds described herein can be co-administered with conventional immunotherapeutic agents including, but not limited to, immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), and radioimmunotherapy (e.g., anti-CD20 monoclonal antibody conjugated to ¹¹¹In, ⁹⁰Y, or ¹³¹I, etc.).

In a further embodiment, the compounds described herein can be co-administered with conventional radiotherapeutic agents including, but not limited to, radionuclides such as ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ^{117m}Sn, ¹⁴⁹Pm, ¹⁵³Sm, ¹⁶⁶Ho, ¹⁷⁷Lu, ¹⁸⁶Re, ¹⁸⁸Re, ²¹¹At, and ²¹²Bi, optionally conjugated to antibodies directed against tumor antigens.

In therapeutic use for the treatment of cancer, compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of cancer diagnosed in a particular patient. The dose administered to a patient, in the context of the present invention, should be sufficient to affect a beneficial therapeutic response in the patient over time. The size of the dose will also be determined by the existence, nature, and extent of any adverse side effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The compounds described herein can be used in combination with one another, with other active agents known to be useful in treating cancer or with adjunctive agents that may not be effective alone, but may contribute to the efficacy of the active agent.

The term “associated” or “associated with” in the context of a substance or substance activity or function associated with a disease (e.g., a protein associated disease, disease associated with a cellular component) means that the disease (e.g., neurodegenerative disease, cancer) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). For example, a neurodegenerative disease associated with a protein aggregate may be a neurodegenerative disease that results (entirely or partially) from aberrant protein aggregation or a neurodegenerative disease wherein a particular symptom of the disease is caused (entirely or partially) by aberrant protein aggregation. As used herein, what is described as being associated with a disease, if a causative agent, could be a target for treatment of the disease. For example, a neurodegenerative disease associated with aberrant protein aggregation or a protein aggregate associated neurodegenerative disease, may be treated with a protein aggregate modulator or protein aggregate targeted autophagy degrader, in the instance where increased protein aggregation causes the neurodegenerative disease.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

“Anti-cancer agent” is used in accordance with its plain ordinary meaning and refers to a composition (e.g., compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer.

“Chemotherapeutic” or “chemotherapeutic agent” is used in accordance with its plain ordinary meaning and refers to a chemical composition or compound having antineoplastic properties or the ability to inhibit the growth or proliferation of cells.

The term “electrophilic” as used herein refers to a chemical group that is capable of accepting electron density. An “electrophilic substituent,” “electrophilic chemical moiety,” or “electrophic moiety” refers to an electron-poor chemical group, substitutent, or moiety (monovalent chemical group), which may react with an electron-donating group, such as a nucleophile, by accepting an electron pair or electron density to form a bond. In some embodiments, the electrophilic substituent of the compound is capable of reacting with a cysteine residue. In some embodiments, the electrophilic substituent is capable of forming a covalent bond with a cysteine residue (e.g., LC3, p62, NBR1, NDP52, or Optineurin cysteine residue) and may be referred to as a “covalent cysteine modifier” or “covalent cysteine modifier moiety” or “covalent cysteine modifier substituent.” The covalent bond formed between the electrophilic substituent and the sulfhydryl group of the cysteine may be a reversible or irreversible bond. In some embodiments, the electrophilic substituent of the compound is capable of reacting with a lysine residue. In some embodiments, the electrophilic substituent of the compound is capable of reacting with a serine residue. In some embodiments, the electrophilic substituent of the compound is capable of reacting with a methionine residue.

“Nucleophilic” as used herein refers to a chemical group that is capable of donating electron density.

An amino acid residue in a protein “corresponds” to a given residue when it occupies the same essential structural position within the protein as the given residue. For example, a selected residue in a selected protein corresponds to C26 of human p62/SQSTM1 protein when the selected residue occupies the same essential spatial or other structural relationship as C26 in human p62/SQSTM1 protein. In some embodiments, where a selected protein is aligned for maximum homology with the human p62/SQSTM1 protein, the position in the aligned selected protein aligning with C26 is said to correspond to C26. Instead of a primary sequence alignment, a three dimensional structural alignment can also be used, e.g., where the structure of the selected protein is aligned for maximum correspondence with the human p62/SQSTM1 protein and the overall structures compared. In this case, an amino acid that occupies the same essential position as C26 in the structural model is said to correspond to the C26 residue.

An amino acid residue in a protein “corresponds” to a given residue when it occupies the same essential structural position within the protein as the given residue. For example, a selected residue in a selected protein corresponds to C27 of human p62/SQSTM1 protein when the selected residue occupies the same essential spatial or other structural relationship as C27 in human p62/SQSTM1 protein. In some embodiments, where a selected protein is aligned for maximum homology with the human p62/SQSTM1 protein, the position in the aligned selected protein aligning with C27 is said to correspond to C27. Instead of a primary sequence alignment, a three dimensional structural alignment can also be used, e.g., where the structure of the selected protein is aligned for maximum correspondence with the human p62/SQSTM1 protein and the overall structures compared. In this case, an amino acid that occupies the same essential position as C27 in the structural model is said to correspond to the C27 residue.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, wherein the polymer may In some embodiments, be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

As used herein, “biomolecule” is used in its customary sense and refers to a molecule found in nature or derivatives thereof, including macromolecules such as proteins, carbohydrates, lipids, and nucleic acids, as well as small molecules such as primary metabolites, secondary metabolites, and natural products. A biomolecule may be present as a moiety attached to the remainder of a compound. A biomolecule includes but is not limited to nucleic acids (e.g., DNA and RNA), peptide nucleic acids, sugars, peptides, proteins, antibodies, aptamers, lipids, small molecule affinity ligands (e.g., inhibitors, biotin, and haptens).

The term “targeted autophagy degrader” refers to a first substance (e.g., compound, biomolecule) capable of binding a targeted second substance (e.g., protein, protein aggregate, cellular component) and also binding a third substance, wherein the third substance is a component of an autophagy pathway or is associated with an autophagosome or with autophagy and wherein the targeted autophagy degrader binding to both the targeted second substance and third substance results in encapsulation of the targeted second substance in an autophagosome and subsequent degradation by autophagy. In some embodiments, a targeted autophagy binder is a compound described herein.

The term “autophagy adapter protein binder” as used herein refers to a substance (e.g., a biomolecule, macromolecule, or compound) which is capable of binding (e.g., covalently binding) an autophagy adapter protein. In some embodiments, autophagy adapter protein binder is a targeted autophagy binder. In some embodiments, autophagy adapter protein binder is a part of a targeted autophagy binder. The term “targeted autophagy binder” refers to a substance (e.g., a biomolecule, macromolecule, or compound) which is capable of contacting a component of an autophagy pathway or component (e.g., protein) of a complex involved in the autophagy and/or formation of the autophagosome. In some embodiments, the targeted autophagy binder is capable of binding (e.g., covalently binding) an autophagy adapter protein.

The term “autophagy adapter protein” as used herein refers to a protein which act as cargo receptor for degradation by autophagy. In some embodiments, the autophagy adapter protein is p62, or a derivative, fragment, or homolog thereof. Additional information and characterization of the mechanisms involved with autophagy adapter proteins may be found in Johansen and Lamark (Johansen T, Lamark T. Selective autophagy mediated by autophagic adapter proteins. Autophagy. 2011; 7(3):279-296. doi:10.4161/auto.7.3.14487), which is incorporated herein by reference in its entirety.

The term “cellular component binder” as used herein refers to a substance (e.g., a biomolecule, macromolecule, or compound) which is capable of binding a cellular component. In some embodiments, the cellular component binder is a compound (e.g., a compound described herein). In some embodiments, the cellular component binder is capable of binding a protein (e.g., BRD4). In some embodiments, the cellular component binder is capable of binding a protein aggregate. In some embodiments, the cellular component binder is a protein (e.g., antibody, antibody fragment, or receptor), nucleic acid (e.g., siRNA, antisense nucleic acid), aptamer, or compound).

The term “cellular component” as used herein refers to matter contained inside a cell (i.e., intracellular). Cellular components include matter naturally inside the cell (i.e., on the interior of the cell's lipid bilayer) as well as originally foreign agents (e.g., microorganisms, viruses, asbestos, or compounds or extracellular origin) that exist inside the cell. Non-limiting examples of a cellular component includes a protein (e.g., LC3, p62, NBR1, NDP52, Optineurin, or a derivative, fragment, or homolog thereof), ion (e.g., Na⁺, Mg⁺, Cu⁺, Cu²⁺, Zn²⁺, Mn²⁺, Fe²⁺, and Co²⁺), polysaccharides, lipid (e.g., fats, waxes, sterols, fat-soluble vitamins such as vitamins A, D, E, and K, monoglycerides, diglycerides, triglycerides, or phospholipids), nucleic acid (e.g., DNA or RNA), nucleotide, amino acid, particle (e.g., nanoparticle), fibers (e.g., asbestos fibers), organelle (e.g., mitochondria, peroxisome, plastid, endoplasmic reticulum, flagellum, or Golgi apparatus), cellular compartment, microorganism (e.g., bacterium, virus, or fungus), virus, vesicle (e.g., lysosome, peroxisome), small molecule, protein complex, protein aggregate, or a macromolecule). In some embodiments, the cellular component is a biomolecule. In some embodiments, the cellular component is a protein aggregate, soluble protein, midbody ring, damaged mitochodria, peroxisomes, intracellular bacteria, phagocytic membrane remnants, or viral capsid proteins. Non-limiting examples of intracellular proteins include BRD4, KRAS, MYC, YAP, TAZ, CTNNB1, APP, HTT, SNCA, NRF2, and MAPT. In some embodiments, the cellular component is a protein aggregate (e.g., HTT, APP, SNCA, or MAPT). In some embodiments, the cellular component is PINK1, ATG32, ESYT, PI3KC3, RAB10, or ATGL. In some embodiments, the cellular component is a microorganism. In some embodiments, the cellular component is a bacterial cell-surface glycan or bacterial cell surface protein.

The term “microorganism” is used in accordance with its plain ordinary meaning and refers to a single-cell organism, or multi-cellular organism (e.g., bacteria, fungi, protozoa) that is not visible to the naked eye. In some embodiments, the microorganism is a bacterium.

The terms “virus” or “virus particle” are used according to their plain ordinary meanings within Virology and refer to a virion including the viral genome (e.g., DNA, RNA, single strand, double strand), viral capsid and associated proteins, and in the case of enveloped viruses (e.g., herpesvirus), an envelope including lipids and optionally components of host cell membranes, and/or viral proteins.

The term “small molecule” is used in accordance with its plain ordinary meaning and refers to a low molecular weight (e.g., with a molecular weight equal to or less than 900 Daltons) compound. In some embodiments, the molecular weight of the small molecule is less than 500 Daltons. In some embodiments, metabolites (e.g., secondary metabolites) are considered small molecules.

The term “protein complex” is used in accordance with its plain ordinary meaning and refers to a protein which is associated with an additional substance (e.g., another protein, protein subunit, or a compound). Protein complexes typically have defined quaternary structure. The association between the protein and the additional substance may be a covalent bond. In some embodiments, the association between the protein and the additional substance (e.g., compound) is via non-covalent interactions. In some embodiments, a protein complex refers to a group of two or more polypeptide chains. Proteins in a protein complex are linked by non-covalent protein-protein interactions. A non-limiting example of a protein complex is the proteasome.

The term “proteasome” is used in accordance with its plain ordinary meaning and refers to a protein complex which degrades proteins by proteolysis. The proteasome is made up of two subcomplexes: a catalytic core particle (also known as the 20S proteasome) and one or two terminal 19S regulatory particle(s) (RP) that serves as a proteasome activator with a molecular mass of approximately 700 kDa (called PA700). In some embodiments, the proteasome degrades proteins thereby generating oligopeptides ranging in length from 3 to 15 amino-acid residues. Further information regarding the proteasome may be found in Tanaka (Tanaka K. The proteasome: Overview of structure and functions. Proceedings of the Japan Academy Series B, Physical and Biological Sciences. 2009; 85(1):12-36. doi:10.2183/pjab.85.12), which is incorporated herein by reference in its entirety for all purposes.

The term “protein aggregate” is used in accordance with its plain ordinary meaning and refers to an aberrant collection or accumulation of proteins (e.g., misfolded proteins). Protein aggregates are often associated with diseases (e.g., amyloidosis). Typically, when a protein misfolds as a result of a change in the amino acid sequence or a change in the native environment which disrupts normal non-covalent interactions, and the misfolded protein is not corrected or degraded, the unfolded/misfolded protein may aggregate. There are three main types of protein aggregates that may form: amorphous aggregates, oligomers, and amyloid fibrils. In some embodiments, protein aggregates are termed aggresomes. In some embodiments, the protein aggregate is HTT, APP, SNCA, or MAPT. In some embodiments, the protein aggregate includes the protein Beta amyloid, Amyloid precursor protein, IAPP (Amylin), Alpha-synuclein, PrPSc, PrPSc, Huntingtin, Calcitonin, Atrial natriuretic factor, Apolipoprotein AI, Serum amyloid A, Medin, Prolactin, Transthyretin, Lysozyme, Beta-2 microglobulin, Gelsolin, Keratoepithelin, Beta amyloid, Cystatin, Immunoglobulin light chain AL, or S-IBM.

The term “amyloid” is used in accordance with its plain ordinary meaning and refers to a protein aggregate wherein the protein is folded into a shape that allows multiple copies of that protein to stick together. In some embodiments, amyloids form fibrils. In some embodiments, the compound described herein binds an amyloid, and is therefore an “amyloid binder”.

The term “macromolecule” is used in accordance with its plain ordinary meaning and refers to a substance (e.g., compound, protein, nucleic acid, carbohydrate, lipid, or macrocycle) of high relative molecular mass, the structure of which may be derived from molecules of low relative molecular mass. In some embodiments, a macromolecule has a molecular weight of greater than 900 Da. In some embodiments, a macromolecule has a molecular weight of greater than 1500 Da. In some embodiments, a macromolecule has a molecular weight of greater than 3000 Da.

A “nanoparticle,” as used herein, is a particle wherein the longest diameter is from 1 to 1000 nanometers. The longest dimension of the nanoparticle may be referred to herein as the length of the nanoparticle. The shortest dimension of the nanoparticle may be referred to herein refer as the width of the nanoparticle. Nanoparticles may be composed of any appropriate material.

The term “vesicle” is used in accordance with its plain ordinary meaning and refers to a small membrane enclosed compartment within a cell. Vesicles are typically involved in transport, buoyancy control, or enzyme storage within a cell. Some vesicles, for example a lysosome, may include enzymes, proteins, polysaccharides, lipids, nucleic acids, or organelles within the compartment. Vesicles are typically formed within cells as a result of exocytosis or phagocytosis, however some vesicles are formed at the Golgi complex and transported to the cell membrane. Vesicles may be unilamellar or multilamellar.

The term “Sequestosome-1” or “SQSTM1” or “p62/SQSTM1” or “ubiquitin-binding protein p62” or “p62” refers to an autophagosome cargo protein (including homologs, isoforms, and functional fragments thereof) that targets other proteins that bind to it for selective autophagy. p62 harbors active nuclear import and export signals and shuttles between the nucleus and cytoplasm. The term “p62” refers to the nucleotide sequences or proteins of human p62. The term “p62” includes both the wild-type form of the nucleotide sequences or proteins as well as any mutants thereof. In some embodiments, “p62” is wild-type p62. In some embodiments, “p62” is one or more mutant forms. The term “p62” XYZ refers to a nucleotide sequence or protein of a mutant p62 wherein the Y numbered amino acid of p62 that has an X amino acid in the wildtype instead has a Z amino acid in the mutant. In some embodiments, p62 is a functional fragment thereof. In some embodiments p62 refers to UniProt C9J6J8, having the sequence:

(SEQ ID NO: 1)
MASLTVKAYLLGKEDAAREIRRFSFCCSPEPEAEAEA
AAGPGPCERLLSRVAALFPALRPGGFQAHYRGGGFR.

In some embodiments, p62 refers to UniProt Q13501, having the sequence:

(SEQ ID NO: 2)
MASLTVKAYLLGKEDAAREIRRFSFCCSPEPEAEAE
AAAGPGPCERLLSRVAALFPALRPGGFQAHYRDED
GDLVAFSSDEELTMAMSYVKDDIFRIYIKEKKECR
RDHRPPCAQEAPRNMVHPNVICDGCNGPVVGTRYK
CSVCPDYDLCSVCEGKGLHRGHTKLAFPSPFGHLS
EGFSHSRWLRKVKHGHFGWPGWEMGPPGNWSPRPP
RAGEARPGPTAESASGPSEDPSVNFLKNVGESVAA
ALSPLGIEVDIDVEHGGKRSRLTPVSPESSSTEEK
SSSQPSSCCSDPSKPGGNVEGATQSLAEQMRKIAL
ESEGRPEEQMESDNCSGGDDDWTHLSSKEVDPSTG
ELQSLQMPESEGPSSLDPSQEGPTGLKEAALYPHL
PPEADPRLIESLSQMLSMGFSDEGGWLTRLLQTKN
YDIGAALDTIQYSKHPPPL.

In some embodiments, p62 refers to the sequence:

(SEQ ID NO: 3)
RFSFCCSPEPEAEAEAAAGPGPCERL

The term “autophagosome” is used in accordance with its plain ordinary meaning and refers to a vesicle that contains a cellular component slated to be degraded by autophagy. In some embodiments, autophagosome formation is a multistep process that includes the biogenesis of the phagophore, followed by its elongation and closure. In some embodiments, more than 15 autophagy-related ATG proteins, as well as class III PI3 kinases, may be required to construct the autophagosome, including the transmembrane ATG protein ATG9, along with membranes from multiple sources cellular sources.

“Nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including, e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amido acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine.; and peptide nucleic acid backbones and linkages. Other analog nucleic acids include those with positive backbones; non-ionic backbones, modified sugars, and non-ribose backbones (e.g., phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In some embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

An “antisense nucleic acid” as referred to herein is a nucleic acid (e.g., DNA or RNA molecule) that is complementary to at least a portion of a specific target nucleic acid (e.g., a nucleic acid coding for one or more amino acids corresponding to C26 of human p62/SQSTM1 protein; C27 of human p62/SQSTM1protein) and is capable of reducing transcription of the target nucleic acid (e.g., mRNA from DNA), reducing the translation of the target nucleic acid (e.g. mRNA), altering transcript splicing (e.g., single stranded morpholino oligo), or interfering with the endogenous activity of the target nucleic acid. See, e.g., Weintraub, Scientific American, 262:40 (1990). Typically, synthetic antisense nucleic acids (e.g., oligonucleotides) are generally between 15 and 25 bases in length. Thus, antisense nucleic acids are capable of hybridizing to (e.g., selectively hybridizing to) a target nucleic acid (e.g., a nucleic acid coding for one or more amino acids corresponding to C26 of human p62/SQSTM1 protein; C27 of human p62/SQSTM1protein). In some embodiments, the antisense nucleic acid hybridizes to the target nucleic acid (e.g., a nucleic acid coding for one or more amino acids corresponding to C26 of human p62/SQSTM1 protein; C27 of human p62/SQSTM1protein) in vitro. In some embodiments, the antisense nucleic acid hybridizes to the target nucleic acid (e.g., a nucleic acid coding for one or more amino acids corresponding to C26 of human p62/SQSTM1 protein; C27 of human p62/SQSTM1protein) in a cell. In some embodiments, the antisense nucleic acid hybridizes to the target nucleic acid (e.g., a nucleic acid coding for one or more amino acids corresponding to C26 of human p62/SQSTM1 protein; C27 of human p62/SQSTM1protein) in an organism. In some embodiments, the antisense nucleic acid hybridizes to the target nucleic acid (e.g., a nucleic acid coding for one or more amino acids corresponding to C26 of human p62/SQSTM1 protein; C27 of human p62/SQSTM1protein) under physiological conditions. Antisense nucleic acids may comprise naturally occurring nucleotides or modified nucleotides such as, e.g., phosphorothioate, methylphosphonate, and -anomeric sugar-phosphate, backbone-modified nucleotides.

In the cell, the antisense nucleic acids hybridize to the corresponding RNA (e.g., a nucleic acid coding for one or more amino acids corresponding to C26 of human p62/SQSTM1 protein; C27 of human p62/SQSTM1protein) forming a double-stranded molecule. The antisense nucleic acids interfere with the endogenous behavior of the RNA (e.g., a nucleic acid coding for one or more amino acids corresponding to C26 of human p62/SQSTM1 protein; C27 of human p62/SQSTM1protein) and inhibit its function relative to the absence of the antisense nucleic acid. Furthermore, the double-stranded molecule may be degraded via the RNAi pathway. The use of antisense methods to inhibit the in vitro translation of genes is well known in the art (Marcus-Sakura, Anal. Biochem., 172:289, (1988)). Further, antisense molecules which bind directly to the DNA may be used. Antisense nucleic acids may be single or double stranded nucleic acids. Non-limiting examples of antisense nucleic acids include siRNAs (including their derivatives or precursors, such as nucleotide analogs), short hairpin RNAs (shRNA), micro RNAs (miRNA), saRNAs (small activating RNAs) and small nucleolar RNAs (snoRNA) or certain of their derivatives or precursors.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanidine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “V_{H},” or “VH” refer to the variable region of an immunoglobulin heavy chain, including an Fv, scFv, dsFv or Fab; while the terms “variable light chain,” “V_{L},” or “VL” refer to the variable region of an immunoglobulin light chain, including of an Fv, scFv, dsFv or Fab.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., FUNDAMENTAL IMMUNOLOGY (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

The term “irreversible covalent bond” is used in accordance with its plain ordinary meaning in the art and refers to the resulting association between atoms or molecules of (e.g., electrophilic chemical moiety and nucleophilic moiety) wherein the probability of dissociation is low. In some embodiments, the irreversible covalent bond does not easily dissociate under normal biological conditions. In some embodiments, the irreversible covalent bond is formed through a chemical reaction between two species (e.g., electrophilic chemical moiety and cysteine).

### II. Compounds

Provided herein are compounds for targeted autophagy protein binding. The compounds described herein, also referred to as targeted autophagy degraders, comprise a monovalent cellular component binder covalently bound to a monovalent targeted autophagy protein binder.

### Targeted Autophagy Protein Binder

In one aspect, provided herein is a compound comprising a monovalent cellular component binder covalently bound to a monovalent targeted autophagy protein binder, wherein the monovalent targeted autophagy protein binder is a monovalent form of the formula:

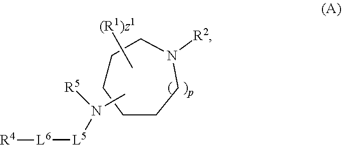

wherein p is 0 or 1, and z1 is an integer from 0-11;

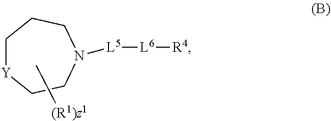

wherein Y is CH₂, NR², O, S, or SO₂; z1 is an integer from 0-12; or

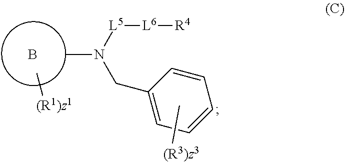

wherein Ring B is (i)

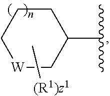

wherein W is O, NH, NR¹, or CH₂; n is 0 or 1; and z1 is an integer from 0-11; or (ii)

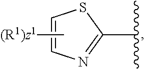

wherein z1 is an integer from 0-2; and z3 is an integer from 0-5;

R¹ is independently oxo, halogen, —CX¹₃, —CHX¹₂, —CH₂X¹, —OCX¹₃, —OCH₂X¹, —OCHX¹₂, —CN, —SOₙ₁R^{1D}, SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or two R¹ substituents are taken together to form a substituted or unsubstituted alkylene, substituted or unsubstituted cycloalkyl, substituted or unsubstituted heterocycloalkyl, substituted or unsubstituted aryl, or substituted or unsubstituted heteroaryl; or one R¹ substituent is taken together with R² to form a substituted or unsubstituted heterocycloalkyl, or substituted or unsubstituted heteroaryl; R² is independently H, oxo, halogen, —CX²₃, —CHX²₂, —CH₂X², —OCX²₃, —OCH₂X², —OCHX²₂, —CN, —SOₙ₂R^{2D}, SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R² is taken together with one R¹ substituent to form a substituted or unsubstituted heterocycloalkyl, or substituted or unsubstituted heteroaryl;

R³ is independently oxo, halogen, —CX³₃, —CHX³₂, —CH₂X³, —OCX³₃, —OCH₂X³, —OCHX³₂, —CN, —SOₙ₃R^{3D}, SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)O R^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or two R³ substituents are taken together to form a substituted or unsubstituted cycloalkyl, substituted or unsubstituted heterocycloalkyl, substituted or unsubstituted aryl, or substituted or unsubstituted heteroaryl;

R⁴ is

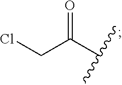 [SMILES: *C(=O)CCl]

L⁵ is a bond, —S(O)₂—, —S(O)—, —NR⁵—, ═N—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R⁵ is hydrogen, —CX⁵₃, —CHX⁵₂, —CH₂X⁵, —OCX⁵₃, —OCH₂X⁵, —OCHX⁵₂, —CN, —C(O)R^{5C}, —C(O)—OR^{5C}, —C(O)NR^{5A}R^{5B}, —OR^{5D} substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L⁶ is a bond, —S(O)₂—, —S(O)—, —NR⁶—, ═N—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R⁶ is hydrogen, —CX⁶₃, —CHX⁶₂, —CH₂X⁶, —OCX⁶₃, —OCH₂X⁶, —OCHX⁶₂, —CN, —C(O)R^{6C}, —C(O)—OR^{6C}, —C(O)NR^{6A}R^{6B}, —OR^{6D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{1A}, R^{1B}, R^{1C}, R^{1D}, R^{2A}, R^{2B}, R^{2C}, R^{2D}, R^{3A}, R^{3B}, R^{3C}, R^{3D}, R^{5A}, R^{5B}, R^{5C}, R^{5D}, R^{6A}, R^{6B}, R^{6C}, and R^{6D} are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom can be taken together to form a substituted or unsubstituted heterocycloalkyl or substituted or unsubstituted heteroaryl; R^{2A} and R^{2B} substituents bonded to the same nitrogen atom can be taken together to form a substituted or unsubstituted heterocycloalkyl or substituted or unsubstituted heteroaryl; R^{3A} and R^{3B} substituents bonded to the same nitrogen atom can be taken together to form a substituted or unsubstituted heterocycloalkyl or substituted or unsubstituted heteroaryl; R^{5A} and R^{5B} substituents bonded to the same nitrogen atom can be taken together to form a substituted or unsubstituted heterocycloalkyl or substituted or unsubstituted heteroaryl; R^{6A} and R^{6B} substituents bonded to the same nitrogen atom can be taken together to form a substituted or unsubstituted heterocycloalkyl or substituted or unsubstituted heteroaryl;

X, X¹, X², X³, X⁵, and X⁶ are independently —F, —Cl, —Br, or —I;

n1, n2, and n3 are independently an integer from 0 to 4; and

m1, m2, m3, v1, v2, and v3 are independently 1 or 2.

In some embodiments, the monovalent targeted autophagy protein binder is a monovalent form of formula (A). In some embodiments, the monovalent targeted autophagy protein binder is a monovalent form of formula (B). In some embodiments, the monovalent targeted autophagy protein binder is a monovalent form of formula (C).

In one aspect, provided herein is a compound comprising a monovalent cellular component binder covalently bound to a monovalent targeted autophagy protein binder, wherein the monovalent targeted autophagy protein binder is a monovalent form of the formula:

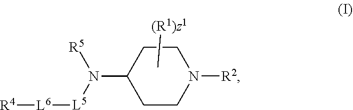

wherein z1 is an integer from 0-9;

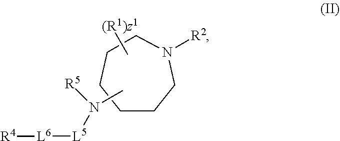

wherein z1 is an integer from 0-11;

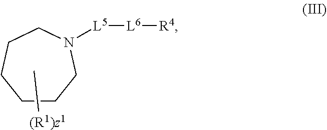

wherein z1 is an integer from 0-12;

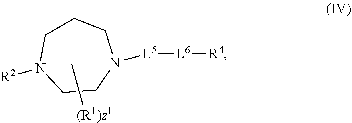

wherein z1 is an integer from 0-10;

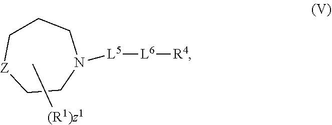

wherein Z is O, S, or SO₂, and z1 is an integer from 0-10;

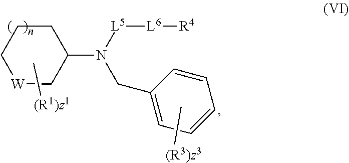

wherein W is O, NH, NR¹, or CH₂; n is 0 or 1; z1 is an integer from 0-11; and z3 is an integer from 0-5;

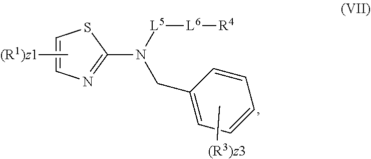

wherein z1 is an integer from 0-2, and z3 is an integer from 0-5;

R¹ is independently oxo, halogen, —CX¹₃, —CHX¹₂, —CH₂X¹, —OCX¹₃, —OCH₂X¹, —OCHX¹₂, —CN, —SOₙ₁R^{1D}, SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or two R¹ substituents are taken together to form a substituted or unsubstituted alkylene, substituted or unsubstituted cycloalkyl, substituted or unsubstituted heterocycloalkyl, substituted or unsubstituted aryl, or substituted or unsubstituted heteroaryl; or one R¹ substituent is taken together with R² to form a substituted or unsubstituted heterocycloalkyl, or substituted or unsubstituted heteroaryl;

R² is independently H, oxo, halogen, —CX²₃, —CHX²₂, —CH₂X², —OCX²₃, —OCH₂X², —OCHX²₂, —CN, —SOₙ₂R^{2D}, —SO^{v2}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R² is taken together with one R¹ substituent to form a substituted or unsubstituted heterocycloalkyl, or substituted or unsubstituted heteroaryl;

R³ is independently oxo, halogen, —CX³₃, —CHX³₂, —CH₂X³, —OCX³₃, —OCH₂X³, —OCHX³₂, —CN, —SOn₃R^{3D}, SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)O R^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or two R³ substituents are taken together to form a substituted or unsubstituted cycloalkyl, substituted or unsubstituted heterocycloalkyl, substituted or unsubstituted aryl, or substituted or unsubstituted heteroaryl;

R⁴ is

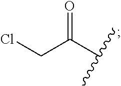 [SMILES: *C(=O)CCl]

L⁵ is a bond, —S(O)₂—, —S(O)—, —NR⁵—, ═N—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R⁵ is hydrogen, —CX⁵₃, —CHX⁵₂, —CH₂X⁵, —OCX⁵₃, —OCH₂X⁵, —OCHX⁵₂, —CN, —C(O)R^{5C}, —C(O)—OR^{5C}, —C(O)NR^{5A}R^{5B}, —OR^{5D} substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L⁶ is a bond, —S(O)₂—, —S(O)—, —NR⁶—, ═N—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R⁶ is hydrogen, —CX⁶₃, —CHX⁶₂, —CH₂X⁶, —OCX⁶₃, —OCH₂X⁶, —OCHX⁶₂, —CN, —C(O)R^{6C}, —C(O)—OR^{6C}, —C(O)NR^{6A}R^{6B}, —OR^{6D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{1A}, R^{1B}, R^{1C}, R^{1D}, R^{2A}, R^{2B}, R^{2C}, R^{2D}, R^{3A}, R^{3B}, R^{3C}, R^{3D}, R^{5A}, R^{5B}, R^{5C}, R^{5D}, R^{6A}, R^{6B}, R^{6C}, and R^{6D} are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom can be taken together to form a substituted or unsubstituted heterocycloalkyl or substituted or unsubstituted heteroaryl; R^{2A} and R^{2B} substituents bonded to the same nitrogen atom can be taken together to form a substituted or unsubstituted heterocycloalkyl or substituted or unsubstituted heteroaryl; R^{3A} and R^{3B} substituents bonded to the same nitrogen atom can be taken together to form a substituted or unsubstituted heterocycloalkyl or substituted or unsubstituted heteroaryl; R^{5A} and R^{5B} substituents bonded to the same nitrogen atom can be taken together to form a substituted or unsubstituted heterocycloalkyl or substituted or unsubstituted heteroaryl; R^{6A} and R^{6B} substituents bonded to the same nitrogen atom can be taken together to form a substituted or unsubstituted heterocycloalkyl or substituted or unsubstituted heteroaryl;

X, X¹, X², X³, X⁵, and X⁶ are independently —F, —Cl, —Br, or —I;

n1, n2, and n3 are independently an integer from 0 to 4; and

m1, m2, m3, v1, v2, and v3 are independently 1 or 2.

In some embodiments, the monovalent targeted autophagy protein binder is a monovalent form of the formula:

wherein z1 is an integer from 0-9. In some embodiments, z1 is 0, 1, or 2. In some embodiments, the monovalent targeted autophagy protein binder is a monovalent form of the formula:

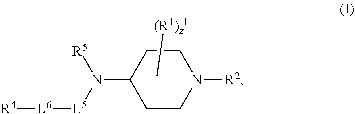

wherein zl is an integer from 0-9. In some embodiments, zl is 0, 1, or 2. In some embodiments, the monovalent targeted autophagy protein binder is a monovalent form of the formula:

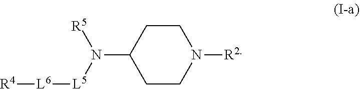

In some embodiments or variations of formula (I) and (I-a), R² is H, —C(O)—OR^{2C}, or substituted or unsubstituted alkyl; and R^{2C} is substituted or unsubstituted alkyl. In some embodiments, R² is H or —C(O)OC(CH₃)₃.

In some embodiments or variations of formula (I) and (I-a), each R¹ is independently halogen, or substituted or unsubstituted alkyl. In some embodiments, each R¹ is independently —F, —Cl, or —CH₃.

In some embodiments or variations of formula (I) and (I-a), R⁵ is hydrogen, substituted or unsubstituted alkyl, substituted or unsubstituted heteroalkyl, substituted or unsubstituted cycloalkyl, or substituted or unsubstituted heterocycloalkyl. In some embodiments, R⁵ is

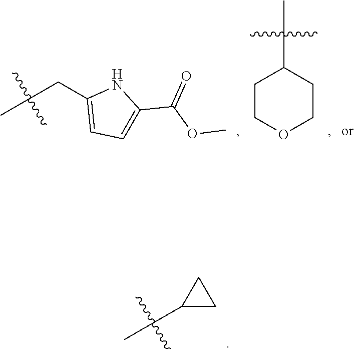

In some embodiments or variations of formula (I) and (I-a), L⁵ is a bond, substituted or unsubstituted alkylene, or substituted or unsubstituted heteroalkylene; and L⁶ is a bond, substituted or unsubstituted alkylene, or substituted or unsubstituted heteroalkylene. In some embodiments, L⁵ and L⁶ are each a bond.

In some embodiments or variations of formula (I) and (I-a), the monovalent targeted autophagy protein binder is a monovalent form of the formula:

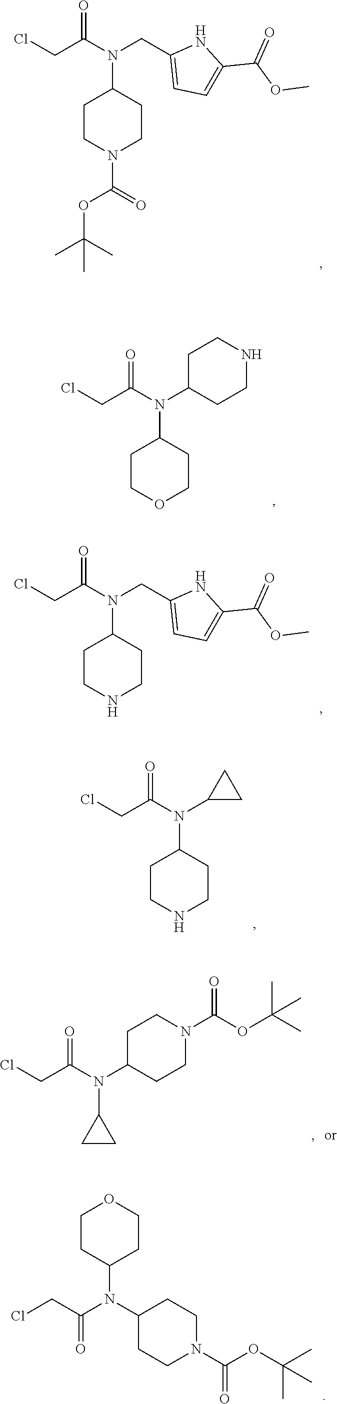 [SMILES: COC(=O)c1ccc(CN(C(=O)CCl)C2CCN(C(=O)OC(C)(C)C)CC2)[nH]1 ; O=C(CCl)N(C1CCNCC1)C1CCOCC1 ; COC(=O)c1ccc(CN(C(=O)CCl)C2CCNCC2)[nH]1 ; O=C(CCl)N(C1CCNCC1)C1CC1 ; CC(C)(C)OC(=O)N1CCC(N(C(=O)CCl)C2CC2)CC1 ; CC(C)(C)OC(=O)N1CCC(N(C(=O)CCl)C2CCOCC2)CC1]

In some embodiments, formula (A) is formula (I) or any variation or embodiment thereof.

In some embodiments, the monovalent targeted autophagy protein binder is a monovalent form of the formula:

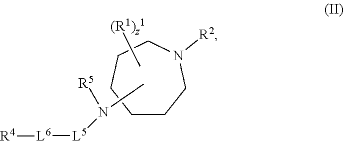

wherein z1 is an integer from 0-11. In some embodiments, z1 is 0, 1, or 2. In some embodiments, the monovalent targeted autophagy protein binder is a monovalent form of the formula:

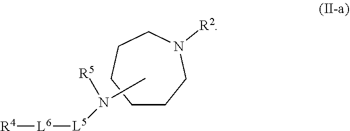

In some embodiments, the monovalent targeted autophagy protein binder is a monovalent form of the formula:

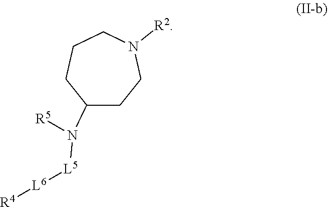

In some embodiments or variations of formula (II), (II-a), and (II-b), R² is H, —C(O)—OR^{2C}, or substituted or unsubstituted alkyl; and R^{2C} is substituted or unsubstituted alkyl. In some embodiments, R² is H or —C(O)OC(CH₃)₃.

In some embodiments or variations of formula (II), (II-a), and (II-b), R⁵ is hydrogen, substituted or unsubstituted alkyl, substituted or unsubstituted heteroalkyl, substituted or unsubstituted cycloalkyl, or substituted or unsubstituted heterocycloalkyl. In some embodiments, R⁵ is isopropyl.

In some embodiments or variations of formula (II), (II-a), and (II-b), L⁵ is a bond, substituted or unsubstituted alkylene, or substituted or unsubstituted heteroalkylene; and L⁶ is a bond, substituted or unsubstituted alkylene, or substituted or unsubstituted heteroalkylene. In some embodiments, L⁵ and L⁶ are each a bond.

In some embodiments or variations of formula (II), (II-a), and (II-b), the monovalent targeted autophagy protein binder is a monovalent form of the formula:

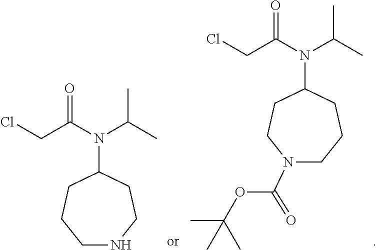 [SMILES: CC(C)N(C(=O)CCl)C1CCCNCC1 ; CC(C)N(C(=O)CCl)C1CCCN(C(=O)OC(C)(C)C)CC1]

In some embodiments, formula (A) is formula (II) or any variation or embodiment thereof.

In some embodiments, the monovalent targeted autophagy protein binder is a monovalent form of the formula:

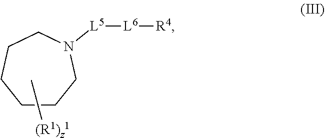

wherein z1 is an integer from 0-12. In some embodiments, z1 is 1, 2, or 3. In some embodiments, the monovalent targeted autophagy protein binder is a monovalent form of the formula:

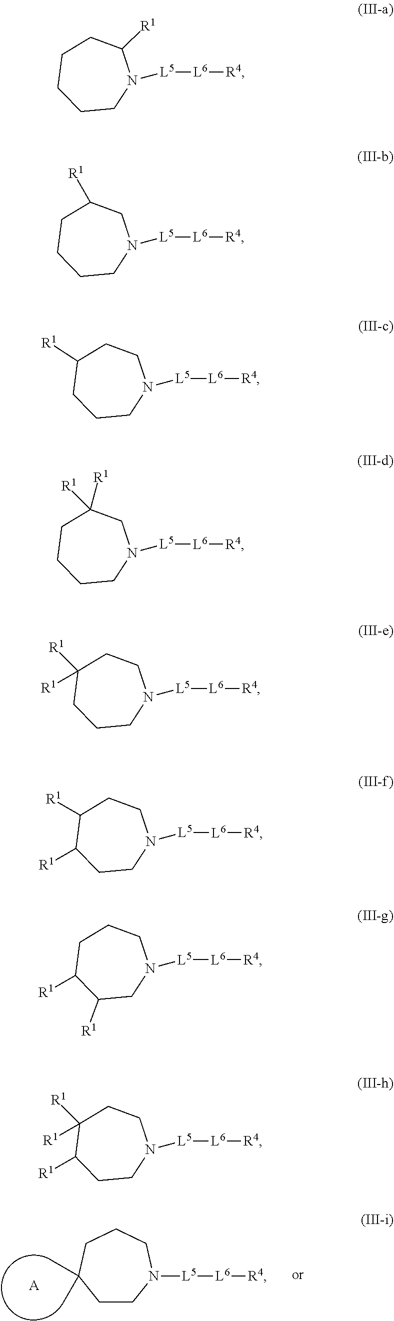

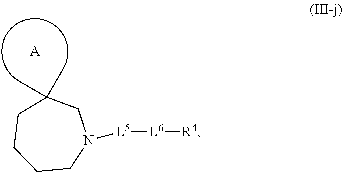

wherein the Ring A moiety is a substituted or unsubstituted cycloalkyl, substituted or unsubstituted heterocycloalkyl, substituted or unsubstituted aryl, or substituted or unsubstituted heteroaryl.

In some embodiments or variations of formula (III) and (III-a)-(III-j), each R¹ is independently oxo, halogen, —CX¹₃, —CHX¹₂, —CH₂X¹, —NR^{1A}R^{1B}, —C(O)—OR^{1C}, —NR^{1A}C(O)OR^{1C}, substituted or unsubstituted alkyl, or substituted or unsubstituted heteroalkyl; or two R¹ substituents are taken together to form a substituted or unsubstituted cycloalkyl, or substituted or unsubstituted heterocycloalkyl; each R^{1A}, R^{1B}, and R^{1C} is independently hydrogen, or substituted or unsubstituted alkyl; and each X¹ is independently —F or —Cl. In some embodiments, each R¹ is independently F, —CH₃, —OH, —CF₃, —CH₂F, —C(O)OCH₂CH₃, —NH₂, oxo, —CH₂N(H)C(O)OCH₂(C₆H₅), or —N(H)C(O)OC(CH₃)₃; or two R¹ groups are taken together to form

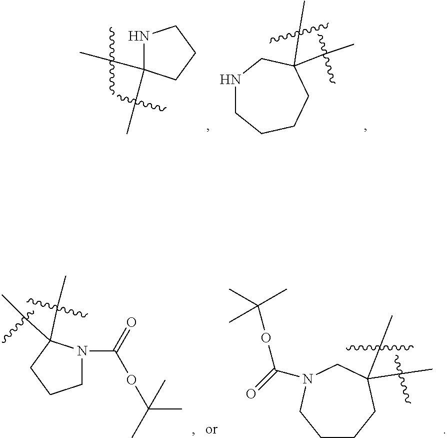 [SMILES: *C1(*)CCCN1 ; *C1(*)CCCCNC1 ; *C1(*)CCCN1C(=O)OC(C)(C)C ; *C1(*)CCCCN(C(=O)OC(C)(C)C)C1]

In some embodiments or variations of formula (III) and (III-a)-(III-j), L⁵ is a bond, substituted or unsubstituted alkylene, or substituted or unsubstituted heteroalkylene; and L⁶ is a bond, substituted or unsubstituted alkylene, or substituted or unsubstituted heteroalkylene. In some embodiments, L⁵ and L⁶ are each a bond.

In some embodiments or variations of formula (III) and (III-a)-(III-j), the monovalent targeted autophagy protein binder is a monovalent form of the formula:

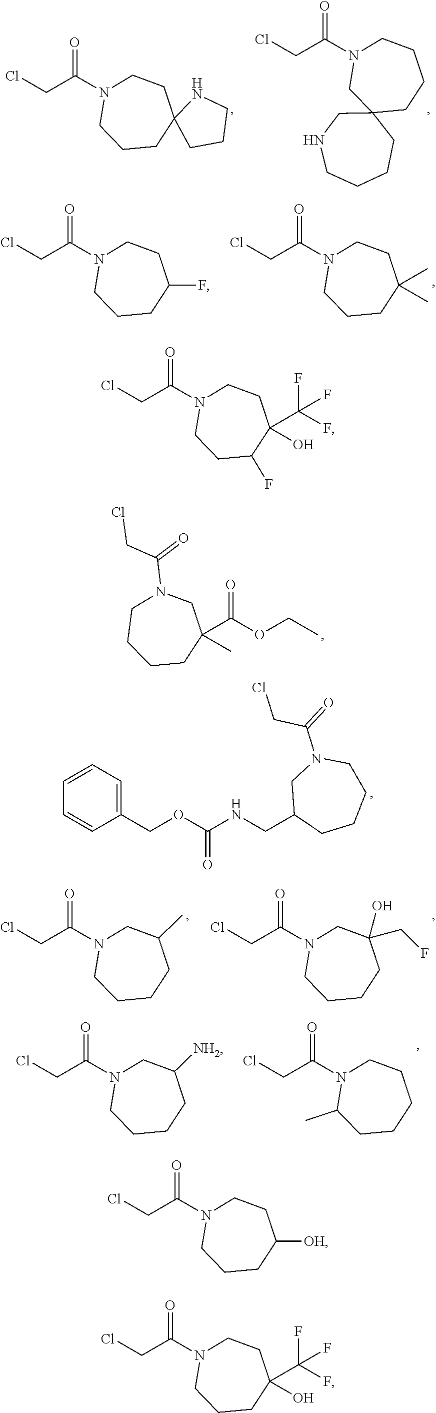

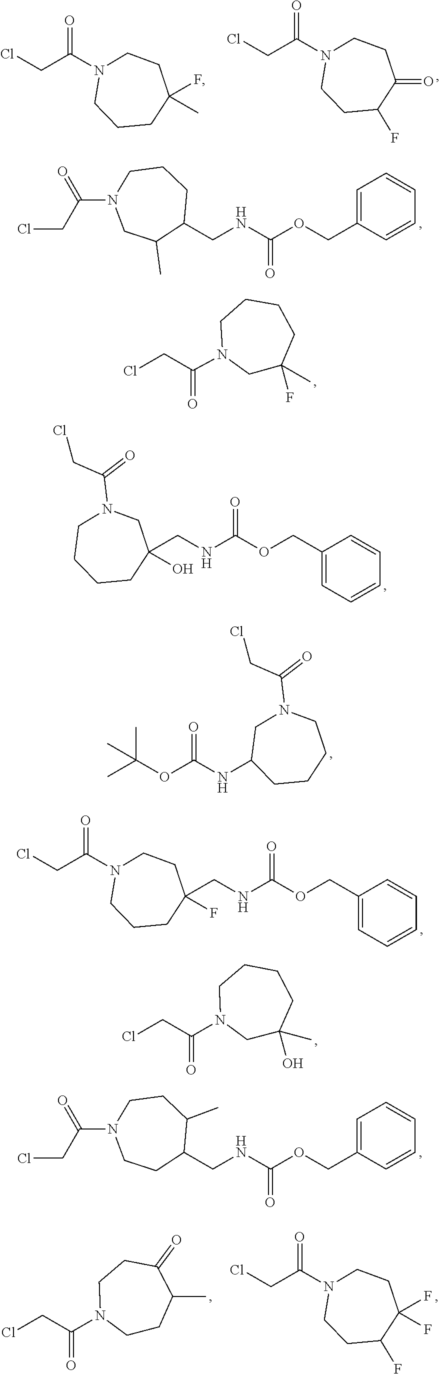

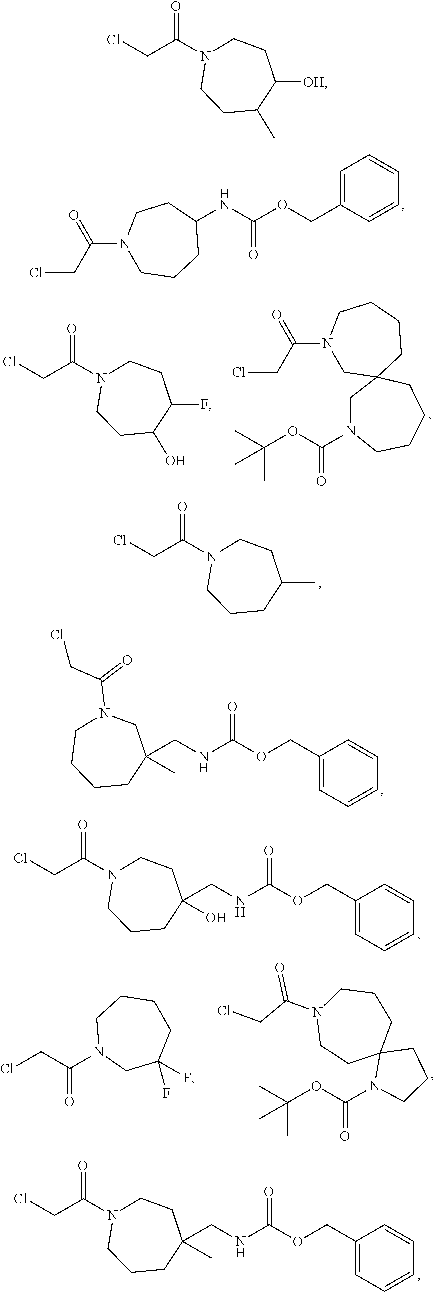

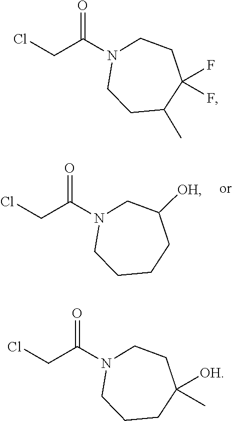

In some embodiments, formula (B) is formula (III) or any variation or embodiment thereof.

In some embodiments, the monovalent targeted autophagy protein binder is a monovalent form of the formula:

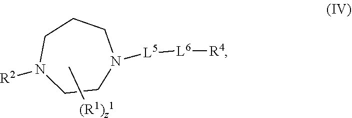

wherein z1 is an integer from 0-10. In some embodiments, z1 is 0, 1, or 2. In some embodiments, the monovalent targeted autophagy protein binder is a monovalent form of the formula:

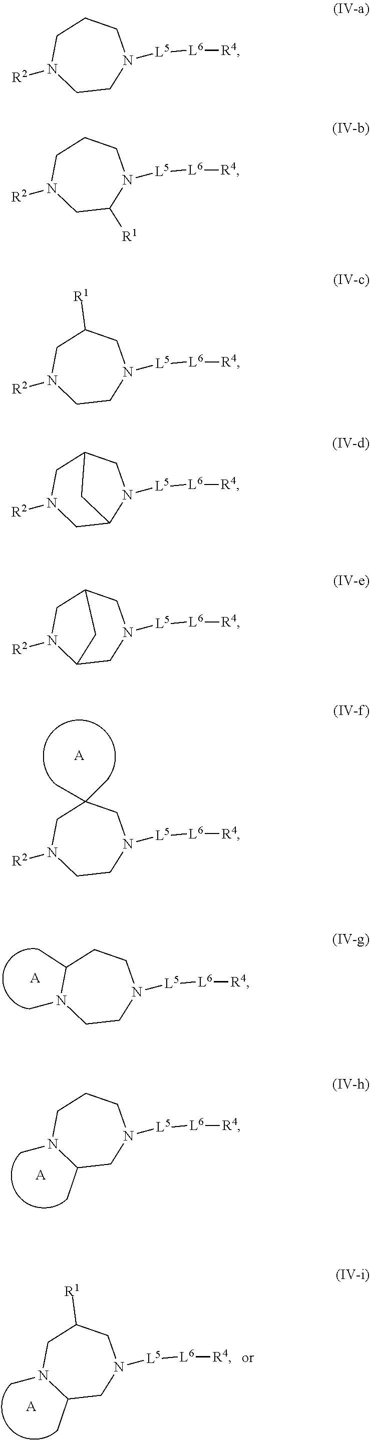

wherein the Ring A moiety is a substituted or unsubstituted cycloalkyl, substituted or unsubstituted heterocycloalkyl, substituted or unsubstituted aryl, or substituted or unsubstituted heteroaryl.

In some embodiments or variations of formula (IV) and (IV-a)-(IV-i), each R¹ is independently halogen, —CX¹₃, —OR^{1D}, substituted or unsubstituted alkyl, or substituted or unsubstituted heteroalkyl; or two R¹ substituents are taken together to form a substituted or unsubstituted alkylene, substituted or unsubstituted cycloalkyl, or substituted or unsubstituted heterocycloalkyl; or one R¹ substituent is taken together with R² to form a substituted or unsubstituted heteroaryl; each R^{1D} is independently hydrogen, or substituted or unsubstituted alkyl; and each X¹ is independently —F or —Cl. In some embodiments, each R¹ is independently —CH₃ or —OH; or two R¹ groups are taken together to form —CH₂—,

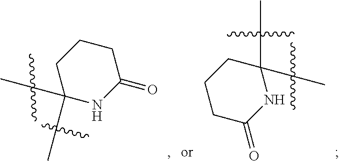 [SMILES: *C1(*)CCCC(=O)N1 ; *C1(*)CCCC(=O)N1]

or R¹ and R² are taken together to form

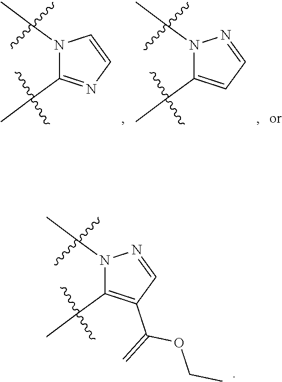 [SMILES: *c1nccn1* ; *c1ccnn1* ; *c1c(C(=C)OCC)cnn1*]

In some embodiments or variations of formula (IV) and (IV-a)-(IV-i), each R² is independently hydrogen, substituted or unsubstituted alkyl, —C(O)OR^{2C}, or substituted or unsubstituted heteroaryl; or R² is taken together with one R¹ substituent to form a substituted or unsubstituted heteroaryl; and each R^{2C} is independently substituted or unsubstituted alkyl. In some embodiments, R² is H, —CH₂CH₃, —C(O)OCH₃, —C(O)OC(CH₃)₃, or

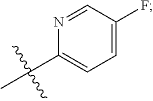 [SMILES: *c1ccc(F)cn1]

or R² and R¹ are taken together to form

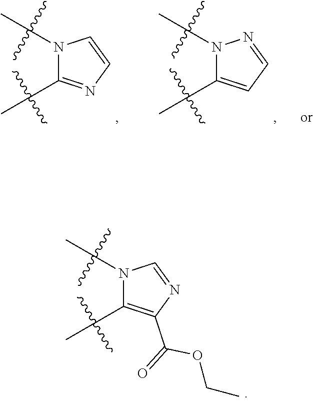 [SMILES: *c1nccn1* ; *c1ccnn1* ; *c1c(C(=O)OCC)ncn1*]

In some embodiments or variations of formula (IV) and (IV-a)-(IV-i), L⁵ is a bond, substituted or unsubstituted alkylene, or substituted or unsubstituted heteroalkylene; and L⁶ is a bond, substituted or unsubstituted alkylene, or substituted or unsubstituted heteroalkylene. In some embodiments, L⁵ and L⁶ are each a bond.

In some embodiments or variations of formula (IV) and (IV-a)-(IV-i), the monovalent targeted autophagy protein binder is a monovalent form of the formula:

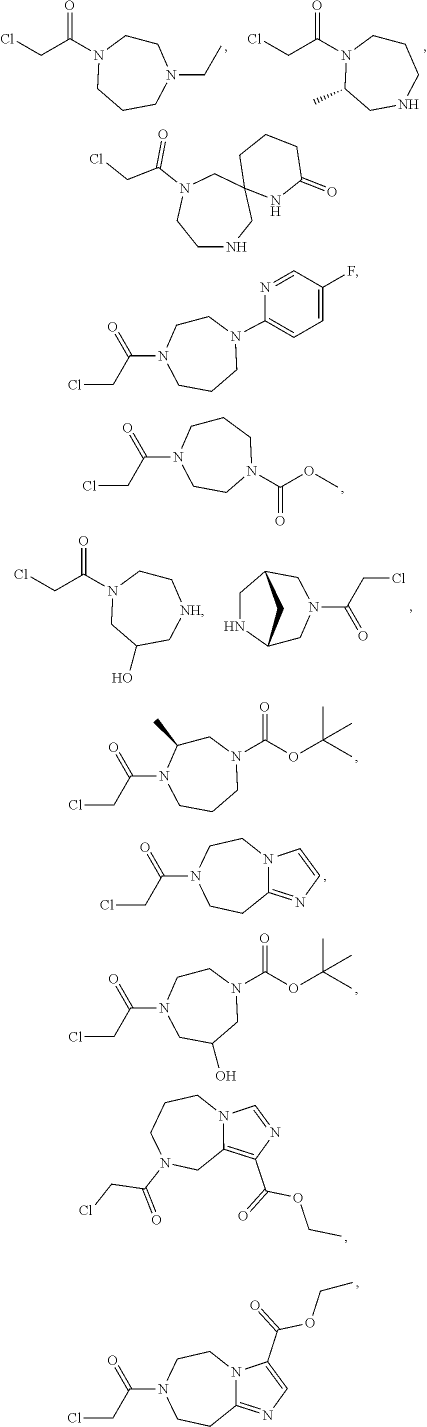

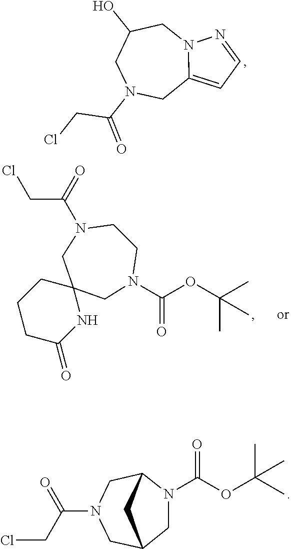

In some embodiments, formula (B) is formula (IV) or any variation or embodiment thereof.

In some embodiments, the monovalent targeted autophagy protein binder is a monovalent form of the formula:

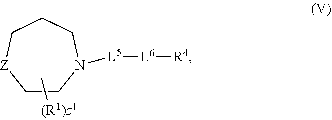

wherein Z is O, S, or SO₂, and z1 is an integer from 0-10. In some embodiments, z1 is 0-3.

In some embodiments, Z is O. In some embodiments, the monovalent targeted autophagy protein binder is a monovalent form of the formula:

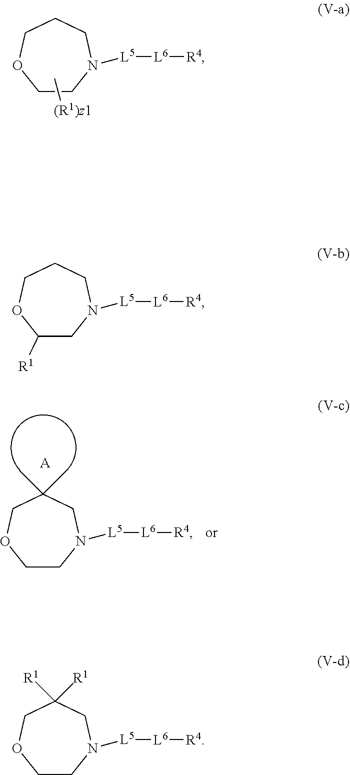

In some embodiments, Z is S. In some embodiments, the monovalent targeted autophagy protein binder is a monovalent form of the formula:

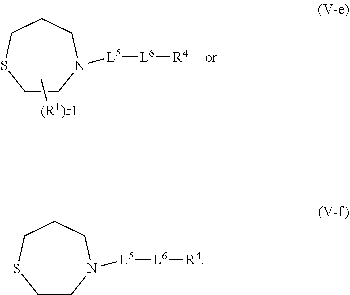

In some embodiments, Z is SO₂. In some embodiments, the monovalent targeted autophagy protein binder is a monovalent form of the formula:

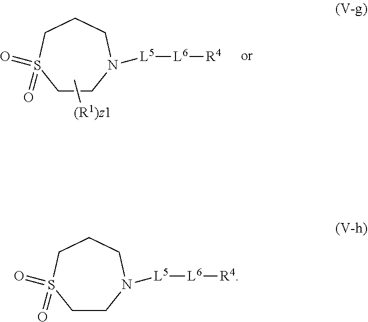

In some embodiments or variations of formula (V) and (V-a)-(V-h), each R¹ is independently halogen, —CX¹₃, substituted or unsubstituted alkyl, or substituted or unsubstituted heteroalkyl; or two R¹ substituents are taken together to form a substituted or unsubstituted cycloalkyl, or substituted or unsubstituted heterocycloalkyl; and each X¹ is independently —F or —Cl. In some embodiments, each R¹ is independently —CH₃ or F;

or two R¹ substituents are taken together to form

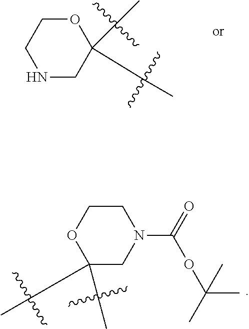 [SMILES: *C1(*)CNCCO1 ; *C1(*)CN(C(=O)OC(C)(C)C)CCO1]

In some embodiments or variations of formula (V) and (V-a)-(V-h), L⁵ is a bond, substituted or unsubstituted alkylene, or substituted or unsubstituted heteroalkylene; and L⁶ is a bond, substituted or unsubstituted alkylene, or substituted or unsubstituted heteroalkylene. In some embodiments, L⁵ and L⁶ are each a bond.

In some embodiments or variations of formula (V) and (V-a)-(V-h), the monovalent targeted autophagy protein binder is a monovalent form of the formula:

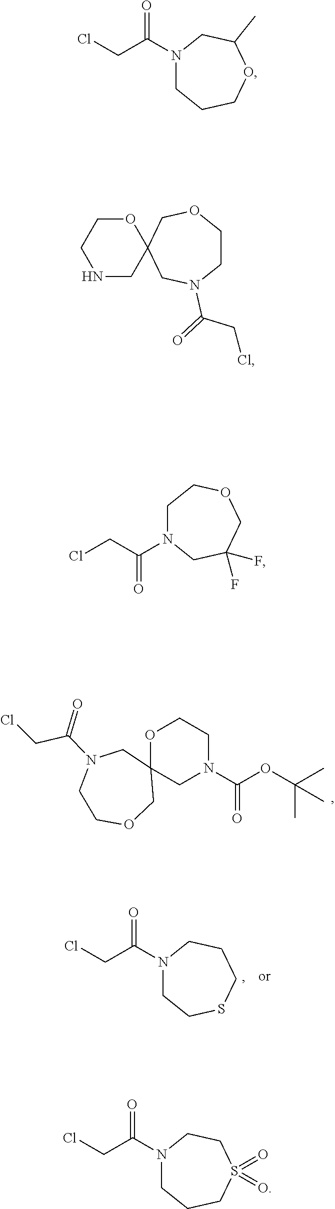 [SMILES: CC1CN(C(=O)CCl)CCCO1 ; O=C(CCl)N1CCOCC2(CNCCO2)C1 ; O=C(CCl)N1CCOCC(F)(F)C1 ; CC(C)(C)OC(=O)N1CCOC2(COCCN(C(=O)CCl)C2)C1 ; O=C(CCl)N1CCCSCC1 ; O=C(CCl)N1CCCS(=O)(=O)CC1]

In some embodiments, formula (B) is formula (V) or any variation or embodiment thereof.

In some embodiments, the monovalent targeted autophagy protein binder is a monovalent form of the formula:

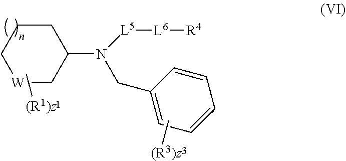

wherein W is O, NH, NR¹, or CH₂; n is 0 or 1; z1 is an integer from 0-11; and z3 is an integer from 0-5. In some embodiments, z1 is 0-3. In some embodiments, z3 is 0-2. In some embodiments, n is 0. In some embodiments, n is 1.

In some embodiments of formula (VI), W is CH₂. In some embodiments, the monovalent targeted autophagy protein binder is a monovalent form of the formula:

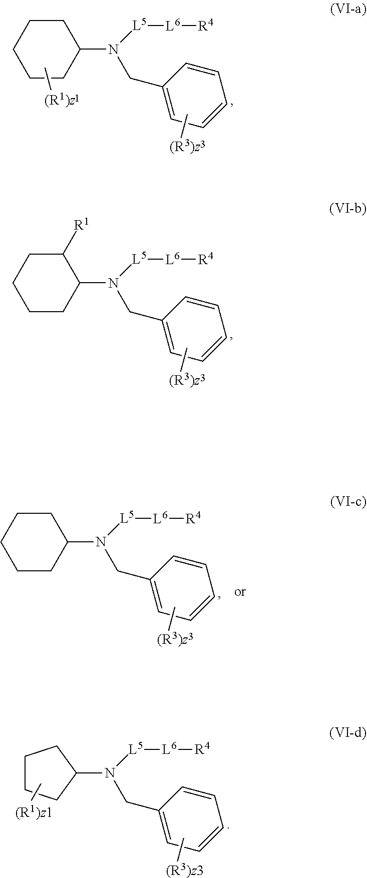

In some embodiments of formula (VI), W is O. In some embodiments, the monovalent targeted autophagy protein binder is a monovalent form of the formula:

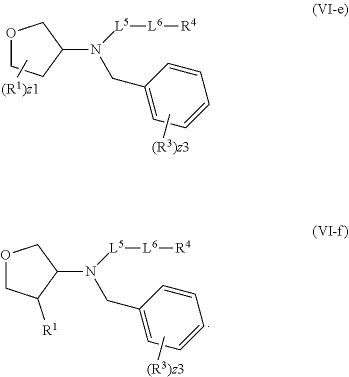

In some embodiments of formula (VI), W is NH or NR¹. In some embodiments, the monovalent targeted autophagy protein binder is a monovalent form of the formula:

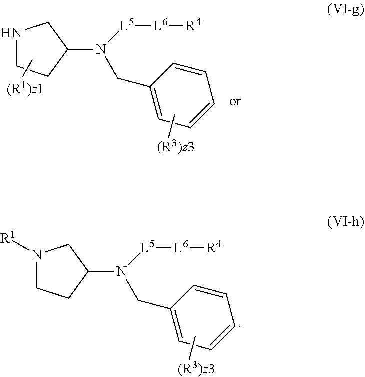

In some embodiments or variations of formula (VI) and (VI-a)-(VI-h), each R¹ is independently oxo, halogen, —OR^{1D}, or substituted or unsubstituted alkyl; and each R^{1D} is independently hydrogen, or substituted or unsubstituted alkyl. In some embodiments, each R¹ is independently —CH₂OH, —OH, oxo, or —CH₂(C₆H₅).

In some embodiments or variations of formula (VI) and (VI-a)-(VI-h), L⁵ is a bond, substituted or unsubstituted alkylene, or substituted or unsubstituted heteroalkylene; and L⁶ is a bond, substituted or unsubstituted alkylene, or substituted or unsubstituted heteroalkylene. In some embodiments, L⁵ and L⁶ are each a bond.

In some embodiments or variations of formula (VI) and (VI-a)-(VI-h), the monovalent targeted autophagy protein binder is a monovalent form of the formula:

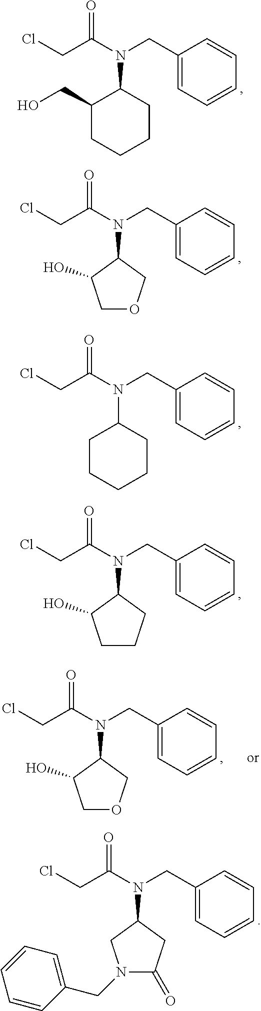

In some embodiments, formula (C) is formula (VI) or any variation or embodiment thereof.

In some embodiments, the monovalent targeted autophagy protein binder is a monovalent form of the formula:

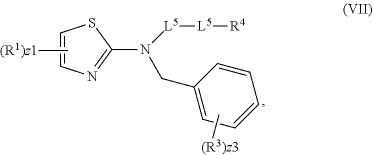

wherein z1 is an integer from 0-2, and z3 is an integer from 0-5. In some embodiments, z1 is an integer from 0 or 1. In some embodiments, z3 is 0, 1, or 2.

In some embodiments of formula (VII), the monovalent targeted autophagy protein binder is a monovalent form of the formula:

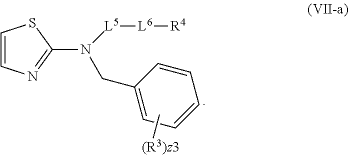

In some embodiments or variations of formula (VII) and (VII-a), each R¹ is independently halogen, —CX¹₃, substituted or unsubstituted alkyl, or substituted or unsubstituted heteroalkyl; and each X¹ s independently —F or —Cl. In some embodiments, each R¹ is independently —F or —CH₃.

In some embodiments or variations of formula (VII) and (VII-a), each R³ is independently halogen, —OR^{3D}, or substituted or unsubstituted alkyl; and each R^{3D} is independently hydrogen, or substituted or unsubstituted alkyl. In some embodiments, each R³ is —OCH₃.

In some embodiments or variations of formula (VII) and (VII-a), L⁵ is a bond, substituted or unsubstituted alkylene, or substituted or unsubstituted heteroalkylene; and L⁶ is a bond, substituted or unsubstituted alkylene, or substituted or unsubstituted heteroalkylene. In some embodiments, L⁵ and L⁶ are each a bond.

In some embodiments or variations of formula (VII) and (VII-a), the monovalent targeted autophagy protein binder is a monovalent form of the formula:

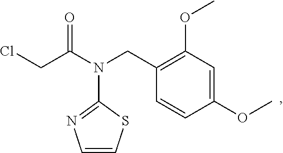 [SMILES: COc1ccc(CN(C(=O)CCl)c2nccs2)c(OC)c1]

It will be understood that floating R-substituents in the formulae described herein (such as R¹ and R³) may be positioned on any ring in a fused or bridged ring system even though the formula may show the floating R-substituent on a single ring.

In some embodiments, formula (C) is formula (VII) or any variation or embodiment thereof.

In some of the embodiments and variations of formula (I), (II), (III), (IV), (V), (VI), and (VII) described herein, R¹ is independently oxo, halogen, —CX¹₃, —CHX¹₂, —CH₂X¹, —OCX¹₃, —OCH₂X¹, —OCHX¹₂, —CN, —SOₙ₁R^{1D}, SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, two R¹ substituents are taken together to form a substituted or unsubstituted alkylene, substituted or unsubstituted cycloalkyl, substituted or unsubstituted heterocycloalkyl, substituted or unsubstituted aryl, or substituted or unsubstituted heteroaryl. In some embodiments, one R¹ substituent is taken together with R² to form a substituted or unsubstituted heterocycloalkyl, or substituted or unsubstituted heteroaryl.

In some of the embodiments and variations of formula (I), (II), (III), (IV), (V), (VI), and (VII) described herein, R² is independently H, oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R² is taken together with one R¹ substituent to form a substituted or unsubstituted heterocycloalkyl, or substituted or unsubstituted heteroaryl.

In some of the embodiments and variations of formula (I), (II), (III), (IV), (V), (VI), and (VII) described herein, R³ is independently oxo, halogen, —CX³₃, —CHX³₂, —CH₂X³, —OCX³₃, —OCH₂X³, —OCHX³₂, —CN, —SOₙ₃R^{3D}, SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In some embodiments, two R³ substituents are taken together to form a substituted or unsubstituted cycloalkyl, substituted or unsubstituted heterocycloalkyl, substituted or unsubstituted aryl, or substituted or unsubstituted heteroaryl.

In some of the embodiments and variations of formula (I), (II), (III), (IV), (V), (VI), and (VII) described herein, R⁴ is

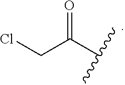 [SMILES: *C(=O)CCl]

In some of the embodiments and variations of formula (I), (II), (III), (IV), (V), (VI), and (VII) described herein, L⁵ is a bond, —S(O)₂—, —S(O)—, —NR⁵—, ═N—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. It will be understood that selection of L⁵ obeys standard rules of chemical valency known in the chemical arts and forms a chemical bond according to standard rules of chemical bonding.

In some of the embodiments and variations of formula (I), (II), (III), (IV), (V), (VI), and (VII) described herein, R⁵ is hydrogen, —CX⁵₃, —CHX⁵₂, —CH₂X⁵, —OCX⁵₃, —OCH₂X⁵, —OCHX⁵₂, —CN, —C(O)R^{5C}, —C(O)—OR^{5C}, —C(O)NR^{5A}R^{5B}, —OR^{5D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In some of the embodiments and variations of formula (I), (II), (III), (IV), (V), (VI), and (VII) described herein, L⁶ is a bond, —S(O)₂—, —S(O)—, —NR⁶—, ═N—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. It will be understood that selection of L⁶ obeys standard rules of chemical valency known in the chemical arts and forms a chemical bond according to standard rules of chemical bonding.

In some of the embodiments and variations of formula (I), (II), (III), (IV), (V), (VI), and (VII) described herein, R⁶ is hydrogen, —CX⁶₃, —CHX⁶₂, —CH₂X⁶, —OCX⁶₃, —OCH₂X⁶, —OCHX⁶₂, —CN, —C(O)R^{6C}, —C(O)—OR^{6C}, —C(O)NR^{6A}R^{6B}, —OR^{6D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In some of the embodiments and variations of formula (I), (II), (III), (IV), (V), (VI), and (VII) described herein, R^{1A}, R^{1B}, R^{1C}, R^{1D}, R^{2A}, R^{2B}, R^{2C}, R^{2D}, R^{3A}, R^{3B}, R^{3C}, R^{3D}, R^{5A}, R^{5B}, R^{5C}, R^{5D}, R^{6A}, R^{6B}, R^{6C}, and R^{6D} are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R^{1A} and R^{1B} substituents bonded to the same nitrogen atom are taken together to form a substituted or unsubstituted heterocycloalkyl or substituted or unsubstituted heteroaryl. In some embodiments, R^{2A} and R^{2B} substituents bonded to the same nitrogen atom are taken together to form a substituted or unsubstituted heterocycloalkyl or substituted or unsubstituted heteroaryl. In some embodiments, R^{3A} and R^{3B} substituents bonded to the same nitrogen atom are taken together to form a substituted or unsubstituted heterocycloalkyl or substituted or unsubstituted heteroaryl. In some embodiments, R^{5A} and R^{5B} substituents bonded to the same nitrogen atom are taken together to form a substituted or unsubstituted heterocycloalkyl or substituted or unsubstituted heteroaryl. In some embodiments, R^{6A} and R^{6B} substituents bonded to the same nitrogen atom are taken together to form a substituted or unsubstituted heterocycloalkyl or substituted or unsubstituted heteroaryl.

In some of the embodiments and variations of formula (I), (II), (III), (IV), (V), (VI), and (VII) described herein, X, X¹, X², X³, X⁵, and X⁶ are independently —F, —Cl, —Br, or —I.

In some of the embodiments and variations of formula (I), (II), (III), (IV), (V), (VI), and (VII) described herein, n1, n2, and n3 are independently an integer from 0 to 4; and m1, m2, m3, v1, v2, and v3 are independently 1 or 2.

In some of the embodiments and variations of formula (I), (II), (III), (IV), (V), (VI), and (VII) described herein, each of L⁵ and L⁶ is a bond and -L⁵-L⁶-R⁴ is —R⁴. In some embodiments, R⁴ is

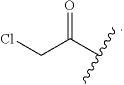 [SMILES: *C(=O)CCl]

In some embodiments, -L⁵-L⁶-R⁴ is

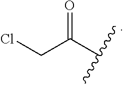 [SMILES: *C(=O)CCl]

In some embodiments, R¹ is independently halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —O CI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N₃, substituted or unsubstituted alkyl (e.g., C₁-C₆ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In some embodiments, R¹ is independently substituted or unsubstituted alkyl (e.g., C₁-C₈ alkyl, C₁-C₆ alkyl, or C₁-C₄ alkyl). In some embodiments, R¹ is independently substituted alkyl (e.g., C₁-C₈ alkyl, C₁-C₆ alkyl, or C₁-C₄ alkyl). In some embodiments, R¹ is independently an unsubstituted alkyl (e.g., C₁-C₈ alkyl, C₁-C₆ alkyl, or C₁-C₄ alkyl). In some embodiments, R¹ is independently substituted or unsubstituted heteroalkyl (e.g., 2 to 8 membered heteroalkyl, 2 to 6 membered heteroalkyl, or 2 to 4 membered heteroalkyl). In some embodiments, R¹ is independently substituted heteroalkyl (e.g., 2 to 8 membered heteroalkyl, 2 to 6 membered heteroalkyl, or 2 to 4 membered heteroalkyl). In some embodiments, R¹ is independently an unsubstituted heteroalkyl (e.g., 2 to 8 membered heteroalkyl, 2 to 6 membered heteroalkyl, or 2 to 4 membered heteroalkyl). In some embodiments, R¹ is independently substituted or unsubstituted cycloalkyl (e.g., C₃-C₈ cycloalkyl, C₃-C₆ cycloalkyl, or C₅-C₆ cycloalkyl). In some embodiments, R¹ is independently substituted cycloalkyl (e.g., C₃-C₈ cycloalkyl, C₃-C₆ cycloalkyl, or C₅-C₆ cycloalkyl). In some embodiments, R¹ is independently an unsubstituted cycloalkyl (e.g., C₃-C₈ cycloalkyl, C₃-C₆ cycloalkyl, or C₅-C₆ cycloalkyl). In some embodiments, R¹ is independently substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R¹ is independently substituted heterocycloalkyl (e.g., 3 to 8 membered heterocycloalkyl, 3 to 6 membered heterocycloalkyl, or 5 to 6 membered heterocycloalkyl). In some embodiments, R¹ is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R¹ is independently substituted or unsubstituted aryl (e.g., C₆-C₁₀ aryl, C₁₀ aryl, or phenyl). In some embodiments, R¹ is independently substituted aryl (e.g., C₆-C₁₀ aryl, C₁₀ aryl, or phenyl). In some embodiments, R¹ is independently an unsubstituted aryl (e.g., C₆-C₁₀ aryl, C₁₀ aryl, or phenyl). In some embodiments, R¹ is independently substituted or unsubstituted heteroaryl (e.g., 5 to 10 membered heteroaryl, 5 to 9 membered heteroaryl, or 5 to 6 membered heteroaryl). In some embodiments, R¹ is independently substituted heteroaryl (e.g., 5 to 10 membered heteroaryl, 5 to 9 membered heteroaryl, or 5 to 6 membered heteroaryl). In some embodiments, R¹ is independently an unsubstituted heteroaryl (e.g., 5 to 10 membered heteroaryl, 5 to 9 membered heteroaryl, or 5 to 6 membered heteroaryl).

In some embodiments, R¹ is independently halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, R²¹-substituted or unsubstituted alkyl (e.g., C₁-C₈ alkyl, C₁-C₆ alkyl, or C₁-C₄ alkyl), R²¹-substituted or unsubstituted heteroalkyl (e.g., 2 to 8 membered heteroalkyl, 2 to 6 membered heteroalkyl, or 2 to 4 membered heteroalkyl), R²¹-substituted or unsubstituted cycloalkyl (e.g., C₃-C₈ cycloalkyl, C₃-C₆ cycloalkyl, or C₅-C₆ cycloalkyl), R²¹-substituted or unsubstituted heterocycloalkyl (e.g., 3 to 8 membered heterocycloalkyl, 3 to 6 membered heterocycloalkyl, or 5 to 6 membered heterocycloalkyl), R²¹-substituted or unsubstituted aryl (e.g., C₆-C₁₀ aryl, C₁₀ aryl, or phenyl), or R²¹-substituted or unsubstituted heteroaryl (e.g., 5 to 10 membered heteroaryl, 5 to 9 membered heteroaryl, or 5 to 6 membered heteroaryl). In some embodiments, R¹ is independently halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R¹ is independently R²¹-substituted or unsubstituted alkyl (e.g., C₁-C₈ alkyl, C₁-C₆ alkyl, or C₁-C₄ alkyl). In some embodiments, R¹ is independently R²¹-substituted alkyl (e.g., C₁-C₈ alkyl, C₁-C₆ alkyl, or C₁-C₄ alkyl). In some embodiments, R¹ is independently an unsubstituted alkyl (e.g., C₁-C₈ alkyl, C₁-C₆ alkyl, or C₁-C₄ alkyl). In some embodiments, R¹ is independently R²¹-substituted or unsubstituted heteroalkyl (e.g., 2 to 8 membered heteroalkyl, 2 to 6 membered heteroalkyl, or 2 to 4 membered heteroalkyl). In some embodiments, R¹ is independently R²¹-substituted heteroalkyl (e.g., 2 to 8 membered heteroalkyl, 2 to 6 membered heteroalkyl, or 2 to 4 membered heteroalkyl). In some embodiments, R¹ is independently an unsubstituted heteroalkyl (e.g., 2 to 8 membered heteroalkyl, 2 to 6 membered heteroalkyl, or 2 to 4 membered heteroalkyl). In some embodiments, R¹ is independently R²¹-substituted or unsubstituted cycloalkyl (e.g., C₃-C₈ cycloalkyl, C₃-C₆ cycloalkyl, or C₅-C₆ cycloalkyl). In some embodiments, R¹ is independently R²¹-substituted cycloalkyl (e.g., C₃-C₈ cycloalkyl, C₃-C₆ cycloalkyl, or C₅-C₆ cycloalkyl). In some embodiments, R¹ is independently an unsubstituted cycloalkyl (e.g., C3-C₈ cycloalkyl, C₃-C₆ cycloalkyl, or C₅-C₆ cycloalkyl). In some embodiments, R¹ is independently R²¹-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R¹ is independently R²¹-substituted heterocycloalkyl (e.g., 3 to 8 membered heterocycloalkyl, 3 to 6 membered heterocycloalkyl, or 5 to 6 membered heterocycloalkyl). In some embodiments, R¹ is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R¹ is independently R²¹-substituted or unsubstituted aryl (e.g., C₆-C₁₀ aryl, C₁₀ aryl, or phenyl). In some embodiments, R¹ is independently R²¹-substituted aryl (e.g., C₆-C₁₀ aryl, C₁₀ aryl, or phenyl). In some embodiments, R¹ is independently an unsubstituted aryl (e.g., C₆-C₁₀ aryl, C₁₀ aryl, or phenyl). In some embodiments, R¹ is independently R²¹-substituted or unsubstituted heteroaryl (e.g., 5 to 10 membered heteroaryl, 5 to 9 membered heteroaryl, or 5 to 6 membered heteroaryl). In some embodiments, R¹ is independently R²¹-substituted heteroaryl (e.g., 5 to 10 membered heteroaryl, 5 to 9 membered heteroaryl, or 5 to 6 membered heteroaryl). In some embodiments, R¹ is independently an unsubstituted heteroaryl (e.g., 5 to 10 membered heteroaryl, 5 to 9 membered heteroaryl, or 5 to 6 membered heteroaryl).

In some embodiments, R¹ is independently —CX¹₃. In some embodiments, R¹ is independently —CHX¹₂. In some embodiments, R¹ is independently —CH₂X¹. In some embodiments, R¹ is independently —OCX¹₃. In some embodiments, R¹ is independently —OCH₂X¹. In some embodiments, R¹ is independently —OCHX¹₂. In some embodiments, R¹ is independently —CN. In some embodiments, R¹ is independently —SR^{1D} In some embodiments, R¹ is independently —SOR^{1D}. In some embodiments, R¹ is independently —SO₂R^{1D}. In some embodiments, R¹ is independently —SO₃R^{1D}. In some embodiments, R¹ is independently —SO₄R^{1D}. In some embodiments, R¹ is independently —SONR^{1A}R^{1B}. In some embodiments, R¹ is independently —SO₂NR^{1A}R^{1B}. In some embodiments, R¹ is independently —NHC(O)NR^{1A}R^{1B}. In some embodiments, R¹ is independently —N(O). In some embodiments, R¹ is independently —N(O)₂. In some embodiments, R¹ is independently —NR^{1A}R^{1B}. In some embodiments, R¹ is independently —C(O)R^{1C}. In some embodiments, R¹ is independently —C(O)—OR^{1C}. In some embodiments, R¹ is independently —C(O)NR^{1A}R^{1B}. In some embodiments, R¹ is independently —OR^{1D}. In some embodiments, R¹ is independently —NR^{1A}SO₂R^{1D}. In some embodiments, R¹ is independently —NR^{1A}C(O)R^{1C}. In some embodiments, R¹ is independently —NR^{1A}C(O)OR^{1C}. In some embodiments, R¹ is independently —NR^{1A}OR^{1C}.

In some embodiments, R¹ is independently oxo. In some embodiments, R¹ is independently halogen. In some embodiments, R¹ is independently —CCl₃. In some embodiments, R¹ is independently —CBr₃. In some embodiments, R¹ is independently —CF₃. In some embodiments, R¹ is independently —CI₃. In some embodiments, R¹ is independently —CHCl₂. In some embodiments, R¹ is independently —CHBr₂. In some embodiments, R¹ is independently —CHF₂. In some embodiments, R¹ is independently —CHI₂. In some embodiments, R¹ is independently —CH₂Cl. In some embodiments, R¹ is independently —CH₂Br. In some embodiments, R¹ is independently —CH₂F. In some embodiments, R¹ is independently —CH₂I. In some embodiments, R¹ is independently —CN. In some embodiments, R¹ is independently —OH. In some embodiments, R¹ is independently —NH₂. In some embodiments, R¹ is independently —COOH. In some embodiments, R¹ is independently —CONH₂. In some embodiments, R¹ is independently —NO₂. In some embodiments, R¹ is independently —SH. In some embodiments, R¹ is independently —SO₃H. In some embodiments, R¹ is independently —SO₄H. In some embodiments, R¹ is independently —SO₂NH₂. In some embodiments, R¹ is independently —NHNH₂. In some embodiments, R¹ is independently —ONH₂. In some embodiments, R¹ is independently —NHC(O)NHNH₂. In some embodiments, R¹ is independently —NHC(O)NH₂. In some embodiments, R¹ is independently —NHSO₂H. In some embodiments, R¹ is independently —NHC(O)H. In some embodiments, R¹ is independently —NHC(O)OH. In some embodiments, R¹ is independently —NHOH. In some embodiments, R¹ is independently —OCCl₃. In some embodiments, R¹ is independently —OCF₃. In some embodiments, R¹ is independently —OCBr₃. In some embodiments, R¹ is independently —OCI₃. In some embodiments, R¹ is independently —OCHCl₂. In some embodiments, R¹ is independently —OCHBr₂. In some embodiments, R¹ is independently —OCHI₂. In some embodiments, R¹ is independently —OCHF₂. In some embodiments, R¹ is independently —OCH₂Cl. In some embodiments, R¹ is independently —OCH₂Br. In some embodiments, R¹ is independently —OCH₂I. In some embodiments, R¹ is independently —OCH₂F. In some embodiments, R¹ is independently —N₃. In some embodiments, R¹ is independently —OCH₃. In some embodiments, R¹ is independently —CH₃. In some embodiments, R¹ is independently —CH₂CH₃. In some embodiments, R¹ is independently unsubstituted propyl. In some embodiments, R¹ is independently unsubstituted isopropyl. In some embodiments, R¹ is independently unsubstituted butyl. In some embodiments, R¹ is independently unsubstituted tert-butyl. In some embodiments, R¹ is independently —F. In some embodiments, R¹ is independently —Cl. In some embodiments, R¹ is independently —Br. In some embodiments, R¹ is independently —I.

In some embodiments, R²¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R²²-substituted or unsubstituted alkyl (e.g., C₁-C₈ alkyl, C₁-C₆ alkyl, or C₁-C₄ alkyl), R²²-substituted or unsubstituted heteroalkyl (e.g., 2 to 8 membered heteroalkyl, 2 to 6 membered heteroalkyl, or 2 to 4 membered heteroalkyl), R²²-substituted or unsubstituted cycloalkyl (e.g., C₃-C₈ cycloalkyl, C₃-C₆ cycloalkyl, or C₅-C₆ cycloalkyl), R²²-substituted or unsubstituted heterocycloalkyl (e.g., 3 to 8 membered heterocycloalkyl, 3 to 6 membered heterocycloalkyl, or 5 to 6 membered heterocycloalkyl), R²²-substituted or unsubstituted aryl (e.g., C₆-C₁₀ aryl, C₁₀ aryl, or phenyl), or R²²-substituted or unsubstituted heteroaryl (e.g., 5 to 10 membered heteroaryl, 5 to 9 membered heteroaryl, or 5 to 6 membered heteroaryl). In some embodiments, R²¹ is independently oxo, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R²¹ is independently oxo. In some embodiments, R²¹ is independently halogen. In some embodiments, R²¹ is independently —CCl₃. In some embodiments, R²¹ is independently —CBr₃. In some embodiments, R²¹ is independently —CF₃. In some embodiments, R²¹ is independently —CI₃. In some embodiments, R²¹ is independently —CHCl₂. In some embodiments, R²¹ is independently —CHBr₂. In some embodiments, R²¹ is independently —CHF₂. In some embodiments, R²¹ is independently —CHI₂. In some embodiments, R²¹ is independently —CH₂Cl. In some embodiments, R²¹ is independently —CH₂Br. In some embodiments, R²¹ is independently —CH₂F. In some embodiments, R²¹ is independently —CH₂I. In some embodiments, R²¹ is independently —CN. In some embodiments, R²¹ is independently —OH. In some embodiments, R²¹ is independently —NH₂. In some embodiments, R²¹ is independently —COOH. In some embodiments, R²¹ is independently —CONH₂. In some embodiments, R²¹ is independently —NO₂. In some embodiments, R²¹ is independently —SH. In some embodiments, R²¹ is independently —SO₃H. In some embodiments, R²¹ is independently —SO₄H. In some embodiments, R²¹ is independently —SO₂NH₂. In some embodiments, R²¹ is independently —NHNH₂. In some embodiments, R²¹ is independently —ONH₂. In some embodiments, R²¹ is independently —NHC(O)NHNH₂. In some embodiments, R²¹ is independently —NHC(O)NH₂. In some embodiments, R²¹ is independently —NHSO₂H. In some embodiments, R²¹ is independently —NHC(O)H. In some embodiments, R²¹ is independently —NHC(O)OH. In some embodiments, R²¹ is independently —NHOH. In some embodiments, R²¹ is independently —OCCl₃. In some embodiments, R²¹ is independently —OCF₃. In some embodiments, R²¹ is independently —OCBr₃. In some embodiments, R²¹ is independently —OCI₃. In some embodiments, R²¹ is independently —OCHCl₂. In some embodiments, R²¹ is independently —OCHBr₂. In some embodiments, R²¹ is independently —OCHI₂. In some embodiments, R²¹ is independently —OCHF₂. In some embodiments, R²¹ is independently —OCH₂Cl. In some embodiments, R²¹ is independently —OCH₂Br. In some embodiments, R²¹ is independently —OCH₂I. In some embodiments, R²¹ is independently —OCH₂F. In some embodiments, R²¹ is independently —N₃. In some embodiments, R²¹ is independently —OCH₃. In some embodiments, R²¹ is independently —CH₃. In some embodiments, R²¹ is independently —CH₂CH₃. In some embodiments, R²¹ is independently unsubstituted propyl.

In some embodiments, R²¹ is independently unsubstituted isopropyl. In some embodiments, R²¹ is independently unsubstituted butyl. In some embodiments, R²¹ is independently unsubstituted tert-butyl. In some embodiments, R²¹ is independently —F. In some embodiments, R²¹ is independently —Cl. In some embodiments, R²¹ is independently —Br. In some embodiments, R²¹ is independently —I.

In some embodiments, R²¹ is independently R²²-substituted or unsubstituted alkyl (e.g., C₁-C₈ alkyl, C₁-C₆ alkyl, or C₁-C₄ alkyl). In some embodiments, R²¹ is independently R²²-substituted alkyl (e.g., C₁-C₈ alkyl, C₁-C₆ alkyl, or C₁-C₄ alkyl). In some embodiments, R²¹ is independently an unsubstituted alkyl (e.g., C₁-C₈ alkyl, C₁-C₆ alkyl, or C₁-C₄ alkyl). In some embodiments, R²¹ is independently R²²-substituted or unsubstituted heteroalkyl (e.g., 2 to 8 membered heteroalkyl, 2 to 6 membered heteroalkyl, or 2 to 4 membered heteroalkyl). In some embodiments, R²¹ is independently R²²-substituted heteroalkyl (e.g., 2 to 8 membered heteroalkyl, 2 to 6 membered heteroalkyl, or 2 to 4 membered heteroalkyl). In some embodiments, R²¹ is independently an unsubstituted heteroalkyl (e.g., 2 to 8 membered heteroalkyl, 2 to 6 membered heteroalkyl, or 2 to 4 membered heteroalkyl). In some embodiments, R²¹ is independently R²²-substituted or unsubstituted cycloalkyl (e.g., C₃-C₈ cycloalkyl, C₃-C₆ cycloalkyl, or C₅-C₆ cycloalkyl). In some embodiments, R²¹ is independently R²²-substituted cycloalkyl (e.g., C₃-C₈ cycloalkyl, C₃-C₆ cycloalkyl, or C₅-C₆ cycloalkyl). In some embodiments, R²¹ is independently an unsubstituted cycloalkyl (e.g., C₃-C₈ cycloalkyl, C₃-C₆ cycloalkyl, or C₅-C₆ cycloalkyl). In some embodiments, R²¹ is independently R²²-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R²¹ is independently R²²-substituted heterocycloalkyl (e.g., 3 to 8 membered heterocycloalkyl, 3 to 6 membered heterocycloalkyl, or 5 to 6 membered heterocycloalkyl). In some embodiments, R²¹ is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R²¹ is independently R²²-substituted or unsubstituted aryl (e.g., C₆-C₁₀ aryl, C₁₀ aryl, or phenyl). In some embodiments, R²¹ is independently R²²-substituted aryl (e.g., C₆-C₁₀ aryl, C₁₀ aryl, or phenyl). In some embodiments, R²¹ is independently an unsubstituted aryl (e.g., C₆-C₁₀ aryl, C₁₀ aryl, or phenyl). In some embodiments, R²¹ is independently R²²-substituted or unsubstituted heteroaryl (e.g., 5 to 10 membered heteroaryl, 5 to 9 membered heteroaryl, or 5 to 6 membered heteroaryl). In some embodiments, R²¹ is independently R²²-substituted heteroaryl (e.g., 5 to 10 membered heteroaryl, 5 to 9 membered heteroaryl, or 5 to 6 membered heteroaryl). In some embodiments, R²¹ is independently an unsubstituted heteroaryl (e.g., 5 to 10 membered heteroaryl, 5 to 9 membered heteroaryl, or 5 to 6 membered heteroaryl).

In some embodiments, R²² is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R²³-substituted or unsubstituted alkyl (e.g., C₁-C₈ alkyl, C₁-C₆ alkyl, or C₁-C₄ alkyl), R²³-substituted or unsubstituted heteroalkyl (e.g., 2 to 8 membered heteroalkyl, 2 to 6 membered heteroalkyl, or 2 to 4 membered heteroalkyl), R²³-substituted or unsubstituted cycloalkyl (e.g., C₃-C₈ cycloalkyl, C₃-C₆ cycloalkyl, or C₅-C₆ cycloalkyl), R²³-substituted or unsubstituted heterocycloalkyl (e.g., 3 to 8 membered heterocycloalkyl, 3 to 6 membered heterocycloalkyl, or 5 to 6 membered heterocycloalkyl), R²³-substituted or unsubstituted aryl (e.g., C₆-C₁₀ aryl, C₁₀ aryl, or phenyl), or R²³-substituted or unsubstituted heteroaryl (e.g., 5 to 10 membered heteroaryl, 5 to 9 membered heteroaryl, or 5 to 6 membered heteroaryl). In some embodiments, R²² is independently oxo, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R²² is independently R²³-substituted or unsubstituted alkyl (e.g., C₁-C₈ alkyl, C1-C₆ alkyl, or C₁-C₄ alkyl). In some embodiments, R²² is independently R²³-substituted alkyl (e.g., C₁-C₈ alkyl, C₁-C₆ alkyl, or C₁-C₄ alkyl). In some embodiments, R²² is independently an unsubstituted alkyl (e.g., C₁-C₈ alkyl, C₁-C₆ alkyl, or C₁-C₄ alkyl). In some embodiments, R²² is independently R²³-substituted or unsubstituted heteroalkyl (e.g., 2 to 8 membered heteroalkyl, 2 to 6 membered heteroalkyl, or 2 to 4 membered heteroalkyl). In some embodiments, R²² is independently R²³-substituted heteroalkyl (e.g., 2 to 8 membered heteroalkyl, 2 to 6 membered heteroalkyl, or 2 to 4 membered heteroalkyl). In some embodiments, R²² is independently an unsubstituted heteroalkyl (e.g., 2 to 8 membered heteroalkyl, 2 to 6 membered heteroalkyl, or 2 to 4 membered heteroalkyl). In some embodiments, R²² is independently R²³-substituted or unsubstituted cycloalkyl (e.g., C₃-C₈ cycloalkyl, C₃-C₆ cycloalkyl, or C₅-C₆ cycloalkyl). In some embodiments, R²² is independently R²³-substituted cycloalkyl (e.g., C₃-C₈ cycloalkyl, C₃-C₆ cycloalkyl, or C₅-C₆ cycloalkyl). In some embodiments, R²² is independently an unsubstituted cycloalkyl (e.g., C₃-C₈ cycloalkyl, C₃-C₆ cycloalkyl, or C₅-C₆ cycloalkyl). In some embodiments, R²² is independently R²³-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R²² is independently R²³-substituted heterocycloalkyl (e.g., 3 to 8 membered heterocycloalkyl, 3 to 6 membered heterocycloalkyl, or 5 to 6 membered heterocycloalkyl). In some embodiments, R²² is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R²² is independently R²³-substituted or unsubstituted aryl (e.g., C₆-C₁₀ aryl, C₁₀ aryl, or phenyl). In some embodiments, R²² is independently R²³-substituted aryl (e.g., C₆-C₁₀ aryl, C₁₀ aryl, or phenyl). In some embodiments, R²² is independently an unsubstituted aryl (e.g., C₆-C₁₀ aryl, C₁₀ aryl, or phenyl). In some embodiments, R²² is independently R²³-substituted or unsubstituted heteroaryl (e.g., 5 to 10 membered heteroaryl, 5 to 9 membered heteroaryl, or 5 to 6 membered heteroaryl). In some embodiments, R²² is independently R²³-substituted heteroaryl (e.g., 5 to 10 membered heteroaryl, 5 to 9 membered heteroaryl, or 5 to 6 membered heteroaryl). In some embodiments, R²² is independently an unsubstituted heteroaryl (e.g., 5 to 10 membered heteroaryl, 5 to 9 membered heteroaryl, or 5 to 6 membered heteroaryl).

In some embodiments, R²² is independently oxo. In some embodiments, R²² is independently halogen. In some embodiments, R²² is independently —CCl₃. In some embodiments, R²² is independently —CBr₃. In some embodiments, R²² is independently —CF₃. In some embodiments, R²² is independently —CI₃. In some embodiments, R²² is independently CHCl₂. In some embodiments, R²² is independently —CHBr₂. In some embodiments, R²² is independently —CHF₂. In some embodiments, R²² is independently —CHI₂. In some embodiments, R²² is independently —CH₂Cl. In some embodiments, R²² is independently —CH₂Br. In some embodiments, R²² is independently —CH₂F. In some embodiments, R²² is independently —CH₂I. In some embodiments, R²² is independently —CN. In some embodiments, R²² is independently —OH. In some embodiments, R²² is independently —NH₂. In some embodiments, R²² is independently —COOH. In some embodiments, R²² is independently —CONH₂. In some embodiments, R²² is independently —NO₂. In some embodiments, R²² is independently —SH. In some embodiments, R²² is independently —SO₃H. In some embodiments, R²² is independently —SO₄H. In some embodiments, R²² is independently —SO₂NH₂. In some embodiments, R²² is independently —NHNH₂. In some embodiments, R²² is independently —ONH₂. In some embodiments, R²² is independently —NHC(O)NHNH₂. In some embodiments, R²² is independently —NHC(O)NH₂. In some embodiments, R²² is independently —NHSO₂H. In some embodiments, R²² is independently —NHC(O)H. In some embodiments, R²² is independently —NHC(O)OH. In some embodiments, R²² is independently —NHOH. In some embodiments, R²² is independently —OCCl₃. In some embodiments, R²² is independently —OCF₃. In some embodiments, R²² is independently —OCBr₃. In some embodiments, R²² is independently —OCI₃. In some embodiments, R²² is independently —OCHCl₂. In some embodiments, R²² is independently —OCHBr₂. In some embodiments, R²² is independently —OCHI₂. In some embodiments, R²² is independently —OCHF₂. In some embodiments, R²² is independently —OCH₂Cl. In some embodiments, R²² is independently —OCH₂Br. In some embodiments, R²² is independently —OCH₂I. In some embodiments, R²² is independently —OCH₂F. In some embodiments, R²² is independently —N₃. In some embodiments, R²² is independently —OCH₃. In some embodiments, R²² is independently —CH₃. In some embodiments, R²² is independently —CH₂CH₃. In some embodiments, R²² is independently unsubstituted propyl. In some embodiments, R²² is independently unsubstituted isopropyl. In some embodiments, R²² is independently unsubstituted butyl. In some embodiments, R²² is independently unsubstituted tert-butyl. In some embodiments, R²² is independently —F. In some embodiments, R²² is independently —Cl. In some embodiments, R²² is independently —Br. In some embodiments, R²² is independently —I.

In some embodiments, R²³ is independently oxo, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R²³ is independently an unsubstituted alkyl (e.g., C₁-C₈ alkyl, C₁-C₆ alkyl, or C₁-C₄ alkyl). In some embodiments, R²³ is independently an unsubstituted heteroalkyl (e.g., 2 to 8 membered heteroalkyl, 2 to 6 membered heteroalkyl, or 2 to 4 membered heteroalkyl). In some embodiments, R²³ is independently an unsubstituted cycloalkyl (e.g., C₃-C₈ cycloalkyl, C₃-C₆ cycloalkyl, or C₅-C₆ cycloalkyl). In some embodiments, R²³ is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R²³ is independently an unsubstituted aryl (e.g., C₆-C₁₀ aryl, C₁₀ aryl, or phenyl). In some embodiments, R²³ is independently an unsubstituted heteroaryl (e.g., 5 to 10 membered heteroaryl, 5 to 9 membered heteroaryl, or 5 to 6 membered heteroaryl).

In some embodiments, R²³ is independently oxo. In some embodiments, R²³ is independently halogen. In some embodiments, R²³ is independently —CCl₃. In some embodiments, R²³ is independently —CBr₃. In some embodiments, R²³ is independently —CF₃. In some embodiments, R²³ is independently —CI₃. In some embodiments, R²³ is independently —CHCl₂. In some embodiments, R²³ is independently —CHBr₂. In some embodiments, R²³ is independently —CHF₂. In some embodiments, R²³ is independently —CHI₂. In some embodiments, R²³ is independently —CH₂Cl. In some embodiments, R²³ is independently —CH₂Br. In some embodiments, R²³ is independently —CH₂F. In some embodiments, R²³ is independently —CH₂I. In some embodiments, R²³ is independently —CN. In some embodiments, R²³ is independently —OH. In some embodiments, R²³ is independently —NH₂. In some embodiments, R²³ is independently —COOH. In some embodiments, R²³ is independently —CONH₂. In some embodiments, R²³ is independently —NO₂. In some embodiments, R²³ is independently —SH. In some embodiments, R²³ is independently —SO₃H. In some embodiments, R²³ is independently —SO₄H. In some embodiments, R²³ is independently —SO₂NH₂. In some embodiments, R²³ is independently —NHNH₂. In some embodiments, R²³ is independently —ONH₂. In some embodiments, R²³ is independently —NHC(O)NHNH₂. In some embodiments, R²³ is independently —NHC(O)NH₂. In some embodiments, R²³ is independently —NHSO₂H. In some embodiments, R²³ is independently —NHC(O)H. In some embodiments, R²³ is independently —NHC(O)OH. In some embodiments, R²³ is independently —NHOH. In some embodiments, R²³ is independently —OCCl₃. In some embodiments, R²³ is independently —OCF₃. In some embodiments, R²³ is independently —OCBr₃. In some embodiments, R²³ is independently —OCI₃. In some embodiments, R²³ is independently —OCHCl₂. In some embodiments, R²³ is independently —OCHBr₂. In some embodiments, R²³ is independently —OCHI₂. In some embodiments, R²³ is independently —OCHF₂. In some embodiments, R²³ is independently —OCH₂Cl. In some embodiments, R²³ is independently —OCH₂Br. In some embodiments, R²³ is independently —OCH₂I. In some embodiments, R²³ is independently —OCH₂F. In some embodiments, R²³ is independently —N₃. In some embodiments, R²³ is independently —OCH₃.

In some embodiments, R²³ is independently —CH₃. In some embodiments, R²³ is independently —CH₂CH₃. In some embodiments, R²³ is independently unsubstituted propyl. In some embodiments, R²³ is independently unsubstituted isopropyl. In some embodiments, R²³ is independently unsubstituted butyl. In some embodiments, R²³ is independently unsubstituted tert-butyl. In some embodiments, R²³ is independently —F. In some embodiments, R²³ is independently —Cl. In some embodiments, R²³ is independently —Br. In some embodiments, R²³ is independently —I.

In some embodiments, two (e.g., adjacent) R¹ substituents are independently joined to form a substituted or unsubstituted alkylene (e.g., —CH₂- or —CH₂CH₂-), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). In some embodiments, two R¹ substituents are independently joined to form a substituted or unsubstituted alkylene (e.g., —CH₂- or —CH₂CH₂-). In some embodiments, two adjacent R¹ substituents are independently joined to form a substituted or unsubstituted alkylene (e.g., —CH₂- or —CH₂CH₂-). In some embodiments, two non-adjacent R¹ substituents are independently joined to form a substituted or unsubstituted alkylene (e.g., —CH₂- or —CH₂CH₂-). In some embodiments, two (e.g., adjacent) R¹ substituents are independently joined to form a substituted or unsubstituted cycloalkyl (e.g., C₃-C₈ cycloalkyl, C₃-C₆ cycloalkyl, or C₅-C₆ cycloalkyl). In some embodiments, two (e.g., adjacent) R¹ substituents are independently joined to form a substituted cycloalkyl (e.g., C₃-C₈ cycloalkyl, C₃-C₆ cycloalkyl, or C₅-C₆ cycloalkyl). In some embodiments, two (e.g., adjacent) R¹ substituents are independently joined to form an unsubstituted cycloalkyl (e.g., C₃-C₈ cycloalkyl, C₃-C₆ cycloalkyl, or C₅-C₆ cycloalkyl). In some embodiments, two (e.g., adjacent) R¹ substituents are independently joined to form a substituted or unsubstituted heterocycloalkyl (e.g., 3 to 8 membered heterocycloalkyl, 3 to 6 membered heterocycloalkyl, or 5 to 6 membered heterocycloalkyl). In some embodiments, two (e.g., adjacent) R¹ substituents are independently joined to form a substituted heterocycloalkyl (e.g., 3 to 8 membered heterocycloalkyl, 3 to 6 membered heterocycloalkyl, or 5 to 6 membered heterocycloalkyl). In some embodiments, two (e.g., adjacent) R¹ substituents are independently joined to form an unsubstituted heterocycloalkyl (e.g., 3 to 8 membered heterocycloalkyl, 3 to 6 membered heterocycloalkyl, or 5 to 6 membered heterocycloalkyl). In some embodiments, two (e.g., adjacent) R¹ substituents are independently joined to form a substituted or unsubstituted aryl (e.g., C₆-C₁₀ aryl, C₁₀ aryl, or phenyl). In some embodiments, two (e.g., adjacent) R¹ substituents are independently joined to form a substituted aryl (e.g., C₆-C₁₀ aryl, C₁₀ aryl, or phenyl). In some embodiments, two (e.g., adjacent) R¹ substituents are independently joined to form an unsubstituted aryl (e.g., C₆-C₁₀ aryl, C₁₀ aryl, or phenyl). In some embodiments, two (e.g., adjacent) R¹ substituents are independently joined to form a substituted or unsubstituted heteroaryl (e.g., 5 to 10 membered heteroaryl, 5 to 9 membered heteroaryl, or 5 to 6 membered heteroaryl). In some embodiments, two (e.g., adjacent) R¹ substituents are independently joined to form a substituted heteroaryl (e.g., 5 to 10 membered heteroaryl, 5 to 9 membered heteroaryl, or 5 to 6 membered heteroaryl). In some embodiments, two (e.g., adjacent) R¹ substituents are independently joined to form an unsubstituted heteroaryl (e.g., 5 to 10 membered heteroaryl, 5 to 9 membered heteroaryl, or 5 to 6 membered heteroaryl). In some embodiments, two (e.g., adjacent) R¹ substituents are independently joined to form an R²¹-substituted or unsubstituted cycloalkyl (e.g., C₃-C₈ cycloalkyl, C₃-C₆ cycloalkyl, or C₅-C₆ cycloalkyl). In some embodiments, two (e.g., adjacent) R¹ substituents are independently joined to form an R²¹-substituted cycloalkyl (e.g., C₃-C₈ cycloalkyl, C₃-C₆ cycloalkyl, or C₅-C₆ cycloalkyl). In some embodiments, two (e.g., adjacent) R¹ substituents are independently joined to form an unsubstituted cycloalkyl (e.g., C₃-C₈ cycloalkyl, C₃-C₆ cycloalkyl, or C₅-C₆ cycloalkyl). In some embodiments, two (e.g., adjacent) R¹ substituents are independently joined to form an R²¹-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, two (e.g., adjacent) R¹ substituents are independently joined to form an R²¹-substituted heterocycloalkyl (e.g., 3 to 8 membered heterocycloalkyl, 3 to 6 membered heterocycloalkyl, or 5 to 6 membered heterocycloalkyl). In some embodiments, two (e.g., adjacent) R¹ substituents are independently joined to form an unsubstituted heterocycloalkyl (e.g., 3 to 8 membered heterocycloalkyl, 3 to 6 membered heterocycloalkyl, or 5 to 6 membered heterocycloalkyl). In some embodiments, two (e.g., adjacent) R¹ substituents are independently joined to form an R²¹-substituted or unsubstituted aryl (e.g., C₆-C₁₀ aryl, C₁₀ aryl, or phenyl). In some embodiments, two (e.g., adjacent) R¹ substituents are independently joined to form an R²¹-substituted aryl (e.g., C₆-C₁₀ aryl, C₁₀ aryl, or phenyl). In some embodiments, two (e.g., adjacent) R¹ substituents are independently joined to form an unsubstituted aryl (e.g., C₆-C₁₀ aryl, C₁₀ aryl, or phenyl). In some embodiments, two (e.g., adjacent) R¹ substituents are independently joined to form an R²¹-substituted or unsubstituted heteroaryl (e.g., 5 to 10 membered heteroaryl, 5 to 9 membered heteroaryl, or 5 to 6 membered heteroaryl). In some embodiments, two (e.g., adjacent) R¹ substituents are independently joined to form an R²¹-substituted heteroaryl (e.g., 5 to 10 membered heteroaryl, 5 to 9 membered heteroaryl, or 5 to 6 membered heteroaryl). In some embodiments, two (e.g., adjacent) R¹ substituents are independently joined to form an unsubstituted heteroaryl (e.g., 5 to 10 membered heteroaryl, 5 to 9 membered heteroaryl, or 5 to 6 membered heteroaryl). It is understood that when two R¹ substituents are taken together to form a ring structure (fused or bridged), the two R¹ substituents can be adjacent or non-adjacent.

In some embodiments, one R¹ substituent is taken together with R² to form a substituted or unsubstituted heterocycloalkyl (e.g., 3 to 8 membered heterocycloalkyl, 3 to 6 membered heterocycloalkyl, or 5 to 6 membered heterocycloalkyl), or substituted or unsubstituted heteroaryl (e.g., 5 to 10 membered heteroaryl, 5 to 9 membered heteroaryl, or 5 to 6 membered heteroaryl). In some embodiments, one R¹ substituent is taken together with R² to form a substituted or unsubstituted heterocycloalkyl (e.g., 3 to 8 membered heterocycloalkyl, 3 to 6 membered heterocycloalkyl, or 5 to 6 membered heterocycloalkyl). In some embodiments, one R¹ substituent is taken together with R² to form a substituted heterocycloalkyl (e.g., 3 to 8 membered heterocycloalkyl, 3 to 6 membered heterocycloalkyl, or 5 to 6 membered heterocycloalkyl). In some embodiments, one R¹ substituent is taken together with R² to form an unsubstituted heterocycloalkyl (e.g., 3 to 8 membered heterocycloalkyl, 3 to 6 membered heterocycloalkyl, or 5 to 6 membered heterocycloalkyl). In some embodiments, one R¹ substituent is taken together with R² to form an R²¹-substituted heterocycloalkyl (e.g., 3 to 8 membered heterocycloalkyl, 3 to 6 membered heterocycloalkyl, or 5 to 6 membered heterocycloalkyl). In some embodiments, one R¹ substituent is taken together with R² to form a substituted or unsubstituted heteroaryl (e.g., 5 to 10 membered heteroaryl, 5 to 9 membered heteroaryl, or 5 to 6 membered heteroaryl). In some embodiments, one R¹ substituent is taken together with R² to form a substituted heteroaryl (e.g., 5 to 10 membered heteroaryl, 5 to 9 membered heteroaryl, or 5 to 6 membered heteroaryl). In some embodiments, one R¹ substituent is taken together with R² to form an unsubstituted heteroaryl (e.g., 5 to 10 membered heteroaryl, 5 to 9 membered heteroaryl, or 5 to 6 membered heteroaryl). In some embodiments, one R¹ substituent is taken together with R² to form an R²¹-substituted heteroaryl (e.g., 5 to 10 membered heteroaryl, 5 to 9 membered heteroaryl, or 5 to 6 membered heteroaryl).

In some embodiments, the divalent linker bonded to both the monovalent cellular component binder and monovalent targeted autophagy protein binder is attached to the monovalent targeted autophagy protein binder at an R¹ position. In some embodiments, when the divalent linker bonded to both the monovalent cellular component binder and monovalent targeted autophagy protein binder is attached to the monovalent targeted autophagy protein binder at an R¹ position, R¹ is replaced with a divalent linker, referred to in this embodiment as L^{R1}.

In some embodiments, L^{R1} is a bond, —S(O)₂—, —S(O)—, —NR^{1A}—, ═N—, —O—, —S—, —C(O)—, —C(O)NR^{1A}—, —NR^{1A}C(O)—, —NR^{1A}C(O)NH—, —NHC(O)NR^{1A}—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. It will be understood that when -L^{R1}- is ═N—, one of the two direct covalent connections to L^{R1} shown in “-L^{R1}-” is a double bond and L^{R1} may equivalently be shown as “=L^{R1}-” and the atom to which the double bond is directly attached must obey standard rules of chemical valency known in the chemical arts and be capable of forming such a double bond.

In some embodiments, L^{R1} is independently a bond, —S(O)₂—, —S(O)—, —NH—, —O—, —S—, —C(O)—, —C(O)NH—, —NHC(O)—, —NHC(O)NH—, —C(O)O—, —OC(O)—, substituted or unsubstituted alkylene (e.g., C₁-C₈ alkylene, C₁-C₆ alkylene, or C₁-C₄ alkylene), substituted or unsubstituted heteroalkylene (e.g., 2 to 8 membered heteroalkylene, 2 to 6 membered heteroalkylene, or 2 to 4 membered heteroalkylene), substituted or unsubstituted cycloalkylene (e.g., C₃-C₈ cycloalkylene, C₃-C₆ cycloalkylene, or C₅-C₆ cycloalkylene), substituted or unsubstituted heterocycloalkylene (e.g., 3 to 8 membered heterocycloalkylene, 3 to 6 membered heterocycloalkylene, or 5 to 6 membered heterocycloalkylene), substituted or unsubstituted arylene (e.g., C₆-C₁₀ arylene, C₁₀ arylene, or phenylene), or substituted or unsubstituted heteroarylene (e.g., 5 to 10 membered heteroarylene, 5 to 9 membered heteroarylene, or 5 to 6 membered heteroarylene). In some embodiments, L^{R1} is independently a —S(O)₂—. In some embodiments, L^{R1} is independently a —S(O)—. In some embodiments, L^{R1} is independently a —NH—. In some embodiments, L^{R1} is independently a —O—. In some embodiments, L^{R1} is independently a —S—. In some embodiments, L^{R1} is independently a —C(O)—. In some embodiments, L^{R1} is independently a —C(O)NH—. In some embodiments, L^{R1} is independently a —NHC(O)—. In some embodiments, L^{R1} is independently a —NHC(O)NH—. In some embodiments, L^{R1} is independently a —C(O)O—. In some embodiments, L^{R1} is independently —OC(O)—. In some embodiments, L^{R1} is independently —NR^{1A}—. In some embodiments, L^{R1} is independently —C(O)NR^{1A}—. In some embodiments, L^{R1} is independently —NR^{1A}C(O)—. In some embodiments, L^{R1} is independently —NR^{1A}C(O)NH—. In some embodiments, L^{R1} is independently —NHC(O)NR^{1A}—. In some embodiments, L^{R1} is independently a bond.

In some embodiments, L^{R1} is substituted or unsubstituted alkylene (e.g., C₁-C₈ alkylene, C₁-C₆ alkylene, or C₁-C₄ alkylene). In some embodiments, L^{R1} is substituted alkylene (e.g., C₁-C₈ alkylene, C₁-C₆ alkylene, or C₁-C₄ alkylene). In some embodiments, L^{R1} is an unsubstituted alkylene (e.g., C₁-C₈ alkylene, C₁-C₆ alkylene, or C₁-C₄ alkylene). In some embodiments, L^{R1} is substituted or unsubstituted heteroalkylene (e.g., 2 to 8 membered heteroalkylene, 2 to 6 membered heteroalkylene, or 2 to 4 membered heteroalkylene). In some embodiments, L^{R1} is substituted heteroalkylene (e.g., 2 to 8 membered heteroalkylene, 2 to 6 membered heteroalkylene, or 2 to 4 membered heteroalkylene). In some embodiments, L^{R1} is an unsubstituted heteroalkylene (e.g., 2 to 8 membered heteroalkylene, 2 to 6 membered heteroalkylene, or 2 to 4 membered heteroalkylene). In some embodiments, L^{R1} is substituted or unsubstituted cycloalkylene (e.g., C₃-C₈ cycloalkylene, C₃-C₆ cycloalkylene, or C₅-C₆ cycloalkylene). In some embodiments, L^{R1} is substituted cycloalkylene (e.g., C3-C8 cycloalkylene, C₃-C₆ cycloalkylene, or C₅-C₆ cycloalkylene). In some embodiments, L^{R1} is an unsubstituted cycloalkylene (e.g., C₃-C₈ cycloalkylene, C₃-C₆ cycloalkylene, or C₅-C₆ cycloalkylene). In some embodiments, L^{R1} is substituted or unsubstituted heterocycloalkylene (e.g., 3 to 8 membered heterocycloalkylene, 3 to 6 membered heterocycloalkylene, or 5 to 6 membered heterocycloalkylene). In some embodiments, L^{R1} is substituted heterocycloalkylene (e.g., 3 to 8 membered heterocycloalkylene, 3 to 6 membered heterocycloalkylene, or 5 to 6 membered heterocycloalkylene). In some embodiments, L^{R1} is an unsubstituted heterocycloalkylene (e.g., 3 to 8 membered heterocycloalkylene, 3 to 6 membered heterocycloalkylene, or 5 to 6 membered heterocycloalkylene). In some embodiments, L^{R1} is substituted or unsubstituted arylene (e.g., C₆-C₁₀ arylene, C₁₀ arylene, or phenylene). In some embodiments, L^{R1} is substituted arylene (e.g., C₆-C₁₀ arylene, C₁₀ arylene, or phenylene). In some embodiments, L^{R1} is an unsubstituted arylene (e.g., C₆-C₁₀ arylene, C₁₀ arylene, or phenylene). In some embodiments, L^{R1} is substituted or unsubstituted heteroarylene (e.g., 5 to 10 membered heteroarylene, 5 to 9 membered heteroarylene, or 5 to 6 membered heteroarylene). In some embodiments, L^{R1} is substituted heteroarylene (e.g., 5 to 10 membered heteroarylene, 5 to 9 membered heteroarylene, or 5 to 6 membered heteroarylene). In some embodiments, L^{R1} is an unsubstituted heteroarylene (e.g., 5 to 10 membered heteroarylene, 5 to 9 membered heteroarylene, or 5 to 6 membered heteroarylene).

In some embodiments, L^{R1} is independently a bond, —S(O)₂—, —S(O)—, —NH—, —O—, —S—, —C(O)—, —C(O)NH—, —NHC(O)—, —NHC(O)NH—, —C(O)O—, —OC(O)—, R²¹-substituted or unsubstituted alkylene (e.g., C₁-C₈ alkylene, C₁-C₆ alkylene, or C₁-C₄ alkylene), R²¹-substituted or unsubstituted heteroalkylene (e.g., 2 to 8 membered heteroalkylene, 2 to 6 membered heteroalkylene, or 2 to 4 membered heteroalkylene), R²¹-substituted or unsubstituted cycloalkylene (e.g., C₃-C₈ cycloalkylene, C₃-C₆ cycloalkylene, or C₅-C₆ cycloalkylene), R²¹-substituted or unsubstituted heterocycloalkylene (e.g., 3 to 8 membered heterocycloalkylene, 3 to 6 membered heterocycloalkylene, or 5 to 6 membered heterocycloalkylene), R²¹-substituted or unsubstituted arylene (e.g., C₆-C₁₀ arylene, C₁₀ arylene, or phenylene), or R²¹-substituted or unsubstituted heteroarylene (e.g., 5 to 10 membered heteroarylene, 5 to 9 membered heteroarylene, or 5 to 6 membered heteroarylene). In some embodiments, L^{R1} is independently a bond —S(O)₂—, —S(O)—, —NH—, —O—, —S—, —C(O)—, —C(O)NH—, —NHC(O)—, —NHC(O)NH—, —C(O)O—, —OC(O)—, unsubstituted alkylene (e.g., C₁-C₈ alkylene, C₁-C₆ alkylene, or C₁-C₄ alkylene), unsubstituted heteroalkylene (e.g., 2 to 8 membered heteroalkylene, 2 to 6 membered heteroalkylene, or 2 to 4 membered heteroalkylene), unsubstituted cycloalkylene (e.g., C₃-C₈ cycloalkylene, C₃-C₆ cycloalkylene, or C₅-C₆ cycloalkylene), unsubstituted heterocycloalkylene (e.g., 3 to 8 membered heterocycloalkylene, 3 to 6 membered heterocycloalkylene, or 5 to 6 membered heterocycloalkylene), unsubstituted arylene (e.g., C₆-C₁₀ arylene, C₁₀ arylene, or phenylene), or unsubstituted heteroarylene (e.g., 5 to 10 membered heteroarylene, 5 to 9 membered heteroarylene, or 5 to 6 membered heteroarylene).

In some embodiments, L^{R1} is R²¹-substituted or unsubstituted alkylene (e.g., C₁-C₈ alkylene, C₁-C₆ alkylene, or C₁-C₄ alkylene). In some embodiments, L^{R1} is R²¹-substituted alkylene (e.g., C₁-C₈ alkylene, C₁-C₆ alkylene, or C₁-C₄ alkylene). In some embodiments, L^{R1} is an unsubstituted alkylene (e.g., C₁-C₈ alkylene, C₁-C₆ alkylene, or C₁-C₄ alkylene). In some embodiments, L^{R1} is R²¹-substituted or unsubstituted heteroalkylene (e.g., 2 to 8 membered heteroalkylene, 2 to 6 membered heteroalkylene, or 2 to 4 membered heteroalkylene). In some embodiments, L^{R1} is R²¹-substituted heteroalkylene (e.g., 2 to 8 membered heteroalkylene, 2 to 6 membered heteroalkylene, or 2 to 4 membered heteroalkylene). In some embodiments, L^{R1} is an unsubstituted heteroalkylene (e.g., 2 to 8 membered heteroalkylene, 2 to 6 membered heteroalkylene, or 2 to 4 membered heteroalkylene). In some embodiments, L^{R1} is R²¹-substituted or unsubstituted cycloalkylene (e.g., C₃-C₈ cycloalkylene, C₃-C₆ cycloalkylene, or C₅-C₆ cycloalkylene). In some embodiments, L^{R1} is R²¹-substituted cycloalkylene (e.g., C₃-C₈ cycloalkylene, C₃-C₆ cycloalkylene, or C₅-C₆ cycloalkylene). In some embodiments, L^{R1} is an unsubstituted cycloalkylene (e.g., C₃-C₈ cycloalkylene, C₃-C₆ cycloalkylene, or C₅-C₆ cycloalkylene). In some embodiments, L^{R1} is R²¹-substituted or unsubstituted heterocycloalkylene (e.g., 3 to 8 membered heterocycloalkylene, 3 to 6 membered heterocycloalkylene, or 5 to 6 membered heterocycloalkylene). In some embodiments, L^{R1} is R²¹-substituted heterocycloalkylene (e.g., 3 to 8 membered heterocycloalkylene, 3 to 6 membered heterocycloalkylene, or 5 to 6 membered heterocycloalkylene). In some embodiments, L^{R1} is an unsubstituted heterocycloalkylene (e.g., 3 to 8 membered heterocycloalkylene, 3 to 6 membered heterocycloalkylene, or 5 to 6 membered heterocycloalkylene). In some embodiments, L^{R1} is R²¹-substituted or unsubstituted arylene (e.g., C₆-C₁₀ arylene, C₁₀ arylene, or phenylene). In some embodiments, L^{R1} is R²¹-substituted arylene (e.g., C₆-C₁₀ arylene, C₁₀ arylene, or phenylene). In some embodiments, L^{R1} is an unsubstituted arylene (e.g., C₆-C₁₀ arylene, C₁₀ arylene, or phenylene). In some embodiments, L^{R1} is R²¹-substituted or unsubstituted heteroarylene (e.g., 5 to 10 membered heteroarylene, 5 to 9 membered heteroarylene, or 5 to 6 membered heteroarylene). In some embodiments, L^{R1} is R²¹-substituted heteroarylene (e.g., 5 to 10 membered heteroarylene, 5 to 9 membered heteroarylene, or 5 to 6 membered heteroarylene). In some embodiments, L^{R1} is an unsubstituted heteroarylene (e.g., 5 to 10 membered heteroarylene, 5 to 9 membered heteroarylene, or 5 to 6 membered heteroarylene).

In some embodiments, R^{1A} is independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In some embodiments, the divalent linker bonded to both the monovalent cellular component binder and monovalent targeted autophagy protein binder is attached to the monovalent targeted autophagy protein binder at an R^{1A} position. In some embodiments, when the divalent linker bonded to both the monovalent cellular component binder and monovalent targeted autophagy protein binder is attached to the monovalent targeted autophagy protein binder at an R^{1A} position, R^{1A} is replaced with a divalent linker, referred to in this embodiment as L^{R1}.

In some embodiments, R^{1B} is independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In some embodiments, the divalent linker bonded to both the monovalent cellular component binder and monovalent targeted autophagy protein binder is attached to the monovalent targeted autophagy protein binder at an R^{1B} position. In some embodiments, when the divalent linker bonded to both the monovalent cellular component binder and monovalent targeted autophagy protein binder is attached to the monovalent targeted autophagy protein binder at an R^{1B} position, R^{1B} is replaced with a divalent linker, referred to in this embodiment as L^{R1}.

In some embodiments, R^{1C} is independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In some embodiments, the divalent linker bonded to both the monovalent cellular component binder and monovalent targeted autophagy protein binder is attached to the monovalent targeted autophagy protein binder at an R^{1C} position. In some embodiments, when the divalent linker bonded to both the monovalent cellular component binder and monovalent targeted autophagy protein binder is attached to the monovalent targeted autophagy protein binder at an R^{1C} position, R^{1C} is replaced with a divalent linker, referred to in this embodiment as L^{R1}.

In some embodiments, R^{1D} is independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In some embodiments, the divalent linker bonded to both the monovalent cellular component binder and monovalent targeted autophagy protein binder is attached to the monovalent targeted autophagy protein binder at an R^{1D} position. In some embodiments, when the divalent linker bonded to both the monovalent cellular component binder and monovalent targeted autophagy protein binder is attached to the monovalent targeted autophagy protein binder at an R^{1D} position, R^{1D} is replaced with a divalent linker, referred to in this embodiment as L^{R1}.

In some embodiments, R^{1A} and R^{1B} substituents bonded to the same nitrogen atom are independently joined to form a substituted or unsubstituted heterocycloalkyl (e.g., 3 to 8 membered heterocycloalkyl, 3 to 6 membered heterocycloalkyl, or 5 to 6 membered heterocycloalkyl) or substituted or unsubstituted heteroaryl (e.g., 5 to 10 membered heteroaryl, 5 to 9 membered heteroaryl, or 5 to 6 membered heteroaryl). In some embodiments, R^{1A} and R^{1B} substituents bonded to the same nitrogen atom are independently joined to form a substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R^{1A} and R^{1B} substituents bonded to the same nitrogen atom are independently joined to form a substituted heterocycloalkyl (e.g., 3 to 8 membered heterocycloalkyl, 3 to 6 membered heterocycloalkyl, or 5 to 6 membered heterocycloalkyl). In some embodiments, R^{1A} and R^{1B} substituents bonded to the same nitrogen atom are independently joined to form an unsubstituted heterocycloalkyl (e.g., 3 to 8 membered heterocycloalkyl, 3 to 6 membered heterocycloalkyl, or 5 to 6 membered heterocycloalkyl). In some embodiments, R^{1A} and R^{1B} substituents bonded to the same nitrogen atom are independently joined to form a substituted or unsubstituted heteroaryl (e.g., 5 to 10 membered heteroaryl, 5 to 9 membered heteroaryl, or 5 to 6 membered heteroaryl). In some embodiments, R^{1A} and R^{1B} substituents bonded to the same nitrogen atom are independently joined to form a substituted heteroaryl (e.g., 5 to 10 membered heteroaryl, 5 to 9 membered heteroaryl, or 5 to 6 membered heteroaryl). In some embodiments, R^{1A} and R^{1B} substituents bonded to the same nitrogen atom are independently joined to form an unsubstituted heteroaryl (e.g., 5 to 10 membered heteroaryl, 5 to 9 membered heteroaryl, or 5 to 6 membered heteroaryl).

In some embodiments, R^{1A}, R^{1B}, R^{1C}, and R^{1D} are independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, R²¹-substituted or unsubstituted alkyl (e.g., C₁-C₈ alkyl, C₁-C₆ alkyl, or C₁-C₄ alkyl), R²¹-substituted or unsubstituted heteroalkyl (e.g., 2 to 8 membered heteroalkyl, 2 to 6 membered heteroalkyl, or 2 to 4 membered heteroalkyl), R²¹-substituted or unsubstituted cycloalkyl (e.g., C₃-C₈ cycloalkyl, C₃-C₆ cycloalkyl, or C₅-C₆ cycloalkyl), R²¹-substituted or unsubstituted heterocycloalkyl (e.g., 3 to 8 membered heterocycloalkyl, 3 to 6 membered heterocycloalkyl, or 5 to 6 membered heterocycloalkyl), R²¹-substituted or unsubstituted aryl (e.g., C₆-C₁₀ aryl, C₁₀ aryl, or phenyl), or R²¹-substituted or unsubstituted heteroaryl (e.g., 5 to 10 membered heteroaryl, 5 to 9 membered heteroaryl, or 5 to 6 membered heteroaryl). In some embodiments, R^{1A} and R^{1B} substituents bonded to the same nitrogen atom are independently joined to form an R²¹-substituted or unsubstituted heterocycloalkyl (e.g., 3 to 8 membered heterocycloalkyl, 3 to 6 membered heterocycloalkyl, or 5 to 6 membered heterocycloalkyl) or R²¹-substituted or unsubstituted heteroaryl (e.g., 5 to 10 membered heteroaryl, 5 to 9 membered heteroaryl, or 5 to 6 membered heteroaryl). In some embodiments, R^{1A} and R^{1B} substituents bonded to the same nitrogen atom are independently joined to form an R²¹-substituted or unsubstituted heterocycloalkyl (e.g., 3 to 8 membered heterocycloalkyl, 3 to 6 membered heterocycloalkyl. In some embodiments, R^{1A} and R^{1B} substituents bonded to the same nitrogen atom are independently joined to form an R²¹-substituted or unsubstituted heteroaryl (e.g., 5 to 10 membered heteroaryl, 5 to 9 membered heteroaryl, or 5 to 6 membered heteroaryl).

In some embodiments, R^{1A} is independently R²¹-substituted or unsubstituted alkyl (e.g., C₁-C₈ alkyl, C₁-C₆ alkyl, or C₁-C₄ alkyl). In some embodiments, R^{1A} is independently R²¹-substituted alkyl (e.g., C₁-C₈ alkyl, C₁-C₆ alkyl, or C₁-C₄ alkyl). In some embodiments, R^{1A} is independently an unsubstituted alkyl (e.g., C₁-C₈ alkyl, C₁-C₆ alkyl, or C₁-C₄ alkyl). In some embodiments, R^{1A} is independently R²¹-substituted or unsubstituted heteroalkyl (e.g., 2 to 8 membered heteroalkyl, 2 to 6 membered heteroalkyl, or 2 to 4 membered heteroalkyl). In some embodiments, R^{1A} is independently R²¹-substituted heteroalkyl (e.g., 2 to 8 membered heteroalkyl, 2 to 6 membered heteroalkyl, or 2 to 4 membered heteroalkyl). In some embodiments, R^{1A} is independently an unsubstituted heteroalkyl (e.g., 2 to 8 membered heteroalkyl, 2 to 6 membered heteroalkyl, or 2 to 4 membered heteroalkyl). In some embodiments, R^{1A} is independently R²¹-substituted or unsubstituted cycloalkyl (e.g., C₃-C₈ cycloalkyl, C₃-C₆ cycloalkyl, or C₅-C₆ cycloalkyl). In some embodiments, R^{1A} is independently R²¹-substituted cycloalkyl (e.g., C₃-C₈ cycloalkyl, C₃-C₆ cycloalkyl, or C₅-C₆ cycloalkyl). In some embodiments, R^{1A} is independently an unsubstituted cycloalkyl (e.g., C₃-C₈ cycloalkyl, C₃-C₆ cycloalkyl, or C₅-C₆ cycloalkyl). In some embodiments, R^{1A} is independently R²¹-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R^{1A} is independently R²¹-substituted heterocycloalkyl (e.g., 3 to 8 membered heterocycloalkyl, 3 to 6 membered heterocycloalkyl, or 5 to 6 membered heterocycloalkyl). In some embodiments, R^{1A} is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R^{1A} is independently R²¹-substituted or unsubstituted aryl (e.g., C₆-C₁₀ aryl, C₁₀ aryl, or phenyl). In some embodiments, R^{1A} is independently R²¹-substituted aryl (e.g., C₆-C₁₀ aryl, C₁₀ aryl, or phenyl). In some embodiments, R^{1A} is independently an unsubstituted aryl (e.g., C₆-C₁₀ aryl, C₁₀ aryl, or phenyl). In some embodiments, R^{1A} is independently R²¹-substituted or unsubstituted heteroaryl (e.g., 5 to 10 membered heteroaryl, 5 to 9 membered heteroaryl, or 5 to 6 membered heteroaryl). In some embodiments, R^{1A} is independently R²¹-substituted heteroaryl (e.g., 5 to 10 membered heteroaryl, 5 to 9 membered heteroaryl, or 5 to 6 membered heteroaryl). In some embodiments, R^{1A} is independently an unsubstituted heteroaryl (e.g., 5 to 10 membered heteroaryl, 5 to 9 membered heteroaryl, or 5 to 6 membered heteroaryl).

In some embodiments, R^{1A} is independently —CCl₃. In some embodiments, R^{1A} is independently —CBr₃. In some embodiments, R^{1A} is independently —CF₃. In some embodiments, R^{1A} is independently —CI₃. In some embodiments, R^{1A} is independently —CHCl₂. In some embodiments, R^{1A} is independently —CHBr₂. In some embodiments, R^{1A} is independently —CHF₂. In some embodiments, R^{1A} is independently —CHI₂. In some embodiments, R^{1A} is independently —CH₂Cl. In some embodiments, R^{1A} is independently —CH₂Br. In some embodiments, R^{1A} is independently —CH₂F. In some embodiments, R^{1A} is independently —CH₂I. In some embodiments, R^{1A} is independently —CN. In some embodiments, R^{1A} is independently —OH. In some embodiments, R^{1A} is independently —COOH. In some embodiments, R^{1A} is independently —CONH₂. In some embodiments, R^{1A} is independently —OCCl₃. In some embodiments, R^{1A} is independently —OCF₃. In some embodiments, R^{1A} is independently —OCBr₃. In some embodiments, R^{1A} is independently —OCI₁₃. In some embodiments, R^{1A} is independently —OCHCl₂. In some embodiments, R^{1A} is independently —OCHBr₂. In some embodiments, R^{1A} is independently —OCHI₂. In some embodiments, R^{1A} is independently —OCHF₂. In some embodiments, R^{1A} is independently —OCH₂Cl. In some embodiments, R^{1A} is independently —OCH₂Br. In some embodiments, R^{1A} is independently —OCH₂I. In some embodiments, R^{1A} is independently —OCH₂F. In some embodiments, R^{1A} is independently —OCH₃. In some embodiments, R^{1A} is independently —CH₃. In some embodiments, R^{1A} is independently —CH₂CH₃. In some embodiments, R^{1A} is independently unsubstituted propyl. In some embodiments, R^{1A} is independently unsubstituted isopropyl. In some embodiments, R^{1A} is independently unsubstituted butyl. In some embodiments, R^{1A} is independently unsubstituted tert-butyl. In some embodiments, R^{1A} is independently hydrogen.

In some embodiments, R^{1B} is independently R²¹-substituted or unsubstituted alkyl (e.g., C₁-C₈ alkyl, C₁-C₆ alkyl, or C₁-C₄ alkyl). In some embodiments, R^{1B} is independently R²¹-substituted alkyl (e.g., C₁-C₈ alkyl, C₁-C₆ alkyl, or C₁-C₄ alkyl). In some embodiments, R^{1B} is independently an unsubstituted alkyl (e.g., C₁-C₈ alkyl, C₁-C₆ alkyl, or C₁-C₄ alkyl). In some embodiments, R^{1B} is independently R²¹-substituted or unsubstituted heteroalkyl (e.g., 2 to 8 membered heteroalkyl, 2 to 6 membered heteroalkyl, or 2 to 4 membered heteroalkyl). In some embodiments, R^{1B} is independently R²¹-substituted heteroalkyl (e.g., 2 to 8 membered heteroalkyl, 2 to 6 membered heteroalkyl, or 2 to 4 membered heteroalkyl). In some embodiments, R^{1B} is independently an unsubstituted heteroalkyl (e.g., 2 to 8 membered heteroalkyl, 2 to 6 membered heteroalkyl, or 2 to 4 membered heteroalkyl). In some embodiments, R^{1B} is independently R²¹-substituted or unsubstituted cycloalkyl (e.g., C₃-C₈ cycloalkyl, C₃-C₆ cycloalkyl, or C₅-C₆ cycloalkyl). In some embodiments, R^{1B} is independently R²¹-substituted cycloalkyl (e.g., C₃-C₈ cycloalkyl, C₃-C₆ cycloalkyl, or C₅-C₆ cycloalkyl). In some embodiments, R^{1B} is independently an unsubstituted cycloalkyl (e.g., C₃-C₈ cycloalkyl, C₃-C₆ cycloalkyl, or C₅-C₆ cycloalkyl). In some embodiments, R^{1B} is independently R²¹-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R^{1B} is independently R²¹-substituted heterocycloalkyl (e.g., 3 to 8 membered heterocycloalkyl, 3 to 6 membered heterocycloalkyl, or 5 to 6 membered heterocycloalkyl). In some embodiments, R^{1B} is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R^{1B} is independently R²¹-substituted or unsubstituted aryl (e.g., C₆-C₁₀ aryl, C₁₀ aryl, or phenyl). In some embodiments, R^{1B} is independently R²¹-substituted aryl (e.g., C₆-C₁₀ aryl, C₁₀ aryl, or phenyl). In some embodiments, R^{1B} is independently an unsubstituted aryl (e.g., C₆-C₁₀ aryl, C₁₀ aryl, or phenyl). In some embodiments, R^{1B} is independently R²¹-substituted or unsubstituted heteroaryl (e.g., 5 to 10 membered heteroaryl, 5 to 9 membered heteroaryl, or 5 to 6 membered heteroaryl). In some embodiments, R^{1B} is independently R²¹-substituted heteroaryl (e.g., 5 to 10 membered heteroaryl, 5 to 9 membered heteroaryl, or 5 to 6 membered heteroaryl). In some embodiments, R^{1B} is independently an unsubstituted heteroaryl (e.g., 5 to 10 membered heteroaryl, 5 to 9 membered heteroaryl, or 5 to 6 membered heteroaryl).

In some embodiments, R^{1B} is independently —CCl₃. In some embodiments, R^{1B} is independently —CBr₃. In some embodiments, R^{1B} is independently —CF₃. In some embodiments, R^{1B} is independently —CI₃. In some embodiments, R^{1B} is independently —CHCl₂. In some embodiments, R^{1B} is independently —CHBr₂. In some embodiments, R^{1B} is independently —CHF₂. In some embodiments, R^{1B} is independently —CHI₂. In some embodiments, R^{1B} is independently —CH₂Cl. In some embodiments, R^{1B} is independently —CH₂Br. In some embodiments, R^{1B} is independently —CH₂F. In some embodiments, R^{1B} is independently —CH₂I. In some embodiments, R^{1B} is independently —CN. In some embodiments, R^{1B} is independently —OH.

In some embodiments, R^{1B} is independently —COOH. In some embodiments, R^{1B} is independently —CONH₂. In some embodiments, R^{1B} is independently —OCCl₃. In some embodiments, R^{1B} is independently —OCF₃. In some embodiments, R^{1B} is independently —OCBr₃. In some embodiments, R^{1B} is independently —OCI₃. In some embodiments, R^{1B} is independently —OCHCl₂. In some embodiments, R^{1B} is independently —OCHBr₂. In some embodiments, R^{1B} is independently —OCHI₂. In some embodiments, R^{1B} is independently —OCHF₂. In some embodiments, R^{1B} is independently —OCH₂Cl. In some embodiments, R^{1B} is independently —OCH₂Br. In some embodiments, R^{1B} is independently —OCH₂I. In some embodiments, R^{1B} is independently —OCH₂F. In some embodiments, R^{1B} is independently —OCH₃. In some embodiments, R^{1B} is independently —CH₃. In some embodiments, R^{1B} is independently —CH₂CH₃. In some embodiments, R^{1B} is independently unsubstituted propyl. In some embodiments, R^{1B} is independently unsubstituted isopropyl. In some embodiments, R^{1B} is independently unsubstituted butyl. In some embodiments, R^{1B} is independently unsubstituted tert-butyl. In some embodiments, R^{1B} is independently hydrogen.

In some embodiments, R^{1C} is independently R²¹-substituted or unsubstituted alkyl (e.g., C₁-C₈ alkyl, C₁-C₆ alkyl, or C₁-C₄ alkyl). In some embodiments, R^{1C} is independently R²¹-substituted alkyl (e.g., C₁-C₈ alkyl, C₁-C₆ alkyl, or C₁-C₄ alkyl). In some embodiments, R^{1C} is independently an unsubstituted alkyl (e.g., C₁-C₈ alkyl, C₁-C₆ alkyl, or C₁-C₄ alkyl). In some embodiments, R^{1C} is independently R²¹-substituted or unsubstituted heteroalkyl (e.g., 2 to 8 membered heteroalkyl, 2 to 6 membered heteroalkyl, or 2 to 4 membered heteroalkyl). In some embodiments, R^{1C} is independently R²¹-substituted heteroalkyl (e.g., 2 to 8 membered heteroalkyl, 2 to 6 membered heteroalkyl, or 2 to 4 membered heteroalkyl). In some embodiments, R^{1C} is independently an unsubstituted heteroalkyl (e.g., 2 to 8 membered heteroalkyl, 2 to 6 membered heteroalkyl, or 2 to 4 membered heteroalkyl). In some embodiments, R^{1C} is independently R²¹-substituted or unsubstituted cycloalkyl (e.g., C₃-C₈ cycloalkyl, C₃-C₆ cycloalkyl, or C₅-C₆ cycloalkyl). In some embodiments, R^{1C} is independently R²¹-substituted cycloalkyl (e.g., C₃-C₈ cycloalkyl, C₃-C₆ cycloalkyl, or C₅-C₆ cycloalkyl). In some embodiments, R^{1C} is independently an unsubstituted cycloalkyl (e.g., C₃-C₈ cycloalkyl, C₃-C₆ cycloalkyl, or C₅-C₆ cycloalkyl). In some embodiments, R^{1C} is independently R²¹-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R^{1C} is independently R²¹-substituted heterocycloalkyl (e.g., 3 to 8 membered heterocycloalkyl, 3 to 6 membered heterocycloalkyl, or 5 to 6 membered heterocycloalkyl). In some embodiments, R^{1C} is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R^{1C} is independently R²¹-substituted or unsubstituted aryl (e.g., C₆-C₁₀ aryl, C₁₀ aryl, or phenyl). In some embodiments, R^{1C} is independently R²¹-substituted aryl (e.g., C₆-C₁₀ aryl, C₁₀ aryl, or phenyl). In some embodiments, R^{1C} is independently an unsubstituted aryl (e.g., C₆-C₁₀ aryl, C₁₀ aryl, or phenyl). In some embodiments, R^{1C} is independently R²¹-substituted or unsubstituted heteroaryl (e.g., 5 to 10 membered heteroaryl, 5 to 9 membered heteroaryl, or 5 to 6 membered heteroaryl). In some embodiments, R^{1C} is independently R²¹-substituted heteroaryl (e.g., 5 to 10 membered heteroaryl, 5 to 9 membered heteroaryl, or 5 to 6 membered heteroaryl). In some embodiments, R^{1C} is independently an unsubstituted heteroaryl (e.g., 5 to 10 membered heteroaryl, 5 to 9 membered heteroaryl, or 5 to 6 membered heteroaryl).

In some embodiments, R^{1C} is independently —CCl₃. In some embodiments, R^{1C} is independently —CBr₃. In some embodiments, R^{1C} is independently —CF₃. In some embodiments, R^{1C} is independently —CI₃. In some embodiments, R^{1C} is independently —CHCl₂. In some embodiments, R^{1C} is independently —CHBr₂. In some embodiments, R^{1C} is independently —CHF₂. In some embodiments, R^{1C} is independently —CHI₂. In some embodiments, R^{1C} is independently —CH₂Cl. In some embodiments, R^{1C} is independently —CH₂Br. In some embodiments, R^{1C} is independently —CH₂F. In some embodiments, R^{1C} is independently —CH₂I. In some embodiments, R^{1C} is independently —CN. In some embodiments, R^{1C} is independently —OH. In some embodiments, R^{1C} is independently —COOH. In some embodiments, R^{1C} is independently —CONH₂. In some embodiments, R^{1C} is independently —OCCl₃. In some embodiments, R^{1C} is independently —OCF₃. In some embodiments, R^{1C} is independently —OCBr₃. In some embodiments, R^{1C} is independently —OCI₃. In some embodiments, R^{1C} is independently —OCHCl₂. In some embodiments, R^{1C} is independently —OCHBr₂. In some embodiments, R^{1C} is independently —OCHI₂. In some embodiments, R^{1C} is independently —OCHF₂. In some embodiments, R^{1C} is independently —OCH₂Cl. In some embodiments, R^{1C} is independently —OCH₂Br. In some embodiments, R^{1C} is independently —OCH₂I. In some embodiments, R^{1C} is independently —OCH₂F. In some embodiments, R^{1C} is independently —OCH₃. In some embodiments, R^{1C} is independently —CH₃. In some embodiments, R^{1C} is independently —CH₂CH₃. In some embodiments, R^{1C} is independently unsubstituted propyl. In some embodiments, R^{1C} is independently unsubstituted isopropyl. In some embodiments, R^{1C} is independently unsubstituted butyl. In some embodiments, R^{1C} is independently unsubstituted tert-butyl. In some embodiments, R^{1C} is independently hydrogen.

In some embodiments, R^{1D} is independently R²¹-substituted or unsubstituted alkyl (e.g., C₁-C₈ alkyl, C₁-C₆ alkyl, or C₁-C₄ alkyl). In some embodiments, R^{1D} is independently R²¹-substituted alkyl (e.g., C₁-C₈ alkyl, C₁-C₆ alkyl, or C₁-C₄ alkyl). In some embodiments, R^{1D} is independently an unsubstituted alkyl (e.g., C₁-C₈ alkyl, C₁-C₆ alkyl, or C₁-C₄ alkyl). In some embodiments, R^{1D} is independently R²¹-substituted or unsubstituted heteroalkyl (e.g., 2 to 8 membered heteroalkyl, 2 to 6 membered heteroalkyl, or 2 to 4 membered heteroalkyl). In some embodiments, R^{1D} is independently R²¹-substituted heteroalkyl (e.g., 2 to 8 membered heteroalkyl, 2 to 6 membered heteroalkyl, or 2 to 4 membered heteroalkyl). In some embodiments, R^{1D} is independently an unsubstituted heteroalkyl (e.g., 2 to 8 membered heteroalkyl, 2 to 6 membered heteroalkyl, or 2 to 4 membered heteroalkyl). In some embodiments, R^{1D} is independently R²¹-substituted or unsubstituted cycloalkyl (e.g., C₃-C₈ cycloalkyl, C₃-C₆ cycloalkyl, or C₅-C₆ cycloalkyl). In some embodiments, R^{1D} is independently R²¹-substituted cycloalkyl (e.g., C₃-C₈ cycloalkyl, C₃-C₆ cycloalkyl, or C₅-C₆ cycloalkyl). In some embodiments, R^{1D} is independently an unsubstituted cycloalkyl (e.g., C₃-C₈ cycloalkyl, C₃-C₆ cycloalkyl, or C₅-C₆ cycloalkyl). In some embodiments, R^{1D} is independently R²¹-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R^{1D} is independently R²¹-substituted heterocycloalkyl (e.g., 3 to 8 membered heterocycloalkyl, 3 to 6 membered heterocycloalkyl, or 5 to 6 membered heterocycloalkyl). In some embodiments, R^{1D} is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R^{1D} is independently R²¹-substituted or unsubstituted aryl (e.g., C₆-C₁₀ aryl, C₁₀ aryl, or phenyl). In some embodiments, R^{1D} is independently R²¹-substituted aryl (e.g., C₆-C₁₀ aryl, C₁₀ aryl, or phenyl). In some embodiments, R^{1D} is independently an unsubstituted aryl (e.g., C₆-C₁₀ aryl, C₁₀ aryl, or phenyl). In some embodiments, R^{1D} is independently R²¹-substituted or unsubstituted heteroaryl (e.g., 5 to 10 membered heteroaryl, 5 to 9 membered heteroaryl, or 5 to 6 membered heteroaryl). In some embodiments, R^{1D} is independently R²¹-substituted heteroaryl (e.g., 5 to 10 membered heteroaryl, 5 to 9 membered heteroaryl, or 5 to 6 membered heteroaryl). In some embodiments, R^{1D} is independently an unsubstituted heteroaryl (e.g., 5 to 10 membered heteroaryl, 5 to 9 membered heteroaryl, or 5 to 6 membered heteroaryl).

In some embodiments, R^{1D} is independently —CCl₃. In some embodiments, R^{1D} is independently —CBr₃. In some embodiments, R^{1D} is independently —CF₃. In some embodiments, R^{1D} is independently —CI₃. In some embodiments, R^{1D} is independently —CHCl₂. In some embodiments, R^{1D} is independently —CHBr₂. In some embodiments, R^{1D} is independently —CHF₂. In some embodiments, R^{1D} is independently —CHI₂. In some embodiments, R^{1D} is independently —CH₂Cl. In some embodiments, R^{1D} is independently —CH₂Br. In some embodiments, R^{1D} is independently —CH₂F. In some embodiments, R^{1D} is independently —CH₂I. In some embodiments, R^{1D} is independently —CN. In some embodiments, R^{1D} is independently —OH. In some embodiments, R^{1D} is independently —COOH. In some embodiments, R^{1D} is independently —CONH₂. In some embodiments, R^{1D} is independently —OCCl₃. In some embodiments, R^{1D} is independently —OCF₃. In some embodiments, R^{1D} is independently —OCBr₃. In some embodiments, R^{1D} is independently —OCI₃. In some embodiments, R^{1D} is independently —OCHCl₂. In some embodiments, R^{1D} is independently —OCHBr₂. In some embodiments, R^{1D} is independently —OCHI₂. In some embodiments, R^{1D} is independently —OCHF₂. In some embodiments, R^{1D} is independently —OCH₂Cl. In some embodiments, R^{1D} is independently —OCH₂Br. In some embodiments, R^{1D} is independently —OCH₂I. In some embodiments, R^{1D} is independently —OCH₂F. In some embodiments, R^{1D} is independently —OCH₃. In some embodiments, R^{1D} is independently —CH₃. In some embodiments, R^{1D} is independently —CH₂CH₃. In some embodiments, R^{1D} is independently unsubstituted propyl. In some embodiments, R^{1D} is independently unsubstituted isopropyl. In some embodiments, R^{1D} is independently unsubstituted butyl. In some embodiments, R^{1D} is independently unsubstituted tert-butyl. In some embodiments, R^{1D} is independently hydrogen.

In some embodiments, R² is independently H, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N₃, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In some embodiments, R² is H. In some embodiments, R² is independently substituted or unsubstituted alkyl (e.g., C₁-C₈ alkyl, C₁-C₆ alkyl, or C₁-C₄ alkyl). In some embodiments, R² is independently substituted alkyl (e.g., C₁-C₈ alkyl, C₁-C₆ alkyl, or C₁-C₄ alkyl). In some embodiments, R² is independently an unsubstituted alkyl (e.g., C₁-C₈ alkyl, C₁-C₆ alkyl, or C₁-C₄ alkyl). In some embodiments, R² is independently substituted or unsubstituted heteroalkyl (e.g., 2 to 8 membered heteroalkyl, 2 to 6 membered heteroalkyl, or 2 to 4 membered heteroalkyl). In some embodiments, R² is independently substituted heteroalkyl (e.g., 2 to 8 membered heteroalkyl, 2 to 6 membered heteroalkyl, or 2 to 4 membered heteroalkyl). In some embodiments, R² is independently an unsubstituted heteroalkyl (e.g., 2 to 8 membered heteroalkyl, 2 to 6 membered heteroalkyl, or 2 to 4 membered heteroalkyl). In some embodiments, R² is independently substituted or unsubstituted cycloalkyl (e.g., C₃-C₈ cycloalkyl, C₃-C₆ cycloalkyl, or C₅-C₆ cycloalkyl). In some embodiments, R² is independently substituted cycloalkyl (e.g., C₃-C₈ cycloalkyl, C₃-C₆ cycloalkyl, or C₅-C₆ cycloalkyl). In some embodiments, R² is independently an unsubstituted cycloalkyl (e.g., C₃-C₈ cycloalkyl, C₃-C₆ cycloalkyl, or C₅-C₆ cycloalkyl). In some embodiments, R² is independently substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R² is independently substituted heterocycloalkyl (e.g., 3 to 8 membered heterocycloalkyl, 3 to 6 membered heterocycloalkyl, or 5 to 6 membered heterocycloalkyl). In some embodiments, R² is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R² is independently substituted or unsubstituted aryl (e.g., C₆-C₁₀ aryl, C₁₀ aryl, or phenyl). In some embodiments, R² is independently substituted aryl (e.g., C₆-C₁₀ aryl, C₁₀ aryl, or phenyl). In some embodiments, R² is independently an unsubstituted aryl (e.g., C₆-C₁₀ aryl, C₁₀ aryl, or phenyl). In some embodiments, R² is independently substituted or unsubstituted heteroaryl (e.g., 5 to 10 membered heteroaryl, 5 to 9 membered heteroaryl, or 5 to 6 membered heteroaryl). In some embodiments, R² is independently substituted heteroaryl (e.g., 5 to 10 membered heteroaryl, 5 to 9 membered heteroaryl, or 5 to 6 membered heteroaryl). In some embodiments, R² is independently an unsubstituted heteroaryl (e.g., 5 to 10 membered heteroaryl, 5 to 9 membered heteroaryl, or 5 to 6 membered heteroaryl).

In some embodiments, R² is independently H, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, R²⁴-substituted or unsubstituted alkyl (e.g., C₁-C₈ alkyl, C₁-C₆ alkyl, or C₁-C₄ alkyl), R²⁴-substituted or unsubstituted heteroalkyl (e.g., 2 to 8 membered heteroalkyl, 2 to 6 membered heteroalkyl, or 2 to 4 membered heteroalkyl), R²⁴-substituted or unsubstituted cycloalkyl (e.g., C₃-C₈ cycloalkyl, C₃-C₆ cycloalkyl, or C₅-C₆ cycloalkyl), R²⁴-substituted or unsubstituted heterocycloalkyl (e.g., 3 to 8 membered heterocycloalkyl, 3 to 6 membered heterocycloalkyl, or 5 to 6 membered heterocycloalkyl), R²⁴-substituted or unsubstituted aryl (e.g., C₆-C₁₀ aryl, C₁₀ aryl, or phenyl), or R²⁴-substituted or unsubstituted heteroaryl (e.g., 5 to 10 membered heteroaryl, 5 to 9 membered heteroaryl, or 5 to 6 membered heteroaryl). In some embodiments, R² is independently halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R² is H. In some embodiments, R² is independently R²⁴-substituted or unsubstituted alkyl (e.g., C₁-C₈ alkyl, C₁-C₆ alkyl, or C₁-C₄ alkyl). In some embodiments, R² is independently R²⁴-substituted alkyl (e.g., C₁-C₈ alkyl, C₁-C₆ alkyl, or C₁-C₄ alkyl). In some embodiments, R² is independently an unsubstituted alkyl (e.g., C₁-C₈ alkyl, C₁-C₆ alkyl, or C₁-C₄ alkyl). In some embodiments, R² is independently R²⁴-substituted or unsubstituted heteroalkyl (e.g., 2 to 8 membered heteroalkyl, 2 to 6 membered heteroalkyl, or 2 to 4 membered heteroalkyl). In some embodiments, R² is independently R²⁴-substituted heteroalkyl (e.g., 2 to 8 membered heteroalkyl, 2 to 6 membered heteroalkyl, or 2 to 4 membered heteroalkyl). In some embodiments, R² is independently an unsubstituted heteroalkyl (e.g., 2 to 8 membered heteroalkyl, 2 to 6 membered heteroalkyl, or 2 to 4 membered heteroalkyl). In some embodiments, R² is independently R²⁴-substituted or unsubstituted cycloalkyl (e.g., C₃-C₈ cycloalkyl, C₃-C₆ cycloalkyl, or C₅-C₆ cycloalkyl). In some embodiments, R² is independently R²⁴-substituted cycloalkyl (e.g., C₃-C₈ cycloalkyl, C₃-C₆ cycloalkyl, or C₅-C₆ cycloalkyl). In some embodiments, R² is independently an unsubstituted cycloalkyl (e.g., C₃-C₈ cycloalkyl, C₃-C₆ cycloalkyl, or C₅-C₆ cycloalkyl). In some embodiments, R² is independently R²⁴-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R² is independently R²⁴-substituted heterocycloalkyl (e.g., 3 to 8 membered heterocycloalkyl, 3 to 6 membered heterocycloalkyl, or 5 to 6 membered heterocycloalkyl). In some embodiments, R² is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R² is independently R²⁴-substituted or unsubstituted aryl (e.g., C₆-C₁₀ aryl, C₁₀ aryl, or phenyl). In some embodiments, R² is independently R²⁴-substituted aryl (e.g., C₆-C₁₀ aryl, C₁₀ aryl, or phenyl). In some embodiments, R² is independently an unsubstituted aryl (e.g., C₆-C₁₀ aryl, C₁₀ aryl, or phenyl). In some embodiments, R² is independently R²⁴-substituted or unsubstituted heteroaryl (e.g., 5 to 10 membered heteroaryl, 5 to 9 membered heteroaryl, or 5 to 6 membered heteroaryl). In some embodiments, R² is independently R²⁴-substituted heteroaryl (e.g., 5 to 10 membered heteroaryl, 5 to 9 membered heteroaryl, or 5 to 6 membered heteroaryl). In some embodiments, R² is independently an unsubstituted heteroaryl (e.g., 5 to 10 membered heteroaryl, 5 to 9 membered heteroaryl, or 5 to 6 membered heteroaryl).

In some embodiments, R² is independently —CX²₃. In some embodiments, R² is independently —CHX²₂. In some embodiments, R² is independently —CH₂X². In some embodiments, R² is independently —OCX²₃. In some embodiments, R² is independently —OCH₂X². In some embodiments, R² is independently —OCHX²₂. In some embodiments, R² is independently —CN. In some embodiments, R² is independently —SR^{2D}. In some embodiments, R² is independently —SOR^{2D}. In some embodiments, R² is independently —SO₂R^{2D}. In some embodiments, R² is independently —SO₃R^{2D}. In some embodiments, R² is independently —SO₄R^{2D}. In some embodiments, R² is independently —SONR^{2A}R^{2B}. In some embodiments, R² is independently —SO₂NR^{2A}R^{2B}. In some embodiments, R² is independently —NHC(O)NR^{2A}R^{2B}. In some embodiments, R² is independently —N(O). In some embodiments, R² is independently —N(O)₂. In some embodiments, R² is independently —NR^{2A}R^{2B}. In some embodiments, R² is independently —C(O)R^{2C}. In some embodiments, R² is independently —C(O)—OR^{2C}. In some embodiments, R² is independently —C(O)NR^{2A}R^{2B}. In some embodiments, R² is independently —OR^{2D}. In some embodiments, R² is independently —NR^{2A}SO₂R^{2D}. In some embodiments, R² is independently —NR^{2A}C(O)R^{2C}. In some embodiments, R² is independently —NR^{2A}C(O)OR^{2C}. In some embodiments, R² is independently —NR^{2A}OR^{2C}.

In some embodiments, R² is independently oxo. In some embodiments, R² is independently halogen. In some embodiments, R² is independently —CCl₃. In some embodiments, R² is independently —CBr₃. In some embodiments, R² is independently —CF₃. In some embodiments, R² is independently —CI₃. In some embodiments, R² is independently —CHCl₂. In some embodiments, R² is independently —CHBr₂. In some embodiments, R² is independently —CHF₂. In some embodiments, R² is independently —CHI₂. In some embodiments, R² is independently —CH₂Cl. In some embodiments, R² is independently —CH₂Br. In some embodiments, R² is independently —CH₂F. In some embodiments, R² is independently —CH₂I. In some embodiments, R² is independently —CN. In some embodiments, R² is independently —OH. In some embodiments, R² is independently —NH₂. In some embodiments, R² is independently —COOH. In some embodiments, R² is independently —CONH₂. In some embodiments, R² is independently —NO₂. In some embodiments, R² is independently —SH. In some embodiments, R² is independently —SO₃H. In some embodiments, R² is independently —SO₄H. In some embodiments, R² is independently —SO₂NH₂. In some embodiments, R² is independently —NHNH₂. In some embodiments, R² is independently —ONH₂. In some embodiments, R² is independently —NHC(O)NHNH₂. In some embodiments, R² is independently —NHC(O)NH₂. In some embodiments, R² is independently —NHSO₂H. In some embodiments, R² is independently —NHC(O)H. In some embodiments, R² is independently —NHC(O)OH. In some embodiments, R² is independently —NHOH. In some embodiments, R² is independently —OCCl₃. In some embodiments, R² is independently —OCF₃. In some embodiments, R² is independently —OCBr₃. In some embodiments, R² is independently —OCI₃. In some embodiments, R² is independently —OCHCl₂. In some embodiments, R² is independently —OCHBr₂. In some embodiments, R² is independently —OCHI₂. In some embodiments, R² is independently —OCHF₂. In some embodiments, R² is independently —OCH₂Cl. In some embodiments, R² is independently —OCH₂Br. In some embodiments, R² is independently —OCH₂I. In some embodiments, R² is independently —OCH₂F. In some embodiments, R² is independently —N₃. In some embodiments, R² is independently —OCH₃. In some embodiments, R² is independently —CH₃. In some embodiments, R² is independently —CH₂CH₃. In some embodiments, R² is independently unsubstituted propyl. In some embodiments, R² is independently unsubstituted isopropyl. In some embodiments, R² is independently unsubstituted butyl. In some embodiments, R² is independently unsubstituted tert-butyl. In some embodiments, R² is independently —F. In some embodiments, R² is independently —Cl. In some embodiments, R² is independently —Br. In some embodiments, R² is independently —I.

In some embodiments, R²⁴ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R²⁵-substituted or unsubstituted alkyl (e.g., C₁-C₈ alkyl, C₁-C₆ alkyl, or C₁-C₄ alkyl), R²⁵-substituted or unsubstituted heteroalkyl (e.g., 2 to 8 membered heteroalkyl, 2 to 6 membered heteroalkyl, or 2 to 4 membered heteroalkyl), R²⁵-substituted or unsubstituted cycloalkyl (e.g., C₃-C₈ cycloalkyl, C₃-C₆ cycloalkyl, or C₅-C₆ cycloalkyl), R²⁵-substituted or unsubstituted heterocycloalkyl (e.g., 3 to 8 membered heterocycloalkyl, 3 to 6 membered heterocycloalkyl, or 5 to 6 membered heterocycloalkyl), R²⁵-substituted or unsubstituted aryl (e.g., C₆-C₁₀ aryl, C₁₀ aryl, or phenyl), or R²⁵-substituted or unsubstituted heteroaryl (e.g., 5 to 10 membered heteroaryl, 5 to 9 membered heteroaryl, or 5 to 6 membered heteroaryl). In some embodiments, R²⁴ is independently oxo, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R²⁴ is independently oxo. In some embodiments, R²⁴ is independently halogen. In some embodiments, R²⁴ is independently —CCl₃. In some embodiments, R²⁴ is independently —CBr₃. In some embodiments, R²⁴ is independently —CF₃. In some embodiments, R²⁴ is independently —CI₃. In some embodiments, R²⁴ is independently —CHCl₂. In some embodiments, R²⁴ is independently —CHBr₂. In some embodiments, R²⁴ is independently —CHF₂. In some embodiments, R²⁴ is independently —CHI₂. In some embodiments, R²⁴ is independently —CH₂Cl. In some embodiments, R²⁴ is independently —CH₂Br. In some embodiments, R²⁴ is independently —CH₂F. In some embodiments, R²⁴ is independently —CH₂I. In some embodiments, R²⁴ is independently —CN. In some embodiments, R²⁴ is independently —OH. In some embodiments, R²⁴ is independently —NH₂. In some embodiments, R²⁴ is independently —COOH. In some embodiments, R²⁴ is independently —CONH₂. In some embodiments, R²⁴ is independently —NO₂. In some embodiments, R²⁴ is independently —SH. In some embodiments, R²⁴ is independently —SO₃H. In some embodiments, R²⁴ is independently —SO₄H. In some embodiments, R²⁴ is independently —SO₂NH₂. In some embodiments, R²⁴ is independently —NHNH₂. In some embodiments, R²⁴ is independently —ONH₂. In some embodiments, R²⁴ is independently —NHC(O)NHNH₂. In some embodiments, R²⁴ is independently —NHC(O)NH₂. In some embodiments, R²⁴ is independently —NHSO₂H. In some embodiments, R²⁴ is independently —NHC(O)H. In some embodiments, R²⁴ is independently —NHC(O)OH. In some embodiments, R²⁴ is independently —NHOH. In some embodiments, R²⁴ is independently —OCCl₃. In some embodiments, R²⁴ is independently —OCF₃. In some embodiments, R²⁴ is independently —OCBr₃. In some embodiments, R²⁴ is independently —OCI₃. In some embodiments, R²⁴ is independently —OCHCl₂. In some embodiments, R²⁴ is independently —OCHBr₂. In some embodiments, R²⁴ is independently —OCHI₂. In some embodiments, R²⁴ is independently —OCHF₂. In some embodiments, R²⁴ is independently —OCH₂Cl. In some embodiments, R²⁴ is independently —OCH₂Br. In some embodiments, R²⁴ is independently —OCH₂I. In some embodiments, R²⁴ is independently —OCH₂F. In some embodiments, R²⁴ is independently —N₃. In some embodiments, R²⁴ is independently —OCH₃. In some embodiments, R²⁴ is independently —CH₃. In some embodiments, R²⁴ is independently —CH₂CH₃. In some embodiments, R²⁴ is independently unsubstituted propyl. In some embodiments, R²⁴ is independently unsubstituted isopropyl. In some embodiments, R²⁴ is independently unsubstituted butyl. In some embodiments, R²⁴ is independently unsubstituted tert-butyl. In some embodiments, R²⁴ is independently —F. In some embodiments, R²⁴ is independently —Cl. In some embodiments, R²⁴ is independently —Br. In some embodiments, R²⁴ is independently —I.

In some embodiments, R²⁴ is independently R²⁵-substituted or unsubstituted alkyl (e.g., C₁-C₈ alkyl, C₁-C₆ alkyl, or C₁-C₄ alkyl). In some embodiments, R²⁴ is independently R²⁵-substituted alkyl (e.g., C₁-C₈ alkyl, C₁-C₆ alkyl, or C₁-C₄ alkyl). In some embodiments, R²⁴ is independently an unsubstituted alkyl (e.g., C₁-C₈ alkyl, C₁-C₆ alkyl, or C₁-C₄ alkyl). In some embodiments, R²⁴ is independently R²⁵-substituted or unsubstituted heteroalkyl (e.g., 2 to 8 membered heteroalkyl, 2 to 6 membered heteroalkyl, or 2 to 4 membered heteroalkyl). In some embodiments, R²⁴ is independently R²⁵-substituted heteroalkyl (e.g., 2 to 8 membered heteroalkyl, 2 to 6 membered heteroalkyl, or 2 to 4 membered heteroalkyl). In some embodiments, R²⁴ is independently an unsubstituted heteroalkyl (e.g., 2 to 8 membered heteroalkyl, 2 to 6 membered heteroalkyl, or 2 to 4 membered heteroalkyl). In some embodiments, R²⁴ is independently R²⁵-substituted or unsubstituted cycloalkyl (e.g., C₃-C₈ cycloalkyl, C₃-C₆ cycloalkyl, or C₅-C₆ cycloalkyl). In some embodiments, R²⁴ is independently R²⁵-substituted cycloalkyl (e.g., C₃-C₈ cycloalkyl, C₃-C₆ cycloalkyl, or C₅-C₆ cycloalkyl). In some embodiments, R²⁴ is independently an unsubstituted cycloalkyl (e.g., C₃-C₈ cycloalkyl, C₃-C₆ cycloalkyl, or C₅-C₆ cycloalkyl). In some embodiments, R²⁴ is independently R²⁵-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R²⁴ is independently R²⁵-substituted heterocycloalkyl (e.g., 3 to 8 membered heterocycloalkyl, 3 to 6 membered heterocycloalkyl, or 5 to 6 membered heterocycloalkyl). In some embodiments, R²⁴ is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R²⁴ is independently R²⁵-substituted or unsubstituted aryl (e.g., C₆-C₁₀ aryl, C₁₀ aryl, or phenyl). In some embodiments, R²⁴ is independently R²⁵-substituted aryl (e.g., C₆-C₁₀ aryl, C₁₀ aryl, or phenyl). In some embodiments, R²⁴ is independently an unsubstituted aryl (e.g., C₆-C₁₀ aryl, C₁₀ aryl, or phenyl). In some embodiments, R²⁴ is independently R²⁵-substituted or unsubstituted heteroaryl (e.g., 5 to 10 membered heteroaryl, 5 to 9 membered heteroaryl, or 5 to 6 membered heteroaryl). In some embodiments, R²⁴ is independently R²⁵-substituted heteroaryl (e.g., 5 to 10 membered heteroaryl, 5 to 9 membered heteroaryl, or 5 to 6 membered heteroaryl). In some embodiments, R²⁴ is independently an unsubstituted heteroaryl (e.g., 5 to 10 membered heteroaryl, 5 to 9 membered heteroaryl, or 5 to 6 membered heteroaryl).

In some embodiments, R²⁵ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R²⁶-substituted or unsubstituted alkyl (e.g., C₁-C₈ alkyl, C₁-C₆ alkyl, or C₁-C₄ alkyl), R²⁶-substituted or unsubstituted heteroalkyl (e.g., 2 to 8 membered heteroalkyl, 2 to 6 membered heteroalkyl, or 2 to 4 membered heteroalkyl), R²⁶-substituted or unsubstituted cycloalkyl (e.g., C₃-C₈ cycloalkyl, C₃-C₆ cycloalkyl, or C₅-C₆ cycloalkyl), R²⁶-substituted or unsubstituted heterocycloalkyl (e.g., 3 to 8 membered heterocycloalkyl, 3 to 6 membered heterocycloalkyl, or 5 to 6 membered heterocycloalkyl), R²⁶-substituted or unsubstituted aryl (e.g., C₆-C₁₀ aryl, C₁₀ aryl, or phenyl), or R²⁶-substituted or unsubstituted heteroaryl (e.g., 5 to 10 membered heteroaryl, 5 to 9 membered heteroaryl, or 5 to 6 membered heteroaryl). In some embodiments, R²⁵ is independently oxo, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R²⁵ is independently R²⁶-substituted or unsubstituted alkyl (e.g., C₁-C₈ alkyl, C₁-C₆ alkyl, or C₁-C₄ alkyl). In some embodiments, R²⁵ is independently R²⁶-substituted alkyl (e.g., C₁-C₈ alkyl, C₁-C₆ alkyl, or C₁-C₄ alkyl). In some embodiments, R²⁵ is independently an unsubstituted alkyl (e.g., C₁-C₈ alkyl, C₁-C₆ alkyl, or C₁-C₄ alkyl). In some embodiments, R²⁵ is independently R²⁶-substituted or unsubstituted heteroalkyl (e.g., 2 to 8 membered heteroalkyl, 2 to 6 membered heteroalkyl, or 2 to 4 membered heteroalkyl). In some embodiments, R²⁵ is independently R²⁶-substituted heteroalkyl (e.g., 2 to 8 membered heteroalkyl, 2 to 6 membered heteroalkyl, or 2 to 4 membered heteroalkyl). In some embodiments, R²⁵ is independently an unsubstituted heteroalkyl (e.g., 2 to 8 membered heteroalkyl, 2 to 6 membered heteroalkyl, or 2 to 4 membered heteroalkyl). In some embodiments, R²⁵ is independently R²⁶-substituted or unsubstituted cycloalkyl (e.g., C₃-C₈ cycloalkyl, C₃-C₆ cycloalkyl, or C₅-C₆ cycloalkyl). In some embodiments, R²⁵ is independently R²⁶-substituted cycloalkyl (e.g., C₃-C₈ cycloalkyl, C₃-C₆ cycloalkyl, or C₅-C₆ cycloalkyl). In some embodiments, R²⁵ is independently an unsubstituted cycloalkyl (e.g., C₃-C₈ cycloalkyl, C₃-C₆ cycloalkyl, or C₅-C₆ cycloalkyl). In some embodiments, R²⁵ is independently R²⁶-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R²⁵ is independently R²⁶-substituted heterocycloalkyl (e.g., 3 to 8 membered heterocycloalkyl, 3 to 6 membered heterocycloalkyl, or 5 to 6 membered heterocycloalkyl). In some embodiments, R²⁵ is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R²⁵ is independently R²⁶-substituted or unsubstituted aryl (e.g., C₆-C₁₀ aryl, C₁₀ aryl, or phenyl). In some embodiments, R²⁵ is independently R²⁶-substituted aryl (e.g., C₆-C₁₀ aryl, C₁₀ aryl, or phenyl). In some embodiments, R²⁵ is independently an unsubstituted aryl (e.g., C₆-C₁₀ aryl, C₁₀ aryl, or phenyl). In some embodiments, R²⁵ is independently R²⁶-substituted or unsubstituted heteroaryl (e.g., 5 to 10 membered heteroaryl, 5 to 9 membered heteroaryl, or 5 to 6 membered heteroaryl). In some embodiments, R²⁵ is independently R²⁶-substituted heteroaryl (e.g., 5 to 10 membered heteroaryl, 5 to 9 membered heteroaryl, or 5 to 6 membered heteroaryl). In some embodiments, R²⁵ is independently an unsubstituted heteroaryl (e.g., 5 to 10 membered heteroaryl, 5 to 9 membered heteroaryl, or 5 to 6 membered heteroaryl).

In some embodiments, R²⁵ is independently oxo. In some embodiments, R²⁵ is independently halogen. In some embodiments, R²⁵ is independently —CCl₃. In some embodiments, R²⁵ is independently —CBr₃. In some embodiments, R²⁵ is independently —CF₃. In some embodiments, R²⁵ is independently —CI₃. In some embodiments, R²⁵ is independently —CHCl₂. In some embodiments, R²⁵ is independently —CHBr₂. In some embodiments, R²⁵ is independently —CHF₂. In some embodiments, R²⁵ is independently —CHI₂. In some embodiments, R²⁵ is independently —CH₂Cl. In some embodiments, R²⁵ is independently —CH₂Br. In some embodiments, R²⁵ is independently —CH₂F. In some embodiments, R²⁵ is independently —CH₂I. In some embodiments, R²⁵ is independently —CN. In some embodiments, R²⁵ is independently —OH. In some embodiments, R²⁵ is independently —NH₂. In some embodiments, R²⁵ is independently —COOH. In some embodiments, R²⁵ is independently —CONH₂. In some embodiments, R²⁵ is independently —NO₂. In some embodiments, R²⁵ is independently —SH. In some embodiments, R²⁵ is independently —SO₃H. In some embodiments, R²⁵ is independently —SO₄H. In some embodiments, R²⁵ is independently —SO₂NH₂. In some embodiments, R²⁵ is independently —NHNH₂. In some embodiments, R²⁵ is independently —ONH₂. In some embodiments, R²⁵ is independently —NHC(O)NHNH₂. In some embodiments, R²⁵ is independently —NHC(O)NH₂. In some embodiments, R²⁵ is independently —NHSO₂H. In some embodiments, R²⁵ is independently —NHC(O)H. In some embodiments, R²⁵ is independently —NHC(O)OH. In some embodiments, R²⁵ is independently —NHOH. In some embodiments, R²⁵ is independently —OCCl₃. In some embodiments, R²⁵ is independently —OCF₃. In some embodiments, R²⁵ is independently —OCBr₃. In some embodiments, R²⁵ is independently —OCI₃. In some embodiments, R²⁵ is independently —OCHCl₂. In some embodiments, R²⁵ is independently —OCHBr₂. In some embodiments, R²⁵ is independently —OCHI₂. In some embodiments, R²⁵ is independently —OCHF₂. In some embodiments, R²⁵ is independently —OCH₂Cl. In some embodiments, R²⁵ is independently —OCH₂Br. In some embodiments, R²⁵ is independently —OCH₂I. In some embodiments, R²⁵ is independently —OCH₂F. In some embodiments, R²⁵ is independently —N₃. In some embodiments, R²⁵ is independently —OCH₃. In some embodiments, R²⁵ is independently —CH₃. In some embodiments, R²⁵ is independently —CH₂CH₃. In some embodiments, R²⁵ is independently unsubstituted propyl. In some embodiments, R²⁵ is independently unsubstituted isopropyl. In some embodiments, R²⁵ is independently unsubstituted butyl. In some embodiments, R²⁵ is independently unsubstituted tert-butyl. In some embodiments, R²⁵ is independently —F. In some embodiments, R²⁵ is independently —Cl. In some embodiments, R²⁵ is independently —Br. In some embodiments, R²⁵ is independently —I.

In some embodiments, R²⁶ is independently oxo, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R²⁶ is independently an unsubstituted alkyl (e.g., C₁-C₈ alkyl, C₁-C₆ alkyl, or C₁-C₄ alkyl). In some embodiments, R²⁶ is independently an unsubstituted heteroalkyl (e.g., 2 to 8 membered heteroalkyl, 2 to 6 membered heteroalkyl, or 2 to 4 membered heteroalkyl). In some embodiments, R²⁶ is independently an unsubstituted cycloalkyl (e.g., C₃-C₈ cycloalkyl, C₃-C₆ cycloalkyl, or C₅-C₆ cycloalkyl). In some embodiments, R²⁶ is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R²⁶ is independently an unsubstituted aryl (e.g., C₆-C₁₀ aryl, C₁₀ aryl, or phenyl). In some embodiments, R²⁶ is independently an unsubstituted heteroaryl (e.g., 5 to 10 membered heteroaryl, 5 to 9 membered heteroaryl, or 5 to 6 membered heteroaryl).

In some embodiments, R²⁶ is independently oxo. In some embodiments, R²⁶ is independently halogen. In some embodiments, R²⁶ is independently —CCl₃. In some embodiments, R²⁶ is independently —CBr₃. In some embodiments, R²⁶ is independently —CF₃. In some embodiments, R²⁶ is independently —CI₃. In some embodiments, R²⁶ is independently —CHCl₂. In some embodiments, R²⁶ is independently —CHBr₂. In some embodiments, R²⁶ is independently —CHF₂. In some embodiments, R²⁶ is independently —CHI₂. In some embodiments, R²⁶ is independently —CH₂Cl. In some embodiments, R²⁶ is independently —CH₂Br. In some embodiments, R²⁶ is independently —CH₂F. In some embodiments, R²⁶ is independently —CH₂I. In some embodiments, R²⁶ is independently —CN. In some embodiments, R²⁶ is independently —OH. In some embodiments, R²⁶ is independently —NH₂. In some embodiments, R²⁶ is independently —COOH. In some embodiments, R²⁶ is independently —CONH₂. In some embodiments, R²⁶ is independently —NO₂. In some embodiments, R²⁶ is independently —SH. In some embodiments, R²⁶ is independently —SO₃H. In some embodiments, R²⁶ is independently —SO₄H. In some embodiments, R²⁶ is independently —SO₂NH₂. In some embodiments, R²⁶ is independently —NHNH₂. In some embodiments, R²⁶ is independently —ONH₂. In some embodiments, R²⁶ is independently —NHC(O)NHNH₂. In some embodiments, R²⁶ is independently —NHC(O)NH₂. In some embodiments, R²⁶ is independently —NHSO₂H. In some embodiments, R²⁶ is independently —NHC(O)H. In some embodiments, R²⁶ is independently —NHC(O)OH. In some embodiments, R²⁶ is independently —NHOH. In some embodiments, R²⁶ is independently —OCCl₃. In some embodiments, R²⁶ is independently —OCF₃. In some embodiments, R²⁶ is independently —OCBr₃. In some embodiments, R²⁶ is independently —OCI₃. In some embodiments, R²⁶ is independently —OCHCl₂. In some embodiments, R²⁶ is independently —OCHBr₂. In some embodiments, R²⁶ is independently —OCHI₂. In some embodiments, R²⁶ is independently —OCHF₂. In some embodiments, R²⁶ is independently —OCH₂Cl. In some embodiments, R²⁶ is independently —OCH₂Br. In some embodiments, R²⁶ is independently —OCH₂I. In some embodiments, R²⁶ is independently —OCH₂F. In some embodiments, R²⁶ is independently —N₃. In some embodiments, R²⁶ is independently —OCH₃. In some embodiments, R²⁶ is independently —CH₃. In some embodiments, R²⁶ is independently —CH₂CH₃. In some embodiments, R²⁶ is independently unsubstituted propyl.

In some embodiments, R²⁶ is independently unsubstituted isopropyl. In some embodiments, R²⁶ is independently unsubstituted butyl. In some embodiments, R²⁶ is independently unsubstituted tert-butyl. In some embodiments, R²⁶ is independently —F. In some embodiments, R²⁶ is independently —Cl. In some embodiments, R²⁶ is independently —Br. In some embodiments, R²⁶ is independently —I.

In some embodiments, R² is taken together with one R¹ substituent to form a substituted or unsubstituted heterocycloalkyl (e.g., 3 to 8 membered heterocycloalkyl, 3 to 6 membered heterocycloalkyl, or 5 to 6 membered heterocycloalkyl), or substituted or unsubstituted heteroaryl (e.g., 5 to 10 membered heteroaryl, 5 to 9 membered heteroaryl, or 5 to 6 membered heteroaryl). In some embodiments, R² is taken together with one R¹ substituent to form a substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R² is taken together with one R¹ substituent to form a substituted heterocycloalkyl (e.g., 3 to 8 membered heterocycloalkyl, 3 to 6 membered heterocycloalkyl, or 5 to 6 membered heterocycloalkyl). In some embodiments, R² is taken together with one R¹ substituent to form an unsubstituted heterocycloalkyl (e.g., 3 to 8 membered heterocycloalkyl, 3 to 6 membered heterocycloalkyl, or 5 to 6 membered heterocycloalkyl). In some embodiments, R² is taken together with one R¹ substituent to form an R²¹-substituted heterocycloalkyl (e.g., 3 to 8 membered heterocycloalkyl, 3 to 6 membered heterocycloalkyl, or 5 to 6 membered heterocycloalkyl). In some embodiments, R² is taken together with one R¹ substituent to form a substituted or unsubstituted heteroaryl (e.g., 5 to 10 membered heteroaryl, 5 to 9 membered heteroaryl, or 5 to 6 membered heteroaryl). In some embodiments, R² is taken together with one R¹ substituent to form a substituted heteroaryl (e.g., 5 to 10 membered heteroaryl, 5 to 9 membered heteroaryl, or 5 to 6 membered heteroaryl). In some embodiments, R² is taken together with one R¹ substituent to form an unsubstituted heteroaryl (e.g., 5 to 10 membered heteroaryl, 5 to 9 membered heteroaryl, or 5 to 6 membered heteroaryl). In some embodiments, R² is taken together with one R¹substituent to form an R²¹-substituted heteroaryl (e.g., 5 to 10 membered heteroaryl, 5 to 9 membered heteroaryl, or 5 to 6 membered heteroaryl).

In some embodiments, the divalent linker bonded to both the monovalent cellular component binder and monovalent targeted autophagy protein binder is attached to the monovalent targeted autophagy protein binder at an R² position. In some embodiments, when the divalent linker bonded to both the monovalent cellular component binder and monovalent targeted autophagy protein binder is attached to the monovalent targeted autophagy protein binder at an R² position, R² is replaced with a divalent linker, referred to in this embodiment as L^{R2}.

In some embodiments, L^{R2} is a bond, —S(O)₂—, —S(O)—, —NR^{2A}—, ═N—, —O—, —S—, —C(O)—, C(O)NR^{2A}—, —NR^{2A}C(O)—, —NR^{2A}C(O)NH—, —NHC(O)NR^{2A}—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. It will be understood that when -L^{R2}- is ═N—, one of the two direct covalent connections to L^{R2} shown in “-L²-” is a double bond and L^{R2} may equivalently be shown as “=L^{R2}-” and the atom to which the double bond is directly attached must obey standard rules of chemical valency known in the chemical arts and be capable of forming such a double bond.

In some embodiments, L^{R2} is independently a bond, —S(O)₂—, —S(O)—, —NH—, —O—, —S—, —C(O)—, —C(O)NH—, —NHC(O)—, —NHC(O)NH—, —C(O)O—, —OC(O)—, substituted or unsubstituted alkylene (e.g., C₁-C₈ alkylene, C₁-C₆ alkylene, or C₁-C₄ alkylene), substituted or unsubstituted heteroalkylene (e.g., 2 to 8 membered heteroalkylene, 2 to 6 membered heteroalkylene, or 2 to 4 membered heteroalkylene), substituted or unsubstituted cycloalkylene (e.g., C₃-C₈ cycloalkylene, C₃-C₆ cycloalkylene, or C₅-C₆ cycloalkylene), substituted or unsubstituted heterocycloalkylene (e.g., 3 to 8 membered heterocycloalkylene, 3 to 6 membered heterocycloalkylene, or 5 to 6 membered heterocycloalkylene), substituted or unsubstituted arylene (e.g., C₆-C₁₀ arylene, C₁₀ arylene, or phenylene), or substituted or unsubstituted heteroarylene (e.g., 5 to 10 membered heteroarylene, 5 to 9 membered heteroarylene, or 5 to 6 membered heteroarylene). In some embodiments, L^{R2} is independently a —S(O)₂—. In some embodiments, L^{R2} is independently a —S(O)—. In some embodiments, L^{R2} is independently a —NH—. In some embodiments, L^{R2} is independently a —O—. In some embodiments, L^{R2} is independently a —S—. In some embodiments, L^{R2} is independently a —C(O)—. In some embodiments, L^{R2} is independently a —C(O)NH—. In some embodiments, L^{R2} is independently a —NHC(O)—. In some embodiments, L^{R2} is independently a —NHC(O)NH—. In some embodiments, L^{R2} is independently a —C(O)O—. In some embodiments, L^{R2} is independently —OC(O)—. In some embodiments, L^{R2} is independently —NR^{2A}—. In some embodiments, L^{R2} is independently —C(O)NR^{2A}—. In some embodiments, L^{R2} is independently —NR^{2A}C(O)—. In some embodiments, L^{R2} is independently —NR^{2A}C(O)NH—. In some embodiments, L^{R2} is independently —NHC(O)NR^{2A}—. In some embodiments, L^{R2} is independently a bond.

In some embodiments, L^{R2} is substituted or unsubstituted alkylene (e.g., C₁-C₈ alkylene, C₁-C₆ alkylene, or C₁-C₄ alkylene). In some embodiments, L^{R2} is substituted alkylene (e.g., C₁-C₈ alkylene, C₁-C₆ alkylene, or C₁-C₄ alkylene). In some embodiments, L^{R2} is an unsubstituted alkylene (e.g., C₁-C₈ alkylene, C₁-C₆ alkylene, or C₁-C₄ alkylene). In some embodiments, L^{R2} is substituted or unsubstituted heteroalkylene (e.g., 2 to 8 membered heteroalkylene, 2 to 6 membered heteroalkylene, or 2 to 4 membered heteroalkylene). In some embodiments, L^{R2} is substituted heteroalkylene (e.g., 2 to 8 membered heteroalkylene, 2 to 6 membered heteroalkylene, or 2 to 4 membered heteroalkylene). In some embodiments, L^{R2} is an unsubstituted heteroalkylene (e.g., 2 to 8 membered heteroalkylene, 2 to 6 membered heteroalkylene, or 2 to 4 membered heteroalkylene). In some embodiments, L^{R2} is substituted or unsubstituted cycloalkylene (e.g., C₃-C₈ cycloalkylene, C₃-C₆ cycloalkylene, or C₅-C₆ cycloalkylene). In some embodiments, L^{R2} is substituted cycloalkylene (e.g., C₃-C₈ cycloalkylene, C₃-C₆ cycloalkylene, or C₅-C₆ cycloalkylene). In some embodiments, L^{R2} is an unsubstituted cycloalkylene (e.g., C₃-C₈ cycloalkylene, C₃-C₆ cycloalkylene, or C₅-C₆ cycloalkylene). In some embodiments, L^{R2} is substituted or unsubstituted heterocycloalkylene (e.g., 3 to 8 membered heterocycloalkylene, 3 to 6 membered heterocycloalkylene, or 5 to 6 membered heterocycloalkylene). In some embodiments, L^{R2} is substituted heterocycloalkylene (e.g., 3 to 8 membered heterocycloalkylene, 3 to 6 membered heterocycloalkylene, or 5 to 6 membered heterocycloalkylene). In some embodiments, L^{R2} is an unsubstituted heterocycloalkylene (e.g., 3 to 8 membered heterocycloalkylene, 3 to 6 membered heterocycloalkylene, or 5 to 6 membered heterocycloalkylene). In some embodiments, L^{R2} is substituted or unsubstituted arylene (e.g., C₆-C₁₀ arylene, C₁₀ arylene, or phenylene). In some embodiments, L^{R2} is substituted arylene (e.g., C₆-C₁₀ arylene, C₁₀ arylene, or phenylene). In some embodiments, L^{R2} is an unsubstituted arylene (e.g., C₆-C₁₀ arylene, C₁₀ arylene, or phenylene). In some embodiments, L^{R2} is substituted or unsubstituted heteroarylene (e.g., 5 to 10 membered heteroarylene, 5 to 9 membered heteroarylene, or 5 to 6 membered heteroarylene). In some embodiments, L^{R2} is substituted heteroarylene (e.g., 5 to 10 membered heteroarylene, 5 to 9 membered heteroarylene, or 5 to 6 membered heteroarylene). In some embodiments, L^{R2} is an unsubstituted heteroarylene (e.g., 5 to 10 membered heteroarylene, 5 to 9 membered heteroarylene, or 5 to 6 membered heteroarylene).

In some embodiments, L^{R2} is independently a bond, —S(O)₂—, —S(O)—, —NH—, —O—, —S—, —C(O)—, —C(O)NH—, —NHC(O)—, —NHC(O)NH—, —C(O)O—, —OC(O)—, R²⁴-substituted or unsubstituted alkylene (e.g., C₁-C₈ alkylene, C₁-C₆ alkylene, or C₁-C₄ alkylene), R²⁴-substituted or unsubstituted heteroalkylene (e.g., 2 to 8 membered heteroalkylene, 2 to 6 membered heteroalkylene, or 2 to 4 membered heteroalkylene), R²⁴-substituted or unsubstituted cycloalkylene (e.g., C₃-C₈ cycloalkylene, C₃-C₆ cycloalkylene, or C₅-C₆ cycloalkylene), R²⁴-substituted or unsubstituted heterocycloalkylene (e.g., 3 to 8 membered heterocycloalkylene, 3 to 6 membered heterocycloalkylene, or 5 to 6 membered heterocycloalkylene), R²⁴-substituted or unsubstituted arylene (e.g., C₆-C₁₀ arylene, C₁₀ arylene, or phenylene), or R²⁴-substituted or unsubstituted heteroarylene (e.g., 5 to 10 membered heteroarylene, 5 to 9 membered heteroarylene, or 5 to 6 membered heteroarylene). In some embodiments, L^{R2} is independently a bond, —S(O)₂—, —S(O)—, —NH—, —O—, —S—, —C(O)—, —C(O)NH—, —NHC(O)—, —NHC(O)NH—, —C(O)O—, —OC(O)—, unsubstituted alkylene (e.g., C₁-C₈ alkylene, C₁-C₆ alkylene, or C₁-C₄ alkylene), unsubstituted heteroalkylene (e.g., 2 to 8 membered heteroalkylene, 2 to 6 membered heteroalkylene, or 2 to 4 membered heteroalkylene), unsubstituted cycloalkylene (e.g., C₃-C₈ cycloalkylene, C₃-C₆ cycloalkylene, or C₅-C₆ cycloalkylene), unsubstituted heterocycloalkylene (e.g., 3 to 8 membered heterocycloalkylene, 3 to 6 membered heterocycloalkylene, or 5 to 6 membered heterocycloalkylene), unsubstituted arylene (e.g., C₆-C₁₀ arylene, C₁₀ arylene, or phenylene), or unsubstituted heteroarylene (e.g., 5 to 10 membered heteroarylene, 5 to 9 membered heteroarylene, or 5 to 6 membered heteroarylene).

In some embodiments, L^{R2} is R²⁴-substituted or unsubstituted alkylene (e.g., C₁-C₈ alkylene, C₁-C₆ alkylene, or C₁-C₄ alkylene). In some embodiments, L^{R2} is R²⁴-substituted alkylene (e.g., C₁-C₈ alkylene, C₁-C₆ alkylene, or C₁-C₄ alkylene). In some embodiments, L^{R2} is an unsubstituted alkylene (e.g., C₁-C₈ alkylene, C₁-C₆ alkylene, or C₁-C₄ alkylene). In some embodiments, L^{R2} is R²⁴-substituted or unsubstituted heteroalkylene (e.g., 2 to 8 membered heteroalkylene, 2 to 6 membered heteroalkylene, or 2 to 4 membered heteroalkylene). In some embodiments, L^{R2} is R²⁴-substituted heteroalkylene (e.g., 2 to 8 membered heteroalkylene, 2 to 6 membered heteroalkylene, or 2 to 4 membered heteroalkylene). In some embodiments, L^{R2} is an unsubstituted heteroalkylene (e.g., 2 to 8 membered heteroalkylene, 2 to 6 membered heteroalkylene, or 2 to 4 membered heteroalkylene). In some embodiments, L^{R2} is R²⁴-substituted or unsubstituted cycloalkylene (e.g., C₃-C₈ cycloalkylene, C₃-C₆ cycloalkylene, or C₅-C₆ cycloalkylene). In some embodiments, L^{R2} is R²⁴-substituted cycloalkylene (e.g., C₃-C₈ cycloalkylene, C₃-C₆ cycloalkylene, or C₅-C₆ cycloalkylene). In some embodiments, L^{R2} is an unsubstituted cycloalkylene (e.g., C₃-C₈ cycloalkylene, C₃-C₆ cycloalkylene, or C₅-C₆ cycloalkylene). In some embodiments, L^{R2} is R²⁴-substituted or unsubstituted heterocycloalkylene (e.g., 3 to 8 membered heterocycloalkylene, 3 to 6 membered heterocycloalkylene, or 5 to 6 membered heterocycloalkylene). In some embodiments, L^{R2} is R²⁴-substituted heterocycloalkylene (e.g., 3 to 8 membered heterocycloalkylene, 3 to 6 membered heterocycloalkylene, or 5 to 6 membered heterocycloalkylene). In some embodiments, L^{R2} is an unsubstituted heterocycloalkylene (e.g., 3 to 8 membered heterocycloalkylene, 3 to 6 membered heterocycloalkylene, or 5 to 6 membered heterocycloalkylene). In some embodiments, L^{R2} is R²⁴-substituted or unsubstituted arylene (e.g., C₆-C₁₀ arylene, C₁₀ arylene, or phenylene). In some embodiments, L^{R2} is R²⁴-substituted arylene (e.g., C₆-C₁₀ arylene, C₁₀ arylene, or phenylene). In some embodiments, L^{R2} is an unsubstituted arylene (e.g., C₆-C₁₀ arylene, C₁₀ arylene, or phenylene). In some embodiments, L^{R2} is R²⁴-substituted or unsubstituted heteroarylene (e.g., 5 to 10 membered heteroarylene, 5 to 9 membered heteroarylene, or 5 to 6 membered heteroarylene). In some embodiments, L^{R2} is R²⁴-substituted heteroarylene (e.g., 5 to 10 membered heteroarylene, 5 to 9 membered heteroarylene, or 5 to 6 membered heteroarylene). In some embodiments, L^{R2} is an unsubstituted heteroarylene (e.g., 5 to 10 membered heteroarylene, 5 to 9 membered heteroarylene, or 5 to 6 membered heteroarylene).

In some embodiments, R^{2A} is independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In some embodiments, the divalent linker bonded to both the monovalent cellular component binder and monovalent targeted autophagy protein binder is attached to the monovalent targeted autophagy protein binder at an R^{2A} position. In some embodiments, when the divalent linker bonded to both the monovalent cellular component binder and monovalent targeted autophagy protein binder is attached to the monovalent targeted autophagy protein binder at an R^{2A} position, R^{2A} is replaced with a divalent linker, referred to in this embodiment as L^{R2}.

In some embodiments, R^{2B} is independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In some embodiments, the divalent linker bonded to both the monovalent cellular component binder and monovalent targeted autophagy protein binder is attached to the monovalent targeted autophagy protein binder at an R^{2B} position. In some embodiments, when the divalent linker bonded to both the monovalent cellular component binder and monovalent targeted autophagy protein binder is attached to the monovalent targeted autophagy protein binder at an R^{2B} position, R^{2B} is replaced with a divalent linker, referred to in this embodiment as L^{R2}.

In some embodiments, R^{2C} is independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In some embodiments, the divalent linker bonded to both the monovalent cellular component binder and monovalent targeted autophagy protein binder is attached to the monovalent targeted autophagy protein binder at an R^{2C} position. In some embodiments, when the divalent linker bonded to both the monovalent cellular component binder and monovalent targeted autophagy protein binder is attached to the monovalent targeted autophagy protein binder at an R^{2C} position, R^{2C} is replaced with a divalent linker, referred to in this embodiment as L^{R2}.

In some embodiments, R^{2D} is independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In some embodiments, the divalent linker bonded to both the monovalent cellular component binder and monovalent targeted autophagy protein binder is attached to the monovalent targeted autophagy protein binder at an R^{2D} position. In some embodiments, when the divalent linker bonded to both the monovalent cellular component binder and monovalent targeted autophagy protein binder is attached to the monovalent targeted autophagy protein binder at an R^{2D} position, R^{2D} is replaced with a divalent linker, referred to in this embodiment as L^{R2}.

In some embodiments, R^{2A} and R^{2B} substituents bonded to the same nitrogen atom are independently joined to form a substituted or unsubstituted heterocycloalkyl (e.g., 3 to 8 membered heterocycloalkyl, 3 to 6 membered heterocycloalkyl, or 5 to 6 membered heterocycloalkyl) or substituted or unsubstituted heteroaryl (e.g., 5 to 10 membered heteroaryl, 5 to 9 membered heteroaryl, or 5 to 6 membered heteroaryl). In some embodiments, R^{2A} and R^{2B} substituents bonded to the same nitrogen atom are independently joined to form a substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R^{2A} and R^{2B} substituents bonded to the same nitrogen atom are independently joined to form a substituted heterocycloalkyl (e.g., 3 to 8 membered heterocycloalkyl, 3 to 6 membered heterocycloalkyl, or 5 to 6 membered heterocycloalkyl). In some embodiments, R^{2A} and R^{2B} substituents bonded to the same nitrogen atom are independently joined to form an unsubstituted heterocycloalkyl (e.g., 3 to 8 membered heterocycloalkyl, 3 to 6 membered heterocycloalkyl, or 5 to 6 membered heterocycloalkyl). In some embodiments, R^{2A} and R^{2B} substituents bonded to the same nitrogen atom are independently joined to form a substituted or unsubstituted heteroaryl (e.g., 5 to 10 membered heteroaryl, 5 to 9 membered heteroaryl, or 5 to 6 membered heteroaryl). In some embodiments, R^{2A} and R^{2B} substituents bonded to the same nitrogen atom are independently joined to form a substituted heteroaryl (e.g., 5 to 10 membered heteroaryl, 5 to 9 membered heteroaryl, or 5 to 6 membered heteroaryl). In some embodiments, R^{2A} and R^{2B} substituents bonded to the same nitrogen atom are independently joined to form an unsubstituted heteroaryl (e.g., 5 to 10 membered heteroaryl, 5 to 9 membered heteroaryl, or 5 to 6 membered heteroaryl).

In some embodiments, R^{2A}, R^{2B}, R^{2C}, and R^{2D} are independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, R²⁴-substituted or unsubstituted alkyl (e.g., C₁-C₈ alkyl, C₁-C₆ alkyl, or C₁-C₄ alkyl), R²⁴-substituted or unsubstituted heteroalkyl (e.g., 2 to 8 membered heteroalkyl, 2 to 6 membered heteroalkyl, or 2 to 4 membered heteroalkyl), R²⁴-substituted or unsubstituted cycloalkyl (e.g., C₃-C₈ cycloalkyl, C₃-C₆ cycloalkyl, or C₅-C₆ cycloalkyl), R²⁴-substituted or unsubstituted heterocycloalkyl (e.g., 3 to 8 membered heterocycloalkyl, 3 to 6 membered heterocycloalkyl, or 5 to 6 membered heterocycloalkyl), R²⁴-substituted or unsubstituted aryl (e.g., C₆-C₁₀ aryl, C₁₀ aryl, or phenyl), or R²⁴-substituted or unsubstituted heteroaryl (e.g., 5 to 10 membered heteroaryl, 5 to 9 membered heteroaryl, or 5 to 6 membered heteroaryl). In some embodiments, R^{2A} and R^{2B} substituents bonded to the same nitrogen atom are independently joined to form an R²⁴-substituted or unsubstituted heterocycloalkyl (e.g., 3 to 8 membered heterocycloalkyl, 3 to 6 membered heterocycloalkyl, or 5 to 6 membered heterocycloalkyl) or R²⁴-substituted or unsubstituted heteroaryl (e.g., 5 to 10 membered heteroaryl, 5 to 9 membered heteroaryl, or 5 to 6 membered heteroaryl). In some embodiments, R^{2A} and R^{2B} substituents bonded to the same nitrogen atom are independently joined to form an R²⁴-substituted or unsubstituted heterocycloalkyl (e.g., 3 to 8 membered heterocycloalkyl, 3 to 6 membered heterocycloalkyl. In some embodiments, R^{2A} and R^{2B} substituents bonded to the same nitrogen atom are independently joined to form an R²⁴-substituted or unsubstituted heteroaryl (e.g., 5 to 10 membered heteroaryl, 5 to 9 membered heteroaryl, or 5 to 6 membered heteroaryl).

In some embodiments, R^{2A} is independently R²⁴-substituted or unsubstituted alkyl (e.g., C₁-C₈ alkyl, C₁-C₆ alkyl, or C₁-C₄ alkyl). In some embodiments, R^{2A} is independently R²⁴-substituted alkyl (e.g., C₁-C₈ alkyl, C₁-C₆ alkyl, or C₁-C₄ alkyl). In some embodiments, R^{2A} is independently an unsubstituted alkyl (e.g., C₁-C₈ alkyl, C₁-C₆ alkyl, or C₁-C₄ alkyl). In some embodiments, R^{2A} is independently R²⁴-substituted or unsubstituted heteroalkyl (e.g., 2 to 8 membered heteroalkyl, 2 to 6 membered heteroalkyl, or 2 to 4 membered heteroalkyl). In some embodiments, R^{2A} is independently R²⁴-substituted heteroalkyl (e.g., 2 to 8 membered heteroalkyl, 2 to 6 membered heteroalkyl, or 2 to 4 membered heteroalkyl). In some embodiments, R^{2A} is independently an unsubstituted heteroalkyl (e.g., 2 to 8 membered heteroalkyl, 2 to 6 membered heteroalkyl, or 2 to 4 membered heteroalkyl). In some embodiments, R^{2A} is independently R²⁴-substituted or unsubstituted cycloalkyl (e.g., C₃-C₈ cycloalkyl, C₃-C₆ cycloalkyl, or C₅-C₆ cycloalkyl). In some embodiments, R^{2A} is independently R²⁴-substituted cycloalkyl (e.g., C₃-C₈ cycloalkyl, C₃-C₆ cycloalkyl, or C₅-C₆ cycloalkyl). In some embodiments, R^{2A} is independently an unsubstituted cycloalkyl (e.g., C₃-C₈ cycloalkyl, C₃-C₆ cycloalkyl, or C₅-C₆ cycloalkyl). In some embodiments, R^{2A} is independently R²⁴-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R^{2A} is independently R²⁴-substituted heterocycloalkyl (e.g., 3 to 8 membered heterocycloalkyl, 3 to 6 membered heterocycloalkyl, or 5 to 6 membered heterocycloalkyl). In some embodiments, R^{2A} is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R^{2A} is independently R²⁴-substituted or unsubstituted aryl (e.g., C₆-C₁₀ aryl, C₁₀ aryl, or phenyl). In some embodiments, R^{2A} is independently R²⁴-substituted aryl (e.g., C₆-C₁₀ aryl, C₁₀ aryl, or phenyl). In some embodiments, R^{2A} is independently an unsubstituted aryl (e.g., C₆-C₁₀ aryl, C₁₀ aryl, or phenyl). In some embodiments, R^{2A} is independently R²⁴-substituted or unsubstituted heteroaryl (e.g., 5 to 10 membered heteroaryl, 5 to 9 membered heteroaryl, or 5 to 6 membered heteroaryl). In some embodiments, R^{2A} is independently R²⁴-substituted heteroaryl (e.g., 5 to 10 membered heteroaryl, 5 to 9 membered heteroaryl, or 5 to 6 membered heteroaryl). In some embodiments, R^{2A} is independently an unsubstituted heteroaryl (e.g., 5 to 10 membered heteroaryl, 5 to 9 membered heteroaryl, or 5 to 6 membered heteroaryl).

In some embodiments, R^{2A} is independently —CCl₃. In some embodiments, R^{2A} is independently —CBr₃. In some embodiments, R^{2A} is independently —CF₃. In some embodiments, R^{2A} is independently —CI₃. In some embodiments, R^{2A} is independently —CHCl₂. In some embodiments, R^{2A} is independently —CHBr₂. In some embodiments, R^{2A} is independently —CHF₂. In some embodiments, R^{2A} is independently —CHI₂. In some embodiments, R^{2A} is independently —CH₂Cl. In some embodiments, R^{2A} is independently —CH₂Br. In some embodiments, R^{2A} is independently —CH₂F. In some embodiments, R^{2A} is independently —CH₂I. In some embodiments, R^{2A} is independently —CN. In some embodiments, R^{2A} is independently —OH. In some embodiments, R^{2A} is independently —COOH. In some embodiments, R^{2A} is independently —CONH₂. In some embodiments, R^{2A} is independently —OCCl₃. In some embodiments, R^{2A} is independently —OCF₃. In some embodiments, R^{2A} is independently —OCBr₃. In some embodiments, R^{2A} is independently —OCI₃. In some embodiments, R^{2A} is independently —OCHCl₂. In some embodiments, R^{2A} is independently —OCHBr₂. In some embodiments, R^{2A} is independently —OCHI₂. In some embodiments, R^{2A} is independently —OCHF₂. In some embodiments, R^{2A} is independently —OCH₂Cl. In some embodiments, R^{2A} is independently —OCH₂Br. In some embodiments, R^{2A} is independently —OCH₂I. In some embodiments, R^{2A} is independently —OCH₂F. In some embodiments, R^{2A} is independently —OCH₃. In some embodiments, R^{2A} is independently —CH₃. In some embodiments, R^{2A} is independently —CH₂CH₃. In some embodiments, R^{2A} is independently unsubstituted propyl. In some embodiments, R^{2A} is independently unsubstituted isopropyl. In some embodiments, R^{2A} is independently unsubstituted butyl. In some embodiments, R^{2A} is independently unsubstituted tert-butyl. In some embodiments, R^{2A} is independently hydrogen.

In some embodiments, R^{2B} is independently R²⁴-substituted or unsubstituted alkyl (e.g., C₁-C₈ alkyl, C₁-C₆ alkyl, or C₁-C₄ alkyl). In some embodiments, R^{2B} is independently R²⁴-substituted alkyl (e.g., C₁-C₈ alkyl, C₁-C₆ alkyl, or C₁-C₄ alkyl). In some embodiments, R^{2B} is independently an unsubstituted alkyl (e.g., C₁-C₈ alkyl, C₁-C₆ alkyl, or C₁-C₄ alkyl). In some embodiments, R^{2B} is independently R²⁴-substituted or unsubstituted heteroalkyl (e.g., 2 to 8 membered heteroalkyl, 2 to 6 membered heteroalkyl, or 2 to 4 membered heteroalkyl). In some embodiments, R^{2B} is independently R²⁴-substituted heteroalkyl (e.g., 2 to 8 membered heteroalkyl, 2 to 6 membered heteroalkyl, or 2 to 4 membered heteroalkyl). In some embodiments, R^{2B} is independently an unsubstituted heteroalkyl (e.g., 2 to 8 membered heteroalkyl, 2 to 6 membered heteroalkyl, or 2 to 4 membered heteroalkyl). In some embodiments, R^{2B} is independently R²⁴-substituted or unsubstituted cycloalkyl (e.g., C₃-C₈ cycloalkyl, C₃-C₆ cycloalkyl, or C₅-C₆ cycloalkyl). In some embodiments, R^{2B} is independently R²⁴-substituted cycloalkyl (e.g., C₃-C₈ cycloalkyl, C₃-C₆ cycloalkyl, or C₅-C₆ cycloalkyl). In some embodiments, R^{2B} is independently an unsubstituted cycloalkyl (e.g., C₃-C₈ cycloalkyl, C₃-C₆ cycloalkyl, or C₅-C₆ cycloalkyl). In some embodiments, R^{2B} is independently R²⁴-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R^{2B} is independently R²⁴-substituted heterocycloalkyl (e.g., 3 to 8 membered heterocycloalkyl, 3 to 6 membered heterocycloalkyl, or 5 to 6 membered heterocycloalkyl). In some embodiments, R^{2B} is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R^{2B} is independently R²⁴-substituted or unsubstituted aryl (e.g., C₆-C₁₀ aryl, C₁₀ aryl, or phenyl). In some embodiments, R^{2B} is independently R²⁴-substituted aryl (e.g., C₆-C₁₀ aryl, C₁₀ aryl, or phenyl). In some embodiments, R^{2B} is independently an unsubstituted aryl (e.g., C₆-C₁₀ aryl, C₁₀ aryl, or phenyl). In some embodiments, R^{2B} is independently R²⁴-substituted or unsubstituted heteroaryl (e.g., 5 to 10 membered heteroaryl, 5 to 9 membered heteroaryl, or 5 to 6 membered heteroaryl). In some embodiments, R^{2B} is independently R²⁴-substituted heteroaryl (e.g., 5 to 10 membered heteroaryl, 5 to 9 membered heteroaryl, or 5 to 6 membered heteroaryl). In some embodiments, R^{2B} is independently an unsubstituted heteroaryl (e.g., 5 to 10 membered heteroaryl, 5 to 9 membered heteroaryl, or 5 to 6 membered heteroaryl).

In some embodiments, R^{2B} is independently —CCl₃. In some embodiments, R^{2B} is independently —CBr₃. In some embodiments, R^{2B} is independently —CF₃. In some embodiments, R^{2B} is independently —CI₃. In some embodiments, R^{2B} is independently —CHCl₂. In some embodiments, R^{2B} is independently —CHBr₂. In some embodiments, R^{2B} is independently —CHF₂. In some embodiments, R^{2B} is independently —CHI₂. In some embodiments, R^{2B} is independently —CH₂Cl. In some embodiments, R^{2B} is independently —CH₂Br. In some embodiments, R^{2B} is independently —CH₂F. In some embodiments, R^{2B} is independently —CH₂I. In some embodiments, R^{2B} is independently —CN. In some embodiments, R^{2B} is independently —OH. In some embodiments, R^{2B} is independently —COOH. In some embodiments, R^{2B} is independently —CONH₂. In some embodiments, R^{2B} is independently —OCCl₃. In some embodiments, R^{2B} is independently —OCF₃. In some embodiments, R^{2B} is independently —OCBr₃. In some embodiments, R^{2B} is independently —OCI₃. In some embodiments, R^{2B} is independently —OCHCl₂. In some embodiments, R^{2B} is independently —OCHBr₂. In some embodiments, R^{2B} is independently —OCHI₂. In some embodiments, R^{2B} is independently —OCHF₂. In some embodiments, R^{2B} is independently —OCH₂Cl. In some embodiments, R^{2B} is independently —OCH₂Br. In some embodiments, R^{2B} is independently —OCH₂I. In some embodiments, R^{2B} is independently —OCH₂F. In some embodiments, R^{2B} is independently —OCH₃. In some embodiments, R^{2B} is independently —CH₃. In some embodiments, R^{2B} is independently —CH₂CH₃. In some embodiments, R^{2B} is independently unsubstituted propyl. In some embodiments, R^{2B} is independently unsubstituted isopropyl. In some embodiments, R^{2B} is independently unsubstituted butyl. In some embodiments, R^{2B} is independently unsubstituted tert-butyl. In some embodiments, R^{2B} is independently hydrogen.

In some embodiments, R^{2C} is independently R²⁴-substituted or unsubstituted alkyl (e.g., C₁-C₈ alkyl, C₁-C₆ alkyl, or C₁-C₄ alkyl). In some embodiments, R^{2C} is independently R²⁴-substituted alkyl (e.g., C₁-C₈ alkyl, C₁-C₆ alkyl, or C₁-C₄ alkyl). In some embodiments, R^{2C} is independently an unsubstituted alkyl (e.g., C₁-C₈ alkyl, C₁-C₆ alkyl, or C₁-C₄ alkyl). In some embodiments, R^{2C} is independently R²⁴-substituted or unsubstituted heteroalkyl (e.g., 2 to 8 membered heteroalkyl, 2 to 6 membered heteroalkyl, or 2 to 4 membered heteroalkyl). In some embodiments, R^{2C} is independently R²⁴-substituted heteroalkyl (e.g., 2 to 8 membered heteroalkyl, 2 to 6 membered heteroalkyl, or 2 to 4 membered heteroalkyl). In some embodiments, R^{2C} is independently an unsubstituted heteroalkyl (e.g., 2 to 8 membered heteroalkyl, 2 to 6 membered heteroalkyl, or 2 to 4 membered heteroalkyl). In some embodiments, R^{2C} is independently R²⁴-substituted or unsubstituted cycloalkyl (e.g., C₃-C₈ cycloalkyl, C₃-C₆ cycloalkyl, or C₅-C₆ cycloalkyl). In some embodiments, R^{2C} is independently R²⁴-substituted cycloalkyl (e.g., C₃-C₈ cycloalkyl, C₃-C₆ cycloalkyl, or C₅-C₆ cycloalkyl). In some embodiments, R^{2C} is independently an unsubstituted cycloalkyl (e.g., C₃-C₈ cycloalkyl, C₃-C₆ cycloalkyl, or C₅-C₆ cycloalkyl). In some embodiments, R^{2C} is independently R²⁴-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R^{2C} is independently R²⁴-substituted heterocycloalkyl (e.g., 3 to 8 membered heterocycloalkyl, 3 to 6 membered heterocycloalkyl, or 5 to 6 membered heterocycloalkyl). In some embodiments, R^{2C} is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R^{2C} is independently R²⁴-substituted or unsubstituted aryl (e.g., C₆-C₁₀ aryl, C₁₀ aryl, or phenyl). In some embodiments, R^{2C} is independently R²⁴-substituted aryl (e.g., C₆-C₁₀ aryl, C₁₀ aryl, or phenyl). In some embodiments, R^{2C} is independently an unsubstituted aryl (e.g., C₆-C₁₀ aryl, C₁₀ aryl, or phenyl). In some embodiments, R^{2C} is independently R²⁴-substituted or unsubstituted heteroaryl (e.g., 5 to 10 membered heteroaryl, 5 to 9 membered heteroaryl, or 5 to 6 membered heteroaryl). In some embodiments, R^{2C} is independently R²⁴-substituted heteroaryl (e.g., 5 to 10 membered heteroaryl, 5 to 9 membered heteroaryl, or 5 to 6 membered heteroaryl). In some embodiments, R^{2C} is independently an unsubstituted heteroaryl (e.g., 5 to 10 membered heteroaryl, 5 to 9 membered heteroaryl, or 5 to 6 membered heteroaryl).

In some embodiments, R^{2C} is independently —CCl₃. In some embodiments, R^{2C} is independently —CBr₃. In some embodiments, R^{2C} is independently —CF₃. In some embodiments, R^{2C} is independently —CI₃. In some embodiments, R^{2C} is independently —CHCl₂. In some embodiments, R^{2C} is independently —CHBr₂. In some embodiments, R^{2C} is independently —CHF₂. In some embodiments, R^{2C} is independently —CHI₂. In some embodiments, R^{2C} is independently —CH₂Cl. In some embodiments, R^{2C} is independently —CH₂Br. In some embodiments, R^{2C} is independently —CH₂F. In some embodiments, R^{2C} is independently —CH₂I. In some embodiments, R^{2C} is independently —CN. In some embodiments, R^{2C} is independently —OH. In some embodiments, R^{2C} is independently —COOH. In some embodiments, R^{2C} is independently —CONH₂. In some embodiments, R^{2C} is independently —OCCl₃. In some embodiments, R^{2C} is independently —OCF₃. In some embodiments, R^{2C} is independently —OCBr₃. In some embodiments, R^{2C} is independently —OCI₃. In some embodiments, R^{2C} is independently —OCHCl₂. In some embodiments, R^{2C} is independently —OCHBr₂. In some embodiments, R^{2C} is independently —OCHI₂. In some embodiments, R^{2C} is independently —OCHF₂. In some embodiments, R^{2C} is independently —OCH₂Cl. In some embodiments, R^{2C} is independently —OCH₂Br. In some embodiments, R^{2C} is independently —OCH₂I. In some embodiments, R^{2C} is independently —OCH₂F. In some embodiments, R^{2C} is independently —OCH₃. In some embodiments, R^{2C} is independently —CH₃. In some embodiments, R^{2C} is independently —CH₂CH₃. In some embodiments, R^{2C} is independently unsubstituted propyl. In some embodiments, R^{2C} is independently unsubstituted isopropyl. In some embodiments, R^{2C} is independently unsubstituted butyl. In some embodiments, R^{2C} is independently unsubstituted tert-butyl. In some embodiments, R^{2C} is independently hydrogen.

In some embodiments, R^{2D} is independently R²⁴-substituted or unsubstituted alkyl (e.g., C₁-C₈ alkyl, C₁-C₆ alkyl, or C₁-C₄ alkyl). In some embodiments, R^{2D} is independently R²⁴-substituted alkyl (e.g., C₁-C₈ alkyl, C₁-C₆ alkyl, or C₁-C₄ alkyl). In some embodiments, R^{2D} is independently an unsubstituted alkyl (e.g., C₁-C₈ alkyl, C₁-C₆ alkyl, or C₁-C₄ alkyl). In some embodiments, R^{2D} is independently R²⁴-substituted or unsubstituted heteroalkyl (e.g., 2 to 8 membered heteroalkyl, 2 to 6 membered heteroalkyl, or 2 to 4 membered heteroalkyl). In some embodiments, R^{2D} is independently R²⁴-substituted heteroalkyl (e.g., 2 to 8 membered heteroalkyl, 2 to 6 membered heteroalkyl, or 2 to 4 membered heteroalkyl). In some embodiments, R^{2D} is independently an unsubstituted heteroalkyl (e.g., 2 to 8 membered heteroalkyl, 2 to 6 membered heteroalkyl, or 2 to 4 membered heteroalkyl). In some embodiments, R^{2D} is independently R²⁴-substituted or unsubstituted cycloalkyl (e.g., C₃-C₈ cycloalkyl, C₃-C₆ cycloalkyl, or C₅-C₆ cycloalkyl). In some embodiments, R^{2D} is independently R²⁴-substituted cycloalkyl (e.g., C₃-C₈ cycloalkyl, C₃-C₆ cycloalkyl, or C₅-C₆ cycloalkyl). In some embodiments, R^{2D} is independently an unsubstituted cycloalkyl (e.g., C₃-C₈ cycloalkyl, C₃-C₆ cycloalkyl, or C₅-C₆ cycloalkyl). In some embodiments, R^{2D} is independently R²⁴-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R^{2D} is independently R²⁴-substituted heterocycloalkyl (e.g., 3 to 8 membered heterocycloalkyl, 3 to 6 membered heterocycloalkyl, or 5 to 6 membered heterocycloalkyl). In some embodiments, R^{2D} is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R^{2D} is independently R²⁴-substituted or unsubstituted aryl (e.g., C₆-C₁₀ aryl, C₁₀ aryl, or phenyl). In some embodiments, R^{2D} is independently R²⁴-substituted aryl (e.g., C₆-C₁₀ aryl, C₁₀ aryl, or phenyl). In some embodiments, R^{2D} is independently an unsubstituted aryl (e.g., C₆-C₁₀ aryl, C₁₀ aryl, or phenyl). In some embodiments, R^{2D} is independently R²⁴-substituted or unsubstituted heteroaryl (e.g., 5 to 10 membered heteroaryl, 5 to 9 membered heteroaryl, or 5 to 6 membered heteroaryl). In some embodiments, R^{2D} is independently R²⁴-substituted heteroaryl (e.g., 5 to 10 membered heteroaryl, 5 to 9 membered heteroaryl, or 5 to 6 membered heteroaryl). In some embodiments, R^{2D} is independently an unsubstituted heteroaryl (e.g., 5 to 10 membered heteroaryl, 5 to 9 membered heteroaryl, or 5 to 6 membered heteroaryl).

In some embodiments, R^{2D} is independently —CCl₃. In some embodiments, R^{2D} is independently —CBr₃. In some embodiments, R^{2D} is independently —CF₃. In some embodiments, R^{2D} is independently —CI₃. In some embodiments, R^{2D} is independently —CHCl₂. In some embodiments, R^{2D} is independently —CHBr₂. In some embodiments, R^{2D} is independently —CHF₂. In some embodiments, R^{2D} is independently —CHI₂. In some embodiments, R^{2D} is independently —CH₂Cl. In some embodiments, R^{2D} is independently —CH₂Br. In some embodiments, R^{2D} is independently —CH₂F. In some embodiments, R^{2D} is independently —CH₂I. In some embodiments, R^{2D} is independently —CN. In some embodiments, R^{2D} is independently —OH. In some embodiments, R^{2D} is independently —COOH. In some embodiments, R^{2D} is independently —CONH₂. In some embodiments, R^{2D} is independently —OCCl₃. In some embodiments, R^{2D} is independently —OCF₃. In some embodiments, R^{2D} is independently —OCBr₃. In some embodiments, R^{2D} is independently —OCI₃. In some embodiments, R^{2D} is independently —OCHCl₂. In some embodiments, R^{2D} is independently —OCHBr₂. In some embodiments, R^{2D} is independently —OCHI₂. In some embodiments, R^{2D} is independently —OCHF₂. In some embodiments, R^{2D} is independently —OCH₂Cl. In some embodiments, R^{2D} is independently —OCH₂Br. In some embodiments, R^{2D} is independently —OCH₂I. In some embodiments, R^{2D} is independently —OCH₂F. In some embodiments, R^{2D} is independently —OCH₃. In some embodiments, R^{2D} is independently —CH₃. In some embodiments, R^{2D} is independently —CH₂CH₃. In some embodiments, R^{2D} is independently unsubstituted propyl. In some embodiments, R^{2D} is independently unsubstituted isopropyl. In some embodiments, R^{2D} is independently unsubstituted butyl. In some embodiments, R^{2D} is independently unsubstituted tert-butyl. In some embodiments, R^{2D} is independently hydrogen.

In some embodiments, R³ is independently halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N₃, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In some embodiments, R³ is independently substituted or unsubstituted alkyl (e.g., C₁-C₈ alkyl, C₁-C₆ alkyl, or C₁-C₄ alkyl). In some embodiments, R³ is independently substituted alkyl (e.g., C₁-C₈ alkyl, C₁-C₆ alkyl, or C₁-C₄ alkyl). In some embodiments, R³ is independently an unsubstituted alkyl (e.g., C₁-C₈ alkyl, C₁-C₆ alkyl, or C₁-C₄ alkyl). In some embodiments, R³ is independently substituted or unsubstituted heteroalkyl (e.g., 2 to 8 membered heteroalkyl, 2 to 6 membered heteroalkyl, or 2 to 4 membered heteroalkyl). In some embodiments, R³ is independently substituted heteroalkyl (e.g., 2 to 8 membered heteroalkyl, 2 to 6 membered heteroalkyl, or 2 to 4 membered heteroalkyl). In some embodiments, R³ is independently an unsubstituted heteroalkyl (e.g., 2 to 8 membered heteroalkyl, 2 to 6 membered heteroalkyl, or 2 to 4 membered heteroalkyl). In some embodiments, R³ is independently substituted or unsubstituted cycloalkyl (e.g., C₃-C₈ cycloalkyl, C₃-C₆ cycloalkyl, or C₅-C₆ cycloalkyl). In some embodiments, R³ is independently substituted cycloalkyl (e.g., C₃-C₈ cycloalkyl, C₃-C₆ cycloalkyl, or C₅-C₆ cycloalkyl). In some embodiments, R³ is independently an unsubstituted cycloalkyl (e.g., C3-C₈ cycloalkyl, C₃-C₆ cycloalkyl, or C₅-C₆ cycloalkyl). In some embodiments, R³ is independently substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R³ is independently substituted heterocycloalkyl (e.g., 3 to 8 membered heterocycloalkyl, 3 to 6 membered heterocycloalkyl, or 5 to 6 membered heterocycloalkyl). In some embodiments, R³ is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R³ is independently substituted or unsubstituted aryl (e.g., C₆-C₁₀ aryl, C₁₀ aryl, or phenyl). In some embodiments, R³ is independently substituted aryl (e.g., C₆-C₁₀ aryl, C₁₀ aryl, or phenyl). In some embodiments, R³ is independently an unsubstituted aryl (e.g., C₆-C₁₀ aryl, C₁₀ aryl, or phenyl). In some embodiments, R³ is independently substituted or unsubstituted heteroaryl (e.g., 5 to 10 membered heteroaryl, 5 to 9 membered heteroaryl, or 5 to 6 membered heteroaryl). In some embodiments, R³ is independently substituted heteroaryl (e.g., 5 to 10 membered heteroaryl, 5 to 9 membered heteroaryl, or 5 to 6 membered heteroaryl). In some embodiments, R³ is independently an unsubstituted heteroaryl (e.g., 5 to 10 membered heteroaryl, 5 to 9 membered heteroaryl, or 5 to 6 membered heteroaryl).

In some embodiments, R³ is independently halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, R²⁷-substituted or unsubstituted alkyl (e.g., C₁-C₈ alkyl, C₁-C₆ alkyl, or C₁-C₄ alkyl), R²⁷-substituted or unsubstituted heteroalkyl (e.g., 2 to 8 membered heteroalkyl, 2 to 6 membered heteroalkyl, or 2 to 4 membered heteroalkyl), R²⁷-substituted or unsubstituted cycloalkyl (e.g., C₃-C₈ cycloalkyl, C₃-C₆ cycloalkyl, or C₅-C₆ cycloalkyl), R²⁷-substituted or unsubstituted heterocycloalkyl (e.g., 3 to 8 membered heterocycloalkyl, 3 to 6 membered heterocycloalkyl, or 5 to 6 membered heterocycloalkyl), R²⁷-substituted or unsubstituted aryl (e.g., C₆-C₁₀ aryl, C₁₀ aryl, or phenyl), or R²⁷-substituted or unsubstituted heteroaryl (e.g., 5 to 10 membered heteroaryl, 5 to 9 membered heteroaryl, or 5 to 6 membered heteroaryl). In some embodiments, R³ is independently halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R³ is independently R²⁷-substituted or unsubstituted alkyl (e.g., C₁-C₈ alkyl, C₁-C₆ alkyl, or C₁-C₄ alkyl). In some embodiments, R³ is independently R²⁷-substituted alkyl (e.g., C₁-C₈ alkyl, C₁-C₆ alkyl, or C₁-C₄ alkyl). In some embodiments, R³ is independently an unsubstituted alkyl (e.g., C₁-C₈ alkyl, C₁-C₆ alkyl, or C₁-C₄ alkyl). In some embodiments, R³ is independently R²⁷-substituted or unsubstituted heteroalkyl (e.g., 2 to 8 membered heteroalkyl, 2 to 6 membered heteroalkyl, or 2 to 4 membered heteroalkyl). In some embodiments, R³ is independently R²⁷-substituted heteroalkyl (e.g., 2 to 8 membered heteroalkyl, 2 to 6 membered heteroalkyl, or 2 to 4 membered heteroalkyl). In some embodiments, R³ is independently an unsubstituted heteroalkyl (e.g., 2 to 8 membered heteroalkyl, 2 to 6 membered heteroalkyl, or 2 to 4 membered heteroalkyl). In some embodiments, R³ is independently R²⁷-substituted or unsubstituted cycloalkyl (e.g., C₃-C₈ cycloalkyl, C₃-C₆ cycloalkyl, or C₅-C₆ cycloalkyl). In some embodiments, R³ is independently R²⁷-substituted cycloalkyl (e.g., C₃-C₈ cycloalkyl, C₃-C₆ cycloalkyl, or C₅-C₆ cycloalkyl). In some embodiments, R³ is independently an unsubstituted cycloalkyl (e.g., C3-C₈ cycloalkyl, C₃-C₆ cycloalkyl, or C₅-C₆ cycloalkyl). In some embodiments, R³ is independently R²⁷-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R³ is independently R²⁷-substituted heterocycloalkyl (e.g., 3 to 8 membered heterocycloalkyl, 3 to 6 membered heterocycloalkyl, or 5 to 6 membered heterocycloalkyl). In some embodiments, R³ is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R³ is independently R²⁷-substituted or unsubstituted aryl (e.g., C₆-C₁₀ aryl, C₁₀ aryl, or phenyl). In some embodiments, R³ is independently R²⁷-substituted aryl (e.g., C₆-C₁₀ aryl, C₁₀ aryl, or phenyl). In some embodiments, R³ is independently an unsubstituted aryl (e.g., C₆-C₁₀ aryl, C₁₀ aryl, or phenyl). In some embodiments, R³ is independently R²⁷-substituted or unsubstituted heteroaryl (e.g., 5 to 10 membered heteroaryl, 5 to 9 membered heteroaryl, or 5 to 6 membered heteroaryl). In some embodiments, R³ is independently R²⁷-substituted heteroaryl (e.g., 5 to 10 membered heteroaryl, 5 to 9 membered heteroaryl, or 5 to 6 membered heteroaryl). In some embodiments, R³ is independently an unsubstituted heteroaryl (e.g., 5 to 10 membered heteroaryl, 5 to 9 membered heteroaryl, or 5 to 6 membered heteroaryl).

In some embodiments, R³ is independently —CX³₃. In some embodiments, R³ is independently —CHX³₂. In some embodiments, R³ is independently —CH₂X³. In some embodiments, R³ is independently —OCX³₃. In some embodiments, R³ is independently —OCH₂X³. In some embodiments, R³ is independently —OCHX³₂. In some embodiments, R³ is independently —CN. In some embodiments, R³ is independently —SR^{3D}In some embodiments, R³ is independently —SOR^{3D}. In some embodiments, R³ is independently —SO₂R^{3D}. In some embodiments, R³ is independently —SO₃R^{3D}. In some embodiments, R³ is independently —SO₄R^{3D}. In some embodiments, R³ is independently —SONR^{3A}R^{3B}. In some embodiments, R³ is independently —SO₂NR^{3A}R^{3B}. In some embodiments, R³ is independently —NHC(O)NR^{3A}R^{3B}. In some embodiments, R³ is independently —N(O). In some embodiments, R³ is independently —N(O)₂. In some embodiments, R³ is independently —NR^{3A}R^{3B}. In some embodiments, R³ is independently —C(O)R^{3C}. In some embodiments, R³ is independently —C(O)—OR^{3C}. In some embodiments, R³ is independently —C(O)NR^{3A}R^{3B}. In some embodiments, R³ is independently —OR^{3D}. In some embodiments, R³ is independently —NR^{3A}SO₂R^{3D}. In some embodiments, R³ is independently —NR^{3A}C(O)R^{3C}. In some embodiments, R³ is independently —NR^{3A}C(O)OR^{3C}. In some embodiments, R³ is independently —NR^{3A}OR^{3C}.

In some embodiments, R³ is independently oxo. In some embodiments, R³ is independently halogen. In some embodiments, R³ is independently —CCl₃. In some embodiments, R³ is independently —CBr₃. In some embodiments, R³ is independently —CF₃. In some embodiments, R³ is independently —CI₃. In some embodiments, R³ is independently —CHCl₂. In some embodiments, R³ is independently —CHBr₂. In some embodiments, R³ is independently —CHF₂. In some embodiments, R³ is independently —CHI₂. In some embodiments, R³ is independently —CH₂Cl. In some embodiments, R³ is independently —CH₂Br. In some embodiments, R³ is independently —CH₂F. In some embodiments, R³ is independently —CH₂I. In some embodiments, R³ is independently —CN. In some embodiments, R³ is independently —OH. In some embodiments, R³ is independently —NH₂. In some embodiments, R³ is independently —COOH. In some embodiments, R³ is independently —CONH₂. In some embodiments, R³ is independently —NO₂. In some embodiments, R³ is independently —SH. In some embodiments, R³ is independently —SO₃H. In some embodiments, R³ is independently —SO₄H. In some embodiments, R³ is independently —SO₂NH₂. In some embodiments, R³ is independently —NHNH₂. In some embodiments, R³ is independently —ONH₂. In some embodiments, R³ is independently —NHC(O)NHNH₂. In some embodiments, R³ is independently —NHC(O)NH₂. In some embodiments, R³ is independently —NHSO₂H. In some embodiments, R³ is independently —NHC(O)H. In some embodiments, R³ is independently —NHC(O)OH. In some embodiments, R³ is independently —NHOH. In some embodiments, R³ is independently —OCCl₃. In some embodiments, R³ is independently —OCF₃. In some embodiments, R³ is independently —OCBr₃. In some embodiments, R³ is independently —OCI₃. In some embodiments, R³ is independently —OCHCl₂. In some embodiments, R³ is independently —OCHBr₂. In some embodiments, R³ is independently —OCHI₂. In some embodiments, R³ is independently —OCHF₂. In some embodiments, R³ is independently —OCH₂Cl. In some embodiments, R³ is independently —OCH₂Br. In some embodiments, R³ is independently —OCH₂I. In some embodiments, R³ is independently —OCH₂F. In some embodiments, R³ is independently —N₃. In some embodiments, R³ is independently —OCH₃. In some embodiments, R³ is independently —CH₃. In some embodiments, R³ is independently —CH₂CH₃. In some embodiments, R³ is independently unsubstituted propyl. In some embodiments, R³ is independently unsubstituted isopropyl. In some embodiments, R³ is independently unsubstituted butyl. In some embodiments, R³ is independently unsubstituted tert-butyl. In some embodiments, R³ is independently —F. In some embodiments, R³ is independently —Cl. In some embodiments, R³ is independently —Br. In some embodiments, R³ is independently —I.

In some embodiments, R²⁷ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R²⁸-substituted or unsubstituted alkyl (e.g., C₁-C₈ alkyl, C₁-C₆ alkyl, or C₁-C₄ alkyl), R²⁸-substituted or unsubstituted heteroalkyl (e.g., 2 to 8 membered heteroalkyl, 2 to 6 membered heteroalkyl, or 2 to 4 membered heteroalkyl), R²⁸-substituted or unsubstituted cycloalkyl (e.g., C₃-C₈ cycloalkyl, C₃-C₆ cycloalkyl, or C₅-C₆ cycloalkyl), R²⁸-substituted or unsubstituted heterocycloalkyl (e.g., 3 to 8 membered heterocycloalkyl, 3 to 6 membered heterocycloalkyl, or 5 to 6 membered heterocycloalkyl), R²⁸-substituted or unsubstituted aryl (e.g., C₆-C₁₀ aryl, C₁₀ aryl, or phenyl), or R²⁸-substituted or unsubstituted heteroaryl (e.g., 5 to 10 membered heteroaryl, 5 to 9 membered heteroaryl, or 5 to 6 membered heteroaryl). In some embodiments, R²⁷ is independently oxo, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R²⁷ is independently oxo. In some embodiments, R²⁷ is independently halogen. In some embodiments, R²⁷ is independently —CCl₃. In some embodiments, R²⁷ is independently —CBr₃. In some embodiments, R²⁷ is independently —CF₃. In some embodiments, R²⁷ is independently —CI₃. In some embodiments, R²⁷ is independently —CHCl₂. In some embodiments, R²⁷ is independently —CHBr₂. In some embodiments, R²⁷ is independently —CHF₂. In some embodiments, R²⁷ is independently —CHI₂. In some embodiments, R²⁷ is independently —CH₂Cl. In some embodiments, R²⁷ is independently —CH₂Br. In some embodiments, R²⁷ is independently —CH₂F. In some embodiments, R²⁷ is independently —CH₂I. In some embodiments, R²⁷ is independently —CN. In some embodiments, R²⁷ is independently —OH. In some embodiments, R²⁷ is independently —NH₂. In some embodiments, R²⁷ is independently —COOH. In some embodiments, R²⁷ is independently —CONH₂. In some embodiments, R²⁷ is independently —NO₂. In some embodiments, R²⁷ is independently —SH. In some embodiments, R²⁷ is independently —SO₃H. In some embodiments, R²⁷ is independently —SO₄H. In some embodiments, R²⁷ is independently —SO₂NH₂. In some embodiments, R²⁷ is independently —NHNH₂. In some embodiments, R²⁷ is independently —ONH₂. In some embodiments, R²⁷ is independently —NHC(O)NHNH₂. In some embodiments, R²⁷ is independently —NHC(O)NH₂. In some embodiments, R²⁷ is independently —NHSO₂H. In some embodiments, R²⁷ is independently —NHC(O)H. In some embodiments, R²⁷ is independently —NHC(O)OH. In some embodiments, R²⁷ is independently —NHOH. In some embodiments, R²⁷ is independently —OCCl₃. In some embodiments, R²⁷ is independently —OCF₃. In some embodiments, R²⁷ is independently —OCBr₃. In some embodiments, R²⁷ is independently —OCI₃. In some embodiments, R²⁷ is independently —OCHCl₂. In some embodiments, R²⁷ is independently —OCHBr₂. In some embodiments, R²⁷ is independently —OCHI₂. In some embodiments, R²⁷ is independently —OCHF₂. In some embodiments, R²⁷ is independently —OCH₂Cl. In some embodiments, R²⁷ is independently —OCH₂Br. In some embodiments, R²⁷ is independently —OCH₂I. In some embodiments, R²⁷ is independently —OCH₂F. In some embodiments, R²⁷ is independently —N₃. In some embodiments, R²⁷ is independently —OCH₃. In some embodiments, R²⁷ is independently —CH₃. In some embodiments, R²⁷ is independently —CH₂CH₃. In some embodiments, R²⁷ is independently unsubstituted propyl. In some embodiments, R²⁷ is independently unsubstituted isopropyl. In some embodiments, R²⁷ is independently unsubstituted butyl. In some embodiments, R²⁷ is independently unsubstituted tert-butyl. In some embodiments, R²⁷ is independently —F. In some embodiments, R²⁷ is independently —Cl. In some embodiments, R²⁷ is independently —Br. In some embodiments, R²⁷ is independently —I.

In some embodiments, R²⁷ is independently R²⁸-substituted or unsubstituted alkyl (e.g., C₁-C₈ alkyl, C₁-C₆ alkyl, or C₁-C₄ alkyl). In some embodiments, R²⁷ is independently R²⁸-substituted alkyl (e.g., C₁-C₈ alkyl, C₁-C₆ alkyl, or C₁-C₄ alkyl). In some embodiments, R²⁷ is independently an unsubstituted alkyl (e.g., C₁-C₈ alkyl, C₁-C₆ alkyl, or C₁-C₄ alkyl). In some embodiments, R²⁷ is independently R²⁸-substituted or unsubstituted heteroalkyl (e.g., 2 to 8 membered heteroalkyl, 2 to 6 membered heteroalkyl, or 2 to 4 membered heteroalkyl). In some embodiments, R²⁷ is independently R²⁸-substituted heteroalkyl (e.g., 2 to 8 membered heteroalkyl, 2 to 6 membered heteroalkyl, or 2 to 4 membered heteroalkyl). In some embodiments, R²⁷ is independently an unsubstituted heteroalkyl (e.g., 2 to 8 membered heteroalkyl, 2 to 6 membered heteroalkyl, or 2 to 4 membered heteroalkyl). In some embodiments, R²⁷ is independently R²⁸-substituted or unsubstituted cycloalkyl (e.g., C₃-C₈ cycloalkyl, C₃-C₆ cycloalkyl, or C₅-C₆ cycloalkyl). In some embodiments, R²⁷ is independently R²⁸-substituted cycloalkyl (e.g., C₃-C₈ cycloalkyl, C₃-C₆ cycloalkyl, or C₅-C₆ cycloalkyl). In some embodiments, R²⁷ is independently an unsubstituted cycloalkyl (e.g., C₃-C₈ cycloalkyl, C₃-C₆ cycloalkyl, or C₅-C₆ cycloalkyl). In some embodiments, R²⁷ is independently R²⁸-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R²⁷ is independently R²⁸-substituted heterocycloalkyl (e.g., 3 to 8 membered heterocycloalkyl, 3 to 6 membered heterocycloalkyl, or 5 to 6 membered heterocycloalkyl). In some embodiments, R²⁷ is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R²⁷ is independently R²⁸-substituted or unsubstituted aryl (e.g., C₆-C₁₀ aryl, C₁₀ aryl, or phenyl). In some embodiments, R²⁷ is independently R²⁸-substituted aryl (e.g., C₆-C₁₀ aryl, C₁₀ aryl, or phenyl). In some embodiments, R²⁷ is independently an unsubstituted aryl (e.g., C₆-C₁₀ aryl, C₁₀ aryl, or phenyl). In some embodiments, R²⁷ is independently R²⁸-substituted or unsubstituted heteroaryl (e.g., 5 to 10 membered heteroaryl, 5 to 9 membered heteroaryl, or 5 to 6 membered heteroaryl). In some embodiments, R²⁷ is independently R²⁸-substituted heteroaryl (e.g., 5 to 10 membered heteroaryl, 5 to 9 membered heteroaryl, or 5 to 6 membered heteroaryl). In some embodiments, R²⁷ is independently an unsubstituted heteroaryl (e.g., 5 to 10 membered heteroaryl, 5 to 9 membered heteroaryl, or 5 to 6 membered heteroaryl).

In some embodiments, R²⁸ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R²⁹-substituted or unsubstituted alkyl (e.g., C₁-C₈ alkyl, C₁-C₆ alkyl, or C₁-C₄ alkyl), R²⁹-substituted or unsubstituted heteroalkyl (e.g., 2 to 8 membered heteroalkyl, 2 to 6 membered heteroalkyl, or 2 to 4 membered heteroalkyl), R²⁹-substituted or unsubstituted cycloalkyl (e.g., C₃-C₈ cycloalkyl, C₃-C₆ cycloalkyl, or C₅-C₆ cycloalkyl), R²⁹-substituted or unsubstituted heterocycloalkyl (e.g., 3 to 8 membered heterocycloalkyl, 3 to 6 membered heterocycloalkyl, or 5 to 6 membered heterocycloalkyl), R²⁹-substituted or unsubstituted aryl (e.g., C₆-C₁₀ aryl, C₁₀ aryl, or phenyl), or R²⁹-substituted or unsubstituted heteroaryl (e.g., 5 to 10 membered heteroaryl, 5 to 9 membered heteroaryl, or 5 to 6 membered heteroaryl). In some embodiments, R²⁸ is independently oxo, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R²⁸ is independently R²⁹-substituted or unsubstituted alkyl (e.g., C₁-C₈ alkyl, C₁-C₆ alkyl, or C₁-C₄ alkyl). In some embodiments, R²⁸ is independently R²⁹-substituted alkyl (e.g., C₁-C₈ alkyl, C₁-C₆ alkyl, or C₁-C₄ alkyl). In some embodiments, R²⁸ is independently an unsubstituted alkyl (e.g., C₁-C₈ alkyl, C₁-C₆ alkyl, or C₁-C₄ alkyl). In some embodiments, R²⁸ is independently R²⁹-substituted or unsubstituted heteroalkyl (e.g., 2 to 8 membered heteroalkyl, 2 to 6 membered heteroalkyl, or 2 to 4 membered heteroalkyl). In some embodiments, R²⁸ is independently R²⁹-substituted heteroalkyl (e.g., 2 to 8 membered heteroalkyl, 2 to 6 membered heteroalkyl, or 2 to 4 membered heteroalkyl). In some embodiments, R²⁸ is independently an unsubstituted heteroalkyl (e.g., 2 to 8 membered heteroalkyl, 2 to 6 membered heteroalkyl, or 2 to 4 membered heteroalkyl). In some embodiments, R²⁸ is independently R²⁹-substituted or unsubstituted cycloalkyl (e.g., C₃-C₈ cycloalkyl, C₃-C₆ cycloalkyl, or C₅-C₆ cycloalkyl). In some embodiments, R²⁸ is independently R²⁹-substituted cycloalkyl (e.g., C₃-C₈ cycloalkyl, C₃-C₆ cycloalkyl, or C₅-C₆ cycloalkyl). In some embodiments, R²⁸ is independently an unsubstituted cycloalkyl (e.g., C₃-C₈ cycloalkyl, C₃-C₆ cycloalkyl, or C₅-C₆ cycloalkyl). In some embodiments, R²⁸ is independently R²⁹-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R²⁸ is independently R²⁹-substituted heterocycloalkyl (e.g., 3 to 8 membered heterocycloalkyl, 3 to 6 membered heterocycloalkyl, or 5 to 6 membered heterocycloalkyl). In some embodiments, R²⁸ is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R²⁸ is independently R²⁹-substituted or unsubstituted aryl (e.g., C₆-C₁₀ aryl, C₁₀ aryl, or phenyl). In some embodiments, R²⁸ is independently R²⁹-substituted aryl (e.g., C₆-C₁₀ aryl, C₁₀ aryl, or phenyl). In some embodiments, R²⁸ is independently an unsubstituted aryl (e.g., C₆-C₁₀ aryl, C₁₀ aryl, or phenyl). In some embodiments, R²⁸ is independently R²⁹-substituted or unsubstituted heteroaryl (e.g., 5 to 10 membered heteroaryl, 5 to 9 membered heteroaryl, or 5 to 6 membered heteroaryl). In some embodiments, R²⁸ is independently R²⁹-substituted heteroaryl (e.g., 5 to 10 membered heteroaryl, 5 to 9 membered heteroaryl, or 5 to 6 membered heteroaryl). In some embodiments, R²⁸ is independently an unsubstituted heteroaryl (e.g., 5 to 10 membered heteroaryl, 5 to 9 membered heteroaryl, or 5 to 6 membered heteroaryl).

In some embodiments, R²⁸ is independently oxo. In some embodiments, R²⁸ is independently halogen. In some embodiments, R²⁸ is independently —CCl₃. In some embodiments, R²⁸ is independently —CBr₃. In some embodiments, R²⁸ is independently —CF₃. In some embodiments, R²⁸ is independently —CI₃. In some embodiments, R²⁸ is independently —CHCl₂. In some embodiments, R²⁸ is independently —CHBr₂. In some embodiments, R²⁸ is independently —CHF₂. In some embodiments, R²⁸ is independently —CHI₂. In some embodiments, R²⁸ is independently —CH₂Cl. In some embodiments, R²⁸ is independently —CH₂Br. In some embodiments, R²⁸ is independently —CH₂F. In some embodiments, R²⁸ is independently —CH₂I. In some embodiments, R²⁸ is independently —CN. In some embodiments, R²⁸ is independently —OH. In some embodiments, R²⁸ is independently —NH₂. In some embodiments, R²⁸ is independently —COOH. In some embodiments, R²⁸ is independently —CONH₂. In some embodiments, R²⁸ is independently —NO₂. In some embodiments, R²⁸ is independently —SH. In some embodiments, R²⁸ is independently —SO₃H. In some embodiments, R²⁸ is independently —SO₄H. In some embodiments, R²⁸ is independently —SO₂NH₂. In some embodiments, R²⁸ is independently —NHNH₂. In some embodiments, R²⁸ is independently —ONH₂. In some embodiments, R²⁸ is independently —NHC(O)NHNH₂. In some embodiments, R²⁸ is independently —NHC(O)NH₂. In some embodiments, R²⁸ is independently —NHSO₂H. In some embodiments, R²⁸ is independently —NHC(O)H. In some embodiments, R²⁸ is independently —NHC(O)OH. In some embodiments, R²⁸ is independently —NHOH. In some embodiments, R²⁸ is independently —OCCl₃. In some embodiments, R²⁸ is independently —OCF₃. In some embodiments, R²⁸ is independently —OCBr₃. In some embodiments, R²⁸ is independently —OCI₃. In some embodiments, R²⁸ is independently —OCHCl₂. In some embodiments, R²⁸ is independently —OCHBr₂. In some embodiments, R²⁸ is independently —OCHI₂. In some embodiments, R²⁸ is independently —OCHF₂. In some embodiments, R²⁸ is independently —OCH₂Cl. In some embodiments, R²⁸ is independently —OCH₂Br. In some embodiments, R²⁸ is independently —OCH₂I. In some embodiments, R²⁸ is independently —OCH₂F. In some embodiments, R²⁸ is independently —N₃. In some embodiments, R²⁸ is independently —OCH₃. In some embodiments, R²⁸ is independently —CH₃. In some embodiments, R²⁸ is independently —CH₂CH₃. In some embodiments, R²⁸ is independently unsubstituted propyl. In some embodiments, R²⁸ is independently unsubstituted isopropyl. In some embodiments, R²⁸ is independently unsubstituted butyl. In some embodiments, R²⁸ is independently unsubstituted tert-butyl. In some embodiments, R²⁸ is independently —F. In some embodiments, R²⁸ is independently —Cl. In some embodiments, R²⁸ is independently —Br. In some embodiments, R²⁸ is independently —I.

In some embodiments, R²⁹ is independently oxo, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R²⁹ is independently an unsubstituted alkyl (e.g., C₁-C₈ alkyl, C₁-C₆ alkyl, or C₁-C₄ alkyl). In some embodiments, R²⁹ is independently an unsubstituted heteroalkyl (e.g., 2 to 8 membered heteroalkyl, 2 to 6 membered heteroalkyl, or 2 to 4 membered heteroalkyl). In some embodiments, R²⁹ is independently an unsubstituted cycloalkyl (e.g., C₃-C₈ cycloalkyl, C₃-C₆ cycloalkyl, or C₅-C₆ cycloalkyl). In some embodiments, R²⁹ is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R²⁹ is independently an unsubstituted aryl (e.g., C₆-C₁₀ aryl, C₁₀ aryl, or phenyl). In some embodiments, R²⁹ is independently an unsubstituted heteroaryl (e.g., 5 to 10 membered heteroaryl, 5 to 9 membered heteroaryl, or 5 to 6 membered heteroaryl).

In some embodiments, R²⁹ is independently oxo. In some embodiments, R²⁹ is independently halogen. In some embodiments, R²⁹ is independently —CCl₃. In some embodiments, R²⁹ is independently —CBr₃. In some embodiments, R²⁹ is independently —CF₃. In some embodiments, R²⁹ is independently —CI₃. In some embodiments, R²⁹ is independently —CHCl₂. In some embodiments, R²⁹ is independently —CHBr₂. In some embodiments, R²⁹ is independently —CHF₂. In some embodiments, R²⁹ is independently —CHI₂. In some embodiments, R²⁹ is independently —CH₂Cl. In some embodiments, R²⁹ is independently —CH₂Br. In some embodiments, R²⁹ is independently —CH₂F. In some embodiments, R²⁹ is independently —CH₂I. In some embodiments, R²⁹ is independently —CN. In some embodiments, R²⁹ is independently —OH. In some embodiments, R²⁹ is independently —NH₂. In some embodiments, R²⁹ is independently —COOH. In some embodiments, R²⁹ is independently —CONH₂. In some embodiments, R²⁹ is independently —NO₂. In some embodiments, R²⁹ is independently —SH. In some embodiments, R²⁹ is independently —SO₃H. In some embodiments, R²⁹ is independently —SO₄H. In some embodiments, R²⁹ is independently —SO₂NH₂. In some embodiments, R²⁹ is independently —NHNH₂. In some embodiments, R²⁹ is independently —ONH₂. In some embodiments, R²⁹ is independently —NHC(O)NHNH₂. In some embodiments, R²⁹ is independently —NHC(O)NH₂. In some embodiments, R²⁹ is independently —NHSO₂H. In some embodiments, R²⁹ is independently —NHC(O)H. In some embodiments, R²⁹ is independently —NHC(O)OH. In some embodiments, R²⁹ is independently —NHOH. In some embodiments, R²⁹ is independently —OCCl₃. In some embodiments, R²⁹ is independently —OCF₃. In some embodiments, R²⁹ is independently —OCBr₃. In some embodiments, R²⁹ is independently —OCI₃. In some embodiments, R²⁹ is independently —OCHCl₂. In some embodiments, R²⁹ is independently —OCHBr₂. In some embodiments, R²⁹ is independently —OCHI₂. In some embodiments, R²⁹ is independently —OCHF₂. In some embodiments, R²⁹ is independently —OCH₂Cl. In some embodiments, R²⁹ is independently —OCH₂Br. In some embodiments, R²⁹ is independently —OCH₂I. In some embodiments, R²⁹ is independently —OCH₂F. In some embodiments, R²⁹ is independently —N₃. In some embodiments, R²⁹ is independently —OCH₃. In some embodiments, R²⁹ is independently —CH₃. In some embodiments, R²⁹ is independently —CH₂CH₃. In some embodiments, R²⁹ is independently unsubstituted propyl.

In some embodiments, R²⁹ is independently unsubstituted isopropyl. In some embodiments, R²⁹ is independently unsubstituted butyl. In some embodiments, R²⁹ is independently unsubstituted tert-butyl. In some embodiments, R²⁹ is independently —F. In some embodiments, R²⁹ is independently —Cl. In some embodiments, R²⁹ is independently —Br. In some embodiments, R²⁹ is independently —I.

In some embodiments, two (e.g., adjacent) R³ substituents are independently joined to form a substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). In some embodiments, two (e.g., adjacent) R³ substituents are independently joined to form a substituted or unsubstituted cycloalkyl (e.g., C₃-C₈ cycloalkyl, C₃-C₆ cycloalkyl, or C₅-C₆ cycloalkyl). In some embodiments, two (e.g., adjacent) R³ substituents are independently joined to form a substituted cycloalkyl (e.g., C₃-C₈ cycloalkyl, C₃-C₆ cycloalkyl, or C₅-C₆ cycloalkyl). In some embodiments, two (e.g., adjacent) R³ substituents are independently joined to form an unsubstituted cycloalkyl (e.g., C₃-C₈ cycloalkyl, C₃-C₆ cycloalkyl, or C₅-C₆ cycloalkyl). In some embodiments, two (e.g., adjacent) R³ substituents are independently joined to form a substituted or unsubstituted heterocycloalkyl (e.g., 3 to 8 membered heterocycloalkyl, 3 to 6 membered heterocycloalkyl, or 5 to 6 membered heterocycloalkyl). In some embodiments, two (e.g., adjacent) R³ substituents are independently joined to form a substituted heterocycloalkyl (e.g., 3 to 8 membered heterocycloalkyl, 3 to 6 membered heterocycloalkyl, or 5 to 6 membered heterocycloalkyl). In some embodiments, two (e.g., adjacent) R³ substituents are independently joined to form an unsubstituted heterocycloalkyl (e.g., 3 to 8 membered heterocycloalkyl, 3 to 6 membered heterocycloalkyl, or 5 to 6 membered heterocycloalkyl). In some embodiments, two (e.g., adjacent) R³ substituents are independently joined to form a substituted or unsubstituted aryl (e.g., C₆-C₁₀ aryl, C₁₀ aryl, or phenyl). In some embodiments, two (e.g., adjacent) R³ substituents are independently joined to form a substituted aryl (e.g., C₆-C₁₀ aryl, C₁₀ aryl, or phenyl). In some embodiments, two (e.g., adjacent) R³ substituents are independently joined to form an unsubstituted aryl (e.g., C₆-C₁₀ aryl, C₁₀ aryl, or phenyl). In some embodiments, two (e.g., adjacent) R³ substituents are independently joined to form a substituted or unsubstituted heteroaryl (e.g., 5 to 10 membered heteroaryl, 5 to 9 membered heteroaryl, or 5 to 6 membered heteroaryl). In some embodiments, two (e.g., adjacent) R³ substituents are independently joined to form a substituted heteroaryl (e.g., 5 to 10 membered heteroaryl, 5 to 9 membered heteroaryl, or 5 to 6 membered heteroaryl). In some embodiments, two (e.g., adjacent) R³ substituents are independently joined to form an unsubstituted heteroaryl (e.g., 5 to 10 membered heteroaryl, 5 to 9 membered heteroaryl, or 5 to 6 membered heteroaryl). In some embodiments, two (e.g., adjacent) R³ substituents are independently joined to form an R²⁷-substituted or unsubstituted cycloalkyl (e.g., C₃-C₈ cycloalkyl, C₃-C₆ cycloalkyl, or C₅-C₆ cycloalkyl). In some embodiments, two (e.g., adjacent) R³ substituents are independently joined to form an R²⁷-substituted cycloalkyl (e.g., C₃-C₈ cycloalkyl, C₃-C₆ cycloalkyl, or C₅-C₆ cycloalkyl). In some embodiments, two (e.g., adjacent) R³ substituents are independently joined to form an unsubstituted cycloalkyl (e.g., C₃-C₈ cycloalkyl, C₃-C₆ cycloalkyl, or C₅-C₆ cycloalkyl). In some embodiments, two (e.g., adjacent) R³ substituents are independently joined to form an R²⁷-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, two (e.g., adjacent) R³ substituents are independently joined to form an R²⁷-substituted heterocycloalkyl (e.g., 3 to 8 membered heterocycloalkyl, 3 to 6 membered heterocycloalkyl, or 5 to 6 membered heterocycloalkyl). In some embodiments, two (e.g., adjacent) R³ substituents are independently joined to form an unsubstituted heterocycloalkyl (e.g., 3 to 8 membered heterocycloalkyl, 3 to 6 membered heterocycloalkyl, or 5 to 6 membered heterocycloalkyl). In some embodiments, two (e.g., adjacent) R³ substituents are independently joined to form an R²⁷-substituted or unsubstituted aryl (e.g., C₆-C₁₀ aryl, C₁₀ aryl, or phenyl). In some embodiments, two (e.g., adjacent) R³ substituents are independently joined to form an R²⁷-substituted aryl (e.g., C₆-C₁₀ aryl, C₁₀ aryl, or phenyl). In some embodiments, two (e.g., adjacent) R³ substituents are independently joined to form an unsubstituted aryl (e.g., C₆-C₁₀ aryl, C₁₀ aryl, or phenyl). In some embodiments, two (e.g., adjacent) R³ substituents are independently joined to form an R²⁷-substituted or unsubstituted heteroaryl (e.g., 5 to 10 membered heteroaryl, 5 to 9 membered heteroaryl, or 5 to 6 membered heteroaryl). In some embodiments, two (e.g., adjacent) R³ substituents are independently joined to form an R²⁷-substituted heteroaryl (e.g., 5 to 10 membered heteroaryl, 5 to 9 membered heteroaryl, or 5 to 6 membered heteroaryl). In some embodiments, two (e.g., adjacent) R³ substituents are independently joined to form an unsubstituted heteroaryl (e.g., 5 to 10 membered heteroaryl, 5 to 9 membered heteroaryl, or 5 to 6 membered heteroaryl).

In some embodiments, the divalent linker bonded to both the monovalent cellular component binder and monovalent targeted autophagy protein binder is attached to the monovalent targeted autophagy protein binder at an R³ position. In some embodiments, when the divalent linker bonded to both the monovalent cellular component binder and monovalent targeted autophagy protein binder is attached to the monovalent targeted autophagy protein binder at an R³ position, R³ is replaced with a divalent linker, referred to in this embodiment as L^{R3}.

In some embodiments, L^{R3} is a bond, —S(O)₂—, —S(O)—, —NR^{1A}—, ═N—, —O—, —S—, —C(O)—, —C(O)NR^{1A}—, —NR^{1A}C(O)-, —NR^{1A}C(O)NH—, —NHC(O)NR^{1A}—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. It will be understood that when -L^{R3}- is ═N—, one of the two direct covalent connections to L^{R3} shown in “-L^{R3}-” is a double bond and L^{R3} may equivalently be shown as “=L^{R3}-” and the atom to which the double bond is directly attached must obey standard rules of chemical valency known in the chemical arts and be capable of forming such a double bond.

In some embodiments, L^{R3} is independently a bond, —S(O)₂—, —S(O)—, —NH—, —O—, —S—, —C(O)—, —C(O)NH—, —NHC(O)—, —NHC(O)NH—, —C(O)O—, —OC(O)—, substituted or unsubstituted alkylene (e.g., C₁-C₈ alkylene, C₁-C₆ alkylene, or C₁-C₄ alkylene), substituted or unsubstituted heteroalkylene (e.g., 2 to 8 membered heteroalkylene, 2 to 6 membered heteroalkylene, or 2 to 4 membered heteroalkylene), substituted or unsubstituted cycloalkylene (e.g., C₃-C₈ cycloalkylene, C₃-C₆ cycloalkylene, or C₅-C₆ cycloalkylene), substituted or unsubstituted heterocycloalkylene (e.g., 3 to 8 membered heterocycloalkylene, 3 to 6 membered heterocycloalkylene, or 5 to 6 membered heterocycloalkylene), substituted or unsubstituted arylene (e.g., C₆-C₁₀ arylene, C₁₀ arylene, or phenylene), or substituted or unsubstituted heteroarylene (e.g., 5 to 10 membered heteroarylene, 5 to 9 membered heteroarylene, or 5 to 6 membered heteroarylene). In some embodiments, L^{R3} is independently a —S(O)₂—. In some embodiments, L^{R3} is independently a —S(O)—. In some embodiments, L^{R3} is independently a —NH—. In some embodiments, L^{R3} is independently a —O—. In some embodiments, L^{R3} is independently a —S—. In some embodiments, L^{R3} is independently a —C(O)—. In some embodiments, L^{R3} is independently a —C(O)NH—. In some embodiments, L^{R3} is independently a —NHC(O)—. In some embodiments, L^{R3} is independently a —NHC(O)NH—. In some embodiments, L^{R3} is independently a —C(O)O—. In some embodiments, L^{R3} is independently —OC(O)—. In some embodiments, L^{R3} is independently —NR^{3A}—. In some embodiments, L^{R3} is independently —C(O)NR^{3A}—. In some embodiments, L^{R3} is independently —NR^{3A}C(O)—. In some embodiments, L^{R3} is independently —NR^{3A}C(O)NH—. In some embodiments, L^{R3} is independently —NHC(O)NR^{3A}—. In some embodiments, L^{R3} is independently a bond.

In some embodiments, L^{R3} is substituted or unsubstituted alkylene (e.g., C₁-C₈ alkylene, C₁-C₆ alkylene, or C₁-C₄ alkylene). In some embodiments, L^{R3} is substituted alkylene (e.g., C₁-C₈ alkylene, C₁-C₆ alkylene, or C₁-C₄ alkylene). In some embodiments, L^{R3} is an unsubstituted alkylene (e.g., C₁-C₈ alkylene, C₁-C₆ alkylene, or C₁-C₄ alkylene). In some embodiments, L^{R3} is substituted or unsubstituted heteroalkylene (e.g., 2 to 8 membered heteroalkylene, 2 to 6 membered heteroalkylene, or 2 to 4 membered heteroalkylene). In some embodiments, L^{R3} is substituted heteroalkylene (e.g., 2 to 8 membered heteroalkylene, 2 to 6 membered heteroalkylene, or 2 to 4 membered heteroalkylene). In some embodiments, L^{R3} is an unsubstituted heteroalkylene (e.g., 2 to 8 membered heteroalkylene, 2 to 6 membered heteroalkylene, or 2 to 4 membered heteroalkylene). In some embodiments, L^{R3} is substituted or unsubstituted cycloalkylene (e.g., C₃-C₈ cycloalkylene, C₃-C₆ cycloalkylene, or C₅-C₆ cycloalkylene). In some embodiments, L^{R1} is substituted cycloalkylene (e.g., C3-C8 cycloalkylene, C₃-C₆ cycloalkylene, or C₅-C₆ cycloalkylene). In some embodiments, L^{R1} is an unsubstituted cycloalkylene (e.g., C₃-C₈ cycloalkylene, C₃-C₆ cycloalkylene, or C₅-C₆ cycloalkylene). In some embodiments, L^{R3} is substituted or unsubstituted heterocycloalkylene (e.g., 3 to 8 membered heterocycloalkylene, 3 to 6 membered heterocycloalkylene, or 5 to 6 membered heterocycloalkylene). In some embodiments, L^{R3} is substituted heterocycloalkylene (e.g., 3 to 8 membered heterocycloalkylene, 3 to 6 membered heterocycloalkylene, or 5 to 6 membered heterocycloalkylene). In some embodiments, L^{R3} is an unsubstituted heterocycloalkylene (e.g., 3 to 8 membered heterocycloalkylene, 3 to 6 membered heterocycloalkylene, or 5 to 6 membered heterocycloalkylene). In some embodiments, L^{R3} is substituted or unsubstituted arylene (e.g., C₆-C₁₀ arylene, C₁₀ arylene, or phenylene). In some embodiments, L^{R3} is substituted arylene (e.g., C₆-C₁₀ arylene, C₁₀ arylene, or phenylene). In some embodiments, L^{R3} is an unsubstituted arylene (e.g., C₆-C₁₀ arylene, C₁₀ arylene, or phenylene). In some embodiments, L^{R3} is substituted or unsubstituted heteroarylene (e.g., 5 to 10 membered heteroarylene, 5 to 9 membered heteroarylene, or 5 to 6 membered heteroarylene). In some embodiments, L^{R3} is substituted heteroarylene (e.g., 5 to 10 membered heteroarylene, 5 to 9 membered heteroarylene, or 5 to 6 membered heteroarylene). In some embodiments, L^{R3} is an unsubstituted heteroarylene (e.g., 5 to 10 membered heteroarylene, 5 to 9 membered heteroarylene, or 5 to 6 membered heteroarylene).

In some embodiments, L^{R3} is independently a bond, —S(O)₂—, —S(O)—, —NH—, —O—, —S—, —C(O)—, —C(O)NH—, —NHC(O)—, —NHC(O)NH—, —C(O)O—, —OC(O)—, R²⁷-substituted or unsubstituted alkylene (e.g., C₁-C₈ alkylene, C₁-C₆ alkylene, or C₁-C₄ alkylene), R²⁷-substituted or unsubstituted heteroalkylene (e.g., 2 to 8 membered heteroalkylene, 2 to 6 membered heteroalkylene, or 2 to 4 membered heteroalkylene), R²⁷-substituted or unsubstituted cycloalkylene (e.g., C₃-C₈ cycloalkylene, C₃-C₆ cycloalkylene, or C₅-C₆ cycloalkylene), R²⁷-substituted or unsubstituted heterocycloalkylene (e.g., 3 to 8 membered heterocycloalkylene, 3 to 6 membered heterocycloalkylene, or 5 to 6 membered heterocycloalkylene), R²⁷-substituted or unsubstituted arylene (e.g., C₆-C₁₀ arylene, C₁₀ arylene, or phenylene), or R²⁷-substituted or unsubstituted heteroarylene (e.g., 5 to 10 membered heteroarylene, 5 to 9 membered heteroarylene, or 5 to 6 membered heteroarylene). In some embodiments, L^{R3} is independently a bond, —S(O)₂—, —S(O)—, —NH—, —O—, —S—, —C(O)—, —C(O)NH—, —NHC(O)—, —NHC(O)NH—, —C(O)O—, —OC(O)—, unsubstituted alkylene (e.g., C₁-C₈ alkylene, C₁-C₆ alkylene, or C₁-C₄ alkylene), unsubstituted heteroalkylene (e.g., 2 to 8 membered heteroalkylene, 2 to 6 membered heteroalkylene, or 2 to 4 membered heteroalkylene), unsubstituted cycloalkylene (e.g., C₃-C₈ cycloalkylene, C₃-C₆ cycloalkylene, or C₅-C₆ cycloalkylene), unsubstituted heterocycloalkylene (e.g., 3 to 8 membered heterocycloalkylene, 3 to 6 membered heterocycloalkylene, or 5 to 6 membered heterocycloalkylene), unsubstituted arylene (e.g., C₆-C₁₀ arylene, C₁₀ arylene, or phenylene), or unsubstituted heteroarylene (e.g., 5 to 10 membered heteroarylene, 5 to 9 membered heteroarylene, or 5 to 6 membered heteroarylene).

In some embodiments, L^{R3} is R²⁷-substituted or unsubstituted alkylene (e.g., C₁-C₈ alkylene, C₁-C₆ alkylene, or C₁-C₄ alkylene). In some embodiments, L^{R3} is R²⁷-substituted alkylene (e.g., C₁-C₈ alkylene, C₁-C₆ alkylene, or C₁-C₄ alkylene). In some embodiments, L^{R3} is an unsubstituted alkylene (e.g., C₁-C₈ alkylene, C₁-C₆ alkylene, or C₁-C₄ alkylene). In some embodiments, L^{R3} is R²⁷-substituted or unsubstituted heteroalkylene (e.g., 2 to 8 membered heteroalkylene, 2 to 6 membered heteroalkylene, or 2 to 4 membered heteroalkylene). In some embodiments, L^{R3} is R²⁷-substituted heteroalkylene (e.g., 2 to 8 membered heteroalkylene, 2 to 6 membered heteroalkylene, or 2 to 4 membered heteroalkylene). In some embodiments, L^{R3} is an unsubstituted heteroalkylene (e.g., 2 to 8 membered heteroalkylene, 2 to 6 membered heteroalkylene, or 2 to 4 membered heteroalkylene). In some embodiments, L^{R3} is R²⁷-substituted or unsubstituted cycloalkylene (e.g., C₃-C₈ cycloalkylene, C₃-C₆ cycloalkylene, or C₅-C₆ cycloalkylene). In some embodiments, L^{R3} is R²⁷-substituted cycloalkylene (e.g., C₃-C₈ cycloalkylene, C₃-C₆ cycloalkylene, or C₅-C₆ cycloalkylene). In some embodiments, L^{R3} is an unsubstituted cycloalkylene (e.g., C₃-C₈ cycloalkylene, C₃-C₆ cycloalkylene, or C₅-C₆ cycloalkylene). In some embodiments, L^{R3} is R²⁷-substituted or unsubstituted heterocycloalkylene (e.g., 3 to 8 membered heterocycloalkylene, 3 to 6 membered heterocycloalkylene, or 5 to 6 membered heterocycloalkylene). In some embodiments, L^{R3} is R²⁷-substituted heterocycloalkylene (e.g., 3 to 8 membered heterocycloalkylene, 3 to 6 membered heterocycloalkylene, or 5 to 6 membered heterocycloalkylene). In some embodiments, L^{R3} is an unsubstituted heterocycloalkylene (e.g., 3 to 8 membered heterocycloalkylene, 3 to 6 membered heterocycloalkylene, or 5 to 6 membered heterocycloalkylene). In some embodiments, L^{R3} is R²⁷-substituted or unsubstituted arylene (e.g., C₆-C₁₀ arylene, C₁₀ arylene, or phenylene). In some embodiments, L^{R3} is R²⁷-substituted arylene (e.g., C₆-C₁₀ arylene, C₁₀ arylene, or phenylene). In some embodiments, L^{R3} is an unsubstituted arylene (e.g., C₆-C₁₀ arylene, C₁₀ arylene, or phenylene). In some embodiments, L^{R3} is R²⁷-substituted or unsubstituted heteroarylene (e.g., 5 to 10 membered heteroarylene, 5 to 9 membered heteroarylene, or 5 to 6 membered heteroarylene). In some embodiments, L^{R3} is R²⁷-substituted heteroarylene (e.g., 5 to 10 membered heteroarylene, 5 to 9 membered heteroarylene, or 5 to 6 membered heteroarylene). In some embodiments, L^{R3} is an unsubstituted heteroarylene (e.g., 5 to 10 membered heteroarylene, 5 to 9 membered heteroarylene, or 5 to 6 membered heteroarylene).

In some embodiments, R^{3A} is independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In some embodiments, the divalent linker bonded to both the monovalent cellular component binder and monovalent targeted autophagy protein binder is attached to the monovalent targeted autophagy protein binder at an R^{3A} position. In some embodiments, when the divalent linker bonded to both the monovalent cellular component binder and monovalent targeted autophagy protein binder is attached to the monovalent targeted autophagy protein binder at an R^{3A} position, R^{3A} is replaced with a divalent linker, referred to in this embodiment as L^{R3}.

In some embodiments, R^{3B} is independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In some embodiments, the divalent linker bonded to both the monovalent cellular component binder and monovalent targeted autophagy protein binder is attached to the monovalent targeted autophagy protein binder at an R^{3B} position. In some embodiments, when the divalent linker bonded to both the monovalent cellular component binder and monovalent targeted autophagy protein binder is attached to the monovalent targeted autophagy protein binder at an R^{3B} position, R^{3B} is replaced with a divalent linker, referred to in this embodiment as L^{R3}.

In some embodiments, R^{3C} is independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In some embodiments, the divalent linker bonded to both the monovalent cellular component binder and monovalent targeted autophagy protein binder is attached to the monovalent targeted autophagy protein binder at an R^{3C} position. In some embodiments, when the divalent linker bonded to both the monovalent cellular component binder and monovalent targeted autophagy protein binder is attached to the monovalent targeted autophagy protein binder at an R^{3C} position, R^{3C} is replaced with a divalent linker, referred to in this embodiment as L^{R3}.

In some embodiments, R^{3D} is independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In some embodiments, the divalent linker bonded to both the monovalent cellular component binder and monovalent targeted autophagy protein binder is attached to the monovalent targeted autophagy protein binder at an R^{3D} position. In some embodiments, when the divalent linker bonded to both the monovalent cellular component binder and monovalent targeted autophagy protein binder is attached to the monovalent targeted autophagy protein binder at an R^{3D} position, R^{3D} is replaced with a divalent linker, referred to in this embodiment as L^{R3}.

In some embodiments, R^{3A} and R^{3B} substituents bonded to the same nitrogen atom are independently joined to form a substituted or unsubstituted heterocycloalkyl (e.g., 3 to 8 membered heterocycloalkyl, 3 to 6 membered heterocycloalkyl, or 5 to 6 membered heterocycloalkyl) or substituted or unsubstituted heteroaryl (e.g., 5 to 10 membered heteroaryl, 5 to 9 membered heteroaryl, or 5 to 6 membered heteroaryl). In some embodiments, R^{3A}and R^{3B} substituents bonded to the same nitrogen atom are independently joined to form a substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R^{3A} and R^{3B} substituents bonded to the same nitrogen atom are independently joined to form a substituted heterocycloalkyl (e.g., 3 to 8 membered heterocycloalkyl, 3 to 6 membered heterocycloalkyl, or 5 to 6 membered heterocycloalkyl). In some embodiments, R^{3A} and R^{3B} substituents bonded to the same nitrogen atom are independently joined to form an unsubstituted heterocycloalkyl (e.g., 3 to 8 membered heterocycloalkyl, 3 to 6 membered heterocycloalkyl, or 5 to 6 membered heterocycloalkyl). In some embodiments, R^{3A} and R^{3B} substituents bonded to the same nitrogen atom are independently joined to form a substituted or unsubstituted heteroaryl (e.g., 5 to 10 membered heteroaryl, 5 to 9 membered heteroaryl, or 5 to 6 membered heteroaryl). In some embodiments, R^{3A} and R^{3B} substituents bonded to the same nitrogen atom are independently joined to form a substituted heteroaryl (e.g., 5 to 10 membered heteroaryl, 5 to 9 membered heteroaryl, or 5 to 6 membered heteroaryl). In some embodiments, R^{3A} and R^{3B} substituents bonded to the same nitrogen atom are independently joined to form an unsubstituted heteroaryl (e.g., 5 to 10 membered heteroaryl, 5 to 9 membered heteroaryl, or 5 to 6 membered heteroaryl).

In some embodiments, R^{3A}, R^{3B}, R^{3C}, and R^{3D} are independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, R²⁷-substituted or unsubstituted alkyl (e.g., C₁-C₈ alkyl, C₁-C₆ alkyl, or C₁-C₄ alkyl), R²⁷-substituted or unsubstituted heteroalkyl (e.g., 2 to 8 membered heteroalkyl, 2 to 6 membered heteroalkyl, or 2 to 4 membered heteroalkyl), R²⁷-substituted or unsubstituted cycloalkyl (e.g., C₃-C₈ cycloalkyl, C₃-C₆ cycloalkyl, or C₅-C₆ cycloalkyl), R²⁷-substituted or unsubstituted heterocycloalkyl (e.g., 3 to 8 membered heterocycloalkyl, 3 to 6 membered heterocycloalkyl, or 5 to 6 membered heterocycloalkyl), R²⁷-substituted or unsubstituted aryl (e.g., C₆-C₁₀ aryl, C₁₀ aryl, or phenyl), or R²⁷-substituted or unsubstituted heteroaryl (e.g., 5 to 10 membered heteroaryl, 5 to 9 membered heteroaryl, or 5 to 6 membered heteroaryl). In some embodiments, R^{3A} and R^{3B} substituents bonded to the same nitrogen atom are independently joined to form an R²⁷-substituted or unsubstituted heterocycloalkyl (e.g., 3 to 8 membered heterocycloalkyl, 3 to 6 membered heterocycloalkyl, or 5 to 6 membered heterocycloalkyl) or R²⁷-substituted or unsubstituted heteroaryl (e.g., 5 to 10 membered heteroaryl, 5 to 9 membered heteroaryl, or 5 to 6 membered heteroaryl). In some embodiments, R^{3A} and R^{3B} substituents bonded to the same nitrogen atom are independently joined to form an R²⁷-substituted or unsubstituted heterocycloalkyl (e.g., 3 to 8 membered heterocycloalkyl, 3 to 6 membered heterocycloalkyl. In some embodiments, R^{3A} and R^{3B} substituents bonded to the same nitrogen atom are independently joined to form an R²⁷-substituted or unsubstituted heteroaryl (e.g., 5 to 10 membered heteroaryl, 5 to 9 membered heteroaryl, or 5 to 6 membered heteroaryl).

In some embodiments, R^{3A} is independently R²⁷-substituted or unsubstituted alkyl (e.g., C₁-C₈ alkyl, C₁-C₆ alkyl, or C₁-C₄ alkyl). In some embodiments, R^{3A} is independently R²⁷-substituted alkyl (e.g., C₁-C₈ alkyl, C₁-C₆ alkyl, or C₁-C₄ alkyl). In some embodiments, R^{3A} is independently an unsubstituted alkyl (e.g., C₁-C₈ alkyl, C₁-C₆ alkyl, or C₁-C₄ alkyl). In some embodiments, R^{3A} is independently R²⁷-substituted or unsubstituted heteroalkyl (e.g., 2 to 8 membered heteroalkyl, 2 to 6 membered heteroalkyl, or 2 to 4 membered heteroalkyl). In some embodiments, R^{3A} is independently R²⁷-substituted heteroalkyl (e.g., 2 to 8 membered heteroalkyl, 2 to 6 membered heteroalkyl, or 2 to 4 membered heteroalkyl). In some embodiments, R^{3A} is independently an unsubstituted heteroalkyl (e.g., 2 to 8 membered heteroalkyl, 2 to 6 membered heteroalkyl, or 2 to 4 membered heteroalkyl). In some embodiments, R^{3A} is independently R²⁷-substituted or unsubstituted cycloalkyl (e.g., C₃-C₈ cycloalkyl, C₃-C₆ cycloalkyl, or C₅-C₆ cycloalkyl). In some embodiments, R^{3A} is independently R²⁷-substituted cycloalkyl (e.g., C₃-C₈ cycloalkyl, C₃-C₆ cycloalkyl, or C₅-C₆ cycloalkyl). In some embodiments, R^{3A} is independently an unsubstituted cycloalkyl (e.g., C₃-C₈ cycloalkyl, C₃-C₆ cycloalkyl, or C₅-C₆ cycloalkyl). In some embodiments, R^{3A} is independently R²⁷-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R^{3A} is independently R²⁷-substituted heterocycloalkyl (e.g., 3 to 8 membered heterocycloalkyl, 3 to 6 membered heterocycloalkyl, or 5 to 6 membered heterocycloalkyl). In some embodiments, R^{3A} is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R^{3A} is independently R²⁷-substituted or unsubstituted aryl (e.g., C₆-C₁₀ aryl, C₁₀ aryl, or phenyl). In some embodiments, R^{3A} is independently R²⁷-substituted aryl (e.g., C₆-C₁₀ aryl, C₁₀ aryl, or phenyl). In some embodiments, R^{3A} is independently an unsubstituted aryl (e.g., C₆-C₁₀ aryl, C₁₀ aryl, or phenyl). In some embodiments, R^{3A} is independently R²⁷-substituted or unsubstituted heteroaryl (e.g., 5 to 10 membered heteroaryl, 5 to 9 membered heteroaryl, or 5 to 6 membered heteroaryl). In some embodiments, R^{3A} is independently R²⁷-substituted heteroaryl (e.g., 5 to 10 membered heteroaryl, 5 to 9 membered heteroaryl, or 5 to 6 membered heteroaryl). In some embodiments, R^{3A} is independently an unsubstituted heteroaryl (e.g., 5 to 10 membered heteroaryl, 5 to 9 membered heteroaryl, or 5 to 6 membered heteroaryl).

In some embodiments, R^{3A} is independently —CCl₃. In some embodiments, R^{3A} is independently —CBr₃. In some embodiments, R^{3A} is independently —CF₃. In some embodiments, R^{3A} is independently —CI₃. In some embodiments, R^{3A} is independently —CHCl₂. In some embodiments, R^{3A} is independently —CHBr₂. In some embodiments, R^{3A} is independently —CHF₂. In some embodiments, R^{3A} is independently —CHI₂. In some embodiments, R^{3A} is independently —CH₂Cl. In some embodiments, R^{3A} is independently —CH₂Br. In some embodiments, R^{3A} is independently —CH₂F. In some embodiments, R^{3A} is independently —CH₂I. In some embodiments, R^{3A} is independently —CN. In some embodiments, R^{3A} is independently —OH. In some embodiments, R^{3A} is independently —COOH. In some embodiments, R^{3A} is independently —CONH₂. In some embodiments, R^{3A} is independently —OCCl₃. In some embodiments, R^{3A} is independently —OCF₃. In some embodiments, R^{3A} is independently —OCBr₃. In some embodiments, R^{3A} is independently —OCI₃. In some embodiments, R^{3A} is independently —OCHCl₂. In some embodiments, R^{3A} is independently —OCHBr₂. In some embodiments, R^{3A} is independently —OCHI₂. In some embodiments, R^{3A} is independently —OCHF₂. In some embodiments, R^{3A} is independently —OCH₂Cl. In some embodiments, R^{3A} is independently —OCH₂Br. In some embodiments, R^{3A} is independently —OCH₂I. In some embodiments, R^{3A} is independently —OCH₂F. In some embodiments, R^{3A} is independently —OCH₃. In some embodiments, R^{3A} is independently —CH₃. In some embodiments, R^{3A} is independently —CH₂CH₃. In some embodiments, R^{3A} is independently unsubstituted propyl. In some embodiments, R^{3A} is independently unsubstituted isopropyl. In some embodiments, R^{3A} is independently unsubstituted butyl. In some embodiments, R^{3A} is independently unsubstituted tert-butyl. In some embodiments, R^{3A} is independently hydrogen.

In some embodiments, R^{3B} is independently R²⁷-substituted or unsubstituted alkyl (e.g., C₁-C₈ alkyl, C₁-C₆ alkyl, or C₁-C₄ alkyl). In some embodiments, R^{3B} is independently R²⁷-substituted alkyl (e.g., C₁-C₈ alkyl, C₁-C₆ alkyl, or C₁-C₄ alkyl). In some embodiments, R^{3B} is independently an unsubstituted alkyl (e.g., C₁-C₈ alkyl, C₁-C₆ alkyl, or C₁-C₄ alkyl). In some embodiments, R^{3B} is independently R²⁷-substituted or unsubstituted heteroalkyl (e.g., 2 to 8 membered heteroalkyl, 2 to 6 membered heteroalkyl, or 2 to 4 membered heteroalkyl). In some embodiments, R^{3B} is independently R²⁷-substituted heteroalkyl (e.g., 2 to 8 membered heteroalkyl, 2 to 6 membered heteroalkyl, or 2 to 4 membered heteroalkyl). In some embodiments, R^{3B} is independently an unsubstituted heteroalkyl (e.g., 2 to 8 membered heteroalkyl, 2 to 6 membered heteroalkyl, or 2 to 4 membered heteroalkyl). In some embodiments, R^{3B} is independently R²⁷-substituted or unsubstituted cycloalkyl (e.g., C₃-C₈ cycloalkyl, C₃-C₆ cycloalkyl, or C₅-C₆ cycloalkyl). In some embodiments, R^{3B} is independently R²⁷-substituted cycloalkyl (e.g., C₃-C₈ cycloalkyl, C₃-C₆ cycloalkyl, or C₅-C₆ cycloalkyl). In some embodiments, R^{3B} is independently an unsubstituted cycloalkyl (e.g., C₃-C₈ cycloalkyl, C₃-C₆ cycloalkyl, or C₅-C₆ cycloalkyl). In some embodiments, R^{3B} is independently R²⁷-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R^{3B} is independently R²⁷-substituted heterocycloalkyl (e.g., 3 to 8 membered heterocycloalkyl, 3 to 6 membered heterocycloalkyl, or 5 to 6 membered heterocycloalkyl). In some embodiments, R^{3B} is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R^{3B} is independently R²⁷-substituted or unsubstituted aryl (e.g., C₆-C₁₀ aryl, C₁₀ aryl, or phenyl). In some embodiments, R^{3B} is independently R²⁷-substituted aryl (e.g., C₆-C₁₀ aryl, C₁₀ aryl, or phenyl). In some embodiments, R^{3B} is independently an unsubstituted aryl (e.g., C₆-C₁₀ aryl, C₁₀ aryl, or phenyl). In some embodiments, R^{3B} is independently R²⁷-substituted or unsubstituted heteroaryl (e.g., 5 to 10 membered heteroaryl, 5 to 9 membered heteroaryl, or 5 to 6 membered heteroaryl). In some embodiments, R^{3B} is independently R²⁷-substituted heteroaryl (e.g., 5 to 10 membered heteroaryl, 5 to 9 membered heteroaryl, or 5 to 6 membered heteroaryl). In some embodiments, R^{3B} is independently an unsubstituted heteroaryl (e.g., 5 to 10 membered heteroaryl, 5 to 9 membered heteroaryl, or 5 to 6 membered heteroaryl).

In some embodiments, R^{3B} is independently —CCl₃. In some embodiments, R^{3B} is independently —CBr₃. In some embodiments, R^{3B} is independently —CF₃. In some embodiments, R^{3B} is independently —CI₃. In some embodiments, R^{3B} is independently —CHCl₂. In some embodiments, R^{3B} is independently —CHBr₂. In some embodiments, R^{3B} is independently —CHF₂. In some embodiments, R^{3B} is independently —CHI₂. In some embodiments, R^{3B} is independently —CH₂Cl. In some embodiments, R^{3B} is independently —CH₂Br. In some embodiments, R^{3B} is independently —CH₂F. In some embodiments, R^{3B} is independently —CH₂I. In some embodiments, R^{3B} is independently —CN. In some embodiments, R^{3B} is independently —OH. In some embodiments, R^{3B} is independently —COOH. In some embodiments, R^{3B} is independently —CONH₂. In some embodiments, R^{3B} is independently —OCCl₃. In some embodiments, R^{3B} is independently —OCF₃. In some embodiments, R^{3B} is independently —OCBr₃. In some embodiments, R^{3B} is independently —OCI₃. In some embodiments, R^{3B} is independently —OCHCl₂. In some embodiments, R^{3B} is independently —OCHBr₂. In some embodiments, R^{3B} is independently —OCHI₂. In some embodiments, R^{3B} is independently —OCHF₂. In some embodiments, R^{3B} is independently —OCH₂Cl. In some embodiments, R^{3B} is independently —OCH₂Br. In some embodiments, R^{3B} is independently —OCH₂I. In some embodiments, R^{3B} is independently —OCH₂F. In some embodiments, R^{3B} is independently —OCH₃. In some embodiments, R^{3B} is independently —CH₃. In some embodiments, R^{3B} is independently —CH₂CH₃. In some embodiments, R^{3B} is independently unsubstituted propyl. In some embodiments, R^{3B} is independently unsubstituted isopropyl. In some embodiments, R^{3B} is independently unsubstituted butyl. In some embodiments, R^{3B} is independently unsubstituted tert-butyl. In some embodiments, R^{3B} is independently hydrogen.

In some embodiments, R^{3C} is independently R²⁷-substituted or unsubstituted alkyl (e.g., C₁-C₈ alkyl, C₁-C₆ alkyl, or C₁-C₄ alkyl). In some embodiments, R^{3C} is independently R²⁷-substituted alkyl (e.g., C₁-C₈ alkyl, C₁-C₆ alkyl, or C₁-C₄ alkyl). In some embodiments, R^{3C} is independently an unsubstituted alkyl (e.g., C₁-C₈ alkyl, C₁-C₆ alkyl, or C₁-C₄ alkyl). In some embodiments, R^{3C} is independently R²⁷-substituted or unsubstituted heteroalkyl (e.g., 2 to 8 membered heteroalkyl, 2 to 6 membered heteroalkyl, or 2 to 4 membered heteroalkyl). In some embodiments, R^{3C} is independently R²⁷-substituted heteroalkyl (e.g., 2 to 8 membered heteroalkyl, 2 to 6 membered heteroalkyl, or 2 to 4 membered heteroalkyl). In some embodiments, R^{3C} is independently an unsubstituted heteroalkyl (e.g., 2 to 8 membered heteroalkyl, 2 to 6 membered heteroalkyl, or 2 to 4 membered heteroalkyl). In some embodiments, R^{3C} is independently R²⁷-substituted or unsubstituted cycloalkyl (e.g., C₃-C₈ cycloalkyl, C₃-C₆ cycloalkyl, or C₅-C₆ cycloalkyl). In some embodiments, R^{3C} is independently R²⁷-substituted cycloalkyl (e.g., C₃-C₈ cycloalkyl, C₃-C₆ cycloalkyl, or C₅-C₆ cycloalkyl). In some embodiments, R^{3C} is independently an unsubstituted cycloalkyl (e.g., C₃-C₈ cycloalkyl, C₃-C₆ cycloalkyl, or C₅-C₆ cycloalkyl). In some embodiments, R^{3C} is independently R²⁷-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R^{3C} is independently R²⁷-substituted heterocycloalkyl (e.g., 3 to 8 membered heterocycloalkyl, 3 to 6 membered heterocycloalkyl, or 5 to 6 membered heterocycloalkyl). In some embodiments, R^{3C} is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R^{3C} is independently R²⁷-substituted or unsubstituted aryl (e.g., C₆-C₁₀ aryl, C₁₀ aryl, or phenyl). In some embodiments, R^{3C} is independently R²⁷-substituted aryl (e.g., C₆-C₁₀ aryl, C₁₀ aryl, or phenyl). In some embodiments, R^{3C} is independently an unsubstituted aryl (e.g., C₆-C₁₀ aryl, C₁₀ aryl, or phenyl). In some embodiments, R^{3C} is independently R²⁷-substituted or unsubstituted heteroaryl (e.g., 5 to 10 membered heteroaryl, 5 to 9 membered heteroaryl, or 5 to 6 membered heteroaryl). In some embodiments, R^{3C} is independently R²⁷-substituted heteroaryl (e.g., 5 to 10 membered heteroaryl, 5 to 9 membered heteroaryl, or 5 to 6 membered heteroaryl). In some embodiments, R^{3C} is independently an unsubstituted heteroaryl (e.g., 5 to 10 membered heteroaryl, 5 to 9 membered heteroaryl, or 5 to 6 membered heteroaryl).

In some embodiments, R^{3C} is independently —CCl₃. In some embodiments, R^{3C} is independently —CBr₃. In some embodiments, R^{3C} is independently —CF₃. In some embodiments, R^{3C} is independently —CI₃. In some embodiments, R^{3C} is independently —CHCl₂. In some embodiments, R^{3C} is independently —CHBr₂. In some embodiments, R^{3C} is independently —CHF₂. In some embodiments, R^{3C} is independently —CHI₂. In some embodiments, R^{3C} is independently —CH₂Cl. In some embodiments, R^{3C} is independently —CH₂Br. In some embodiments, R^{3C} is independently —CH₂F. In some embodiments, R^{3C} is independently —CH₂I. In some embodiments, R^{3C} is independently —CN. In some embodiments, R^{3C} is independently —OH. In some embodiments, R^{3C} is independently —COOH. In some embodiments, R^{3C} is independently —CONH₂. In some embodiments, R^{3C} is independently —OCCl₃. In some embodiments, R^{3C} is independently —OCF₃. In some embodiments, R^{3C} is independently —OCBr₃. In some embodiments, R^{3C} is independently —OCI₃. In some embodiments, R^{3C} is independently —OCHCl₂. In some embodiments, R^{3C} is independently —OCHBr₂. In some embodiments, R^{3C} is independently —OCHI₂. In some embodiments, R^{3C} is independently —OCHF₂. In some embodiments, R^{3C} is independently —OCH₂Cl. In some embodiments, R^{3C} is independently —OCH₂Br. In some embodiments, R^{3C} is independently —OCH₂I. In some embodiments, R^{3C} is independently —OCH₂F. In some embodiments, R^{3C} is independently —OCH₃. In some embodiments, R^{3C} is independently —CH₃. In some embodiments, R^{3C} is independently —CH₂CH₃. In some embodiments, R^{3C} is independently unsubstituted propyl. In some embodiments, R^{3C} is independently unsubstituted isopropyl. In some embodiments, R^{3C} is independently unsubstituted butyl. In some embodiments, R^{3C} is independently unsubstituted tert-butyl. In some embodiments, R^{3C} is independently hydrogen.

In some embodiments, R^{3D} is independently R²⁷-substituted or unsubstituted alkyl (e.g., C₁-C₈ alkyl, C₁-C₆ alkyl, or C₁-C₄ alkyl). In some embodiments, R^{3D} is independently R²⁷-substituted alkyl (e.g., C₁-C₈ alkyl, C₁-C₆ alkyl, or C₁-C₄ alkyl). In some embodiments, R^{3D} is independently an unsubstituted alkyl (e.g., C₁-C₈ alkyl, C₁-C₆ alkyl, or C₁-C₄ alkyl). In some embodiments, R^{3D} is independently R²⁷-substituted or unsubstituted heteroalkyl (e.g., 2 to 8 membered heteroalkyl, 2 to 6 membered heteroalkyl, or 2 to 4 membered heteroalkyl). In some embodiments, R^{3D} is independently R²⁷-substituted heteroalkyl (e.g., 2 to 8 membered heteroalkyl, 2 to 6 membered heteroalkyl, or 2 to 4 membered heteroalkyl). In some embodiments, R^{3D} is independently an unsubstituted heteroalkyl (e.g., 2 to 8 membered heteroalkyl, 2 to 6 membered heteroalkyl, or 2 to 4 membered heteroalkyl). In some embodiments, R^{3D} is independently R²⁷-substituted or unsubstituted cycloalkyl (e.g., C₃-C₈ cycloalkyl, C₃-C₆ cycloalkyl, or C₅-C₆ cycloalkyl). In some embodiments, R^{3D} is independently R²⁷-substituted cycloalkyl (e.g., C₃-C₈ cycloalkyl, C₃-C₆ cycloalkyl, or C₅-C₆ cycloalkyl). In some embodiments, R^{3D} is independently an unsubstituted cycloalkyl (e.g., C₃-C₈ cycloalkyl, C₃-C₆ cycloalkyl, or C₅-C₆ cycloalkyl). In some embodiments, R^{3D} is independently R²⁷-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R^{3D} is independently R²⁷-substituted heterocycloalkyl (e.g., 3 to 8 membered heterocycloalkyl, 3 to 6 membered heterocycloalkyl, or 5 to 6 membered heterocycloalkyl). In some embodiments, R^{3D} is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R^{3D} is independently p27-substituted or unsubstituted aryl (e.g., C₆-C₁₀ aryl, C₁₀ aryl, or phenyl). In some embodiments, R^{3D} is independently R²⁷-substituted aryl (e.g., C₆-C₁₀ aryl, C₁₀ aryl, or phenyl). In some embodiments, R^{3D} is independently an unsubstituted aryl (e.g., C₆-C₁₀ aryl, C₁₀ aryl, or phenyl). In some embodiments, R^{3D} is independently R²⁷-substituted or unsubstituted heteroaryl (e.g., 5 to 10 membered heteroaryl, 5 to 9 membered heteroaryl, or 5 to 6 membered heteroaryl). In some embodiments, R^{3D} is independently R²⁷-substituted heteroaryl (e.g., 5 to 10 membered heteroaryl, 5 to 9 membered heteroaryl, or 5 to 6 membered heteroaryl). In some embodiments, R^{3D} is independently an unsubstituted heteroaryl (e.g., 5 to 10 membered heteroaryl, 5 to 9 membered heteroaryl, or 5 to 6 membered heteroaryl).

In some embodiments, R^{3D} is independently —CCl₃. In some embodiments, R^{3D} is independently —CBr₃. In some embodiments, R^{3D} is independently —CF₃. In some embodiments, R^{3D} is independently —CI₃. In some embodiments, R^{3D} is independently —CHCl₂. In some embodiments, R^{3D} is independently —CHBr₂. In some embodiments, R^{3D} is independently —CHF₂. In some embodiments, R^{3D} is independently —CHI₂. In some embodiments, R^{3D} is independently —CH₂Cl. In some embodiments, R^{3D} is independently —CH₂Br. In some embodiments, R^{3D} is independently —CH₂F. In some embodiments, R^{3D} is independently —CH₂I. In some embodiments, R^{3D} is independently —CN. In some embodiments, R^{3D} is independently —OH. In some embodiments, R^{3D} is independently —COOH. In some embodiments, R^{3D} is independently —CONH₂. In some embodiments, R^{3D} is independently —OCCl₃. In some embodiments, R^{3D} is independently —OCF₃. In some embodiments, R^{3D} is independently —OCBr₃. In some embodiments, R^{3D} is independently —OCI₃. In some embodiments, R^{3D} is independently —OCHCl₂. In some embodiments, R^{3D} is independently —OCHBr₂. In some embodiments, R^{3D} is independently —OCHI₂. In some embodiments, R^{3D} is independently —OCHF₂. In some embodiments, R^{3D} is independently —OCH₂Cl. In some embodiments, R^{3D} is independently —OCH₂Br. In some embodiments, R^{3D} is independently —OCH₂I. In some embodiments, R^{3D} is independently —OCH₂F. In some embodiments, R^{3D} is independently —OCH₃. In some embodiments, R^{3D} is independently —CH₃. In some embodiments, R^{3D} is independently —CH₂CH₃. In some embodiments, R^{3D} is independently unsubstituted propyl. In some embodiments, R^{3D} is independently unsubstituted isopropyl. In some embodiments, R^{3D} is independently unsubstituted butyl. In some embodiments, R^{3D} is independently unsubstituted tert-butyl. In some embodiments, R^{3D} is independently hydrogen.

In some embodiments, R⁴ is

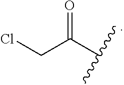 [SMILES: *C(=O)CCl]

In some embodiments, R⁵ is independently hydrogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —COOH, —CONH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In some embodiments, R⁵ is independently substituted or unsubstituted alkyl (e.g., C₁-C₈ alkyl, C₁-C₆ alkyl, or C₁-C₄ alkyl). In some embodiments, R⁵ is independently substituted alkyl (e.g., C₁-C₈ alkyl, C₁-C₆ alkyl, or C₁-C₄ alkyl). In some embodiments, R⁵ is independently an unsubstituted alkyl (e.g., C₁-C₈ alkyl, C₁-C₆ alkyl, or C₁-C₄ alkyl). In some embodiments, R⁵ is independently substituted or unsubstituted heteroalkyl (e.g., 2 to 8 membered heteroalkyl, 2 to 6 membered heteroalkyl, or 2 to 4 membered heteroalkyl). In some embodiments, R⁵ is independently substituted heteroalkyl (e.g., 2 to 8 membered heteroalkyl, 2 to 6 membered heteroalkyl, or 2 to 4 membered heteroalkyl). In some embodiments, R⁵ is independently an unsubstituted heteroalkyl (e.g., 2 to 8 membered heteroalkyl, 2 to 6 membered heteroalkyl, or 2 to 4 membered heteroalkyl). In some embodiments, R⁵ is independently substituted or unsubstituted cycloalkyl (e.g., C₃-C₈ cycloalkyl, C₃-C₆ cycloalkyl, or C₅-C₆ cycloalkyl). In some embodiments, R⁵ is independently substituted cycloalkyl (e.g., C₃-C₈ cycloalkyl, C₃-C₆ cycloalkyl, or C₅-C₆ cycloalkyl). In some embodiments, R⁵ is independently an unsubstituted cycloalkyl (e.g., C3-C₈ cycloalkyl, C₃-C₆ cycloalkyl, or C₅-C₆ cycloalkyl). In some embodiments, R⁵ is independently substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R⁵ is independently substituted heterocycloalkyl (e.g., 3 to 8 membered heterocycloalkyl, 3 to 6 membered heterocycloalkyl, or 5 to 6 membered heterocycloalkyl). In some embodiments, R⁵ is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R⁵ is independently substituted or unsubstituted aryl (e.g., C₆-C₁₀ aryl, C₁₀ aryl, or phenyl). In some embodiments, R⁵ is independently substituted aryl (e.g., C₆-C₁₀ aryl, C₁₀ aryl, or phenyl). In some embodiments, R⁵ is independently an unsubstituted aryl (e.g., C₆-C₁₀ aryl, C₁₀ aryl, or phenyl). In some embodiments, R⁵ is independently substituted or unsubstituted heteroaryl (e.g., 5 to 10 membered heteroaryl, 5 to 9 membered heteroaryl, or 5 to 6 membered heteroaryl). In some embodiments, R⁵ is independently substituted heteroaryl (e.g., 5 to 10 membered heteroaryl, 5 to 9 membered heteroaryl, or 5 to 6 membered heteroaryl). In some embodiments, R⁵ is independently an unsubstituted heteroaryl (e.g., 5 to 10 membered heteroaryl, 5 to 9 membered heteroaryl, or 5 to 6 membered heteroaryl).

In some embodiments, R⁵ is independently hydrogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —COOH, —CONH₂, R³³-substituted or unsubstituted alkyl (e.g., C₁-C₈ alkyl, C₁-C₆ alkyl, or C₁-C₄ alkyl), R³³-substituted or unsubstituted heteroalkyl (e.g., 2 to 8 membered heteroalkyl, 2 to 6 membered heteroalkyl, or 2 to 4 membered heteroalkyl), R³³-substituted or unsubstituted cycloalkyl (e.g., C₃-C₈ cycloalkyl, C₃-C₆ cycloalkyl, or C₅-C₆ cycloalkyl), R³³-substituted or unsubstituted heterocycloalkyl (e.g., 3 to 8 membered heterocycloalkyl, 3 to 6 membered heterocycloalkyl, or 5 to 6 membered heterocycloalkyl), R³³-substituted or unsubstituted aryl (e.g., C₆-C₁₀ aryl, C₁₀ aryl, or phenyl), or R³³-substituted or unsubstituted heteroaryl (e.g., 5 to 10 membered heteroaryl, 5 to 9 membered heteroaryl, or 5 to 6 membered heteroaryl). In some embodiments, R⁵ is independently hydrogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —COOH, —CONH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R⁵ is independently R³³-substituted or unsubstituted alkyl (e.g., C₁-C₈ alkyl, C₁-C₆ alkyl, or C₁-C₄ alkyl). In some embodiments, R⁵ is independently R³³-substituted alkyl (e.g., C₁-C₈ alkyl, C₁-C₆ alkyl, or C₁-C₄ alkyl). In some embodiments, R⁵ is independently an unsubstituted alkyl (e.g., C₁-C₈ alkyl, C₁-C₆ alkyl, or C₁-C₄ alkyl). In some embodiments, R⁵ is independently R³³-substituted or unsubstituted heteroalkyl (e.g., 2 to 8 membered heteroalkyl, 2 to 6 membered heteroalkyl, or 2 to 4 membered heteroalkyl). In some embodiments, R⁵ is independently R³³-substituted heteroalkyl (e.g., 2 to 8 membered heteroalkyl, 2 to 6 membered heteroalkyl, or 2 to 4 membered heteroalkyl). In some embodiments, R⁵ is independently an unsubstituted heteroalkyl (e.g., 2 to 8 membered heteroalkyl, 2 to 6 membered heteroalkyl, or 2 to 4 membered heteroalkyl). In some embodiments, R⁵ is independently R³³-substituted or unsubstituted cycloalkyl (e.g., C₃-C₈ cycloalkyl, C₃-C₆ cycloalkyl, or C₅-C₆ cycloalkyl). In some embodiments, R⁵ is independently R³³-substituted cycloalkyl (e.g., C₃-C₈ cycloalkyl, C₃-C₆ cycloalkyl, or C₅-C₆ cycloalkyl). In some embodiments, R⁵ is independently an unsubstituted cycloalkyl (e.g., C3-C₈ cycloalkyl, C₃-C₆ cycloalkyl, or C₅-C₆ cycloalkyl). In some embodiments, R⁵ is independently R³³-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R⁵ is independently R³³-substituted heterocycloalkyl (e.g., 3 to 8 membered heterocycloalkyl, 3 to 6 membered heterocycloalkyl, or 5 to 6 membered heterocycloalkyl). In some embodiments, R⁵ is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R⁵ is independently R³³-substituted or unsubstituted aryl (e.g., C₆-C₁₀ aryl, C₁₀ aryl, or phenyl). In some embodiments, R⁵ is independently R³³-substituted aryl (e.g., C₆-C₁₀ aryl, C₁₀ aryl, or phenyl). In some embodiments, R⁵ is independently an unsubstituted aryl (e.g., C₆-C₁₀ aryl, C₁₀ aryl, or phenyl). In some embodiments, R⁵ is independently R³³-substituted or unsubstituted heteroaryl (e.g., 5 to 10 membered heteroaryl, 5 to 9 membered heteroaryl, or 5 to 6 membered heteroaryl). In some embodiments, R⁵ is independently R³³-substituted heteroaryl (e.g., 5 to 10 membered heteroaryl, 5 to 9 membered heteroaryl, or 5 to 6 membered heteroaryl). In some embodiments, R⁵ is independently an unsubstituted heteroaryl (e.g., 5 to 10 membered heteroaryl, 5 to 9 membered heteroaryl, or 5 to 6 membered heteroaryl).

In some embodiments, R⁵ is independently —CX⁵₃. In some embodiments, R⁵ is independently —CHX⁵₂. In some embodiments, R⁵ is independently —CH₂X⁵. In some embodiments, R⁵ is independently —OCX⁵₃. In some embodiments, R⁵ is independently —OCH₂X⁵. In some embodiments, R⁵ is independently —OCHX⁵₂. In some embodiments, R⁵ is independently —CN. In some embodiments, R⁵ is independently —C(O)R^{5C}. In some embodiments, R⁵ is independently —C(O)—OR^{5C}. In some embodiments, R⁵ is independently —C(O)NR^{5A}R^{5B}. In some embodiments, R⁵ is independently —OR^{5D}. In some embodiments, R⁵ is independently hydrogen. X⁵ is independently halogen.

In some embodiments, R⁵ is independently —CCl₃. In some embodiments, R⁵ is independently —CBr₃. In some embodiments, R⁵ is independently —CF₃. In some embodiments, R⁵ is independently —CI₃. In some embodiments, R⁵ is independently —CHCl₂. In some embodiments, R⁵ is independently —CHBr₂. In some embodiments, R⁵ is independently —CHF₂. In some embodiments, R⁵ is independently —CHI₂. In some embodiments, R⁵ is independently —CH₂Cl. In some embodiments, R⁵ is independently —CH₂Br. In some embodiments, R⁵ is independently —CH₂F. In some embodiments, R⁵ is independently —CH₂I. In some embodiments, R⁵ is independently —CN. In some embodiments, R⁵ is independently —OH. In some embodiments, R⁵ is independently —COOH. In some embodiments, R⁵ is independently —CONH₂. In some embodiments, R⁵ is independently —OCCl₃. In some embodiments, R⁵ is independently —OCF₃. In some embodiments, R⁵ is independently —OCBr₃. In some embodiments, R⁵ is independently —OCI₃. In some embodiments, R⁵ is independently —OCHCl₂. In some embodiments, R⁵ is independently —OCHBr₂. In some embodiments, R⁵ is independently —OCHI₂. In some embodiments, R⁵ is independently —OCHF₂. In some embodiments, R⁵ is independently —OCH₂Cl. In some embodiments, R⁵ is independently —OCH₂Br. In some embodiments, R⁵ is independently —OCH₂I. In some embodiments, R⁵ is independently —OCH₂F. In some embodiments, R⁵ is independently —OCH₃. In some embodiments, R⁵ is independently —CH₃. In some embodiments, R⁵ is independently —CH₂CH₃. In some embodiments, R⁵ is independently unsubstituted propyl. In some embodiments, R⁵ is independently unsubstituted isopropyl. In some embodiments, R⁵ is independently unsubstituted butyl. In some embodiments, R⁵ is independently unsubstituted tert-butyl. In some embodiments, X⁵ is independently —F. In some embodiments, X⁵ is independently —Cl. In some embodiments, X⁵ is independently —Br. In some embodiments, X⁵ is independently —I.

In some embodiments, R³³ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R³⁴-substituted or unsubstituted alkyl (e.g., C₁-C₈ alkyl, C₁-C₆ alkyl, or C₁-C₄ alkyl), R³⁴-substituted or unsubstituted heteroalkyl (e.g., 2 to 8 membered heteroalkyl, 2 to 6 membered heteroalkyl, or 2 to 4 membered heteroalkyl), R³⁴-substituted or unsubstituted cycloalkyl (e.g., C₃-C₈ cycloalkyl, C₃-C₆ cycloalkyl, or C₅-C₆ cycloalkyl), R³⁴-substituted or unsubstituted heterocycloalkyl (e.g., 3 to 8 membered heterocycloalkyl, 3 to 6 membered heterocycloalkyl, or 5 to 6 membered heterocycloalkyl), R³⁴-substituted or unsubstituted aryl (e.g., C₆-C₁₀ aryl, C₁₀ aryl, or phenyl), or R³⁴-substituted or unsubstituted heteroaryl (e.g., 5 to 10 membered heteroaryl, 5 to 9 membered heteroaryl, or 5 to 6 membered heteroaryl). In some embodiments, R³³ is independently oxo, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R³³ is independently oxo. In some embodiments, R³³ is independently halogen. In some embodiments, R³³ is independently —CCl₃. In some embodiments, R³³ is independently —CBr₃. In some embodiments, R³³ is independently —CF₃.

In some embodiments, R³³ is independently —CI₃. In some embodiments, R³³ is independently —CHCl₂. In some embodiments, R³³ is independently —CHBr₂. In some embodiments, R³³ is independently —CHF₂. In some embodiments, R³³ is independently —CHI₂. In some embodiments, R³³ is independently —CH₂Cl. In some embodiments, R³³ is independently —CH₂Br. In some embodiments, R³³ is independently —CH₂F. In some embodiments, R³³ is independently —CH₂I. In some embodiments, R³³ is independently —CN. In some embodiments, R³³ is independently —OH. In some embodiments, R³³ is independently —NH₂. In some embodiments, R³³ is independently —COOH. In some embodiments, R³³ is independently —CONH₂. In some embodiments, R³³ is independently —NO₂. In some embodiments, R³³ is independently —SH. In some embodiments, R³³ is independently —SO₃H. In some embodiments, R³³ is independently —SO₄H. In some embodiments, R³³ is independently —SO₂NH₂. In some embodiments, R³³ is independently —NHNH₂. In some embodiments, R³³ is independently —ONH₂. In some embodiments, R³³ is independently —NHC(O)NHNH₂. In some embodiments, R³³ is independently —NHC(O)NH₂. In some embodiments, R³³ is independently —NHSO₂H. In some embodiments, R³³ is independently —NHC(O)H. In some embodiments, R³³ is independently —NHC(O)OH. In some embodiments, R³³ is independently —NHOH. In some embodiments, R³³ is independently —OCCl₃. In some embodiments, R³³ is independently —OCF₃. In some embodiments, R³³ is independently —OCBr₃. In some embodiments, R³³ is independently —OCI₃. In some embodiments, R³³ is independently —OCHCl₂. In some embodiments, R³³ is independently —OCHBr₂. In some embodiments, R³³ is independently —OCHI₂. In some embodiments, R³³ is independently —OCHF₂. In some embodiments, R³³ is independently —OCH₂Cl. In some embodiments, R³³ is independently —OCH₂Br. In some embodiments, R³³ is independently —OCH₂I. In some embodiments, R³³ is independently —OCH₂F. In some embodiments, R³³ is independently —N₃. In some embodiments, R³³ is independently —OCH₃. In some embodiments, R³³ is independently —CH₃. In some embodiments, R³³ is independently —CH₂CH₃. In some embodiments, R³³ is independently unsubstituted propyl. In some embodiments, R³³ is independently unsubstituted isopropyl. In some embodiments, R³³ is independently unsubstituted butyl. In some embodiments, R³³ is independently unsubstituted tert-butyl. In some embodiments, R³³ is independently —F. In some embodiments, R³³ is independently —Cl. In some embodiments, R³³ is independently —Br. In some embodiments, R³³ is independently —I.

In some embodiments, R³³ is independently R³⁴-substituted or unsubstituted alkyl (e.g., C₁-C₈ alkyl, C₁-C₆ alkyl, or C₁-C₄ alkyl). In some embodiments, R³³ is independently R³⁴-substituted alkyl (e.g., C₁-C₈ alkyl, C₁-C₆ alkyl, or C₁-C₄ alkyl). In some embodiments, R³³ is independently an unsubstituted alkyl (e.g., C₁-C₈ alkyl, C₁-C₆ alkyl, or C₁-C₄ alkyl). In some embodiments, R³³ is independently R³⁴-substituted or unsubstituted heteroalkyl (e.g., 2 to 8 membered heteroalkyl, 2 to 6 membered heteroalkyl, or 2 to 4 membered heteroalkyl). In some embodiments, R³³ is independently R³⁴-substituted heteroalkyl (e.g., 2 to 8 membered heteroalkyl, 2 to 6 membered heteroalkyl, or 2 to 4 membered heteroalkyl). In some embodiments, R³³ is independently an unsubstituted heteroalkyl (e.g., 2 to 8 membered heteroalkyl, 2 to 6 membered heteroalkyl, or 2 to 4 membered heteroalkyl). In some embodiments, R³³ is independently R³⁴-substituted or unsubstituted cycloalkyl (e.g., C₃-C₈ cycloalkyl, C₃-C₆ cycloalkyl, or C₅-C₆ cycloalkyl). In some embodiments, R³³ is independently R³⁴-substituted cycloalkyl (e.g., C₃-C₈ cycloalkyl, C₃-C₆ cycloalkyl, or C₅-C₆ cycloalkyl). In some embodiments, R³³ is independently an unsubstituted cycloalkyl (e.g., C₃-C₈ cycloalkyl, C₃-C₆ cycloalkyl, or C₅-C₆ cycloalkyl). In some embodiments, R³³ is independently R³⁴-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R³³ is independently R³⁴-substituted heterocycloalkyl (e.g., 3 to 8 membered heterocycloalkyl, 3 to 6 membered heterocycloalkyl, or 5 to 6 membered heterocycloalkyl). In some embodiments, R³³ is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R³³ is independently R³⁴-substituted or unsubstituted aryl (e.g., C₆-C₁₀ aryl, C₁₀ aryl, or phenyl). In some embodiments, R³³ is independently R³⁴-substituted aryl (e.g., C₆-C₁₀ aryl, C₁₀ aryl, or phenyl). In some embodiments, R³³ is independently an unsubstituted aryl (e.g., C₆-C₁₀ aryl, C₁₀ aryl, or phenyl). In some embodiments, R³³ is independently R³⁴-substituted or unsubstituted heteroaryl (e.g., 5 to 10 membered heteroaryl, 5 to 9 membered heteroaryl, or 5 to 6 membered heteroaryl). In some embodiments, R³³ is independently R³⁴-substituted heteroaryl (e.g., 5 to 10 membered heteroaryl, 5 to 9 membered heteroaryl, or 5 to 6 membered heteroaryl). In some embodiments, R³³ is independently an unsubstituted heteroaryl (e.g., 5 to 10 membered heteroaryl, 5 to 9 membered heteroaryl, or 5 to 6 membered heteroaryl).

In some embodiments, R³⁴ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R³⁵-substituted or unsubstituted alkyl (e.g., C₁-C₈ alkyl, C₁-C₆ alkyl, or C₁-C₄ alkyl), R³⁵-substituted or unsubstituted heteroalkyl (e.g., 2 to 8 membered heteroalkyl, 2 to 6 membered heteroalkyl, or 2 to 4 membered heteroalkyl), R³⁵-substituted or unsubstituted cycloalkyl (e.g., C₃-C₈ cycloalkyl, C₃-C₆ cycloalkyl, or C₅-C₆ cycloalkyl), R³⁵-substituted or unsubstituted heterocycloalkyl (e.g., 3 to 8 membered heterocycloalkyl, 3 to 6 membered heterocycloalkyl, or 5 to 6 membered heterocycloalkyl), R³⁵-substituted or unsubstituted aryl (e.g., C₆-C₁₀ aryl, C₁₀ aryl, or phenyl), or R³⁵-substituted or unsubstituted heteroaryl (e.g., 5 to 10 membered heteroaryl, 5 to 9 membered heteroaryl, or 5 to 6 membered heteroaryl). In some embodiments, R³⁴ is independently oxo, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R³⁴ is independently R³⁵-substituted or unsubstituted alkyl (e.g., C₁-C₈ alkyl, C₁-C₆ alkyl, or C₁-C₄ alkyl). In some embodiments, R³⁴ is independently R³⁵-substituted alkyl (e.g., C₁-C₈ alkyl, C₁-C₆ alkyl, or C₁-C₄ alkyl). In some embodiments, R³⁴ is independently an unsubstituted alkyl (e.g., C₁-C₈ alkyl, C₁-C₆ alkyl, or C₁-C₄ alkyl). In some embodiments, R³⁴ is independently R³⁵-substituted or unsubstituted heteroalkyl (e.g., 2 to 8 membered heteroalkyl, 2 to 6 membered heteroalkyl, or 2 to 4 membered heteroalkyl). In some embodiments, R³⁴ is independently R³⁵-substituted heteroalkyl (e.g., 2 to 8 membered heteroalkyl, 2 to 6 membered heteroalkyl, or 2 to 4 membered heteroalkyl). In some embodiments, R³⁴ is independently an unsubstituted heteroalkyl (e.g., 2 to 8 membered heteroalkyl, 2 to 6 membered heteroalkyl, or 2 to 4 membered heteroalkyl). In some embodiments, R³⁴ is independently R³⁵-substituted or unsubstituted cycloalkyl (e.g., C₃-C₈ cycloalkyl, C₃-C₆ cycloalkyl, or C₅-C₆ cycloalkyl). In some embodiments, R³⁴ is independently R³⁵-substituted cycloalkyl (e.g., C₃-C₈ cycloalkyl, C₃-C₆ cycloalkyl, or C₅-C₆ cycloalkyl). In some embodiments, R³⁴ is independently an unsubstituted cycloalkyl (e.g., C₃-C₈ cycloalkyl, C₃-C₆ cycloalkyl, or C₅-C₆ cycloalkyl). In some embodiments, R³⁴ is independently R³⁵-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R³⁴ is independently R³⁵-substituted heterocycloalkyl (e.g., 3 to 8 membered heterocycloalkyl, 3 to 6 membered heterocycloalkyl, or 5 to 6 membered heterocycloalkyl). In some embodiments, R³⁴ is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R³⁴ is independently R³⁵-substituted or unsubstituted aryl (e.g., C₆-C₁₀ aryl, C₁₀ aryl, or phenyl). In some embodiments, R³⁴ is independently R³⁵-substituted aryl (e.g., C₆-C₁₀ aryl, C₁₀ aryl, or phenyl). In some embodiments, R³⁴ is independently an unsubstituted aryl (e.g., C₆-C₁₀ aryl, C₁₀ aryl, or phenyl). In some embodiments, R³⁴ is independently R³⁵-substituted or unsubstituted heteroaryl (e.g., 5 to 10 membered heteroaryl, 5 to 9 membered heteroaryl, or 5 to 6 membered heteroaryl). In some embodiments, R³⁴ is independently R³⁵-substituted heteroaryl (e.g., 5 to 10 membered heteroaryl, 5 to 9 membered heteroaryl, or 5 to 6 membered heteroaryl). In some embodiments, R³⁴ is independently an unsubstituted heteroaryl (e.g., 5 to 10 membered heteroaryl, 5 to 9 membered heteroaryl, or 5 to 6 membered heteroaryl).

In some embodiments, R³⁴ is independently oxo. In some embodiments, R³⁴ is independently halogen. In some embodiments, R³⁴ is independently —CCl₃. In some embodiments, R³⁴ is independently —CBr₃. In some embodiments, R³⁴ is independently —CF₃. In some embodiments, R³⁴ is independently —CI₃. In some embodiments, R³⁴ is independently —CHCl₂. In some embodiments, R³⁴ is independently —CHBr₂. In some embodiments, R³⁴ is independently —CHF₂. In some embodiments, R³⁴ is independently —CHI₂. In some embodiments, R³⁴ is independently —CH₂Cl. In some embodiments, R³⁴ is independently —CH₂Br. In some embodiments, R³⁴ is independently —CH₂F. In some embodiments, R³⁴ is independently —CH₂I. In some embodiments, R³⁴ is independently —CN. In some embodiments, R³⁴ is independently —OH. In some embodiments, R³⁴ is independently —NH₂. In some embodiments, R³⁴ is independently —COOH. In some embodiments, R³⁴ is independently —CONH₂. In some embodiments, R³⁴ is independently —NO₂. In some embodiments, R³⁴ is independently —SH. In some embodiments, R³⁴ is independently —SO₃H. In some embodiments, R³⁴ is independently —SO₄H. In some embodiments, R³⁴ is independently —SO₂NH₂. In some embodiments, R³⁴ is independently —NHNH₂. In some embodiments, R³⁴ is independently —ONH₂. In some embodiments, R³⁴ is independently —NHC(O)NHNH₂. In some embodiments, R³⁴ is independently —NHC(O)NH₂. In some embodiments, R³⁴ is independently —NHSO₂H. In some embodiments, R³⁴ is independently —NHC(O)H. In some embodiments, R³⁴ is independently —NHC(O)OH. In some embodiments, R³⁴ is independently —NHOH. In some embodiments, R³⁴ is independently —OCCl₃. In some embodiments, R³⁴ is independently —OCF₃. In some embodiments, R³⁴ is independently —OCBr₃. In some embodiments, R³⁴ is independently —OCI₃. In some embodiments, R³⁴ is independently —OCHCl₂. In some embodiments, R³⁴ is independently —OCHBr₂. In some embodiments, R³⁴ is independently —OCHI₂. In some embodiments, R³⁴ is independently —OCHF₂. In some embodiments, R³⁴ is independently —OCH₂Cl. In some embodiments, R³⁴ is independently —OCH₂Br. In some embodiments, R³⁴ is independently —OCH₂I. In some embodiments, R³⁴ is independently —OCH₂F. In some embodiments, R³⁴ is independently —N₃. In some embodiments, R³⁴ is independently —OCH₃. In some embodiments, R³⁴ is independently —CH₃. In some embodiments, R³⁴ is independently —CH₂CH₃. In some embodiments, R³⁴ is independently unsubstituted propyl. In some embodiments, R³⁴ is independently unsubstituted isopropyl. In some embodiments, R³⁴ is independently unsubstituted butyl. In some embodiments, R³⁴ is independently unsubstituted tert-butyl. In some embodiments, R³⁴ is independently —F. In some embodiments, R³⁴ is independently —Cl. In some embodiments, R³⁴ is independently —Br. In some embodiments, R³⁴ is independently —I.

In some embodiments, R³⁵ is independently oxo, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R³⁵ is independently an unsubstituted alkyl (e.g., C₁-C₈ alkyl, C₁-C₆ alkyl, or C₁-C₄ alkyl). In some embodiments, R³⁵ is independently an unsubstituted heteroalkyl (e.g., 2 to 8 membered heteroalkyl, 2 to 6 membered heteroalkyl, or 2 to 4 membered heteroalkyl). In some embodiments, R³⁵ is independently an unsubstituted cycloalkyl (e.g., C₃-C₈ cycloalkyl, C₃-C₆ cycloalkyl, or C₅-C₆ cycloalkyl). In some embodiments, R³⁵ is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R³⁵ is independently an unsubstituted aryl (e.g., C₆-C₁₀ aryl, C₁₀ aryl, or phenyl). In some embodiments, R³⁵ is independently an unsubstituted heteroaryl (e.g., 5 to 10 membered heteroaryl, 5 to 9 membered heteroaryl, or 5 to 6 membered heteroaryl).

In some embodiments, R³⁵ is independently oxo. In some embodiments, R³⁵ is independently halogen. In some embodiments, R³⁵ is independently —CCl₃. In some embodiments, R³⁵ is independently —CBr₃. In some embodiments, R³⁵ is independently —CF₃. In some embodiments, R³⁵ is independently —CI₃. In some embodiments, R³⁵ is independently —CHCl₂. In some embodiments, R³⁵ is independently —CHBr₂. In some embodiments, R³⁵ is independently —CHF₂. In some embodiments, R³⁵ is independently —CHI₂. In some embodiments, R³⁵ is independently —CH₂Cl. In some embodiments, R³⁵ is independently —CH₂Br. In some embodiments, R³⁵ is independently —CH₂F. In some embodiments, R³⁵ is independently —CH₂I. In some embodiments, R³⁵ is independently —CN. In some embodiments, R³⁵ is independently —OH. In some embodiments, R³⁵ is independently —NH₂. In some embodiments, R³⁵ is independently —COOH. In some embodiments, R³⁵ is independently —CONH₂. In some embodiments, R³⁵ is independently —NO₂. In some embodiments, R³⁵ is independently —SH. In some embodiments, R³⁵ is independently —SO₃H. In some embodiments, R³⁵ is independently —SO₄H. In some embodiments, R³⁵ is independently —SO₂NH₂. In some embodiments, R³⁵ is independently —NHNH₂. In some embodiments, R³⁵ is independently —ONH₂. In some embodiments, R³⁵ is independently —NHC(O)NHNH₂. In some embodiments, R³⁵ is independently —NHC(O)NH₂. In some embodiments, R³⁵ is independently —NHSO₂H. In some embodiments, R³⁵ is independently —NHC(O)H. In some embodiments, R³⁵ is independently —NHC(O)OH. In some embodiments, R³⁵ is independently —NHOH. In some embodiments, R³⁵ is independently —OCCl₃. In some embodiments, R³⁵ is independently —OCF₃. In some embodiments, R³⁵ is independently —OCBr₃. In some embodiments, R³⁵ is independently —OCI₃. In some embodiments, R³⁵ is independently —OCHCl₂. In some embodiments, R³⁵ is independently —OCHBr₂. In some embodiments, R³⁵ is independently —OCHI₂. In some embodiments, R³⁵ is independently —OCHF₂. In some embodiments, R³⁵ is independently —OCH₂Cl. In some embodiments, R³⁵ is independently —OCH₂Br. In some embodiments, R³⁵ is independently —OCH₂I. In some embodiments, R³⁵ is independently —OCH₂F. In some embodiments, R³⁵ is independently —N₃. In some embodiments, R³⁵ is independently —OCH₃. In some embodiments, R³⁵ is independently —CH₃. In some embodiments, R³⁵ is independently —CH₂CH₃. In some embodiments, R³⁵ is independently unsubstituted propyl. In some embodiments, R³⁵ is independently unsubstituted isopropyl. In some embodiments, R³⁵ is independently unsubstituted butyl. In some embodiments, R³⁵ is independently unsubstituted tert-butyl. In some embodiments, R³⁵ is independently —F. In some embodiments, R³⁵ is independently —Cl. In some embodiments, R³⁵ is independently —Br. In some embodiments, R³⁵ is independently —I.

In some embodiments, R^{5A} is independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In some embodiments, R^{5B} is independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In some embodiments, R^{5C} is independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In some embodiments, R^{5D} is independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In some embodiments, R^{5A} and R^{5B} substituents bonded to the same nitrogen atom are independently joined to form a substituted or unsubstituted heterocycloalkyl (e.g., 3 to 8 membered heterocycloalkyl, 3 to 6 membered heterocycloalkyl, or 5 to 6 membered heterocycloalkyl) or substituted or unsubstituted heteroaryl (e.g., 5 to 10 membered heteroaryl, 5 to 9 membered heteroaryl, or 5 to 6 membered heteroaryl). In some embodiments, R^{5A} and R^{5B} substituents bonded to the same nitrogen atom are independently joined to form a substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R^{5A} and R^{5B} substituents bonded to the same nitrogen atom are independently joined to form a substituted heterocycloalkyl (e.g., 3 to 8 membered heterocycloalkyl, 3 to 6 membered heterocycloalkyl, or 5 to 6 membered heterocycloalkyl). In some embodiments, R^{5A} and R^{5B} substituents bonded to the same nitrogen atom are independently joined to form an unsubstituted heterocycloalkyl (e.g., 3 to 8 membered heterocycloalkyl, 3 to 6 membered heterocycloalkyl, or 5 to 6 membered heterocycloalkyl). In some embodiments, R^{5A} and R^{5B} substituents bonded to the same nitrogen atom are independently joined to form a substituted or unsubstituted heteroaryl (e.g., 5 to 10 membered heteroaryl, 5 to 9 membered heteroaryl, or 5 to 6 membered heteroaryl). In some embodiments, R^{5A} and R^{5B} substituents bonded to the same nitrogen atom are independently joined to form a substituted heteroaryl (e.g., 5 to 10 membered heteroaryl, 5 to 9 membered heteroaryl, or 5 to 6 membered heteroaryl). In some embodiments, R^{5A} and R^{5B} substituents bonded to the same nitrogen atom are independently joined to form an unsubstituted heteroaryl (e.g., 5 to 10 membered heteroaryl, 5 to 9 membered heteroaryl, or 5 to 6 membered heteroaryl).

In some embodiments, R^{5A}, R^{5B}, R^{5C}, and R^{5D} are independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, R³³-substituted or unsubstituted alkyl (e.g., C₁-C₈ alkyl, C₁-C₆ alkyl, or C₁-C₄ alkyl), R³³-substituted or unsubstituted heteroalkyl (e.g., 2 to 8 membered heteroalkyl, 2 to 6 membered heteroalkyl, or 2 to 4 membered heteroalkyl), R³³-substituted or unsubstituted cycloalkyl (e.g., C₃-C₈ cycloalkyl, C₃-C₆ cycloalkyl, or C₅-C₆ cycloalkyl), R³³-substituted or unsubstituted heterocycloalkyl (e.g., 3 to 8 membered heterocycloalkyl, 3 to 6 membered heterocycloalkyl, or 5 to 6 membered heterocycloalkyl), R³³-substituted or unsubstituted aryl (e.g., C₆-C₁₀ aryl, C₁₀ aryl, or phenyl), or R³³-substituted or unsubstituted heteroaryl (e.g., 5 to 10 membered heteroaryl, 5 to 9 membered heteroaryl, or 5 to 6 membered heteroaryl). In some embodiments, R^{5A} and R^{5B} substituents bonded to the same nitrogen atom are independently joined to form an R³³-substituted or unsubstituted heterocycloalkyl (e.g., 3 to 8 membered heterocycloalkyl, 3 to 6 membered heterocycloalkyl, or 5 to 6 membered heterocycloalkyl) or R³³-substituted or unsubstituted heteroaryl (e.g., 5 to 10 membered heteroaryl, 5 to 9 membered heteroaryl, or 5 to 6 membered heteroaryl). In some embodiments, R^{5A} and R^{5B} substituents bonded to the same nitrogen atom are independently joined to form an R³³-substituted or unsubstituted heterocycloalkyl (e.g., 3 to 8 membered heterocycloalkyl, 3 to 6 membered heterocycloalkyl. In some embodiments, R^{5A} and R^{5B} substituents bonded to the same nitrogen atom are independently joined to form an R³³-substituted or unsubstituted heteroaryl (e.g., 5 to 10 membered heteroaryl, 5 to 9 membered heteroaryl, or 5 to 6 membered heteroaryl).

In some embodiments, R^{5A} is independently R³³-substituted or unsubstituted alkyl (e.g., C₁-C₈ alkyl, C₁-C₆ alkyl, or C₁-C₄ alkyl). In some embodiments, R^{5A} is independently R³³-substituted alkyl (e.g., C₁-C₈ alkyl, C₁-C₆ alkyl, or C₁-C₄ alkyl). In some embodiments, R^{5A} is independently an unsubstituted alkyl (e.g., C₁-C₈ alkyl, C₁-C₆ alkyl, or C₁-C₄ alkyl). In some embodiments, R^{5A} is independently R³³-substituted or unsubstituted heteroalkyl (e.g., 2 to 8 membered heteroalkyl, 2 to 6 membered heteroalkyl, or 2 to 4 membered heteroalkyl). In some embodiments, R^{5A} is independently R³³-substituted heteroalkyl (e.g., 2 to 8 membered heteroalkyl, 2 to 6 membered heteroalkyl, or 2 to 4 membered heteroalkyl). In some embodiments, R^{5A} is independently an unsubstituted heteroalkyl (e.g., 2 to 8 membered heteroalkyl, 2 to 6 membered heteroalkyl, or 2 to 4 membered heteroalkyl). In some embodiments, R^{5A} is independently R³³-substituted or unsubstituted cycloalkyl (e.g., C₃-C₈ cycloalkyl, C₃-C₆ cycloalkyl, or C₅-C₆ cycloalkyl). In some embodiments, R^{5A} is independently R³³-substituted cycloalkyl (e.g., C₃-C₈ cycloalkyl, C₃-C₆ cycloalkyl, or C₅-C₆ cycloalkyl). In some embodiments, R^{5A} is independently an unsubstituted cycloalkyl (e.g., C₃-C₈ cycloalkyl, C₃-C₆ cycloalkyl, or C₅-C₆ cycloalkyl). In some embodiments, R^{5A} is independently R³³-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R^{5A} is independently R³³-substituted heterocycloalkyl (e.g., 3 to 8 membered heterocycloalkyl, 3 to 6 membered heterocycloalkyl, or 5 to 6 membered heterocycloalkyl). In some embodiments, R^{5A} is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R^{5A} is independently R³³-substituted or unsubstituted aryl (e.g., C₆-C₁₀ aryl, C₁₀ aryl, or phenyl). In some embodiments, R^{5A} is independently R³³-substituted aryl (e.g., C₆-C₁₀ aryl, C₁₀ aryl, or phenyl). In some embodiments, R^{5A} is independently an unsubstituted aryl (e.g., C₆-C₁₀ aryl, C₁₀ aryl, or phenyl). In some embodiments, R^{5A} is independently R³³-substituted or unsubstituted heteroaryl (e.g., 5 to 10 membered heteroaryl, 5 to 9 membered heteroaryl, or 5 to 6 membered heteroaryl). In some embodiments, R^{5A} is independently R³³-substituted heteroaryl (e.g., 5 to 10 membered heteroaryl, 5 to 9 membered heteroaryl, or 5 to 6 membered heteroaryl). In some embodiments, R^{5A} is independently an unsubstituted heteroaryl (e.g., 5 to 10 membered heteroaryl, 5 to 9 membered heteroaryl, or 5 to 6 membered heteroaryl).

In some embodiments, R^{5A} is independently —CCl₃. In some embodiments, R^{5A} is independently —CBr₃. In some embodiments, R^{5A} is independently —CF₃. In some embodiments, R^{5A} is independently —CI₃. In some embodiments, R^{5A} is independently —CHCl₂. In some embodiments, R^{5A} is independently —CHBr₂. In some embodiments, R^{5A} is independently —CHF₂. In some embodiments, R^{5A} is independently —CHI₂. In some embodiments, R^{5A} is independently —CH₂Cl. In some embodiments, R^{5A} is independently —CH₂Br. In some embodiments, R^{5A} is independently —CH₂F. In some embodiments, R^{5A} is independently —CH₂I. In some embodiments, R^{5A} is independently —CN. In some embodiments, R^{5A} is independently —OH. In some embodiments, R^{5A} is independently —COOH. In some embodiments, R^{5A} is independently —CONH₂. In some embodiments, R^{5A} is independently —OCCl₃. In some embodiments, R^{5A} is independently —OCF₃. In some embodiments, R^{5A} is independently —OCBr₃. In some embodiments, R^{5A} is independently —OCI₃. In some embodiments, R^{5A} is independently —OCHCl₂. In some embodiments, R^{5A} is independently —OCHBr₂. In some embodiments, R^{5A} is independently —OCHI₂. In some embodiments, R^{5A} is independently —OCHF₂. In some embodiments, R^{5A} is independently —OCH₂Cl. In some embodiments, R^{5A} is independently —OCH₂Br. In some embodiments, R^{5A} is independently —OCH₂I. In some embodiments, R^{5A} is independently —OCH₂F. In some embodiments, R^{5A} is independently —OCH₃. In some embodiments, R^{5A} is independently —CH₃. In some embodiments, R^{5A} is independently —CH₂CH₃. In some embodiments, R^{5A} is independently unsubstituted propyl. In some embodiments, R^{5A} is independently unsubstituted isopropyl. In some embodiments, R^{5A} is independently unsubstituted butyl. In some embodiments, R^{5A} is independently unsubstituted tert-butyl. In some embodiments, R^{5A} is independently hydrogen.

In some embodiments, R^{5B} is independently R³³-substituted or unsubstituted alkyl (e.g., C₁-C₈ alkyl, C₁-C₆ alkyl, or C₁-C₄ alkyl). In some embodiments, R^{5B} is independently R³³-substituted alkyl (e.g., C₁-C₈ alkyl, C₁-C₆ alkyl, or C₁-C₄ alkyl). In some embodiments, R^{5B} is independently an unsubstituted alkyl (e.g., C₁-C₈ alkyl, C₁-C₆ alkyl, or C₁-C₄ alkyl). In some embodiments, R^{5B} is independently R³³-substituted or unsubstituted heteroalkyl (e.g., 2 to 8 membered heteroalkyl, 2 to 6 membered heteroalkyl, or 2 to 4 membered heteroalkyl). In some embodiments, R^{5B} is independently R³³-substituted heteroalkyl (e.g., 2 to 8 membered heteroalkyl, 2 to 6 membered heteroalkyl, or 2 to 4 membered heteroalkyl). In some embodiments, R^{5B} is independently an unsubstituted heteroalkyl (e.g., 2 to 8 membered heteroalkyl, 2 to 6 membered heteroalkyl, or 2 to 4 membered heteroalkyl). In some embodiments, R^{5B} is independently R³³-substituted or unsubstituted cycloalkyl (e.g., C₃-C₈ cycloalkyl, C₃-C₆ cycloalkyl, or C₅-C₆ cycloalkyl). In some embodiments, R^{5B} is independently R³³-substituted cycloalkyl (e.g., C₃-C₈ cycloalkyl, C₃-C₆ cycloalkyl, or C₅-C₆ cycloalkyl). In some embodiments, R^{5B} is independently an unsubstituted cycloalkyl (e.g., C₃-C₈ cycloalkyl, C₃-C₆ cycloalkyl, or C₅-C₆ cycloalkyl). In some embodiments, R^{5B} is independently R³³-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R^{5B} is independently R³³-substituted heterocycloalkyl (e.g., 3 to 8 membered heterocycloalkyl, 3 to 6 membered heterocycloalkyl, or 5 to 6 membered heterocycloalkyl). In some embodiments, R^{5B} is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R^{5B} is independently R³³-substituted or unsubstituted aryl (e.g., C₆-C₁₀ aryl, C₁₀ aryl, or phenyl). In some embodiments, R^{5B} is independently R³³-substituted aryl (e.g., C₆-C₁₀ aryl, C₁₀ aryl, or phenyl). In some embodiments, R^{5B} is independently an unsubstituted aryl (e.g., C₆-C₁₀ aryl, C₁₀ aryl, or phenyl). In some embodiments, R^{5B} is independently R³³-substituted or unsubstituted heteroaryl (e.g., 5 to 10 membered heteroaryl, 5 to 9 membered heteroaryl, or 5 to 6 membered heteroaryl). In some embodiments, R^{5B} is independently R³³-substituted heteroaryl (e.g., 5 to 10 membered heteroaryl, 5 to 9 membered heteroaryl, or 5 to 6 membered heteroaryl). In some embodiments, R^{5B} is independently an unsubstituted heteroaryl (e.g., 5 to 10 membered heteroaryl, 5 to 9 membered heteroaryl, or 5 to 6 membered heteroaryl).

In some embodiments, R^{5B} is independently —CCl₃. In some embodiments, R^{5B} is independently —CBr₃. In some embodiments, R^{5B} is independently —CF₃. In some embodiments, R^{5B} is independently —CI₃. In some embodiments, R^{5B} is independently —CHCl₂. In some embodiments, R^{5B} is independently —CHBr₂. In some embodiments, R^{5B} is independently —CHF₂. In some embodiments, R^{5B} is independently —CHI₂. In some embodiments, R^{5B} is independently —CH₂Cl. In some embodiments, R^{5B} is independently —CH₂Br. In some embodiments, R^{5B} is independently —CH₂F. In some embodiments, R^{5B} is independently —CH₂I. In some embodiments, R^{5B} is independently —CN. In some embodiments, R^{5B} is independently —OH. In some embodiments, R^{5B} is independently —COOH. In some embodiments, R^{5B} is independently —CONH₂. In some embodiments, R^{5B} is independently —OCCl₃. In some embodiments, R^{5B} is independently —OCF₃. In some embodiments, R^{5B} is independently —OCBr₃. In some embodiments, R^{5B} is independently —OCI₃. In some embodiments, R^{5B} is independently —OCHCl₂. In some embodiments, R^{5B} is independently —OCHBr₂. In some embodiments, R^{5B} is independently —OCHI₂. In some embodiments, R^{5B} is independently —OCHF₂. In some embodiments, R^{5B} is independently —OCH₂Cl. In some embodiments, R^{5B} is independently —OCH₂Br. In some embodiments, R^{5B} is independently —OCH₂I. In some embodiments, R^{5B} is independently —OCH₂F. In some embodiments, R^{5B} is independently —OCH₃. In some embodiments, R^{5B} is independently —CH₃. In some embodiments, R^{5B} is independently —CH₂CH₃. In some embodiments, R^{5B} is independently unsubstituted propyl. In some embodiments, R^{5B} is independently unsubstituted isopropyl. In some embodiments, R^{5B} is independently unsubstituted butyl. In some embodiments, R^{5B} is independently unsubstituted tert-butyl. In some embodiments, R^{5B} is independently hydrogen.

In some embodiments, R^{5C} is independently R³³-substituted or unsubstituted alkyl (e.g., C₁-C₈ alkyl, C₁-C₆ alkyl, or C₁-C₄ alkyl). In some embodiments, R^{5C} is independently R³³-substituted alkyl (e.g., C₁-C₈ alkyl, C₁-C₆ alkyl, or C₁-C₄ alkyl). In some embodiments, R^{5C} is independently an unsubstituted alkyl (e.g., C₁-C₈ alkyl, C₁-C₆ alkyl, or C₁-C₄ alkyl). In some embodiments, R^{5C} is independently R³³-substituted or unsubstituted heteroalkyl (e.g., 2 to 8 membered heteroalkyl, 2 to 6 membered heteroalkyl, or 2 to 4 membered heteroalkyl). In some embodiments, R^{5C} is independently R³³-substituted heteroalkyl (e.g., 2 to 8 membered heteroalkyl, 2 to 6 membered heteroalkyl, or 2 to 4 membered heteroalkyl). In some embodiments, R^{5C} is independently an unsubstituted heteroalkyl (e.g., 2 to 8 membered heteroalkyl, 2 to 6 membered heteroalkyl, or 2 to 4 membered heteroalkyl). In some embodiments, R^{5C} is independently R³³-substituted or unsubstituted cycloalkyl (e.g., C₃-C₈ cycloalkyl, C₃-C₆ cycloalkyl, or C₅-C₆ cycloalkyl). In some embodiments, R^{5C} is independently R³³-substituted cycloalkyl (e.g., C₃-C₈ cycloalkyl, C₃-C₆ cycloalkyl, or C₅-C₆ cycloalkyl). In some embodiments, R^{5C} is independently an unsubstituted cycloalkyl (e.g., C₃-C₈ cycloalkyl, C₃-C₆ cycloalkyl, or C₅-C₆ cycloalkyl). In some embodiments, R^{5C} is independently R³³-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R^{5C} is independently R³³-substituted heterocycloalkyl (e.g., 3 to 8 membered heterocycloalkyl, 3 to 6 membered heterocycloalkyl, or 5 to 6 membered heterocycloalkyl). In some embodiments, R^{5C} is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R^{5C} is independently R³³-substituted or unsubstituted aryl (e.g., C₆-C₁₀ aryl, C₁₀ aryl, or phenyl). In some embodiments, R^{5C} is independently R³³-substituted aryl (e.g., C₆-C₁₀ aryl, C₁₀ aryl, or phenyl). In some embodiments, R^{5C} is independently an unsubstituted aryl (e.g., C₆-C₁₀ aryl, C₁₀ aryl, or phenyl). In some embodiments, R^{5C} is independently R³³-substituted or unsubstituted heteroaryl (e.g., 5 to 10 membered heteroaryl, 5 to 9 membered heteroaryl, or 5 to 6 membered heteroaryl). In some embodiments, R^{5C} is independently R³³-substituted heteroaryl (e.g., 5 to 10 membered heteroaryl, 5 to 9 membered heteroaryl, or 5 to 6 membered heteroaryl). In some embodiments, R^{5C} is independently an unsubstituted heteroaryl (e.g., 5 to 10 membered heteroaryl, 5 to 9 membered heteroaryl, or 5 to 6 membered heteroaryl).

In some embodiments, R^{5C} is independently —CCl₃. In some embodiments, R^{5C} is independently —CBr₃. In some embodiments, R^{5C} is independently —CF₃. In some embodiments, R^{5C} is independently —CI₃. In some embodiments, R^{5C} is independently —CHCl₂. In some embodiments, R^{5C} is independently —CHBr₂. In some embodiments, R^{5C} is independently —CHF₂. In some embodiments, R^{5C} is independently —CHI₂. In some embodiments, R^{5C} is independently —CH₂Cl. In some embodiments, R^{5C} is independently —CH₂Br. In some embodiments, R^{5C} is independently —CH₂F. In some embodiments, R^{5C} is independently —CH₂I. In some embodiments, R^{5C} is independently —CN. In some embodiments, R^{5C} is independently —OH. In some embodiments, R^{5C} is independently —COOH. In some embodiments, R^{5C} is independently —CONH₂. In some embodiments, R^{5C} is independently —OCCl₃. In some embodiments, R^{5C} is independently —OCF₃. In some embodiments, R^{5C} is independently —OCBr₃. In some embodiments, R^{5C} is independently —OCI₃. In some embodiments, R^{5C} is independently —OCHCl₂. In some embodiments, R^{5C} is independently —OCHBr₂. In some embodiments, R^{5C} is independently —OCHI₂. In some embodiments, R^{5C} is independently —OCHF₂. In some embodiments, R^{5C} is independently —OCH₂Cl. In some embodiments, R^{5C} is independently —OCH₂Br. In some embodiments, R^{5C} is independently —OCH₂I. In some embodiments, R^{5C} is independently —OCH₂F. In some embodiments, R^{5C} is independently —OCH₃. In some embodiments, R^{5C} is independently —CH₃. In some embodiments, R^{5C} is independently —CH₂CH₃. In some embodiments, R^{5C} is independently unsubstituted propyl. In some embodiments, R^{5C} is independently unsubstituted isopropyl. In some embodiments, R^{5C} is independently unsubstituted butyl. In some embodiments, R^{5C} is independently unsubstituted tert-butyl. In some embodiments, R^{5C} is independently hydrogen.

In some embodiments, R^{5D} is independently R³³-substituted or unsubstituted alkyl (e.g., C₁-C₈ alkyl, C₁-C₆ alkyl, or C₁-C₄ alkyl). In some embodiments, R^{5D} is independently R³³-substituted alkyl (e.g., C₁-C₈ alkyl, C₁-C₆ alkyl, or C₁-C₄ alkyl). In some embodiments, R^{5D} is independently an unsubstituted alkyl (e.g., C₁-C₈ alkyl, C₁-C₆ alkyl, or C₁-C₄ alkyl). In some embodiments, R^{5D} is independently R³³-substituted or unsubstituted heteroalkyl (e.g., 2 to 8 membered heteroalkyl, 2 to 6 membered heteroalkyl, or 2 to 4 membered heteroalkyl). In some embodiments, R^{5D} is independently R³³-substituted heteroalkyl (e.g., 2 to 8 membered heteroalkyl, 2 to 6 membered heteroalkyl, or 2 to 4 membered heteroalkyl). In some embodiments, R^{5D} is independently an unsubstituted heteroalkyl (e.g., 2 to 8 membered heteroalkyl, 2 to 6 membered heteroalkyl, or 2 to 4 membered heteroalkyl). In some embodiments, R^{5D} is independently R³³-substituted or unsubstituted cycloalkyl (e.g., C₃-C₈ cycloalkyl, C₃-C₆ cycloalkyl, or C₅-C₆ cycloalkyl). In some embodiments, R^{5D} is independently R³³-substituted cycloalkyl (e.g., C₃-C₈ cycloalkyl, C₃-C₆ cycloalkyl, or C₅-C₆ cycloalkyl). In some embodiments, R^{5D} is independently an unsubstituted cycloalkyl (e.g., C₃-C₈ cycloalkyl, C₃-C₆ cycloalkyl, or C₅-C₆ cycloalkyl). In some embodiments, R^{5D} is independently R³³-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R^{5D} is independently R³³-substituted heterocycloalkyl (e.g., 3 to 8 membered heterocycloalkyl, 3 to 6 membered heterocycloalkyl, or 5 to 6 membered heterocycloalkyl). In some embodiments, R^{5D} is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R^{5D} is independently R³³-substituted or unsubstituted aryl (e.g., C₆-C₁₀ aryl, C₁₀ aryl, or phenyl). In some embodiments, R^{5D} is independently R³³-substituted aryl (e.g., C₆-C₁₀ aryl, C₁₀ aryl, or phenyl). In some embodiments, R^{5D} is independently an unsubstituted aryl (e.g., C₆-C₁₀ aryl, C₁₀ aryl, or phenyl). In some embodiments, R^{5D} is independently R³³-substituted or unsubstituted heteroaryl (e.g., 5 to 10 membered heteroaryl, 5 to 9 membered heteroaryl, or 5 to 6 membered heteroaryl). In some embodiments, R^{5D} is independently R³³-substituted heteroaryl (e.g., 5 to 10 membered heteroaryl, 5 to 9 membered heteroaryl, or 5 to 6 membered heteroaryl). In some embodiments, R^{5D} is independently an unsubstituted heteroaryl (e.g., 5 to 10 membered heteroaryl, 5 to 9 membered heteroaryl, or 5 to 6 membered heteroaryl).

In some embodiments, R^{5D} is independently —CCl₃. In some embodiments, R^{5D} is independently —CBr₃. In some embodiments, R^{5D} is independently —CF₃. In some embodiments, R^{5D} is independently —CI₃. In some embodiments, R^{5D} is independently —CHCl₂. In some embodiments, R^{5D} is independently —CHBr₂. In some embodiments, R^{5D} is independently —CHF₂. In some embodiments, R^{5D} is independently —CHI₂. In some embodiments, R^{5D} is independently —CH₂Cl. In some embodiments, R^{5D} is independently —CH₂Br. In some embodiments, R^{5D} is independently —CH₂F. In some embodiments, R^{5D} is independently —CH₂I. In some embodiments, R^{5D} is independently —CN. In some embodiments, R^{5D} is independently —OH. In some embodiments, R^{5D} is independently —COOH. In some embodiments, R^{5D} is independently —CONH₂. In some embodiments, R^{5D} is independently —OCCl₃. In some embodiments, R^{5D} is independently —OCF₃. In some embodiments, R^{5D} is independently —OCBr₃. In some embodiments, R^{5D} is independently —OCI₃. In some embodiments, R^{5D} is independently —OCHCl₂. In some embodiments, R^{5D} is independently —OCHBr₂. In some embodiments, R^{5D} is independently —OCHI₂. In some embodiments, R^{5D} is independently —OCHF₂. In some embodiments, R^{5D} is independently —OCH₂Cl. In some embodiments, R^{5D} is independently —OCH₂Br. In some embodiments, R^{5D} is independently —OCH₂I. In some embodiments, R^{5D} is independently —OCH₂F. In some embodiments, R^{5D} is independently —OCH₃. In some embodiments, R^{5D} is independently —CH₃. In some embodiments, R^{5D} is independently —CH₂CH₃. In some embodiments, R^{5D} is independently unsubstituted propyl. In some embodiments, R^{5D} is independently unsubstituted isopropyl. In some embodiments, R^{5D} is independently unsubstituted butyl. In some embodiments, R^{5D} is independently unsubstituted tert-butyl. In some embodiments, R^{5D} is independently hydrogen.

In some embodiments, R⁶ is independently hydrogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —COOH, —CONH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In some embodiments, R⁶ is independently substituted or unsubstituted alkyl (e.g., C₁-C₈ alkyl, C₁-C₆ alkyl, or C₁-C₄ alkyl). In some embodiments, R⁶ is independently substituted alkyl (e.g., C₁-C₈ alkyl, C₁-C₆ alkyl, or C₁-C₄ alkyl). In some embodiments, R⁶ is independently an unsubstituted alkyl (e.g., C₁-C₈ alkyl, C₁-C₆ alkyl, or C₁-C₄ alkyl). In some embodiments, R⁶ is independently substituted or unsubstituted heteroalkyl (e.g., 2 to 8 membered heteroalkyl, 2 to 6 membered heteroalkyl, or 2 to 4 membered heteroalkyl). In some embodiments, R⁶ is independently substituted heteroalkyl (e.g., 2 to 8 membered heteroalkyl, 2 to 6 membered heteroalkyl, or 2 to 4 membered heteroalkyl). In some embodiments, R⁶ is independently an unsubstituted heteroalkyl (e.g., 2 to 8 membered heteroalkyl, 2 to 6 membered heteroalkyl, or 2 to 4 membered heteroalkyl). In some embodiments, R⁶ is independently substituted or unsubstituted cycloalkyl (e.g., C₃-C₈ cycloalkyl, C₃-C₆ cycloalkyl, or C₅-C₆ cycloalkyl). In some embodiments, R⁶ is independently substituted cycloalkyl (e.g., C₃-C₈ cycloalkyl, C₃-C₆ cycloalkyl, or C₅-C₆ cycloalkyl). In some embodiments, R⁶ is independently an unsubstituted cycloalkyl (e.g., C3-C₈ cycloalkyl, C₃-C₆ cycloalkyl, or C₅-C₆ cycloalkyl). In some embodiments, R⁶ is independently substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R⁶ is independently substituted heterocycloalkyl (e.g., 3 to 8 membered heterocycloalkyl, 3 to 6 membered heterocycloalkyl, or 5 to 6 membered heterocycloalkyl). In some embodiments, R⁶ is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R⁶ is independently substituted or unsubstituted aryl (e.g., C₆-C₁₀ aryl, C₁₀ aryl, or phenyl). In some embodiments, R⁶ is independently substituted aryl (e.g., C₆-C₁₀ aryl, C₁₀ aryl, or phenyl). In some embodiments, R⁶ is independently an unsubstituted aryl (e.g., C₆-C₁₀ aryl, C₁₀ aryl, or phenyl). In some embodiments, R⁶ is independently substituted or unsubstituted heteroaryl (e.g., 5 to 10 membered heteroaryl, 5 to 9 membered heteroaryl, or 5 to 6 membered heteroaryl). In some embodiments, R⁶ is independently substituted heteroaryl (e.g., 5 to 10 membered heteroaryl, 5 to 9 membered heteroaryl, or 5 to 6 membered heteroaryl). In some embodiments, R⁶ is independently an unsubstituted heteroaryl (e.g., 5 to 10 membered heteroaryl, 5 to 9 membered heteroaryl, or 5 to 6 membered heteroaryl).

In some embodiments, R⁶ is independently hydrogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —COOH, —CONH₂, R³⁶-substituted or unsubstituted alkyl (e.g., C₁-C₈ alkyl, C₁-C₆ alkyl, or C₁-C₄ alkyl), R³⁶-substituted or unsubstituted heteroalkyl (e.g., 2 to 8 membered heteroalkyl, 2 to 6 membered heteroalkyl, or 2 to 4 membered heteroalkyl), R³⁶-substituted or unsubstituted cycloalkyl (e.g., C₃-C₈ cycloalkyl, C₃-C₆ cycloalkyl, or C₅-C₆ cycloalkyl), R³⁶-substituted or unsubstituted heterocycloalkyl (e.g., 3 to 8 membered heterocycloalkyl, 3 to 6 membered heterocycloalkyl, or 5 to 6 membered heterocycloalkyl), R³⁶-substituted or unsubstituted aryl (e.g., C₆-C₁₀ aryl, C₁₀ aryl, or phenyl), or R³⁶-substituted or unsubstituted heteroaryl (e.g., 5 to 10 membered heteroaryl, 5 to 9 membered heteroaryl, or 5 to 6 membered heteroaryl). In some embodiments, R⁶ is independently hydrogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —COOH, —CONH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R⁶ is independently R³⁶-substituted or unsubstituted alkyl (e.g., C₁-C₈ alkyl, C₁-C₆ alkyl, or C₁-C₄ alkyl). In some embodiments, R⁶ is independently R³⁶-substituted alkyl (e.g., C₁-C₈ alkyl, C₁-C₆ alkyl, or C₁-C₄ alkyl). In some embodiments, R⁶ is independently an unsubstituted alkyl (e.g., C₁-C₈ alkyl, C₁-C₆ alkyl, or C₁-C₄ alkyl). In some embodiments, R⁶ is independently R³⁶-substituted or unsubstituted heteroalkyl (e.g., 2 to 8 membered heteroalkyl, 2 to 6 membered heteroalkyl, or 2 to 4 membered heteroalkyl). In some embodiments, R⁶ is independently R³⁶-substituted heteroalkyl (e.g., 2 to 8 membered heteroalkyl, 2 to 6 membered heteroalkyl, or 2 to 4 membered heteroalkyl). In some embodiments, R⁶ is independently an unsubstituted heteroalkyl (e.g., 2 to 8 membered heteroalkyl, 2 to 6 membered heteroalkyl, or 2 to 4 membered heteroalkyl). In some embodiments, R⁶ is independently R³⁶-substituted or unsubstituted cycloalkyl (e.g., C₃-C₈ cycloalkyl, C₃-C₆ cycloalkyl, or C₅-C₆ cycloalkyl). In some embodiments, R⁶ is independently R³⁶-substituted cycloalkyl (e.g., C₃-C₈ cycloalkyl, C₃-C₆ cycloalkyl, or C₅-C₆ cycloalkyl). In some embodiments, R⁶ is independently an unsubstituted cycloalkyl (e.g., C₃-C₈ cycloalkyl, C₃-C₆ cycloalkyl, or C₅-C₆ cycloalkyl). In some embodiments, R⁶ is independently R³⁶-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R⁶ is independently R³⁶-substituted heterocycloalkyl (e.g., 3 to 8 membered heterocycloalkyl, 3 to 6 membered heterocycloalkyl, or 5 to 6 membered heterocycloalkyl). In some embodiments, R⁶ is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R⁶ is independently R³⁶-substituted or unsubstituted aryl (e.g., C₆-C₁₀ aryl, C₁₀ aryl, or phenyl). In some embodiments, R⁶ is independently R³⁶-substituted aryl (e.g., C₆-C₁₀ aryl, C₁₀ aryl, or phenyl). In some embodiments, R⁶ is independently an unsubstituted aryl (e.g., C₆-C₁₀ aryl, C₁₀ aryl, or phenyl). In some embodiments, R⁶ is independently R³⁶-substituted or unsubstituted heteroaryl (e.g., 5 to 10 membered heteroaryl, 5 to 9 membered heteroaryl, or 5 to 6 membered heteroaryl). In some embodiments, R⁶ is independently R³⁶-substituted heteroaryl (e.g., 5 to 10 membered heteroaryl, 5 to 9 membered heteroaryl, or 5 to 6 membered heteroaryl). In some embodiments, R⁶ is independently an unsubstituted heteroaryl (e.g., 5 to 10 membered heteroaryl, 5 to 9 membered heteroaryl, or 5 to 6 membered heteroaryl).

In some embodiments, R⁶ is independently —CX⁶₃. In some embodiments, R⁶ is independently —CHX⁶₂. In some embodiments, R⁶ is independently —CH₂X⁶. In some embodiments, R⁶ is independently —OCX⁶₃. In some embodiments, R⁶ is independently —OCH₂X⁶. In some embodiments, R⁶ is independently —OCHX⁶₂. In some embodiments, R⁶ is independently —CN. In some embodiments, R⁶ is independently —C(O)R^{6C}. In some embodiments, R⁶ is independently —C(O)—OR^{6C}. In some embodiments, R⁶ is independently —C(O)NR^{6A}R^{6B}. In some embodiments, R⁶ is independently —OR^{6D}. In some embodiments, R⁶ is independently hydrogen. X⁶ is independently halogen.

In some embodiments, R⁶ is independently —CCl₃. In some embodiments, R⁶ is independently —CBr₃. In some embodiments, R⁶ is independently —CF₃. In some embodiments, R⁶ is independently —CI₃. In some embodiments, R⁶ is independently —CHCl₂. In some embodiments, R⁶ is independently —CHBr₂. In some embodiments, R⁶ is independently —CHF₂. In some embodiments, R⁶ is independently —CHI₂. In some embodiments, R⁶ is independently —CH₂Cl. In some embodiments, R⁶ is independently —CH₂Br. In some embodiments, R⁶ is independently —CH₂F. In some embodiments, R⁶ is independently —CH₂I. In some embodiments, R⁶ is independently —CN. In some embodiments, R⁶ is independently —OH. In some embodiments, R⁶ is independently —COOH. In some embodiments, R⁶ is independently —CONH₂. In some embodiments, R⁶ is independently —OCCl₃. In some embodiments, R⁶ is independently —OCF₃. In some embodiments, R⁶ is independently —OCBr₃. In some embodiments, R⁶ is independently —OCI₃. In some embodiments, R⁶ is independently —OCHCl₂. In some embodiments, R⁶ is independently —OCHBr₂. In some embodiments, R⁶ is independently —OCHI₂. In some embodiments, R⁶ is independently —OCHF₂. In some embodiments, R⁶ is independently —OCH₂Cl. In some embodiments, R⁶ is independently —OCH₂Br. In some embodiments, R⁶ is independently —OCH₂I. In some embodiments, R⁶ is independently —OCH₂F. In some embodiments, R⁶ is independently —OCH₃. In some embodiments, R⁶ is independently —CH₃. In some embodiments, R⁶ is independently —CH₂CH₃. In some embodiments, R⁶ is independently unsubstituted propyl. In some embodiments, R⁶ is independently unsubstituted isopropyl. In some embodiments, R⁶ is independently unsubstituted butyl. In some embodiments, R⁶ is independently unsubstituted tert-butyl. In some embodiments, X⁶ is independently —F. In some embodiments, X⁶ is independently —Cl. In some embodiments, X⁶ is independently —Br. In some embodiments, X⁶ is independently —I.

In some embodiments, R³⁶ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R³⁷-substituted or unsubstituted alkyl (e.g., C₁-C₈ alkyl, C₁-C₆ alkyl, or C₁-C₄ alkyl), R³⁷-substituted or unsubstituted heteroalkyl (e.g., 2 to 8 membered heteroalkyl, 2 to 6 membered heteroalkyl, or 2 to 4 membered heteroalkyl), R³⁷-substituted or unsubstituted cycloalkyl (e.g., C₃-C₈ cycloalkyl, C₃-C₆ cycloalkyl, or C₅-C₆ cycloalkyl), R³⁷-substituted or unsubstituted heterocycloalkyl (e.g., 3 to 8 membered heterocycloalkyl, 3 to 6 membered heterocycloalkyl, or 5 to 6 membered heterocycloalkyl), R³⁷-substituted or unsubstituted aryl (e.g., C₆-C₁₀ aryl, C₁₀ aryl, or phenyl), or R³⁷-substituted or unsubstituted heteroaryl (e.g., 5 to 10 membered heteroaryl, 5 to 9 membered heteroaryl, or 5 to 6 membered heteroaryl). In some embodiments, R³⁶ is independently oxo, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R³⁶ is independently oxo. In some embodiments, R³⁶ is independently halogen. In some embodiments, R³⁶ is independently —CCl₃. In some embodiments, R³⁶ is independently —CBr₃. In some embodiments, R³⁶ is independently —CF₃. In some embodiments, R³⁶ is independently —CI₃. In some embodiments, R³⁶ is independently —CHCl₂. In some embodiments, R³⁶ is independently —CHBr₂. In some embodiments, R³⁶ is independently —CHF₂. In some embodiments, R³⁶ is independently —CHI₂. In some embodiments, R³⁶ is independently —CH₂Cl. In some embodiments, R³⁶ is independently —CH₂Br. In some embodiments, R³⁶ is independently —CH₂F. In some embodiments, R³⁶ is independently —CH₂I. In some embodiments, R³⁶ is independently —CN. In some embodiments, R³⁶ is independently —OH. In some embodiments, R³⁶ is independently —NH₂. In some embodiments, R³⁶ is independently —COOH. In some embodiments, R³⁶ is independently —CONH₂. In some embodiments, R³⁶ is independently —NO₂. In some embodiments, R³⁶ is independently —SH. In some embodiments, R³⁶ is independently —SO₃H. In some embodiments, R³⁶ is independently —SO₄H. In some embodiments, R³⁶ is independently —SO₂NH₂. In some embodiments, R³⁶ is independently —NHNH₂. In some embodiments, R³⁶ is independently —ONH₂. In some embodiments, R³⁶ is independently —NHC(O)NHNH₂. In some embodiments, R³⁶ is independently —NHC(O)NH₂. In some embodiments, R³⁶ is independently —NHSO₂H. In some embodiments, R³⁶ is independently —NHC(O)H. In some embodiments, R³⁶ is independently —NHC(O)OH. In some embodiments, R³⁶ is independently —NHOH. In some embodiments, R³⁶ is independently —OCCl₃. In some embodiments, R³⁶ is independently —OCF₃. In some embodiments, R³⁶ is independently —OCBr₃. In some embodiments, R³⁶ is independently —OCI₃. In some embodiments, R³⁶ is independently —OCHCl₂. In some embodiments, R³⁶ is independently —OCHBr₂. In some embodiments, R³⁶ is independently —OCHI₂. In some embodiments, R³⁶ is independently —OCHF₂. In some embodiments, R³⁶ is independently —OCH₂Cl. In some embodiments, R³⁶ is independently —OCH₂Br. In some embodiments, R³⁶ is independently —OCH₂I. In some embodiments, R³⁶ is independently —OCH₂F. In some embodiments, R³⁶ is independently —N₃. In some embodiments, R³⁶ is independently —OCH₃. In some embodiments, R³⁶ is independently —CH₃. In some embodiments, R³⁶ is independently —CH₂CH₃. In some embodiments, R³⁶ is independently unsubstituted propyl. In some embodiments, R³⁶ is independently unsubstituted isopropyl. In some embodiments, R³⁶ is independently unsubstituted butyl. In some embodiments, R³⁶ is independently unsubstituted tert-butyl. In some embodiments, R³⁶ is independently —F. In some embodiments, R³⁶ is independently —Cl. In some embodiments, R³⁶ is independently —Br. In some embodiments, R³⁶ is independently —I.

In some embodiments, R³⁶ is independently R³⁷-substituted or unsubstituted alkyl (e.g., C₁-C₈ alkyl, C₁-C₆ alkyl, or C₁-C₄ alkyl). In some embodiments, R³⁶ is independently R³⁷-substituted alkyl (e.g., C₁-C₈ alkyl, C₁-C₆ alkyl, or C₁-C₄ alkyl). In some embodiments, R³⁶ is independently an unsubstituted alkyl (e.g., C₁-C₈ alkyl, C₁-C₆ alkyl, or C₁-C₄ alkyl). In some embodiments, R³⁶ is independently R³⁷-substituted or unsubstituted heteroalkyl (e.g., 2 to 8 membered heteroalkyl, 2 to 6 membered heteroalkyl, or 2 to 4 membered heteroalkyl). In some embodiments, R³⁶ is independently R³⁷-substituted heteroalkyl (e.g., 2 to 8 membered heteroalkyl, 2 to 6 membered heteroalkyl, or 2 to 4 membered heteroalkyl). In some embodiments, R³⁶ is independently an unsubstituted heteroalkyl (e.g., 2 to 8 membered heteroalkyl, 2 to 6 membered heteroalkyl, or 2 to 4 membered heteroalkyl). In some embodiments, R³⁶ is independently R³⁷-substituted or unsubstituted cycloalkyl (e.g., C₃-C₈ cycloalkyl, C₃-C₆ cycloalkyl, or C₅-C₆ cycloalkyl). In some embodiments, R³⁶ is independently R³⁷-substituted cycloalkyl (e.g., C₃-C₈ cycloalkyl, C₃-C₆ cycloalkyl, or C₅-C₆ cycloalkyl). In some embodiments, R³⁶ is independently an unsubstituted cycloalkyl (e.g., C₃-C₈ cycloalkyl, C₃-C₆ cycloalkyl, or C₅-C₆ cycloalkyl). In some embodiments, R³⁶ is independently R³⁷-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R³⁶ is independently R³⁷-substituted heterocycloalkyl (e.g., 3 to 8 membered heterocycloalkyl, 3 to 6 membered heterocycloalkyl, or 5 to 6 membered heterocycloalkyl). In some embodiments, R³⁶ is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R³⁶ is independently R³⁷-substituted or unsubstituted aryl (e.g., C₆-C₁₀ aryl, C₁₀ aryl, or phenyl). In some embodiments, R³⁶ is independently R³⁷-substituted aryl (e.g., C₆-C₁₀ aryl, C₁₀ aryl, or phenyl). In some embodiments, R³⁶ is independently an unsubstituted aryl (e.g., C₆-C₁₀ aryl, C₁₀ aryl, or phenyl). In some embodiments, R³⁶ is independently R³⁷-substituted or unsubstituted heteroaryl (e.g., 5 to 10 membered heteroaryl, 5 to 9 membered heteroaryl, or 5 to 6 membered heteroaryl). In some embodiments, R³⁶ is independently R³⁷-substituted heteroaryl (e.g., 5 to 10 membered heteroaryl, 5 to 9 membered heteroaryl, or 5 to 6 membered heteroaryl). In some embodiments, R³⁶ is independently an unsubstituted heteroaryl (e.g., 5 to 10 membered heteroaryl, 5 to 9 membered heteroaryl, or 5 to 6 membered heteroaryl).

In some embodiments, R³⁷ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R³⁸-substituted or unsubstituted alkyl (e.g., C₁-C₈ alkyl, C₁-C₆ alkyl, or C₁-C₄ alkyl), R³⁸-substituted or unsubstituted heteroalkyl (e.g., 2 to 8 membered heteroalkyl, 2 to 6 membered heteroalkyl, or 2 to 4 membered heteroalkyl), R³⁸-substituted or unsubstituted cycloalkyl (e.g., C₃-C₈ cycloalkyl, C₃-C₆ cycloalkyl, or C₅-C₆ cycloalkyl), R³⁸-substituted or unsubstituted heterocycloalkyl (e.g., 3 to 8 membered heterocycloalkyl, 3 to 6 membered heterocycloalkyl, or 5 to 6 membered heterocycloalkyl), R³⁸-substituted or unsubstituted aryl (e.g., C₆-C₁₀ aryl, C₁₀ aryl, or phenyl), or R³⁸-substituted or unsubstituted heteroaryl (e.g., 5 to 10 membered heteroaryl, 5 to 9 membered heteroaryl, or 5 to 6 membered heteroaryl). In some embodiments, R³⁷ is independently oxo, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂C1, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R³⁷ is independently R³⁸-substituted or unsubstituted alkyl (e.g., C₁-C₈ alkyl, C₁-C₆ alkyl, or C₁-C₄ alkyl). In some embodiments, R³⁷ is independently R³⁸-substituted alkyl (e.g., C₁-C₈ alkyl, C₁-C₆ alkyl, or C₁-C₄ alkyl). In some embodiments, R³⁷ is independently an unsubstituted alkyl (e.g., C₁-C₈ alkyl, C₁-C₆ alkyl, or C₁-C₄ alkyl). In some embodiments, R³⁷ is independently R³⁸-substituted or unsubstituted heteroalkyl (e.g., 2 to 8 membered heteroalkyl, 2 to 6 membered heteroalkyl, or 2 to 4 membered heteroalkyl). In some embodiments, R³⁷ is independently R³⁸-substituted heteroalkyl (e.g., 2 to 8 membered heteroalkyl, 2 to 6 membered heteroalkyl, or 2 to 4 membered heteroalkyl). In some embodiments, R³⁷ is independently an unsubstituted heteroalkyl (e.g., 2 to 8 membered heteroalkyl, 2 to 6 membered heteroalkyl, or 2 to 4 membered heteroalkyl). In some embodiments, R³⁷ is independently R³⁸-substituted or unsubstituted cycloalkyl (e.g., C₃-C₈ cycloalkyl, C₃-C₆ cycloalkyl, or C₅-C₆ cycloalkyl). In some embodiments, R³⁷ is independently R³⁸-substituted cycloalkyl (e.g., C₃-C₈ cycloalkyl, C₃-C₆ cycloalkyl, or C₅-C₆ cycloalkyl). In some embodiments, R³⁷ is independently an unsubstituted cycloalkyl (e.g., C₃-C₈ cycloalkyl, C₃-C₆ cycloalkyl, or C₅-C₆ cycloalkyl). In some embodiments, R³⁷ is independently R³⁸-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R³⁷ is independently R³⁸-substituted heterocycloalkyl (e.g., 3 to 8 membered heterocycloalkyl, 3 to 6 membered heterocycloalkyl, or 5 to 6 membered heterocycloalkyl). In some embodiments, R³⁷ is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R³⁷ is independently R³⁸-substituted or unsubstituted aryl (e.g., C₆-C₁₀ aryl, C₁₀ aryl, or phenyl). In some embodiments, R³⁷ is independently R³⁸-substituted aryl (e.g., C₆-C₁₀ aryl, C₁₀ aryl, or phenyl). In some embodiments, R³⁷ is independently an unsubstituted aryl (e.g., C₆-C₁₀ aryl, C₁₀ aryl, or phenyl). In some embodiments, R³⁷ is independently R³⁸-substituted or unsubstituted heteroaryl (e.g., 5 to 10 membered heteroaryl, 5 to 9 membered heteroaryl, or 5 to 6 membered heteroaryl). In some embodiments, R³⁷ is independently R³⁸-substituted heteroaryl (e.g., 5 to 10 membered heteroaryl, 5 to 9 membered heteroaryl, or 5 to 6 membered heteroaryl). In some embodiments, R³⁷ is independently an unsubstituted heteroaryl (e.g., 5 to 10 membered heteroaryl, 5 to 9 membered heteroaryl, or 5 to 6 membered heteroaryl).

In some embodiments, R³⁷ is independently oxo. In some embodiments, R³⁷ is independently halogen. In some embodiments, R³⁷ is independently —CCl₃. In some embodiments, R³⁷ is independently —CBr₃. In some embodiments, R³⁷ is independently —CF₃. In some embodiments, R³⁷ is independently —CI₃. In some embodiments, R³⁷ is independently —CHCl₂. In some embodiments, R³⁷ is independently —CHBr₂. In some embodiments, R³⁷ is independently —CHF₂. In some embodiments, R³⁷ is independently —CHI₂. In some embodiments, R³⁷ is independently —CH₂Cl. In some embodiments, R³⁷ is independently —CH₂Br. In some embodiments, R³⁷ is independently —CH₂F. In some embodiments, R³⁷ is independently —CH₂I. In some embodiments, R³⁷ is independently —CN. In some embodiments, R³⁷ is independently —OH. In some embodiments, R³⁷ is independently —NH₂. In some embodiments, R³⁷ is independently —COOH. In some embodiments, R³⁷ is independently —CONH₂. In some embodiments, R³⁷ is independently —NO₂. In some embodiments, R³⁷ is independently —SH. In some embodiments, R³⁷ is independently —SO₃H. In some embodiments, R³⁷ is independently —SO₄H. In some embodiments, R³⁷ is independently —SO₂NH₂. In some embodiments, R³⁷ is independently —NHNH₂. In some embodiments, R³⁷ is independently —ONH₂. In some embodiments, R³⁷ is independently —NHC(O)NHNH₂. In some embodiments, R³⁷ is independently —NHC(O)NH₂. In some embodiments, R³⁷ is independently —NHSO₂H. In some embodiments, R³⁷ is independently —NHC(O)H. In some embodiments, R³⁷ is independently —NHC(O)OH. In some embodiments, R³⁷ is independently —NHOH. In some embodiments, R³⁷ is independently —OCCl₃. In some embodiments, R³⁷ is independently —OCF₃. In some embodiments, R³⁷ is independently —OCBr₃. In some embodiments, R³⁷ is independently —OCI₃. In some embodiments, R³⁷ is independently —OCHCl₂. In some embodiments, R³⁷ is independently —OCHBr₂. In some embodiments, R³⁷ is independently —OCHI₂. In some embodiments, R³⁷ is independently —OCHF₂. In some embodiments, R³⁷ is independently —OCH₂Cl. In some embodiments, R³⁷ is independently —OCH₂Br. In some embodiments, R³⁷ is independently —OCH₂I. In some embodiments, R³⁷ is independently —OCH₂F. In some embodiments, R³⁷ is independently —N₃. In some embodiments, R³⁷ is independently —OCH₃. In some embodiments, R³⁷ is independently —CH₃. In some embodiments, R³⁷ is independently —CH₂CH₃. In some embodiments, R³⁷ is independently unsubstituted propyl. In some embodiments, R³⁷ is independently unsubstituted isopropyl. In some embodiments, R³⁷ is independently unsubstituted butyl. In some embodiments, R³⁷ is independently unsubstituted tert-butyl. In some embodiments, R³⁷ is independently —F. In some embodiments, R³⁷ is independently —Cl. In some embodiments, R³⁷ is independently —Br. In some embodiments, R³⁷ is independently —I.

In some embodiments, R³⁸ is independently oxo, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R³⁸ is independently an unsubstituted alkyl (e.g., C₁-C₈ alkyl, C₁-C₆ alkyl, or C₁-C₄ alkyl). In some embodiments, R³⁸ is independently an unsubstituted heteroalkyl (e.g., 2 to 8 membered heteroalkyl, 2 to 6 membered heteroalkyl, or 2 to 4 membered heteroalkyl). In some embodiments, R³⁸ is independently an unsubstituted cycloalkyl (e.g., C₃-C₈ cycloalkyl, C₃-C₆ cycloalkyl, or C₅-C₆ cycloalkyl). In some embodiments, R³⁸ is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R³⁸ is independently an unsubstituted aryl (e.g., C₆-C₁₀ aryl, C₁₀ aryl, or phenyl). In some embodiments, R³⁸ is independently an unsubstituted heteroaryl (e.g., 5 to 10 membered heteroaryl, 5 to 9 membered heteroaryl, or 5 to 6 membered heteroaryl).

In some embodiments, R³⁸ is independently oxo. In some embodiments, R³⁸ is independently halogen. In some embodiments, R³⁸ is independently —CCl₃. In some embodiments, R³⁸ is independently —CBr₃. In some embodiments, R³⁸ is independently —CF₃. In some embodiments, R³⁸ is independently —CI₃. In some embodiments, R³⁸ is independently —CHCl₂. In some embodiments, R³⁸ is independently —CHBr₂. In some embodiments, R³⁸ is independently —CHF₂. In some embodiments, R³⁸ is independently —CHI₂. In some embodiments, R³⁸ is independently —CH₂Cl. In some embodiments, R³⁸ is independently —CH₂Br. In some embodiments, R³⁸ is independently —CH₂F. In some embodiments, R³⁸ is independently —CH₂I. In some embodiments, R³⁸ is independently —CN. In some embodiments, R³⁸ is independently —OH. In some embodiments, R³⁸ is independently —NH₂. In some embodiments, R³⁸ is independently —COOH. In some embodiments, R³⁸ is independently —CONH₂. In some embodiments, R³⁸ is independently —NO₂. In some embodiments, R³⁸ is independently —SH. In some embodiments, R³⁸ is independently —SO₃H. In some embodiments, R³⁸ is independently —SO₄H. In some embodiments, R³⁸ is independently —SO₂NH₂. In some embodiments, R³⁸ is independently —NHNH₂. In some embodiments, R³⁸ is independently —ONH₂. In some embodiments, R³⁸ is independently —NHC(O)NHNH₂. In some embodiments, R³⁸ is independently —NHC(O)NH₂. In some embodiments, R³⁸ is independently —NHSO₂H. In some embodiments, R³⁸ is independently —NHC(O)H. In some embodiments, R³⁸ is independently —NHC(O)OH. In some embodiments, R³⁸ is independently —NHOH. In some embodiments, R³⁸ is independently —OCCl₃. In some embodiments, R³⁸ is independently —OCF₃. In some embodiments, R³⁸ is independently —OCBr₃. In some embodiments, R³⁸ is independently —OCI₃. In some embodiments, R³⁸ is independently —OCHCl₂. In some embodiments, R³⁸ is independently —OCHBr₂. In some embodiments, R³⁸ is independently —OCHI₂. In some embodiments, R³⁸ is independently —OCHF₂. In some embodiments, R³⁸ is independently —OCH₂Cl. In some embodiments, R³⁸ is independently —OCH₂Br. In some embodiments, R³⁸ is independently —OCH₂I. In some embodiments, R³⁸ is independently —OCH₂F. In some embodiments, R³⁸ is independently —N₃. In some embodiments, R³⁸ is independently —OCH₃. In some embodiments, R³⁸ is independently —CH₃. In some embodiments, R³⁸ is independently —CH₂CH₃. In some embodiments, R³⁸ is independently unsubstituted propyl. In some embodiments, R³⁸ is independently unsubstituted isopropyl. In some embodiments, R³⁸ is independently unsubstituted butyl. In some embodiments, R³⁸ is independently unsubstituted tert-butyl. In some embodiments, R³⁸ is independently —F. In some embodiments, R³⁸ is independently —Cl. In some embodiments, R³⁸ is independently —Br. In some embodiments, R³⁸ is independently —I.

In some embodiments, R^{6A} is independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In some embodiments, R^{6B} is independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In some embodiments, R^{6C} is independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In some embodiments, R^{6D} is independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In some embodiments, R^{6A} and R^{6B} substituents bonded to the same nitrogen atom are independently joined to form a substituted or unsubstituted heterocycloalkyl (e.g., 3 to 8 membered heterocycloalkyl, 3 to 6 membered heterocycloalkyl, or 5 to 6 membered heterocycloalkyl) or substituted or unsubstituted heteroaryl (e.g., 5 to 10 membered heteroaryl, 5 to 9 membered heteroaryl, or 5 to 6 membered heteroaryl). In some embodiments, R^{6A} and R^{6B} substituents bonded to the same nitrogen atom are independently joined to form a substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R^{6A} and R^{6B} substituents bonded to the same nitrogen atom are independently joined to form a substituted heterocycloalkyl (e.g., 3 to 8 membered heterocycloalkyl, 3 to 6 membered heterocycloalkyl, or 5 to 6 membered heterocycloalkyl). In some embodiments, R^{6A} and R^{6B} substituents bonded to the same nitrogen atom are independently joined to form an unsubstituted heterocycloalkyl (e.g., 3 to 8 membered heterocycloalkyl, 3 to 6 membered heterocycloalkyl, or 5 to 6 membered heterocycloalkyl). In some embodiments, R^{6A} and R^{6B} substituents bonded to the same nitrogen atom are independently joined to form a substituted or unsubstituted heteroaryl (e.g., 5 to 10 membered heteroaryl, 5 to 9 membered heteroaryl, or 5 to 6 membered heteroaryl). In some embodiments, R^{6A} and R^{6B} substituents bonded to the same nitrogen atom are independently joined to form a substituted heteroaryl (e.g., 5 to 10 membered heteroaryl, 5 to 9 membered heteroaryl, or 5 to 6 membered heteroaryl). In some embodiments, R^{6A} and R^{6B} substituents bonded to the same nitrogen atom are independently joined to form an unsubstituted heteroaryl (e.g., 5 to 10 membered heteroaryl, 5 to 9 membered heteroaryl, or 5 to 6 membered heteroaryl).

In some embodiments, R^{6A}, R^{6B}, R^{6C}, and R^{6D} are independently hydrogen, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, R³⁶-substituted or unsubstituted alkyl (e.g., C₁-C₈ alkyl, C₁-C₆ alkyl, or C₁-C₄ alkyl), R³⁶-substituted or unsubstituted heteroalkyl (e.g., 2 to 8 membered heteroalkyl, 2 to 6 membered heteroalkyl, or 2 to 4 membered heteroalkyl), R³⁶-substituted or unsubstituted cycloalkyl (e.g., C₃-C₈ cycloalkyl, C₃-C₆ cycloalkyl, or C₅-C₆ cycloalkyl), R³⁶-substituted or unsubstituted heterocycloalkyl (e.g., 3 to 8 membered heterocycloalkyl, 3 to 6 membered heterocycloalkyl, or 5 to 6 membered heterocycloalkyl), R³⁶-substituted or unsubstituted aryl (e.g., C₆-C₁₀ aryl, C₁₀ aryl, or phenyl), or R³⁶-substituted or unsubstituted heteroaryl (e.g., 5 to 10 membered heteroaryl, 5 to 9 membered heteroaryl, or 5 to 6 membered heteroaryl). In some embodiments, R^{6A} and R^{6B} substituents bonded to the same nitrogen atom are independently joined to form an R³⁶-substituted or unsubstituted heterocycloalkyl (e.g., 3 to 8 membered heterocycloalkyl, 3 to 6 membered heterocycloalkyl, or 5 to 6 membered heterocycloalkyl) or R³⁶-substituted or unsubstituted heteroaryl (e.g., 5 to 10 membered heteroaryl, 5 to 9 membered heteroaryl, or 5 to 6 membered heteroaryl). In some embodiments, R^{6A} and R^{6B} substituents bonded to the same nitrogen atom are independently joined to form an R³⁶-substituted or unsubstituted heterocycloalkyl (e.g., 3 to 8 membered heterocycloalkyl, 3 to 6 membered heterocycloalkyl. In some embodiments, R^{6A} and R^{6B} substituents bonded to the same nitrogen atom are independently joined to form an R³⁶-substituted or unsubstituted heteroaryl (e.g., 5 to 10 membered heteroaryl, 5 to 9 membered heteroaryl, or 5 to 6 membered heteroaryl).

In some embodiments, R^{6A} is independently R³⁶-substituted or unsubstituted alkyl (e.g., C₁-C₈ alkyl, C₁-C₆ alkyl, or C₁-C₄ alkyl). In some embodiments, R^{6A} is independently R³⁶-substituted alkyl (e.g., C₁-C₈ alkyl, C₁-C₆ alkyl, or C₁-C₄ alkyl). In some embodiments, R^{6A} is independently an unsubstituted alkyl (e.g., C₁-C₈ alkyl, C₁-C₆ alkyl, or C₁-C₄ alkyl). In some embodiments, R^{6A} is independently R³⁶-substituted or unsubstituted heteroalkyl (e.g., 2 to 8 membered heteroalkyl, 2 to 6 membered heteroalkyl, or 2 to 4 membered heteroalkyl). In some embodiments, R^{6A} is independently R³⁶-substituted heteroalkyl (e.g., 2 to 8 membered heteroalkyl, 2 to 6 membered heteroalkyl, or 2 to 4 membered heteroalkyl). In some embodiments, R^{6A} is independently an unsubstituted heteroalkyl (e.g., 2 to 8 membered heteroalkyl, 2 to 6 membered heteroalkyl, or 2 to 4 membered heteroalkyl). In some embodiments, R^{6A} is independently R³⁶-substituted or unsubstituted cycloalkyl (e.g., C₃-C₈ cycloalkyl, C₃-C₆ cycloalkyl, or C₅-C₆ cycloalkyl). In some embodiments, R^{6A} is independently R³⁶-substituted cycloalkyl (e.g., C₃-C₈ cycloalkyl, C₃-C₆ cycloalkyl, or C₅-C₆ cycloalkyl). In some embodiments, R^{6A} is independently an unsubstituted cycloalkyl (e.g., C₃-C₈ cycloalkyl, C₃-C₆ cycloalkyl, or C₅-C₆ cycloalkyl). In some embodiments, R^{6A} is independently R³⁶-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R^{6A} is independently R³⁶-substituted heterocycloalkyl (e.g., 3 to 8 membered heterocycloalkyl, 3 to 6 membered heterocycloalkyl, or 5 to 6 membered heterocycloalkyl). In some embodiments, R^{6A} is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R^{6A} is independently R³⁶-substituted or unsubstituted aryl (e.g., C₆-C₁₀ aryl, C₁₀ aryl, or phenyl). In some embodiments, R^{6A} is independently R³⁶-substituted aryl (e.g., C₆-C₁₀ aryl, C₁₀ aryl, or phenyl). In some embodiments, R^{6A} is independently an unsubstituted aryl (e.g., C₆-C₁₀ aryl, C₁₀ aryl, or phenyl). In some embodiments, R^{6A} is independently R³⁶-substituted or unsubstituted heteroaryl (e.g., 5 to 10 membered heteroaryl, 5 to 9 membered heteroaryl, or 5 to 6 membered heteroaryl). In some embodiments, R^{6A} is independently R³⁶-substituted heteroaryl (e.g., 5 to 10 membered heteroaryl, 5 to 9 membered heteroaryl, or 5 to 6 membered heteroaryl). In some embodiments, R^{6A} is independently an unsubstituted heteroaryl (e.g., 5 to 10 membered heteroaryl, 5 to 9 membered heteroaryl, or 5 to 6 membered heteroaryl).

In some embodiments, R^{6A} is independently —CCl₃. In some embodiments, R^{6A} is independently —CBr₃. In some embodiments, R^{6A} is independently —CF₃. In some embodiments, R^{6A} is independently —CI₃. In some embodiments, R^{6A} is independently —CHCl₂. In some embodiments, R^{6A} is independently —CHBr₂. In some embodiments, R^{6A} is independently —CHF₂. In some embodiments, R^{6A} is independently —CHI₂. In some embodiments, R^{6A} is independently —CH₂Cl. In some embodiments, R^{6A} is independently —CH₂Br. In some embodiments, R^{6A} is independently —CH₂F. In some embodiments, R^{6A} is independently —CH₂I. In some embodiments, R^{6A} is independently —CN. In some embodiments, R^{6A} is independently —OH. In some embodiments, R^{6A} is independently —COOH. In some embodiments, R^{6A} is independently —CONH₂. In some embodiments, R^{6A} is independently —OCCl₃. In some embodiments, R^{6A} is independently —OCF₃. In some embodiments, R^{6A} is independently —OCBr₃. In some embodiments, R^{6A} is independently —OCI₃. In some embodiments, R^{6A} is independently —OCHCl₂. In some embodiments, R^{6A} is independently —OCHBr₂. In some embodiments, R^{6A} is independently —OCHI₂. In some embodiments, R^{6A} is independently —OCHF₂. In some embodiments, R^{6A} is independently —OCH₂Cl. In some embodiments, R^{6A} is independently —OCH₂Br. In some embodiments, R^{6A} is independently —OCH₂I. In some embodiments, R^{6A} is independently —OCH₂F. In some embodiments, R^{6A} is independently —OCH₃. In some embodiments, R^{6A} is independently —CH₃. In some embodiments, R^{6A} is independently —CH₂CH₃. In some embodiments, R^{6A} is independently unsubstituted propyl. In some embodiments, R^{6A} is independently unsubstituted isopropyl. In some embodiments, R^{6A} is independently unsubstituted butyl. In some embodiments, R^{6A} is independently unsubstituted tert-butyl. In some embodiments, R^{6A} is independently hydrogen.

In some embodiments, R^{6B} is independently R³⁶-substituted or unsubstituted alkyl (e.g., C₁-C₈ alkyl, C₁-C₆ alkyl, or C₁-C₄ alkyl). In some embodiments, R^{6B} is independently R³⁶-substituted alkyl (e.g., C₁-C₈ alkyl, C₁-C₆ alkyl, or C₁-C₄ alkyl). In some embodiments, R^{6B} is independently an unsubstituted alkyl (e.g., C₁-C₈ alkyl, C₁-C₆ alkyl, or C₁-C₄ alkyl). In some embodiments, R^{6B} is independently R³⁶-substituted or unsubstituted heteroalkyl (e.g., 2 to 8 membered heteroalkyl, 2 to 6 membered heteroalkyl, or 2 to 4 membered heteroalkyl). In some embodiments, R^{6B} is independently R³⁶-substituted heteroalkyl (e.g., 2 to 8 membered heteroalkyl, 2 to 6 membered heteroalkyl, or 2 to 4 membered heteroalkyl). In some embodiments, R^{6B} is independently an unsubstituted heteroalkyl (e.g., 2 to 8 membered heteroalkyl, 2 to 6 membered heteroalkyl, or 2 to 4 membered heteroalkyl). In some embodiments, R^{6B} is independently R³⁶-substituted or unsubstituted cycloalkyl (e.g., C₃-C₈ cycloalkyl, C₃-C₆ cycloalkyl, or C₅-C₆ cycloalkyl). In some embodiments, R^{6B} is independently R³⁶-substituted cycloalkyl (e.g., C₃-C₈ cycloalkyl, C₃-C₆ cycloalkyl, or C₅-C₆ cycloalkyl). In some embodiments, R^{6B} is independently an unsubstituted cycloalkyl (e.g., C₃-C₈ cycloalkyl, C₃-C₆ cycloalkyl, or C₅-C₆ cycloalkyl). In some embodiments, R^{6B} is independently R³⁶-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R^{6B} is independently R³⁶-substituted heterocycloalkyl (e.g., 3 to 8 membered heterocycloalkyl, 3 to 6 membered heterocycloalkyl, or 5 to 6 membered heterocycloalkyl). In some embodiments, R^{6B} is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R^{6B} is independently R³⁶-substituted or unsubstituted aryl (e.g., C₆-C₁₀ aryl, C₁₀ aryl, or phenyl). In some embodiments, R^{6B} is independently R³⁶-substituted aryl (e.g., C₆-C₁₀ aryl, C₁₀ aryl, or phenyl). In some embodiments, R^{6B} is independently an unsubstituted aryl (e.g., C₆-C₁₀ aryl, C₁₀ aryl, or phenyl). In some embodiments, R^{6B} is independently R³⁶-substituted or unsubstituted heteroaryl (e.g., 5 to 10 membered heteroaryl, 5 to 9 membered heteroaryl, or 5 to 6 membered heteroaryl). In some embodiments, R^{6B} is independently R³⁶-substituted heteroaryl (e.g., 5 to 10 membered heteroaryl, 5 to 9 membered heteroaryl, or 5 to 6 membered heteroaryl). In some embodiments, R^{6B} is independently an unsubstituted heteroaryl (e.g., 5 to 10 membered heteroaryl, 5 to 9 membered heteroaryl, or 5 to 6 membered heteroaryl).

In some embodiments, R^{6B} is independently —CCl₃. In some embodiments, R^{6B} is independently —CBr₃. In some embodiments, R^{6B} is independently —CF₃. In some embodiments, R^{6B} is independently —CI₃. In some embodiments, R^{6B} is independently —CHCl₂. In some embodiments, R^{6B} is independently —CHBr₂. In some embodiments, R^{6B} is independently —CHF₂. In some embodiments, R^{6B} is independently —CHI₂. In some embodiments, R^{6B} is independently —CH₂Cl. In some embodiments, R^{6B} is independently —CH₂Br. In some embodiments, R^{6B} is independently —CH₂F. In some embodiments, R^{6B} is independently —CH₂I. In some embodiments, R^{6B} is independently —CN. In some embodiments, R^{6B} is independently —OH. In some embodiments, R^{6B} is independently —COOH. In some embodiments, R^{6B} is independently —CONH₂. In some embodiments, R^{6B} is independently —OCCl₃. In some embodiments, R^{6B} is independently —OCF₃. In some embodiments, R^{6B} is independently —OCBr₃. In some embodiments, R^{6B} is independently —OCI₃. In some embodiments, R^{6B} is independently —OCHCl₂. In some embodiments, R^{6B} is independently —OCHBr₂. In some embodiments, R^{6B} is independently —OCHI₂. In some embodiments, R^{6B} is independently —OCHF₂. In some embodiments, R^{6B} is independently —OCH₂Cl. In some embodiments, R^{6B} is independently —OCH₂Br. In some embodiments, R^{6B} is independently —OCH₂I. In some embodiments, R^{6B} is independently —OCH₂F. In some embodiments, R^{6B} is independently —OCH₃. In some embodiments, R^{6B} is independently —CH₃. In some embodiments, R^{6B} is independently —CH₂CH₃. In some embodiments, R^{6B} is independently unsubstituted propyl. In some embodiments, R^{6B} is independently unsubstituted isopropyl. In some embodiments, R^{6B} is independently unsubstituted butyl. In some embodiments, R^{6B} is independently unsubstituted tert-butyl. In some embodiments, R^{6B} is independently hydrogen.

In some embodiments, R^{6C} is independently R³⁶-substituted or unsubstituted alkyl (e.g., C₁-C₈ alkyl, C₁-C₆ alkyl, or C₁-C₄ alkyl). In some embodiments, R^{6C} is independently R³⁶-substituted alkyl (e.g., C₁-C₈ alkyl, C₁-C₆ alkyl, or C₁-C₄ alkyl). In some embodiments, R^{6C} is independently an unsubstituted alkyl (e.g., C₁-C₈ alkyl, C₁-C₆ alkyl, or C₁-C₄ alkyl). In some embodiments, R^{6C} is independently R³⁶-substituted or unsubstituted heteroalkyl (e.g., 2 to 8 membered heteroalkyl, 2 to 6 membered heteroalkyl, or 2 to 4 membered heteroalkyl). In some embodiments, R^{6C} is independently R³⁶-substituted heteroalkyl (e.g., 2 to 8 membered heteroalkyl, 2 to 6 membered heteroalkyl, or 2 to 4 membered heteroalkyl). In some embodiments, R^{6C} is independently an unsubstituted heteroalkyl (e.g., 2 to 8 membered heteroalkyl, 2 to 6 membered heteroalkyl, or 2 to 4 membered heteroalkyl). In some embodiments, R^{6C} is independently R³⁶-substituted or unsubstituted cycloalkyl (e.g., C₃-C₈ cycloalkyl, C₃-C₆ cycloalkyl, or C₅-C₆ cycloalkyl). In some embodiments, R^{6C} is independently R³⁶-substituted cycloalkyl (e.g., C₃-C₈ cycloalkyl, C₃-C₆ cycloalkyl, or C₅-C₆ cycloalkyl). In some embodiments, R^{6C} is independently an unsubstituted cycloalkyl (e.g., C₃-C₈ cycloalkyl, C₃-C₆ cycloalkyl, or C₅-C₆ cycloalkyl). In some embodiments, R^{6C} is independently R³⁶-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R^{6C} is independently R³⁶-substituted heterocycloalkyl (e.g., 3 to 8 membered heterocycloalkyl, 3 to 6 membered heterocycloalkyl, or 5 to 6 membered heterocycloalkyl). In some embodiments, R^{6C} is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R^{6C} is independently R³⁶-substituted or unsubstituted aryl (e.g., C₆-C₁₀ aryl, C₁₀ aryl, or phenyl). In some embodiments, R^{6C} is independently R³⁶-substituted aryl (e.g., C₆-C₁₀ aryl, C₁₀ aryl, or phenyl). In some embodiments, R^{6C} is independently an unsubstituted aryl (e.g., C₆-C₁₀ aryl, C₁₀ aryl, or phenyl). In some embodiments, R^{6C} is independently R³⁶-substituted or unsubstituted heteroaryl (e.g., 5 to 10 membered heteroaryl, 5 to 9 membered heteroaryl, or 5 to 6 membered heteroaryl). In some embodiments, R^{6C} is independently R³⁶-substituted heteroaryl (e.g., 5 to 10 membered heteroaryl, 5 to 9 membered heteroaryl, or 5 to 6 membered heteroaryl). In some embodiments, R^{6C} is independently an unsubstituted heteroaryl (e.g., 5 to 10 membered heteroaryl, 5 to 9 membered heteroaryl, or 5 to 6 membered heteroaryl).

In some embodiments, R^{6C} is independently —CCl₃. In some embodiments, R^{6C} is independently —CBr₃. In some embodiments, R^{6C} is independently —CF₃. In some embodiments, R^{6C} is independently —CI₃. In some embodiments, R^{6C} is independently —CHCl₂. In some embodiments, R^{6C} is independently —CHBr₂. In some embodiments, R^{6C} is independently —CHF₂. In some embodiments, R^{6C} is independently —CHI₂. In some embodiments, R^{6C} is independently —CH₂Cl. In some embodiments, R^{6C} is independently —CH₂Br. In some embodiments, R^{6C} is independently —CH₂F. In some embodiments, R^{6C} is independently —CH₂I. In some embodiments, R^{6C} is independently —CN. In some embodiments, R^{6C} is independently —OH. In some embodiments, R^{6C} is independently —COOH. In some embodiments, R^{6C} is independently —CONH₂. In some embodiments, R^{6C} is independently —OCCl₃. In some embodiments, R^{6C} is independently —OCF₃. In some embodiments, R^{6C} is independently —OCBr₃. In some embodiments, R^{6C} is independently —OCI₃. In some embodiments, R^{6C} is independently —OCHCl₂. In some embodiments, R^{6C} is independently —OCHBr₂. In some embodiments, R^{6C} is independently —OCHI₂. In some embodiments, R^{6C} is independently —OCHF₂. In some embodiments, R^{6C} is independently —OCH₂Cl. In some embodiments, R^{6C} is independently —OCH₂Br. In some embodiments, R^{6C} is independently —OCH₂I. In some embodiments, R^{6C} is independently —OCH₂F. In some embodiments, R^{6C} is independently —OCH₃. In some embodiments, R^{6C} is independently —CH₃. In some embodiments, R^{6C} is independently —CH₂CH₃. In some embodiments, R^{6C} is independently unsubstituted propyl. In some embodiments, R^{6C} is independently unsubstituted isopropyl. In some embodiments, R^{6C} is independently unsubstituted butyl. In some embodiments, R^{6C} is independently unsubstituted tert-butyl. In some embodiments, R^{6C} is independently hydrogen.

In some embodiments, R^{6D} is independently R³⁶-substituted or unsubstituted alkyl (e.g., C₁-C₈ alkyl, C₁-C₆ alkyl, or C₁-C₄ alkyl). In some embodiments, R^{6D} is independently R³⁶-substituted alkyl (e.g., C₁-C₈ alkyl, C₁-C₆ alkyl, or C₁-C₄ alkyl). In some embodiments, R^{6D} is independently an unsubstituted alkyl (e.g., C₁-C₈ alkyl, C₁-C₆ alkyl, or C₁-C₄ alkyl). In some embodiments, R^{6D} is independently R³⁶-substituted or unsubstituted heteroalkyl (e.g., 2 to 8 membered heteroalkyl, 2 to 6 membered heteroalkyl, or 2 to 4 membered heteroalkyl). In some embodiments, R^{6D} is independently R³⁶-substituted heteroalkyl (e.g., 2 to 8 membered heteroalkyl, 2 to 6 membered heteroalkyl, or 2 to 4 membered heteroalkyl). In some embodiments, R^{6D} is independently an unsubstituted heteroalkyl (e.g., 2 to 8 membered heteroalkyl, 2 to 6 membered heteroalkyl, or 2 to 4 membered heteroalkyl). In some embodiments, R^{6D} is independently R³⁶-substituted or unsubstituted cycloalkyl (e.g., C₃-C₈ cycloalkyl, C₃-C₆ cycloalkyl, or C₅-C₆ cycloalkyl). In some embodiments, R^{6D} is independently R³⁶-substituted cycloalkyl (e.g., C₃-C₈ cycloalkyl, C₃-C₆ cycloalkyl, or C₅-C₆ cycloalkyl). In some embodiments, R^{6D} is independently an unsubstituted cycloalkyl (e.g., C₃-C₈ cycloalkyl, C₃-C₆ cycloalkyl, or C₅-C₆ cycloalkyl). In some embodiments, R^{6D} is independently R³⁶-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R^{6D} is independently R³⁶-substituted heterocycloalkyl (e.g., 3 to 8 membered heterocycloalkyl, 3 to 6 membered heterocycloalkyl, or 5 to 6 membered heterocycloalkyl). In some embodiments, R^{6D} is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R^{6D} is independently R³⁶-substituted or unsubstituted aryl (e.g., C₆-C₁₀ aryl, C₁₀ aryl, or phenyl). In some embodiments, R^{6D} is independently R³⁶-substituted aryl (e.g., C₆-C₁₀ aryl, C₁₀ aryl, or phenyl). In some embodiments, R^{6D} is independently an unsubstituted aryl (e.g., C₆-C₁₀ aryl, C₁₀ aryl, or phenyl). In some embodiments, R^{6D} is independently R³⁶-substituted or unsubstituted heteroaryl (e.g., 5 to 10 membered heteroaryl, 5 to 9 membered heteroaryl, or 5 to 6 membered heteroaryl). In some embodiments, R^{6D} is independently R³⁶-substituted heteroaryl (e.g., 5 to 10 membered heteroaryl, 5 to 9 membered heteroaryl, or 5 to 6 membered heteroaryl). In some embodiments, R^{6D} is independently an unsubstituted heteroaryl (e.g., 5 to 10 membered heteroaryl, 5 to 9 membered heteroaryl, or 5 to 6 membered heteroaryl).

In some embodiments, R^{6D} is independently —CCl₃. In some embodiments, R^{6D} is independently —CBr₃. In some embodiments, R^{6D} is independently —CF₃. In some embodiments, R^{6D} is independently —CI₃. In some embodiments, R^{6D} is independently —CHCl₂. In some embodiments, R^{6D} is independently —CHBr₂. In some embodiments, R^{6D} is independently —CHF₂. In some embodiments, R^{6D} is independently —CHI₂. In some embodiments, R^{6D} is independently —CH₂Cl. In some embodiments, R^{6D} is independently —CH₂Br. In some embodiments, R^{6D} is independently —CH₂F. In some embodiments, R^{6D} is independently —CH₂I. In some embodiments, R^{6D} is independently —CN. In some embodiments, R^{6D} is independently —OH. In some embodiments, R^{6D} is independently —COOH. In some embodiments, R^{6D} is independently —CONH₂. In some embodiments, R^{6D} is independently —OCCl₃. In some embodiments, R^{6D} is independently —OCF₃. In some embodiments, R^{6D} is independently —OCBr₃. In some embodiments, R^{6D} is independently —OCI₃. In some embodiments, R^{6D} is independently —OCHCl₂. In some embodiments, R^{6D} is independently —OCHBr₂. In some embodiments, R^{6D} is independently —OCHI₂. In some embodiments, R^{6D} is independently —OCHF₂. In some embodiments, R^{6D} is independently —OCH₂Cl. In some embodiments, R^{6D} is independently —OCH₂Br. In some embodiments, R^{6D} is independently —OCH₂I. In some embodiments, R^{6D} is independently —OCH₂F. In some embodiments, R^{6D} is independently —OCH₃. In some embodiments, R^{6D} is independently —CH₃. In some embodiments, R^{6D} is independently —CH₂CH₃. In some embodiments, R^{6D} is independently unsubstituted propyl. In some embodiments, R^{6D} is independently unsubstituted isopropyl. In some embodiments, R^{6D} is independently unsubstituted butyl. In some embodiments, R^{6D} is independently unsubstituted tert-butyl. In some embodiments, R^{6D} is independently hydrogen.

In some embodiments, L⁵ is independently a bond, —S(O)₂—, —S(O)—, ═N—, —NH—, —O—, —S—, —C(O)—, —C(O)NH—, —NHC(O)—, —NHC(O)NH—, —C(O)O—, —OC(O)—, substituted or unsubstituted alkylene (e.g., C₁-C₈ alkylene, C₁-C₆ alkylene, or C₁-C₄ alkylene), substituted or unsubstituted heteroalkylene (e.g., 2 to 8 membered heteroalkylene, 2 to 6 membered heteroalkylene, or 2 to 4 membered heteroalkylene), substituted or unsubstituted cycloalkylene (e.g., C₃-C₈ cycloalkylene, C₃-C₆ cycloalkylene, or C₅-C₆ cycloalkylene), substituted or unsubstituted heterocycloalkylene (e.g., 3 to 8 membered heterocycloalkylene, 3 to 6 membered heterocycloalkylene, or 5 to 6 membered heterocycloalkylene), substituted or unsubstituted arylene (e.g., C₆-C₁₀ arylene, C₁₀ arylene, or phenylene), or substituted or unsubstituted heteroarylene (e.g., 5 to 10 membered heteroarylene, 5 to 9 membered heteroarylene, or 5 to 6 membered heteroarylene). In some embodiments, L⁵ is a bond. In some embodiments, L⁵ is independently a —S(O)₂—. In some embodiments, L⁵ is independently a —S(O)—. In some embodiments, L⁵ is independently a —NH—. In some embodiments, L⁵ is independently a —O—. In some embodiments, L⁵ is independently a —S—. In some embodiments, L⁵ is independently a —C(O)—. In some embodiments, L⁵ is independently a —C(O)NH—. In some embodiments, L⁵ is independently a —NHC(O)—. In some embodiments, L⁵ is independently a —NHC(O)NH—. In some embodiments, L⁵ is independently a —C(O)O—. In some embodiments, L⁵ is independently —OC(O)—. In some embodiments, L⁵ is independently a bond. In some embodiments, L⁵ is independently —NR⁵—.

In some embodiments, L⁵ is independently ═N—. In some embodiments, L⁵ is independently —C(O)NR⁵—. In some embodiments, L⁵ is independently —NR⁵C(O)—. In some embodiments, L⁵ is independently —NR⁵C(O)NH—. In some embodiments, L⁵ is independently —NHC(O)NR⁵—.

In some embodiments, L⁵ is substituted or unsubstituted alkylene (e.g., C₁-C₈ alkylene, C₁-C₆ alkylene, or C₁-C₄ alkylene). In some embodiments, L⁵ is substituted alkylene (e.g., C₁-C₈ alkylene, C₁-C₆ alkylene, or C₁-C₄ alkylene). In some embodiments, L⁵ is an unsubstituted alkylene (e.g., C₁-C₈ alkylene, C₁-C₆ alkylene, or C₁-C₄ alkylene). In some embodiments, L⁵ is substituted or unsubstituted heteroalkylene (e.g., 2 to 8 membered heteroalkylene, 2 to 6 membered heteroalkylene, or 2 to 4 membered heteroalkylene). In some embodiments, L⁵ is substituted heteroalkylene (e.g., 2 to 8 membered heteroalkylene, 2 to 6 membered heteroalkylene, or 2 to 4 membered heteroalkylene). In some embodiments, L⁵ is an unsubstituted heteroalkylene (e.g., 2 to 8 membered heteroalkylene, 2 to 6 membered heteroalkylene, or 2 to 4 membered heteroalkylene). In some embodiments, L⁵ is substituted or unsubstituted cycloalkylene (e.g., C₃-C₈ cycloalkylene, C₃-C₆ cycloalkylene, or C₅-C₆ cycloalkylene). In some embodiments, L⁵ is substituted cycloalkylene (e.g., C₃-C₈ cycloalkylene, C₃-C₆ cycloalkylene, or C₅-C₆ cycloalkylene). In some embodiments, L⁵ is an unsubstituted cycloalkylene (e.g., C₃-C₈ cycloalkylene, C₃-C₆ cycloalkylene, or C₅-C₆ cycloalkylene). In some embodiments, L⁵ is substituted or unsubstituted heterocycloalkylene (e.g., 3 to 8 membered heterocycloalkylene, 3 to 6 membered heterocycloalkylene, or 5 to 6 membered heterocycloalkylene). In some embodiments, L⁵ is substituted heterocycloalkylene (e.g., 3 to 8 membered heterocycloalkylene, 3 to 6 membered heterocycloalkylene, or 5 to 6 membered heterocycloalkylene). In some embodiments, L⁵ is an unsubstituted heterocycloalkylene (e.g., 3 to 8 membered heterocycloalkylene, 3 to 6 membered heterocycloalkylene, or 5 to 6 membered heterocycloalkylene). In some embodiments, L⁵ is substituted or unsubstituted arylene (e.g., C₆-C₁₀ arylene, C₁₀ arylene, or phenylene). In some embodiments, L⁵ is substituted arylene (e.g., C₆-C₁₀ arylene, C₁₀ arylene, or phenylene). In some embodiments, L⁵ is an unsubstituted arylene (e.g., C₆-C₁₀ arylene, C₁₀ arylene, or phenylene). In some embodiments, L⁵ is substituted or unsubstituted heteroarylene (e.g., 5 to 10 membered heteroarylene, 5 to 9 membered heteroarylene, or 5 to 6 membered heteroarylene). In some embodiments, L⁵ is substituted heteroarylene (e.g., 5 to 10 membered heteroarylene, 5 to 9 membered heteroarylene, or 5 to 6 membered heteroarylene). In some embodiments, L⁵ is an unsubstituted heteroarylene (e.g., 5 to 10 membered heteroarylene, 5 to 9 membered heteroarylene, or 5 to 6 membered heteroarylene).

In some embodiments, L⁵ is independently a bond, —S(O)₂—, —S(O)—, ═N—, —NH—, —O—, —S—, —C(O)—, —C(O)NH—, —NHC(O)—, —NHC(O)NH—, —C(O)O—, —OC(O)—, R⁴⁷-substituted or unsubstituted alkylene (e.g., C₁-C₈ alkylene, C₁-C₆ alkylene, or C₁-C₄ alkylene), R⁴⁷-substituted or unsubstituted heteroalkylene (e.g., 2 to 8 membered heteroalkylene, 2 to 6 membered heteroalkylene, or 2 to 4 membered heteroalkylene), R⁴⁷-substituted or unsubstituted cycloalkylene (e.g., C₃-C₈ cycloalkylene, C₃-C₆ cycloalkylene, or C₅-C₆ cycloalkylene), R⁴⁷-substituted or unsubstituted heterocycloalkylene (e.g., 3 to 8 membered heterocycloalkylene, 3 to 6 membered heterocycloalkylene, or 5 to 6 membered heterocycloalkylene), R⁴⁷-substituted or unsubstituted arylene (e.g., C₆-C₁₀ arylene, C₁₀ arylene, or phenylene), or R⁴⁷-substituted or unsubstituted heteroarylene (e.g., 5 to 10 membered heteroarylene, 5 to 9 membered heteroarylene, or 5 to 6 membered heteroarylene). In some embodiments, L⁵ is independently a bond, —S(O)₂—, —S(O)—, —NH—, ═N—, —O—, —S—, —C(O)—, —C(O)NH—, —NHC(O)—, —NHC(O)NH—, —C(O)O—, —OC(O)—, unsubstituted alkylene (e.g., C₁-C₈ alkylene, C₁-C₆ alkylene, or C₁-C₄ alkylene), unsubstituted heteroalkylene (e.g., 2 to 8 membered heteroalkylene, 2 to 6 membered heteroalkylene, or 2 to 4 membered heteroalkylene), unsubstituted cycloalkylene (e.g., C₃-C₈ cycloalkylene, C₃-C₆ cycloalkylene, or C₅-C₆ cycloalkylene), unsubstituted heterocycloalkylene (e.g., 3 to 8 membered heterocycloalkylene, 3 to 6 membered heterocycloalkylene, or 5 to 6 membered heterocycloalkylene), unsubstituted arylene (e.g., C₆-C₁₀ arylene, C₁₀ arylene, or phenylene), or unsubstituted heteroarylene (e.g., 5 to 10 membered heteroarylene, 5 to 9 membered heteroarylene, or 5 to 6 membered heteroarylene).

In some embodiments, L⁵ is R⁴⁷-substituted or unsubstituted alkylene (e.g., C₁-C₈ alkylene, C₁-C₆ alkylene, or C₁-C₄ alkylene). In some embodiments, L⁵ is R⁴⁷-substituted alkylene (e.g., C₁-C₈ alkylene, C₁-C₆ alkylene, or C₁-C₄ alkylene). In some embodiments, L⁵ is an unsubstituted alkylene (e.g., C₁-C₈ alkylene, C₁-C₆ alkylene, or C₁-C₄ alkylene). In some embodiments, L⁵ is R⁴⁷-substituted or unsubstituted heteroalkylene (e.g., 2 to 8 membered heteroalkylene, 2 to 6 membered heteroalkylene, or 2 to 4 membered heteroalkylene). In some embodiments, L⁵ is R⁴⁷-substituted heteroalkylene (e.g., 2 to 8 membered heteroalkylene, 2 to 6 membered heteroalkylene, or 2 to 4 membered heteroalkylene). In some embodiments, L⁵ is an unsubstituted heteroalkylene (e.g., 2 to 8 membered heteroalkylene, 2 to 6 membered heteroalkylene, or 2 to 4 membered heteroalkylene). In some embodiments, L⁵ is R⁴⁷-substituted or unsubstituted cycloalkylene (e.g., C₃-C₈ cycloalkylene, C₃-C₆ cycloalkylene, or C₅-C₆ cycloalkylene). In some embodiments, L⁵ is R⁴⁷-substituted cycloalkylene (e.g., C₃-C₈ cycloalkylene, C₃-C₆ cycloalkylene, or C₅-C₆ cycloalkylene). In some embodiments, L⁵ is an unsubstituted cycloalkylene (e.g., C₃-C₈ cycloalkylene, C₃-C₆ cycloalkylene, or C₅-C₆ cycloalkylene). In some embodiments, L⁵ is R⁴⁷-substituted or unsubstituted heterocycloalkylene (e.g., 3 to 8 membered heterocycloalkylene, 3 to 6 membered heterocycloalkylene, or 5 to 6 membered heterocycloalkylene). In some embodiments, L⁵ is R⁴⁷-substituted heterocycloalkylene (e.g., 3 to 8 membered heterocycloalkylene, 3 to 6 membered heterocycloalkylene, or 5 to 6 membered heterocycloalkylene). In some embodiments, L⁵ is an unsubstituted heterocycloalkylene (e.g., 3 to 8 membered heterocycloalkylene, 3 to 6 membered heterocycloalkylene, or 5 to 6 membered heterocycloalkylene). In some embodiments, L⁵ is R⁴⁷-substituted or unsubstituted arylene (e.g., C₆-C₁₀ arylene, C₁₀ arylene, or phenylene). In some embodiments, L⁵ is R⁴⁷-substituted arylene (e.g., C₆-C₁₀ arylene, C₁₀ arylene, or phenylene). In some embodiments, L⁵ is an unsubstituted arylene (e.g., C₆-C₁₀ arylene, C₁₀ arylene, or phenylene). In some embodiments, L⁵ is R⁴⁷-substituted or unsubstituted heteroarylene (e.g., 5 to 10 membered heteroarylene, 5 to 9 membered heteroarylene, or 5 to 6 membered heteroarylene). In some embodiments, L⁵ is R⁴⁷-substituted heteroarylene (e.g., 5 to 10 membered heteroarylene, 5 to 9 membered heteroarylene, or 5 to 6 membered heteroarylene). In some embodiments, L⁵ is an unsubstituted heteroarylene (e.g., 5 to 10 membered heteroarylene, 5 to 9 membered heteroarylene, or 5 to 6 membered heteroarylene).

In some embodiments, R⁴⁷ is independently oxo, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R⁴⁷ is independently oxo. In some embodiments, R⁴⁷ is independently halogen. In some embodiments, R⁴⁷ is independently —CCl₃. In some embodiments, R⁴⁷ is independently —CBr₃. In some embodiments, R⁴⁷ is independently —CF₃.

In some embodiments, R⁴⁷ is independently —CI₃. In some embodiments, R⁴⁷ is independently —CHCl₂. In some embodiments, R⁴⁷ is independently —CHBr₂. In some embodiments, R⁴⁷ is independently —CHF₂. In some embodiments, R⁴⁷ is independently —CHI₂. In some embodiments, R⁴⁷ is independently —CH₂Cl. In some embodiments, R⁴⁷ is independently —CH₂Br. In some embodiments, R⁴⁷ is independently —CH₂F. In some embodiments, R⁴⁷ is independently —CH₂I. In some embodiments, R⁴⁷ is independently —CN. In some embodiments, R⁴⁷ is independently —OH. In some embodiments, R⁴⁷ is independently —NH₂. In some embodiments, R⁴⁷ is independently —COOH. In some embodiments, R⁴⁷ is independently —CONH₂. In some embodiments, R⁴⁷ is independently —NO₂. In some embodiments, R⁴⁷ is independently —SH. In some embodiments, R⁴⁷ is independently —SO₃H. In some embodiments, R⁴⁷ is independently —SO₄H. In some embodiments, R⁴⁷ is independently —SO₂NH₂. In some embodiments, R⁴⁷ is independently —NHNH₂. In some embodiments, R⁴⁷ is independently —ONH₂. In some embodiments, R⁴⁷ is independently —NHC(O)NHNH₂. In some embodiments, R⁴⁷ is independently —NHC(O)NH₂. In some embodiments, R⁴⁷ is independently —NHSO₂H. In some embodiments, R⁴⁷ is independently —NHC(O)H. In some embodiments, R⁴⁷ is independently —NHC(O)OH. In some embodiments, R⁴⁷ is independently —NHOH. In some embodiments, R⁴⁷ is independently —OCCl₃. In some embodiments, R⁴⁷ is independently —OCF₃. In some embodiments, R⁴⁷ is independently —OCBr₃. In some embodiments, R⁴⁷ is independently —OCI₃. In some embodiments, R⁴⁷ is independently —OCHCl₂. In some embodiments, R⁴⁷ is independently —OCHBr₂. In some embodiments, R⁴⁷ is independently —OCHI₂. In some embodiments, R⁴⁷ is independently —OCHF₂. In some embodiments, R⁴⁷ is independently —OCH₂Cl. In some embodiments, R⁴⁷ is independently —OCH₂Br. In some embodiments, R⁴⁷ is independently —OCH₂I. In some embodiments, R⁴⁷ is independently —OCH₂F. In some embodiments, R⁴⁷ is independently —N₃. In some embodiments, R⁴⁷ is independently —OCH₃. In some embodiments, R⁴⁷ is independently —CH₃. In some embodiments, R⁴⁷ is independently —CH₂CH₃. In some embodiments, R⁴⁷ is independently unsubstituted propyl. In some embodiments, R⁴⁷ is independently unsubstituted isopropyl. In some embodiments, R⁴⁷ is independently unsubstituted butyl. In some embodiments, R⁴⁷ is independently unsubstituted tert-butyl. In some embodiments, R⁴⁷ is independently —F. In some embodiments, R⁴⁷ is independently —Cl. In some embodiments, R⁴⁷ is independently —Br. In some embodiments, R⁴⁷ is independently —I.

In some embodiments, L⁶ is independently a bond, —S(O)₂—, —S(O)—, ═N—, —NH—, —O—, —S—, —C(O)—, —C(O)NH—, —NHC(O)—, —NHC(O)NH—, —C(O)O—, —OC(O)—, substituted or unsubstituted alkylene (e.g., C₁-C₈ alkylene, C₁-C₆ alkylene, or C₁-C₄ alkylene), substituted or unsubstituted heteroalkylene (e.g., 2 to 8 membered heteroalkylene, 2 to 6 membered heteroalkylene, or 2 to 4 membered heteroalkylene), substituted or unsubstituted cycloalkylene (e.g., C₃-C₈ cycloalkylene, C₃-C₆ cycloalkylene, or C₅-C₆ cycloalkylene), substituted or unsubstituted heterocycloalkylene (e.g., 3 to 8 membered heterocycloalkylene, 3 to 6 membered heterocycloalkylene, or 5 to 6 membered heterocycloalkylene), substituted or unsubstituted arylene (e.g., C₆-C₁₀ arylene, C₁₀ arylene, or phenylene), or substituted or unsubstituted heteroarylene (e.g., 5 to 10 membered heteroarylene, 5 to 9 membered heteroarylene, or 5 to 6 membered heteroarylene). In some embodiments, L⁶ is a bond. In some embodiments, L⁶ is independently a —S(O)₂—. In some embodiments, L⁶ is independently a —S(O)—. In some embodiments, L⁶ is independently a —NH—. In some embodiments, L⁶ is independently a —O—. In some embodiments, L⁶ is independently a —S—. In some embodiments, L⁶ is independently a —C(O)—. In some embodiments, L⁶ is independently a —C(O)NH—. In some embodiments, L⁶ is independently a —NHC(O)—. In some embodiments, L⁶ is independently a —NHC(O)NH—. In some embodiments, L⁶ is independently a —C(O)O—. In some embodiments, L⁶ is independently —OC(O)—. In some embodiments, L⁶ is independently a bond. In some embodiments, L⁶ is independently —NR⁶—.

In some embodiments, L⁶ is independently ═N—. In some embodiments, L⁶ is independently —C(O)NR⁶—. In some embodiments, L⁶ is independently —NR⁶C(O)—. In some embodiments, L⁶ is independently —NR⁶C(O)NH—. In some embodiments, L⁶ is independently —NHC(O)NR⁶—.

In some embodiments, L⁶ is substituted or unsubstituted alkylene (e.g., C₁-C₈ alkylene, C₁-C₆ alkylene, or C₁-C₄ alkylene). In some embodiments, L⁶ is substituted alkylene (e.g., C₁-C₈ alkylene, C₁-C₆ alkylene, or C₁-C₄ alkylene). In some embodiments, L⁶ is an unsubstituted alkylene (e.g., C₁-C₈ alkylene, C₁-C₆ alkylene, or C₁-C₄ alkylene). In some embodiments, L⁶ is substituted or unsubstituted heteroalkylene (e.g., 2 to 8 membered heteroalkylene, 2 to 6 membered heteroalkylene, or 2 to 4 membered heteroalkylene). In some embodiments, L⁶ is substituted heteroalkylene (e.g., 2 to 8 membered heteroalkylene, 2 to 6 membered heteroalkylene, or 2 to 4 membered heteroalkylene). In some embodiments, L⁶ is an unsubstituted heteroalkylene (e.g., 2 to 8 membered heteroalkylene, 2 to 6 membered heteroalkylene, or 2 to 4 membered heteroalkylene). In some embodiments, L⁶ is substituted or unsubstituted cycloalkylene (e.g., C₃-C₈ cycloalkylene, C₃-C₆ cycloalkylene, or C₅-C₆ cycloalkylene). In some embodiments, L⁶ is substituted cycloalkylene (e.g., C₃-C₈ cycloalkylene, C₃-C₆ cycloalkylene, or C₅-C₆ cycloalkylene). In some embodiments, L⁶ is an unsubstituted cycloalkylene (e.g., C₃-C₈ cycloalkylene, C₃-C₆ cycloalkylene, or C₅-C₆ cycloalkylene). In some embodiments, L⁶ is substituted or unsubstituted heterocycloalkylene (e.g., 3 to 8 membered heterocycloalkylene, 3 to 6 membered heterocycloalkylene, or 5 to 6 membered heterocycloalkylene). In some embodiments, L⁶ is substituted heterocycloalkylene (e.g., 3 to 8 membered heterocycloalkylene, 3 to 6 membered heterocycloalkylene, or 5 to 6 membered heterocycloalkylene). In some embodiments, L⁶ is an unsubstituted heterocycloalkylene (e.g., 3 to 8 membered heterocycloalkylene, 3 to 6 membered heterocycloalkylene, or 5 to 6 membered heterocycloalkylene). In some embodiments, L⁶ is substituted or unsubstituted arylene (e.g., C₆-C₁₀ arylene, C₁₀ arylene, or phenylene). In some embodiments, L⁶ is substituted arylene (e.g., C₆-C₁₀ arylene, C₁₀ arylene, or phenylene). In some embodiments, L⁶ is an unsubstituted arylene (e.g., C₆-C₁₀ arylene, C₁₀ arylene, or phenylene). In some embodiments, L⁶ is substituted or unsubstituted heteroarylene (e.g., 5 to 10 membered heteroarylene, 5 to 9 membered heteroarylene, or 5 to 6 membered heteroarylene). In some embodiments, L⁶ is substituted heteroarylene (e.g., 5 to 10 membered heteroarylene, 5 to 9 membered heteroarylene, or 5 to 6 membered heteroarylene). In some embodiments, L⁶ is an unsubstituted heteroarylene (e.g., 5 to 10 membered heteroarylene, 5 to 9 membered heteroarylene, or 5 to 6 membered heteroarylene).

In some embodiments, L⁶ is independently a bond, —S(O)₂—, —S(O)—, ═N—, —NH—, —O—, —S—, —C(O)—, —C(O)NH—, —NHC(O)—, —NHC(O)NH—, —C(O)O—, —OC(O)—, R⁴⁸-substituted or unsubstituted alkylene (e.g., C₁-C₈ alkylene, C₁-C₆ alkylene, or C₁-C₄ alkylene), R⁴⁸-substituted or unsubstituted heteroalkylene (e.g., 2 to 8 membered heteroalkylene, 2 to 6 membered heteroalkylene, or 2 to 4 membered heteroalkylene), R⁴⁸-substituted or unsubstituted cycloalkylene (e.g., C₃-C₈ cycloalkylene, C₃-C₆ cycloalkylene, or C₅-C₆ cycloalkylene), R⁴⁸-substituted or unsubstituted heterocycloalkylene (e.g., 3 to 8 membered heterocycloalkylene, 3 to 6 membered heterocycloalkylene, or 5 to 6 membered heterocycloalkylene), R⁴⁸-substituted or unsubstituted arylene (e.g., C₆-C₁₀ arylene, C₁₀ arylene, or phenylene), or R⁴⁸-substituted or unsubstituted heteroarylene (e.g., 5 to 10 membered heteroarylene, 5 to 9 membered heteroarylene, or 5 to 6 membered heteroarylene). In some embodiments, L⁶ is independently a bond, —S(O)₂—, —S(O)—, —NH—, ═N—, —O—, —S—, —C(O)—, —C(O)NH—, —NHC(O)—, —NHC(O)NH—, —C(O)O—, —OC(O)—, unsubstituted alkylene (e.g., C₁-C₈ alkylene, C₁-C₆ alkylene, or C₁-C₄ alkylene), unsubstituted heteroalkylene (e.g., 2 to 8 membered heteroalkylene, 2 to 6 membered heteroalkylene, or 2 to 4 membered heteroalkylene), unsubstituted cycloalkylene (e.g., C₃-C₈ cycloalkylene, C₃-C₆ cycloalkylene, or C₅-C₆ cycloalkylene), unsubstituted heterocycloalkylene (e.g., 3 to 8 membered heterocycloalkylene, 3 to 6 membered heterocycloalkylene, or 5 to 6 membered heterocycloalkylene), unsubstituted arylene (e.g., C₆-C₁₀ arylene, C₁₀ arylene, or phenylene), or unsubstituted heteroarylene (e.g., 5 to 10 membered heteroarylene, 5 to 9 membered heteroarylene, or 5 to 6 membered heteroarylene).

In some embodiments, L⁶ is R⁴⁸-substituted or unsubstituted alkylene (e.g., C₁-C₈ alkylene, C₁-C₆ alkylene, or C₁-C₄ alkylene). In some embodiments, L⁶ is R⁴⁸-substituted alkylene (e.g., C₁-C₈ alkylene, C₁-C₆ alkylene, or C₁-C₄ alkylene). In some embodiments, L⁶ is an unsubstituted alkylene (e.g., C₁-C₈ alkylene, C₁-C₆ alkylene, or C₁-C₄ alkylene). In some embodiments, L⁶ is R⁴⁸-substituted or unsubstituted heteroalkylene (e.g., 2 to 8 membered heteroalkylene, 2 to 6 membered heteroalkylene, or 2 to 4 membered heteroalkylene). In some embodiments, L⁶ is R⁴⁸-substituted heteroalkylene (e.g., 2 to 8 membered heteroalkylene, 2 to 6 membered heteroalkylene, or 2 to 4 membered heteroalkylene). In some embodiments, L⁶ is an unsubstituted heteroalkylene (e.g., 2 to 8 membered heteroalkylene, 2 to 6 membered heteroalkylene, or 2 to 4 membered heteroalkylene). In some embodiments, L⁶ is R⁴⁸-substituted or unsubstituted cycloalkylene (e.g., C₃-C₈ cycloalkylene, C₃-C₆ cycloalkylene, or C₅-C₆ cycloalkylene). In some embodiments, L⁶ is R⁴⁸-substituted cycloalkylene (e.g., C₃-C₈ cycloalkylene, C₃-C₆ cycloalkylene, or C₅-C₆ cycloalkylene). In some embodiments, L⁶ is an unsubstituted cycloalkylene (e.g., C₃-C₈ cycloalkylene, C₃-C₆ cycloalkylene, or C₅-C₆ cycloalkylene). In some embodiments, L⁶ is R⁴⁸-substituted or unsubstituted heterocycloalkylene (e.g., 3 to 8 membered heterocycloalkylene, 3 to 6 membered heterocycloalkylene, or 5 to 6 membered heterocycloalkylene). In some embodiments, L⁶ is R⁴⁸-substituted heterocycloalkylene (e.g., 3 to 8 membered heterocycloalkylene, 3 to 6 membered heterocycloalkylene, or 5 to 6 membered heterocycloalkylene). In some embodiments, L⁶ is an unsubstituted heterocycloalkylene (e.g., 3 to 8 membered heterocycloalkylene, 3 to 6 membered heterocycloalkylene, or 5 to 6 membered heterocycloalkylene). In some embodiments, L⁶ is R⁴⁸-substituted or unsubstituted arylene (e.g., C₆-C₁₀ arylene, C₁₀ arylene, or phenylene). In some embodiments, L⁶ is R⁴⁸-substituted arylene (e.g., C₆-C₁₀ arylene, C₁₀ arylene, or phenylene). In some embodiments, L⁶ is an unsubstituted arylene (e.g., C₆-C₁₀ arylene, C₁₀ arylene, or phenylene). In some embodiments, L⁶ is R⁴⁸-substituted or unsubstituted heteroarylene (e.g., 5 to 10 membered heteroarylene, 5 to 9 membered heteroarylene, or 5 to 6 membered heteroarylene). In some embodiments, L⁶ is R⁴⁸-substituted heteroarylene (e.g., 5 to 10 membered heteroarylene, 5 to 9 membered heteroarylene, or 5 to 6 membered heteroarylene). In some embodiments, L⁶ is an unsubstituted heteroarylene (e.g., 5 to 10 membered heteroarylene, 5 to 9 membered heteroarylene, or 5 to 6 membered heteroarylene).

In some embodiments, R⁴⁸ is independently oxo, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R⁴⁸ is independently oxo. In some embodiments, R⁴⁸ is independently halogen. In some embodiments, R⁴⁸ is independently —CCl₃. In some embodiments, R⁴⁸ is independently —CBr₃. In some embodiments, R⁴⁸ is independently —CF₃. In some embodiments, R⁴⁸ is independently —CI₃. In some embodiments, R⁴⁸ is independently —CHCl₂. In some embodiments, R⁴⁸ is independently —CHBr₂. In some embodiments, R⁴⁸ is independently —CHF₂. In some embodiments, R⁴⁸ is independently —CHI₂. In some embodiments, R⁴⁸ is independently —CH₂Cl. In some embodiments, R⁴⁸ is independently —CH₂Br. In some embodiments, R⁴⁸ is independently —CH₂F. In some embodiments, R⁴⁸ is independently —CH₂I. In some embodiments, R⁴⁸ is independently —CN. In some embodiments, R⁴⁸ is independently —OH. In some embodiments, R⁴⁸ is independently —NH₂. In some embodiments, R⁴⁸ is independently —COOH. In some embodiments, R⁴⁸ is independently —CONH₂. In some embodiments, R⁴⁸ is independently —NO₂. In some embodiments, R⁴⁸ is independently —SH. In some embodiments, R⁴⁸ is independently —SO₃H. In some embodiments, R⁴⁸ is independently —SO₄H. In some embodiments, R⁴⁸ is independently —SO₂NH₂. In some embodiments, R⁴⁸ is independently —NHNH₂. In some embodiments, R⁴⁸ is independently —ONH₂. In some embodiments, R⁴⁸ is independently —NHC(O)NHNH₂. In some embodiments, R⁴⁸ is independently —NHC(O)NH₂. In some embodiments, R⁴⁸ is independently —NHSO₂H. In some embodiments, R⁴⁸ is independently —NHC(O)H. In some embodiments, R⁴⁸ is independently —NHC(O)OH. In some embodiments, R⁴⁸ is independently —NHOH. In some embodiments, R⁴⁸ is independently —OCCl₃. In some embodiments, R⁴⁸ is independently —OCF₃. In some embodiments, R⁴⁸ is independently —OCBr₃. In some embodiments, R⁴⁸ is independently —OCI₃. In some embodiments, R⁴⁸ is independently —OCHCl₂. In some embodiments, R⁴⁸ is independently —OCHBr₂. In some embodiments, R⁴⁸ is independently —OCHI₂. In some embodiments, R⁴⁸ is independently —OCHF₂. In some embodiments, R⁴⁸ is independently —OCH₂Cl. In some embodiments, R⁴⁸ is independently —OCH₂Br. In some embodiments, R⁴⁸ is independently —OCH₂I. In some embodiments, R⁴⁸ is independently —OCH₂F. In some embodiments, R⁴⁸ is independently —N₃. In some embodiments, R⁴⁸ is independently —OCH₃. In some embodiments, R⁴⁸ is independently —CH₃. In some embodiments, R⁴⁸ is independently —CH₂CH₃. In some embodiments, R⁴⁸ is independently unsubstituted propyl. In some embodiments, R⁴⁸ is independently unsubstituted isopropyl. In some embodiments, R⁴⁸ is independently unsubstituted butyl. In some embodiments, R⁴⁸ is independently unsubstituted tert-butyl. In some embodiments, R⁴⁸ is independently —F. In some embodiments, R⁴⁸ is independently —Cl. In some embodiments, R⁴⁸ is independently —Br. In some embodiments, R⁴⁸ is independently —I.

In some embodiments, X is independently —F. In some embodiments, X is independently —Cl. In some embodiments, X is independently —Br. In some embodiments, X is independently —I. In some embodiments, X¹ is independently —F. In some embodiments, X¹ is independently —Cl. In some embodiments, X¹ is independently —Br. In some embodiments, X¹ is independently —I. In some embodiments, X² is independently —F. In some embodiments, X² is independently —Cl. In some embodiments, X² is independently —Br. In some embodiments, X² is independently —I. In some embodiments, X³ is independently —F. In some embodiments, X³ is independently —Cl. In some embodiments, X³ is independently —Br. In some embodiments, X³ is independently —I. In some embodiments, X⁴ is independently —F. In some embodiments, X⁴ is independently —Cl. In some embodiments, X⁴ is independently —Br. In some embodiments, X⁴ is independently —I. In some embodiments, X⁵ is independently —F. In some embodiments, X⁵ is independently —Cl. In some embodiments, X⁵ is independently —Br. In some embodiments, X⁵ is independently —I. In some embodiments, X⁶ is independently —F. In some embodiments, X⁶ is independently —Cl. In some embodiments, X⁶ is independently —Br. In some embodiments, X⁶ is independently —I.

In some embodiments, n1 is independently 0. In some embodiments, n1 is independently 1. In some embodiments, n1 is independently 2. In some embodiments, n1 is independently 3. In some embodiments, n1 is independently 4. In some embodiments, n2 is independently 0. In some embodiments, n2 is independently 1. In some embodiments, n2 is independently 2. In some embodiments, n2 is independently 3. In some embodiments, n2 is independently 4. In some embodiments, n3 is independently 0. In some embodiments, n3 is independently 1. In some embodiments, n3 is independently 2. In some embodiments, n3 is independently 3. In some embodiments, n3 is independently 4.

In some embodiments, m1 is independently 1. In some embodiments, m1 is independently 2. In some embodiments, m2 is independently 1. In some embodiments, m2 is independently 2. In some embodiments, m3 is independently 1. In some embodiments, m3 is independently 2.

In some embodiments, v1 is independently 1. In some embodiments, v1 is independently 2. In some embodiments, v2 is independently 1. In some embodiments, v2 is independently 2. In some embodiments, v3 is independently 1. In some embodiments, v3 is independently 2.

In some embodiments, z1 is independently 0. In some embodiments, z1 is independently 1. In some embodiments, z1 is independently 2. In some embodiments, z1 is independently 3. In some embodiments, z1 is independently 4. In some embodiments, z1 is independently 5. In some embodiments, z1 is independently 6. In some embodiments, z1 is independently 7. In some embodiments, z1 is independently 8. In some embodiments, z1 is independently 9. In some embodiments, z1 is independently 10. In some embodiments, z1 is independently 11. In some embodiments, z1 is independently 12.

In some embodiments, z3 is independently 0. In some embodiments, z3 is independently 1. In some embodiments, z3 is independently 2. In some embodiments, z3 is independently 3. In some embodiments, z3 is independently 4.

In some embodiments, Z is O, S, or SO₂. In some embodiments, Z is O. In some embodiments, Z is S. In some embodiments, Z is SO₂.

In some embodiments, W is O, NH, NR¹, or CH₂. In some embodiments, W is O. In some embodiments, W is NH. In some embodiments, W is NR¹. In some embodiments, W is CH₂. It is understood that when W is CH₂, W is optionally substituted by 1-2 R¹ substituents. In some embodiments, W is CHR¹. In some embodiments, W is CR¹R¹.

In some embodiments, n is 0 or 1. In some embodiments, n is 0. In some embodiments, n is 1.

If a substituent (e.g., R¹ or R³) is floating for a cycloalkyl ring, a heterocyclic ring, or an aromatic ring (e.g., aryl, heteroaryl, arylene, or heteroarylene), it is understood to obey the rules of chemical valency.

A person of ordinary skill in the art will understand when a compound or a compound genus (e.g., a genus described herein) is described by a name or formula of a standalone compound with all valencies filled, the valencie(s) will be dictated by the context in which the compound is used. For example, when a compound (e.g., cellular component binder or targeted autophagy protein binder) as described herein is connected (e.g., bonded) through a linker, it is understood the compound represents a monovalent form of the standalone compound. The compounds provided herein may be depicted as standalone compounds with all valencies filled. However, when it is intended to be a monovalent compound (e.g., monovalent targeted autophagy protein binder) it is understood that a substituent (e.g., hydrogen, halogen, methyl, R¹, R², or R³) may be removed to accommodate the linker.

It is understood that when a compound as shown anywhere in the specification is connected (e.g., bonded) to another moiety through a linker, the compound is intended to be a monovalent form of the standalone compound at any attachment point following the replacement of a substituent (e.g., hydrogen or halogen) with a bond to the linker connected to the other moiety.

It is understood that when a compound as shown anywhere in the specification (e.g., in Table 1) is connected (e.g., bonded) to a linker, it is understood the compound is intended to be a monovalent form of the standalone compound at any attachment point following the replacement of a substituent (e.g., hydrogen or halogen) with the bond to the linker. In some embodiments, the targeted autophagy binder is a compound in Table 1. In some embodiments, the monovalent targeted autophagy binder is a monovalent form of a compound in Table 1.

TABLE 1

| Compound | Structure | Compound | Structure |
|---|---|---|---|
| 1 | 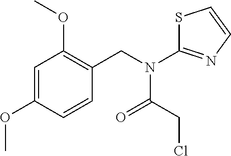 [SMILES: COc1ccc(CN(C(=O)CCl)c2nccs2)c(OC)c1] | 2 | 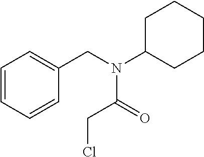 [SMILES: O=C(CCl)N(Cc1ccccc1)C1CCCCC1] |
| 3 | 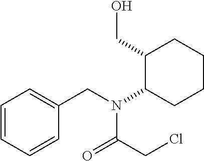 | 4 | 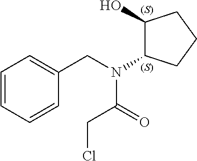 |
| 5 | 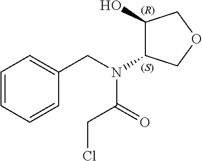 | 6 | 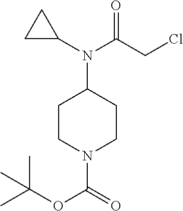 [SMILES: CC(C)(C)OC(=O)N1CCC(N(C(=O)CCl)C2CC2)CC1] |
| 7 | 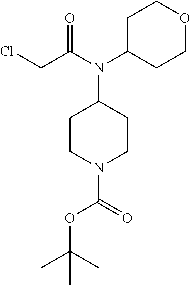 [SMILES: CC(C)(C)OC(=O)N1CCC(N(C(=O)CCl)C2CCOCC2)CC1] | 8 | 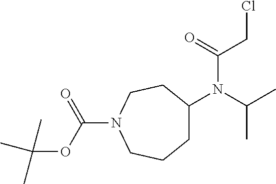 [SMILES: CC(C)N(C(=O)CCl)C1CCCN(C(=O)OC(C)(C)C)CC1] |
| 9 | 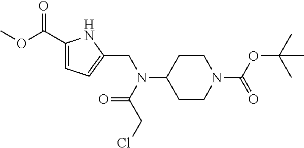 [SMILES: COC(=O)c1ccc(CN(C(=O)CCl)C2CCN(C(=O)OC(C)(C)C)CC2)[nH]1] | 10 | 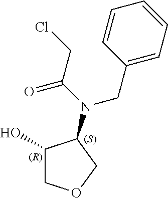 |
| 11 | 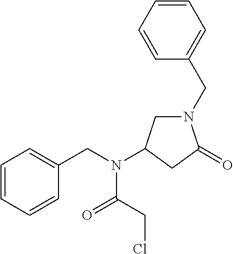 [SMILES: O=C1CC(N(Cc2ccccc2)C(=O)CCl)CN1Cc1ccccc1] | 12 | 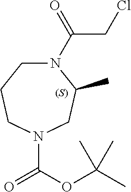 |
| 13 | 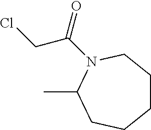 | 14 | 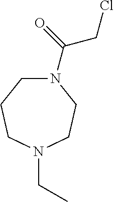 |
| 15 | 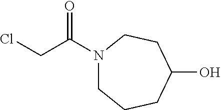 [SMILES: O=C(CCl)N1CCCC(O)CC1] | 16 | 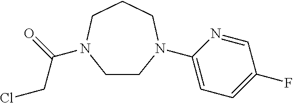 [SMILES: O=C(CCl)N1CCCN(c2ccc(F)cn2)CC1] |
| 17 | 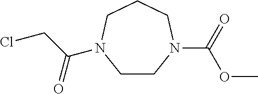 | 18 | 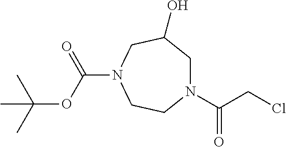 [SMILES: CC(C)(C)OC(=O)N1CCN(C(=O)CCl)CC(O)C1] |
| 19 | 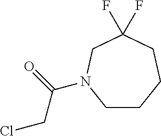 [SMILES: O=C(CCl)N1CCCCC(F)(F)C1] | 20 | 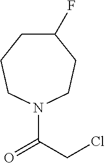 [SMILES: O=C(CCl)N1CCCC(F)CC1] |
| 21 | 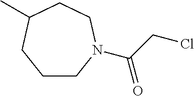 | 22 | 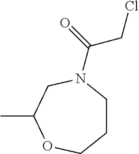 |
| 23 | 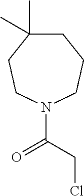 | 24 | 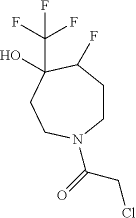 [SMILES: O=C(CCl)N1CCC(F)C(O)(C(F)(F)F)CC1] |
| 25 | 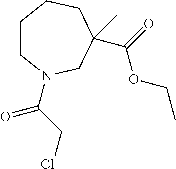 [SMILES: CCOC(=O)C1(C)CCCCN(C(=O)CCl)C1] | 26 | 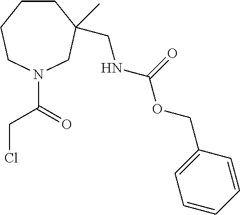 [SMILES: CC1(CNC(=O)OCc2ccccc2)CCCCN(C(=O)CCl)C1] |
| 27 | 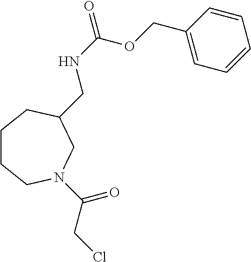 [SMILES: O=C(NCC1CCCCN(C(=O)CCl)C1)OCc1ccccc1] | 28 | 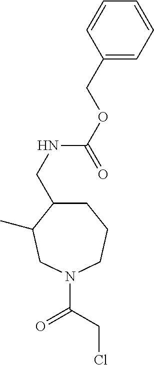 [SMILES: CC1CN(C(=O)CCl)CCCC1CNC(=O)OCc1ccccc1] |
| 29 | 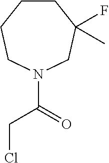 | 30 | 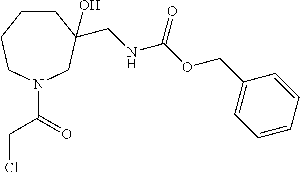 [SMILES: O=C(NCC1(O)CCCCN(C(=O)CCl)C1)OCc1ccccc1] |
| 31 | 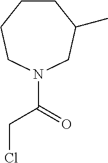 | 32 | 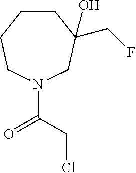 [SMILES: O=C(CCl)N1CCCCC(O)(CF)C1] |
| 33 | 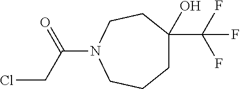 [SMILES: O=C(CCl)N1CCCC(O)(C(F)(F)F)CC1] | 34 | 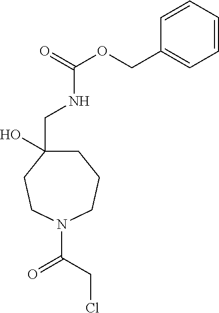 [SMILES: O=C(NCC1(O)CCCN(C(=O)CCl)CC1)OCc1ccccc1] |
| 35 | 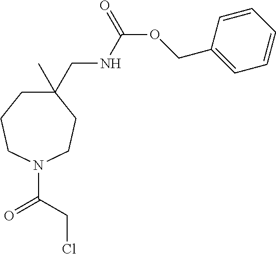 [SMILES: CC1(CNC(=O)OCc2ccccc2)CCCN(C(=O)CCl)CC1] | 36 | 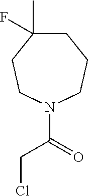 |
| 37 | 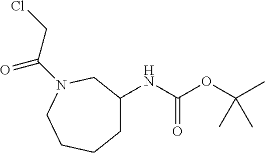 [SMILES: CC(C)(C)OC(=O)NC1CCCCN(C(=O)CCl)C1] | 38 | 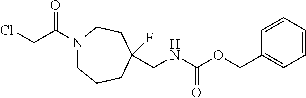 [SMILES: O=C(NCC1(F)CCCN(C(=O)CCl)CC1)OCc1ccccc1] |
| 39 | 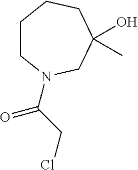 | 40 | 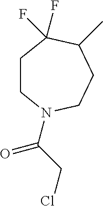 |
| 41 | 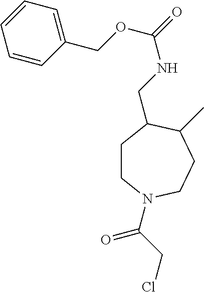 [SMILES: CC1CCN(C(=O)CCl)CCC1CNC(=O)OCc1ccccc1] | 42 | 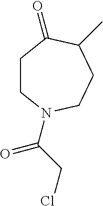 |
| 43 | 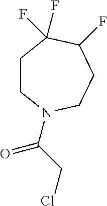 [SMILES: O=C(CCl)N1CCC(F)C(F)(F)CC1] | 44 | 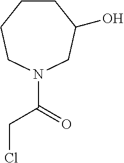 [SMILES: O=C(CCl)N1CCCCC(O)C1] |
| 45 | 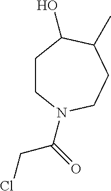 | 46 | 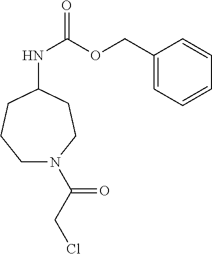 [SMILES: O=C(NC1CCCN(C(=O)CCl)CC1)OCc1ccccc1] |
| 47 | 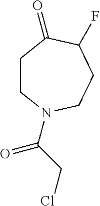 [SMILES: O=C1CCN(C(=O)CCl)CCC1F] | 48 | 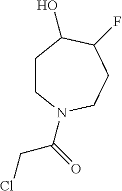 [SMILES: O=C(CCl)N1CCC(O)C(F)CC1] |
| 49 | 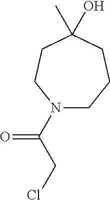 | 50 | 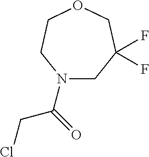 [SMILES: O=C(CCl)N1CCOCC(F)(F)C1] |
| 51 | 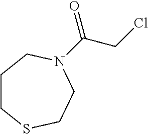 [SMILES: O=C(CCl)N1CCCSCC1] | 52 | 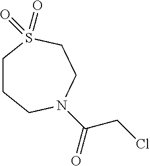 [SMILES: O=C(CCl)N1CCCS(=O)(=O)CC1] |
| 53 | 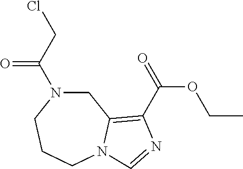 [SMILES: CCOC(=O)c1ncn2c1CN(C(=O)CCl)CCC2] | 54 | 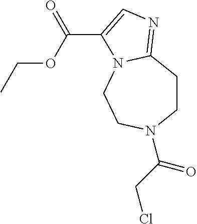 [SMILES: CCOC(=O)c1cnc2n1CCN(C(=O)CCl)CC2] |
| 55 | 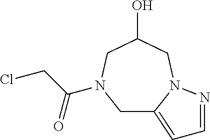 [SMILES: O=C(CCl)N1Cc2ccnn2CC(O)C1] | 56 | 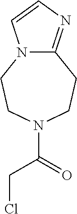 [SMILES: O=C(CCl)N1CCc2nccn2CC1] |
| 57 | 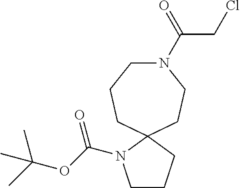 [SMILES: CC(C)(C)OC(=O)N1CCCC12CCCN(C(=O)CCl)CC2] | 58 | 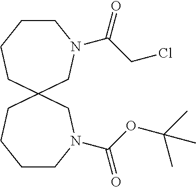 [SMILES: CC(C)(C)OC(=O)N1CCCCC2(CCCCN(C(=O)CCl)C2)C1] |
| 59 | 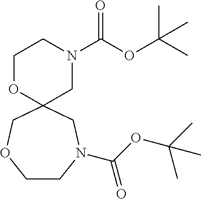 [SMILES: CC(C)(C)OC(=O)N1CCOCC2(C1)CN(C(=O)OC(C)(C)C)CCO2] | 60 | 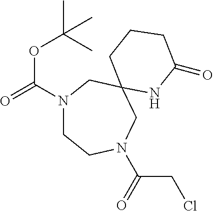 [SMILES: CC(C)(C)OC(=O)N1CCN(C(=O)CCl)CC2(CCCC(=O)N2)C1] |
| 61 | 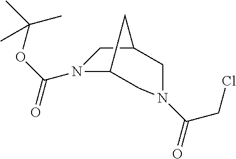 [SMILES: CC(C)(C)OC(=O)N1CC2CC1CN(C(=O)CCl)C2] | 62 | 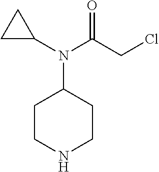 [SMILES: O=C(CCl)N(C1CCNCC1)C1CC1] |
| 63 | 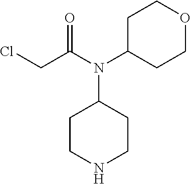 [SMILES: O=C(CCl)N(C1CCNCC1)C1CCOCC1] | 64 | 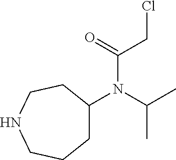 |
| 65 | 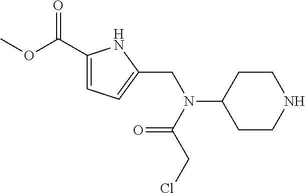 [SMILES: COC(=O)c1ccc(CN(C(=O)CCl)C2CCNCC2)[nH]1] | 66 | 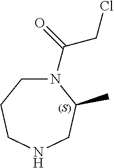 |
| 67 | 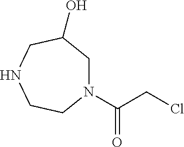 [SMILES: O=C(CCl)N1CCNCC(O)C1] | 68 | 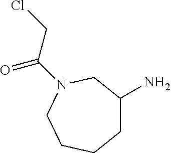 [SMILES: NC1CCCCN(C(=O)CCl)C1] |
| 69 | 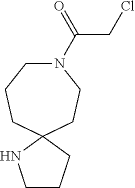 [SMILES: O=C(CCl)N1CCCC2(CCCN2)CC1] | 70 | 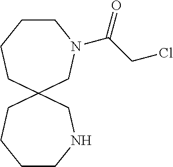 [SMILES: O=C(CCl)N1CCCCC2(CCCCNC2)C1] |
| 71 | 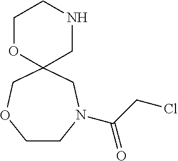 [SMILES: O=C(CCl)N1CCOCC2(CNCCO2)C1] | 72 | 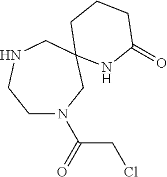 [SMILES: O=C1CCCC2(CNCCN(C(=O)CCl)C2)N1] |
| 73 | 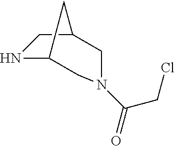 [SMILES: O=C(CCl)N1CC2CNC(C2)C1] | | |

In some embodiments, the compound is a compound described herein. In some embodiments, the compound is a derivative, analogue, or prodrug of a compound described herein. In some embodiments, the compound is a derivative of a compound described herein. In some embodiments, the compound is an analogue of a compound described herein. In some embodiments, the compound is a prodrug of a compound described herein.

It will be understood that unless stereochemistry is explicitly indicated in a chemical structure or name, the structure or name is intended to embrace all possible stereoisomers of a compound depicted. Similarly, if stereochemistry is explicitly indicated in a chemical structure or name, it is understood that the present disclosure embraces all stereoisomers of the indicated compound unless indicated otherwise.

In some embodiments, the targeted autophagy protein binder is capable of contacting an autophagy adapter protein. In some embodiments, the targeted autophagy binder is capable of binding (e.g., covalently binding) an autophagy adapter protein. In some embodiments, the monovalent targeted autophagy binder is capable of contacting an autophagy adapter protein. In some embodiments, the monovalent targeted autophagy binder is capable of binding (e.g., covalently binding) an autophagy adapter protein.

In some embodiments, the targeted autophagy protein binder (e.g., autophagy adapter protein binder) is capable of contacting an autophagy adapter protein. In some embodiments, the monovalent targeted autophagy protein binder (e.g., monovalent autophagy adapter protein binder) is capable of contacting an autophagy adapter protein. In some embodiments, the autophagy adapter protein is p62/SQSTM1, or an analog, derivative, fragment, or homolog thereof. In some embodiments, the autophagy adapter protein is human p62/SQSTM1.

In some embodiments, the targeted autophagy protein binder (e.g., autophagy adapter protein binder) is capable of contacting an amino acid corresponding to C26 of human p62/SQSTM1 protein. In some embodiments, the targeted autophagy protein binder (e.g., autophagy adapter protein binder) is capable of contacting an amino acid corresponding to C27 of human p62/SQSTM1protein. In some embodiments, the targeted autophagy protein binder (e.g., autophagy adapter protein binder) is capable of contacting an amino acid corresponding to C113 of human p62/SQSTM1 protein. In some embodiments, the monovalent targeted autophagy protein binder (e.g., monovalent autophagy adapter protein binder) is capable of contacting an amino acid corresponding to C26 of human p62/SQSTM1 protein. In some embodiments, the monovalent targeted autophagy protein binder (e.g., monovalent autophagy adapter protein binder) is capable of contacting an amino acid corresponding to C27 of human p62/SQSTM1protein. In some embodiments, the monovalent targeted autophagy protein binder (e.g., monovalent autophagy adapter protein binder) is capable of contacting an amino acid corresponding to C113 of human p62/SQSTM1 protein.

In some embodiments, the targeted autophagy protein binder (e.g., autophagy adapter protein binder) is capable of forming a covalent bond with an amino acid corresponding to C26 of human p62/SQSTM1 protein. In some embodiments, the targeted autophagy protein binder (e.g., autophagy adapter protein binder) is capable of forming a covalent bond with an amino acid corresponding to C27 of human p62/SQSTM1protein. In some embodiments, the targeted autophagy protein binder (e.g., autophagy adapter protein binder) is capable of forming a covalent bond with an amino acid corresponding to C113 of human p62/SQSTM1 protein. In some embodiments, the monovalent targeted autophagy protein binder (e.g., monovalent autophagy adapter protein binder) is capable of forming a covalent bond with an amino acid corresponding to C26 of human p62/SQSTM1 protein. In some embodiments, the monovalent targeted autophagy protein binder (e.g., monovalent autophagy adapter protein binder) is capable of forming a covalent bond with an amino acid corresponding to C27 of human p62/SQSTM1protein. In some embodiments, the monovalent targeted autophagy protein binder (e.g., monovalent autophagy adapter protein binder) is capable of forming a covalent bond with an amino acid corresponding to C113 of human p62/SQSTM1protein.

### Linker

In some embodiments, a divalent linker binds the monovalent cellular component binder to the monovalent targeted autophagy protein binder (e.g., monovalent autophagy adapter protein binder). It is understood that the monovalent cellular component binder is connected to the divalent linker at any position provided the resulting covalent bond is constructed according to the standard rules of chemical valency known in the chemical arts.

It is understood that the monovalent targeted autophagy protein binder is connected to the divalent linker at any position provided the resulting covalent bond is constructed according to the standard rules of chemical valency known in the chemical arts.

In some embodiments, the divalent linker has the formula -L¹-L²-L³-L⁴-;

In some embodiments, L¹ is connected directly to the monovalent targeted autophagy protein binder (e.g., monovalent autophagy adapter protein binder). In some embodiments, L¹ is —S(O)₂—, —S(O)—, —NH—, —O—, —S—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or a bioconugate linker. In some embodiments, L¹ is —S(O)₂—, —S(O)—, —NH—, —O—, —S—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In some embodiments, L¹ is a bioconjugate linker.

In some embodiments, L² is a bond, —S(O)₂—, —S(O)—, —NH—, —O—, —S—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or a bioconjugate linker. In some embodiments, L² is a bond, —S(O)₂—, —S(O)—, —NH—, —O—, —S—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In some embodiments, L² is a bioconjugate linker.

In some embodiments, L³ is a bond, —S(O)₂—, —S(O)—, —NH—, —O—, —S—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or a bioconugate linker. In some embodiments, L³ is a bond, —S(O)₂—, —S(O)—, —NH—, —O—, —S—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In some embodiments, L³ is a bioconjugate linker.

In some embodiments, L⁴ is a bond, —S(O)₂—, —S(O)—, —NH—, —O—, —S—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or a bioconugate linker. In some embodiments, L⁴ is a bond, —S(O)₂—, —S(O)—, —NH—, —O—, —S—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In some embodiments, L⁴ is a bioconjugate linker.

In some embodiments, the divalent linker -L¹-L²-L³-L⁴- has the formula —O-L²-L³-L⁴- and L², L³, and L⁴ are as described herein. In some embodiments, the divalent linker has the formula —O-L²-L³-O— and L² and L³ are as described herein. In some embodiments, the divalent linker -L¹-L²-L³-L⁴- has the formula -L¹-L²-L³-O— and L¹, L², and L³ are as described herein. In some embodiments, the divalent linker -L¹-L²-L³-L⁴- has the formula —O-L²-L³-O—, L² is R⁴⁴-substituted or unsubstituted alkylene (e.g., C₁-C₈ alkylene, C₁-C₆ alkylene, or C₁-C₄ alkylene), L³ is R⁴⁵-substituted or unsubstituted alkylene (e.g., C₁-C₈ alkylene, C₁-C₆ alkylene, or C₁-C₄ alkylene), and R⁴⁴ and R⁴⁵ are as described herein. In some embodiments, the divalent linker -L¹-L²-L³-L⁴- has the formula —O-L²-L³-O—, L² is unsubstituted alkylene (e.g., C₁-C₈ alkylene, C₁-C₆ alkylene, or C₁-C₄ alkylene), and L³ is unsubstituted alkylene (e.g., C₁-C₈ alkylene, C₁-C₆ alkylene, or C₁-C₄ alkylene). In some embodiments, the divalent linker -L¹-L²-L³-L⁴- has the formula —O-L²-O—, L² is R⁴⁴-substituted or unsubstituted alkylene (e.g., C₁-C₈ alkylene, C₁-C₆ alkylene, or C₁-C₄ alkylene), and R⁴⁴ is as described herein. In some embodiments, the divalent linker -L¹-L²-L³-L⁴- has the formula —O-L²-L³-O—, L³ is a bond, L² is unsubstituted alkylene (e.g., C₁-C₈ alkylene, C₁-C₆ alkylene, or C₁-C₄ alkylene). In some embodiments, of the divalent linker of formula -L¹-L²-L³-L⁴-; L¹, L³, and L⁴ are a bond; L² is R⁴⁴-substituted or unsubstituted heteroalkylene (e.g., 2 to 8 membered heteroalkylene, 2 to 6 membered heteroalkylene, or 2 to 4 membered heteroalkylene); and R⁴⁴ is as described herein. In some embodiments, of the divalent linker of formula -L¹-L²-L³-L⁴-; L¹, L³, and L⁴ are a bond; L² is R⁴⁴-substituted heteroalkylene (e.g., 2 to 8 membered heteroalkylene, 2 to 6 membered heteroalkylene, or 2 to 4 membered heteroalkylene); and R⁴⁴ is as described herein. In some embodiments, of the divalent linker of formula -L-L²-L³-L⁴-; L¹, L³, and L⁴ are a bond; L² is R⁴⁴-substituted heteroalkylene (e.g., 2 to 8 membered heteroalkylene, 2 to 6 membered heteroalkylene, or 2 to 4 membered heteroalkylene); and R⁴⁴ is oxo. In some embodiments, of the divalent linker of formula -L¹-L²-L³-L⁴-; L¹, L³, and L⁴ are a bond; L² is unsubstituted heteroalkylene (e.g., 2 to 8 membered heteroalkylene, 2 to 6 membered heteroalkylene, or 2 to 4 membered heteroalkylene). In some embodiments, of the divalent linker of formula -L¹-L²-L³-L⁴-; L¹, L³, and L⁴ are a bond; L² is unsubstituted 2 to 8 membered heteroalkylene. In some embodiments, of the divalent linker of formula -L¹-L²-L³-L⁴-; L¹, L³, and L⁴ are a bond; L² is unsubstituted 2 to 6 membered heteroalkylene. In some embodiments, of the divalent linker of formula -L¹-L²-L³-L⁴-; L¹, L³, and L⁴ are a bond; L² is unsubstituted 2 to 4 membered heteroalkylene. In some embodiments, of the divalent linker of formula -L¹-L²-L³-L⁴-; L¹, L³, and L⁴ are a bond; L² is unsubstituted 2 to 12 membered heteroalkylene. In some embodiments, of the divalent linker of formula -L¹-L²-L³-L⁴-; L¹, L³, and L⁴ are a bond; L² is unsubstituted 2 to 10 membered heteroalkylene. In some embodiments, of the divalent linker of formula -L¹-L²-L³-L⁴-; L¹, L³, and L⁴ are a bond; L² is unsubstituted 2 to 8 membered heteroalkylene. In some embodiments, of the divalent linker of formula -L¹-L²-L³-L⁴-; L¹, L³, and L⁴ are a bond; L² is unsubstituted 4 to 12 membered heteroalkylene. In some embodiments, of the divalent linker of formula -L¹-L²-L³-L⁴-; L¹, L³, and L⁴ are a bond; L² is unsubstituted 4 to 10 membered heteroalkylene. In some embodiments, of the divalent linker of formula -L¹-L²-L³-L⁴-; L¹, L³, and L⁴ are a bond; L² is unsubstituted 6 to 12 membered heteroalkylene. In some embodiments, of the divalent linker of formula -L¹-L²-L³-L⁴-; L¹, L³, and L⁴ are a bond; L² is unsubstituted 8 to 12 membered heteroalkylene.

In some embodiments, the linker is a linker described in US20160272639A1, WO2017079723A1, US20130190340A1, or WO2013106643A2 which are incorporated herein by reference in their entirety for all purposes. In some embodiments, the linker is

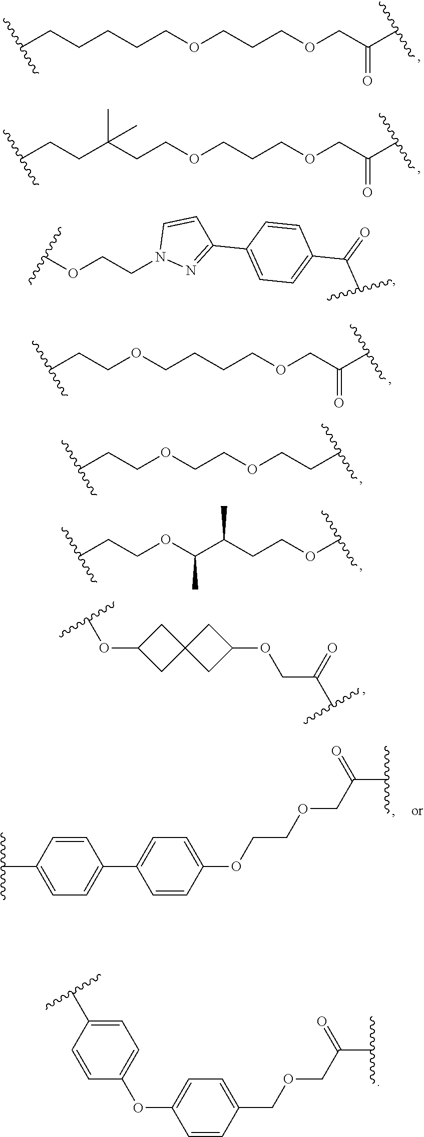

In some embodiments, the linker is:

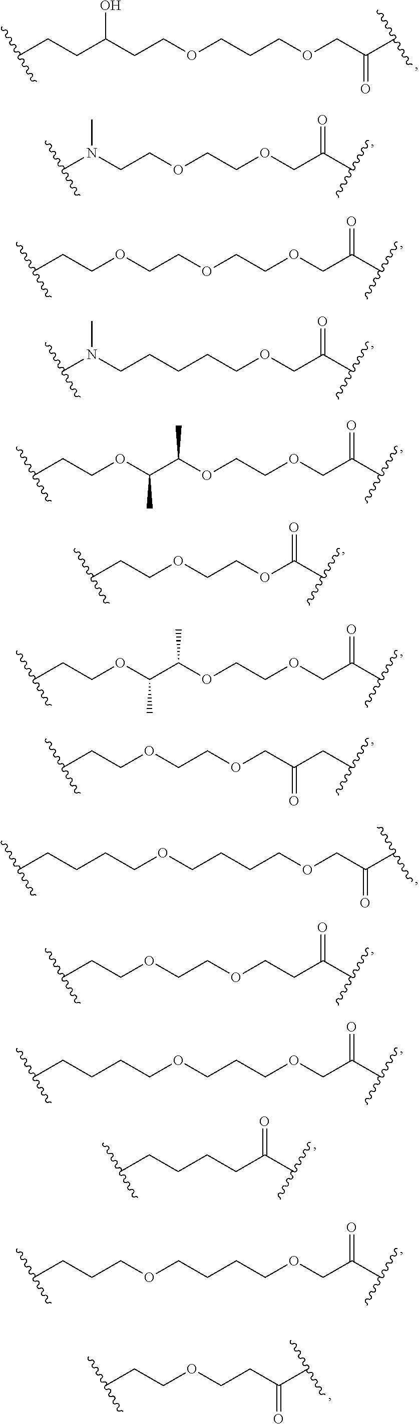

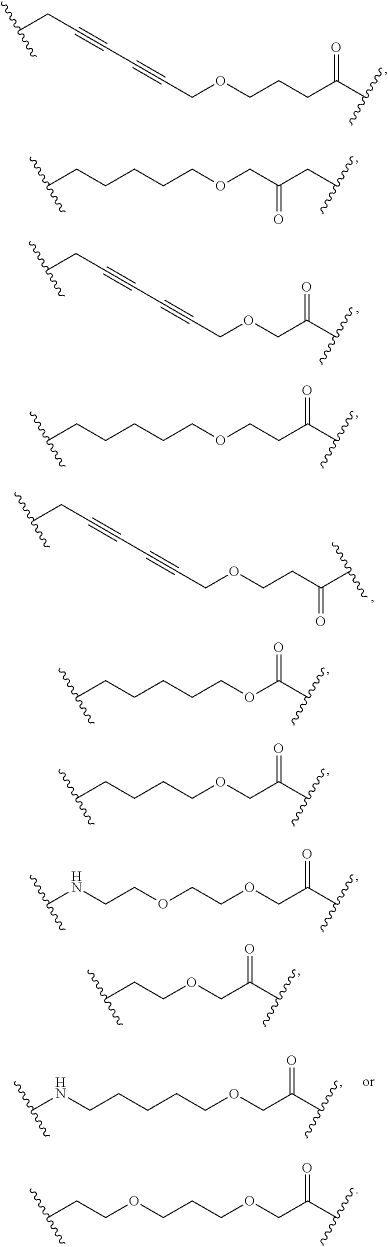

In some embodiments, the linker is

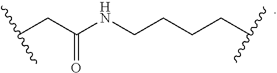 [SMILES: *CCCCNC(=O)C*]

In some embodiments, L¹ is independently a bond, —S(O)₂—, —S(O)—, —NH—, —O—, —S—, —C(O)—, —C(O)NH—, —NHC(O)—, —NHC(O)NH—, —C(O)O—, —OC(O)—, substituted or unsubstituted alkylene (e.g., C₁-C₈ alkylene, C₁-C₆ alkylene, or C₁-C₄ alkylene), substituted or unsubstituted heteroalkylene (e.g., 2 to 8 membered heteroalkylene, 2 to 6 membered heteroalkylene, or 2 to 4 membered heteroalkylene), substituted or unsubstituted cycloalkylene (e.g., C₃-C₈ cycloalkylene, C₃-C₆ cycloalkylene, or C₅-C₆ cycloalkylene), substituted or unsubstituted heterocycloalkylene (e.g., 3 to 8 membered heterocycloalkylene, 3 to 6 membered heterocycloalkylene, or 5 to 6 membered heterocycloalkylene), substituted or unsubstituted arylene (e.g., C₆-C₁₀ arylene, C₁₀ arylene, or phenylene), or substituted or unsubstituted heteroarylene (e.g., 5 to 10 membered heteroarylene, 5 to 9 membered heteroarylene, or 5 to 6 membered heteroarylene). In some embodiments, L¹ is independently a —S(O)₂—. In some embodiments, L¹ is independently a —S(O)—. In some embodiments, L¹ is independently a —NH—. In some embodiments, L¹ is independently a —O—. In some embodiments, L¹ is independently a —S—. In some embodiments, L¹ is independently a —C(O)—. In some embodiments, L¹ is independently a —C(O)NH—. In some embodiments, L¹ is independently a —NHC(O)—. In some embodiments, L¹ is independently a —NHC(O)NH—. In some embodiments, L¹ is independently a —C(O)O—. In some embodiments, L¹ is independently —OC(O)—.

In some embodiments, L¹ is substituted or unsubstituted alkylene (e.g., C₁-C₈ alkylene, C₁-C₆ alkylene, or C₁-C₄ alkylene). In some embodiments, L¹ is substituted alkylene (e.g., C₁-C₈ alkylene, C₁-C₆ alkylene, or C₁-C₄ alkylene). In some embodiments, Li is an unsubstituted alkylene (e.g., C₁-C₈ alkylene, C₁-C₆ alkylene, or C₁-C₄ alkylene). In some embodiments, L¹ is substituted or unsubstituted heteroalkylene (e.g., 2 to 8 membered heteroalkylene, 2 to 6 membered heteroalkylene, or 2 to 4 membered heteroalkylene). In some embodiments, L¹ is substituted heteroalkylene (e.g., 2 to 8 membered heteroalkylene, 2 to 6 membered heteroalkylene, or 2 to 4 membered heteroalkylene). In some embodiments, L¹ is an unsubstituted heteroalkylene (e.g., 2 to 8 membered heteroalkylene, 2 to 6 membered heteroalkylene, or 2 to 4 membered heteroalkylene). In some embodiments, L¹ is substituted or unsubstituted cycloalkylene (e.g., C₃-C₈ cycloalkylene, C₃-C₆ cycloalkylene, or C₅-C₆ cycloalkylene). In some embodiments, L¹ is substituted cycloalkylene (e.g., C₃-C₈ cycloalkylene, C₃-C₆ cycloalkylene, or C₅-C₆ cycloalkylene). In some embodiments, L¹ is an unsubstituted cycloalkylene (e.g., C₃-C₈ cycloalkylene, C₃-C₆ cycloalkylene, or C₅-C₆ cycloalkylene). In some embodiments, L¹ is substituted or unsubstituted heterocycloalkylene (e.g., 3 to 8 membered heterocycloalkylene, 3 to 6 membered heterocycloalkylene, or 5 to 6 membered heterocycloalkylene). In some embodiments, L¹ is substituted heterocycloalkylene (e.g., 3 to 8 membered heterocycloalkylene, 3 to 6 membered heterocycloalkylene, or 5 to 6 membered heterocycloalkylene). In some embodiments, L¹ is an unsubstituted heterocycloalkylene (e.g., 3 to 8 membered heterocycloalkylene, 3 to 6 membered heterocycloalkylene, or 5 to 6 membered heterocycloalkylene). In some embodiments, L¹ is substituted or unsubstituted arylene (e.g., C₆-C₁₀ arylene, C₁₀ arylene, or phenylene). In some embodiments, L¹ is substituted arylene (e.g., C₆-C₁₀ arylene, C₁₀ arylene, or phenylene). In some embodiments, L¹ is an unsubstituted arylene (e.g., C₆-C₁₀ arylene, C₁₀ arylene, or phenylene). In some embodiments, L¹ is substituted or unsubstituted heteroarylene (e.g., 5 to 10 membered heteroarylene, 5 to 9 membered heteroarylene, or 5 to 6 membered heteroarylene). In some embodiments, L¹ is substituted heteroarylene (e.g., 5 to 10 membered heteroarylene, 5 to 9 membered heteroarylene, or 5 to 6 membered heteroarylene). In some embodiments, L¹ is an unsubstituted heteroarylene (e.g., 5 to 10 membered heteroarylene, 5 to 9 membered heteroarylene, or 5 to 6 membered heteroarylene).

In some embodiments, L¹ is independently —S(O)₂—, —S(O)—, —NH—, —O—, —S—, —C(O)—, —C(O)NH—, —NHC(O)—, —NHC(O)NH—, —C(O)O—, —OC(O)—, R⁴³-substituted or unsubstituted alkylene (e.g., C₁-C₈ alkylene, C₁-C₆ alkylene, or C₁-C₄ alkylene), R⁴³-substituted or unsubstituted heteroalkylene (e.g., 2 to 8 membered heteroalkylene, 2 to 6 membered heteroalkylene, or 2 to 4 membered heteroalkylene), R⁴³-substituted or unsubstituted cycloalkylene (e.g., C₃-C₈ cycloalkylene, C₃-C₆ cycloalkylene, or C₅-C₆ cycloalkylene), R⁴³-substituted or unsubstituted heterocycloalkylene (e.g., 3 to 8 membered heterocycloalkylene, 3 to 6 membered heterocycloalkylene, or 5 to 6 membered heterocycloalkylene), R⁴³-substituted or unsubstituted arylene (e.g., C₆-C₁₀ arylene, C₁₀ arylene, or phenylene), or R⁴³-substituted or unsubstituted heteroarylene (e.g., 5 to 10 membered heteroarylene, 5 to 9 membered heteroarylene, or 5 to 6 membered heteroarylene). In some embodiments, L¹ is independently —S(O)₂—, —S(O)—, —NH—, —O—, —S—, —C(O)—, —C(O)NH—, —NHC(O)—, —NHC(O)NH—, —C(O)O—, —OC(O)—, unsubstituted alkylene (e.g., C₁-C₈ alkylene, C₁-C₆ alkylene, or C₁-C₄ alkylene), unsubstituted heteroalkylene (e.g., 2 to 8 membered heteroalkylene, 2 to 6 membered heteroalkylene, or 2 to 4 membered heteroalkylene), unsubstituted cycloalkylene (e.g., C₃-C₈ cycloalkylene, C₃-C₆ cycloalkylene, or C₅-C₆ cycloalkylene), unsubstituted heterocycloalkylene (e.g., 3 to 8 membered heterocycloalkylene, 3 to 6 membered heterocycloalkylene, or 5 to 6 membered heterocycloalkylene), unsubstituted arylene (e.g., C₆-C₁₀ arylene, C₁₀ arylene, or phenylene), or unsubstituted heteroarylene (e.g., 5 to 10 membered heteroarylene, 5 to 9 membered heteroarylene, or 5 to 6 membered heteroarylene).

In some embodiments, L¹ is R⁴³-substituted or unsubstituted alkylene (e.g., C₁-C₈ alkylene, C₁-C₆ alkylene, or C₁-C₄ alkylene). In some embodiments, L¹ is R⁴³-substituted alkylene (e.g., C₁-C₈ alkylene, C₁-C₆ alkylene, or C₁-C₄ alkylene). In some embodiments, Li is an unsubstituted alkylene (e.g., C₁-C₈ alkylene, C₁-C₆ alkylene, or C₁-C₄ alkylene). In some embodiments, L¹ is R⁴³-substituted or unsubstituted heteroalkylene (e.g., 2 to 8 membered heteroalkylene, 2 to 6 membered heteroalkylene, or 2 to 4 membered heteroalkylene). In some embodiments, L¹ is R⁴³-substituted heteroalkylene (e.g., 2 to 8 membered heteroalkylene, 2 to 6 membered heteroalkylene, or 2 to 4 membered heteroalkylene). In some embodiments, L¹ is an unsubstituted heteroalkylene (e.g., 2 to 8 membered heteroalkylene, 2 to 6 membered heteroalkylene, or 2 to 4 membered heteroalkylene). In some embodiments, L¹ is R⁴³-substituted or unsubstituted cycloalkylene (e.g., C₃-C₈ cycloalkylene, C₃-C₆ cycloalkylene, or C₅-C₆ cycloalkylene). In some embodiments, L¹ is R⁴³-substituted cycloalkylene (e.g., C₃-C₈ cycloalkylene, C₃-C₆ cycloalkylene, or C₅-C₆ cycloalkylene). In some embodiments, L¹ is an unsubstituted cycloalkylene (e.g., C₃-C₈ cycloalkylene, C₃-C₆ cycloalkylene, or C₅-C₆ cycloalkylene). In some embodiments, L¹ is R⁴³-substituted or unsubstituted heterocycloalkylene (e.g., 3 to 8 membered heterocycloalkylene, 3 to 6 membered heterocycloalkylene, or 5 to 6 membered heterocycloalkylene). In some embodiments, L¹ is R⁴³-substituted heterocycloalkylene (e.g., 3 to 8 membered heterocycloalkylene, 3 to 6 membered heterocycloalkylene, or 5 to 6 membered heterocycloalkylene). In some embodiments, L¹ is an unsubstituted heterocycloalkylene (e.g., 3 to 8 membered heterocycloalkylene, 3 to 6 membered heterocycloalkylene, or 5 to 6 membered heterocycloalkylene). In some embodiments, L¹ is R⁴³-substituted or unsubstituted arylene (e.g., C₆-C₁₀ arylene, C₁₀ arylene, or phenylene). In some embodiments, L¹ is R⁴³-substituted arylene (e.g., C₆-C₁₀ arylene, C₁₀ arylene, or phenylene). In some embodiments, L¹ is an unsubstituted arylene (e.g., C₆-C₁₀ arylene, C₁₀ arylene, or phenylene). In some embodiments, L¹ is R⁴³-substituted or unsubstituted heteroarylene (e.g., 5 to 10 membered heteroarylene, 5 to 9 membered heteroarylene, or 5 to 6 membered heteroarylene). In some embodiments, L¹ is R⁴³-substituted heteroarylene (e.g., 5 to 10 membered heteroarylene, 5 to 9 membered heteroarylene, or 5 to 6 membered heteroarylene). In some embodiments, L¹ is an unsubstituted heteroarylene (e.g., 5 to 10 membered heteroarylene, 5 to 9 membered heteroarylene, or 5 to 6 membered heteroarylene).

R⁴³ is independently oxo, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R⁴³ is independently oxo. In some embodiments, R⁴³ is independently halogen. In some embodiments, R⁴³ is independently —CCl₃. In some embodiments, R⁴³ is independently —CBr₃. In some embodiments, R⁴³ is independently —CF₃.

In some embodiments, R⁴³ is independently —CI₃. In some embodiments, R⁴³ is independently —CHCl₂. In some embodiments, R⁴³ is independently —CHBr₂. In some embodiments, R⁴³ is independently —CHF₂. In some embodiments, R⁴³ is independently —CHI₂. In some embodiments, R⁴³ is independently —CH₂Cl. In some embodiments, R⁴³ is independently —CH₂Br. In some embodiments, R⁴³ is independently —CH₂F. In some embodiments, R⁴³ is independently —CH₂I. In some embodiments, R⁴³ is independently —CN. In some embodiments, R⁴³ is independently —OH. In some embodiments, R⁴³ is independently —NH₂. In some embodiments, R⁴³ is independently —COOH. In some embodiments, R⁴³ is independently —CONH₂. In some embodiments, R⁴³ is independently —NO₂. In some embodiments, R⁴³ is independently —SH. In some embodiments, R⁴³ is independently —SO₃H. In some embodiments, R⁴³ is independently —SO₄H. In some embodiments, R⁴³ is independently —SO₂NH₂. In some embodiments, R⁴³ is independently —NHNH₂. In some embodiments, R⁴³ is independently —ONH₂. In some embodiments, R⁴³ is independently —NHC(O)NHNH₂. In some embodiments, R⁴³ is independently —NHC(O)NH₂. In some embodiments, R⁴³ is independently —NHSO₂H. In some embodiments, R⁴³ is independently —NHC(O)H. In some embodiments, R⁴³ is independently —NHC(O)OH. In some embodiments, R⁴³ is independently —NHOH. In some embodiments, R⁴³ is independently —OCCl₃. In some embodiments, R⁴³ is independently —OCF₃. In some embodiments, R⁴³ is independently —OCBr₃. In some embodiments, R⁴³ is independently —OCI₃. In some embodiments, R⁴³ is independently —OCHCl₂. In some embodiments, R⁴³ is independently —OCHBr₂. In some embodiments, R⁴³ is independently —OCHI₂. In some embodiments, R⁴³ is independently —OCHF₂. In some embodiments, R⁴³ is independently —OCH₂Cl. In some embodiments, R⁴³ is independently —OCH₂Br. In some embodiments, R⁴³ is independently —OCH₂I. In some embodiments, R⁴³ is independently —OCH₂F. In some embodiments, R⁴³ is independently —N₃. In some embodiments, R⁴³ is independently —OCH₃. In some embodiments, R⁴³ is independently —CH₃. In some embodiments, R⁴³ is independently —CH₂CH₃. In some embodiments, R⁴³ is independently unsubstituted propyl. In some embodiments, R⁴³ is independently unsubstituted isopropyl. In some embodiments, R⁴³ is independently unsubstituted butyl. In some embodiments, R⁴³ is independently unsubstituted tert-butyl. In some embodiments, R⁴³ is independently —F. In some embodiments, R⁴³ is independently —Cl. In some embodiments, R⁴³ is independently —Br. In some embodiments, R⁴³ is independently —I.

In some embodiments, L² is independently a bond, —S(O)₂—, —S(O)—, —NH—, —O—, —S—, —C(O)—, —C(O)NH—, —NHC(O)—, —NHC(O)NH—, —C(O)O—, —OC(O)—, substituted or unsubstituted alkylene (e.g., C₁-C₈ alkylene, C₁-C₆ alkylene, or C₁-C₄ alkylene), substituted or unsubstituted heteroalkylene (e.g., 2 to 8 membered heteroalkylene, 2 to 6 membered heteroalkylene, or 2 to 4 membered heteroalkylene), substituted or unsubstituted cycloalkylene (e.g., C₃-C₈ cycloalkylene, C₃-C₆ cycloalkylene, or C₅-C₆ cycloalkylene), substituted or unsubstituted heterocycloalkylene (e.g., 3 to 8 membered heterocycloalkylene, 3 to 6 membered heterocycloalkylene, or 5 to 6 membered heterocycloalkylene), substituted or unsubstituted arylene (e.g., C₆-C₁₀ arylene, C₁₀ arylene, or phenylene), or substituted or unsubstituted heteroarylene (e.g., 5 to 10 membered heteroarylene, 5 to 9 membered heteroarylene, or 5 to 6 membered heteroarylene). In some embodiments, L² is independently a —S(O)₂—. In some embodiments, L² is independently a —S(O)—. In some embodiments, L² is independently a —NH—. In some embodiments, L² is independently a —O—. In some embodiments, L² is independently a —S—. In some embodiments, L² is independently a —C(O)—. In some embodiments, L² is independently a —C(O)NH—. In some embodiments, L² is independently a —NHC(O)—. In some embodiments, L² is independently a —NHC(O)NH—. In some embodiments, L² is independently a —C(O)O—. In some embodiments, L² is independently —OC(O)—. In some embodiments, L² is independently a bond.

In some embodiments, L² is substituted or unsubstituted alkylene (e.g., C₁-C₈ alkylene, C₁-C₆ alkylene, or C₁-C₄ alkylene). In some embodiments, L² is substituted alkylene (e.g., C₁-C₈ alkylene, C₁-C₆ alkylene, or C₁-C₄ alkylene). In some embodiments, L² is an unsubstituted alkylene (e.g., C₁-C₈ alkylene, C₁-C₆ alkylene, or C₁-C₄ alkylene). In some embodiments, L² is substituted or unsubstituted heteroalkylene (e.g., 2 to 8 membered heteroalkylene, 2 to 6 membered heteroalkylene, or 2 to 4 membered heteroalkylene). In some embodiments, L² is substituted heteroalkylene (e.g., 2 to 8 membered heteroalkylene, 2 to 6 membered heteroalkylene, or 2 to 4 membered heteroalkylene). In some embodiments, L² is an unsubstituted heteroalkylene (e.g., 2 to 8 membered heteroalkylene, 2 to 6 membered heteroalkylene, or 2 to 4 membered heteroalkylene). In some embodiments, L² is substituted or unsubstituted cycloalkylene (e.g., C₃-C₈ cycloalkylene, C₃-C₆ cycloalkylene, or C₅-C₆ cycloalkylene). In some embodiments, L² is substituted cycloalkylene (e.g., C₃-C₈ cycloalkylene, C₃-C₆ cycloalkylene, or C₅-C₆ cycloalkylene). In some embodiments, L² is an unsubstituted cycloalkylene (e.g., C₃-C₈ cycloalkylene, C₃-C₆ cycloalkylene, or C₅-C₆ cycloalkylene). In some embodiments, L² is substituted or unsubstituted heterocycloalkylene (e.g., 3 to 8 membered heterocycloalkylene, 3 to 6 membered heterocycloalkylene, or 5 to 6 membered heterocycloalkylene). In some embodiments, L² is substituted heterocycloalkylene (e.g., 3 to 8 membered heterocycloalkylene, 3 to 6 membered heterocycloalkylene, or 5 to 6 membered heterocycloalkylene). In some embodiments, L² is an unsubstituted heterocycloalkylene (e.g., 3 to 8 membered heterocycloalkylene, 3 to 6 membered heterocycloalkylene, or 5 to 6 membered heterocycloalkylene). In some embodiments, L² is substituted or unsubstituted arylene (e.g., C₆-C₁₀ arylene, C₁₀ arylene, or phenylene). In some embodiments, L² is substituted arylene (e.g., C₆-C₁₀ arylene, C₁₀ arylene, or phenylene). In some embodiments, L² is an unsubstituted arylene (e.g., C₆-C₁₀ arylene, C₁₀ arylene, or phenylene). In some embodiments, L² is substituted or unsubstituted heteroarylene (e.g., 5 to 10 membered heteroarylene, 5 to 9 membered heteroarylene, or 5 to 6 membered heteroarylene). In some embodiments, L² is substituted heteroarylene (e.g., 5 to 10 membered heteroarylene, 5 to 9 membered heteroarylene, or 5 to 6 membered heteroarylene). In some embodiments, L² is an unsubstituted heteroarylene (e.g., 5 to 10 membered heteroarylene, 5 to 9 membered heteroarylene, or 5 to 6 membered heteroarylene).

In some embodiments, L² is independently a bond, —S(O)₂—, —S(O)—, —NH—, —O—, —S—, —C(O)—, —C(O)NH—, —NHC(O)—, —NHC(O)NH—, —C(O)O—, —OC(O)—, R⁴⁴-substituted or unsubstituted alkylene (e.g., C₁-C₈ alkylene, C₁-C₆ alkylene, or C₁-C₄ alkylene), R⁴⁴-substituted or unsubstituted heteroalkylene (e.g., 2 to 8 membered heteroalkylene, 2 to 6 membered heteroalkylene, or 2 to 4 membered heteroalkylene), R⁴⁴-substituted or unsubstituted cycloalkylene (e.g., C₃-C₈ cycloalkylene, C₃-C₆ cycloalkylene, or C₅-C₆ cycloalkylene), R⁴⁴-substituted or unsubstituted heterocycloalkylene (e.g., 3 to 8 membered heterocycloalkylene, 3 to 6 membered heterocycloalkylene, or 5 to 6 membered heterocycloalkylene), R⁴⁴-substituted or unsubstituted arylene (e.g., C₆-C₁₀ arylene, C₁₀ arylene, or phenylene), or R⁴⁴-substituted or unsubstituted heteroarylene (e.g., 5 to 10 membered heteroarylene, 5 to 9 membered heteroarylene, or 5 to 6 membered heteroarylene). In some embodiments, L² is independently a bond, —S(O)₂—, —S(O)—, —NH—, —O—, —S—, —C(O)—, —C(O)NH—, —NHC(O)—, —NHC(O)NH—, —C(O)O—, —OC(O)—, unsubstituted alkylene (e.g., C₁-C₈ alkylene, C₁-C₆ alkylene, or C₁-C₄ alkylene), unsubstituted heteroalkylene (e.g., 2 to 8 membered heteroalkylene, 2 to 6 membered heteroalkylene, or 2 to 4 membered heteroalkylene), unsubstituted cycloalkylene (e.g., C₃-C₈ cycloalkylene, C₃-C₆ cycloalkylene, or C₅-C₆ cycloalkylene), unsubstituted heterocycloalkylene (e.g., 3 to 8 membered heterocycloalkylene, 3 to 6 membered heterocycloalkylene, or 5 to 6 membered heterocycloalkylene), unsubstituted arylene (e.g., C₆-C₁₀ arylene, C₁₀ arylene, or phenylene), or unsubstituted heteroarylene (e.g., 5 to 10 membered heteroarylene, 5 to 9 membered heteroarylene, or 5 to 6 membered heteroarylene).

In some embodiments, L² is R⁴⁴-substituted or unsubstituted alkylene (e.g., C₁-C₈ alkylene, C₁-C₆ alkylene, or C₁-C₄ alkylene). In some embodiments, L² is R⁴⁴-substituted alkylene (e.g., C₁-C₈ alkylene, C₁-C₆ alkylene, or C₁-C₄ alkylene). In some embodiments, L² is an unsubstituted alkylene (e.g., C₁-C₈ alkylene, C₁-C₆ alkylene, or C₁-C₄ alkylene). In some embodiments, L² is R⁴⁴-substituted or unsubstituted heteroalkylene (e.g., 2 to 8 membered heteroalkylene, 2 to 6 membered heteroalkylene, or 2 to 4 membered heteroalkylene). In some embodiments, L² is R⁴⁴-substituted heteroalkylene (e.g., 2 to 8 membered heteroalkylene, 2 to 6 membered heteroalkylene, or 2 to 4 membered heteroalkylene). In some embodiments, L² is an unsubstituted heteroalkylene (e.g., 2 to 8 membered heteroalkylene, 2 to 6 membered heteroalkylene, or 2 to 4 membered heteroalkylene). In some embodiments, L² is R⁴⁴-substituted or unsubstituted cycloalkylene (e.g., C₃-C₈ cycloalkylene, C₃-C₆ cycloalkylene, or C₅-C₆ cycloalkylene). In some embodiments, L² is R⁴⁴-substituted cycloalkylene (e.g., C₃-C₈ cycloalkylene, C₃-C₆ cycloalkylene, or C₅-C₆ cycloalkylene). In some embodiments, L² is an unsubstituted cycloalkylene (e.g., C₃-C₈ cycloalkylene, C₃-C₆ cycloalkylene, or C₅-C₆ cycloalkylene). In some embodiments, L² is R⁴⁴-substituted or unsubstituted heterocycloalkylene (e.g., 3 to 8 membered heterocycloalkylene, 3 to 6 membered heterocycloalkylene, or 5 to 6 membered heterocycloalkylene). In some embodiments, L² is R⁴⁴-substituted heterocycloalkylene (e.g., 3 to 8 membered heterocycloalkylene, 3 to 6 membered heterocycloalkylene, or 5 to 6 membered heterocycloalkylene). In some embodiments, L² is an unsubstituted heterocycloalkylene (e.g., 3 to 8 membered heterocycloalkylene, 3 to 6 membered heterocycloalkylene, or 5 to 6 membered heterocycloalkylene). In some embodiments, L² is R⁴⁴-substituted or unsubstituted arylene (e.g., C₆-C₁₀ arylene, C₁₀ arylene, or phenylene). In some embodiments, L² is R⁴⁴-substituted arylene (e.g., C₆-C₁₀ arylene, C₁₀ arylene, or phenylene). In some embodiments, L² is an unsubstituted arylene (e.g., C₆-C₁₀ arylene, C₁₀ arylene, or phenylene). In some embodiments, L² is R⁴⁴-substituted or unsubstituted heteroarylene (e.g., 5 to 10 membered heteroarylene, 5 to 9 membered heteroarylene, or 5 to 6 membered heteroarylene). In some embodiments, L² is R⁴⁴-substituted heteroarylene (e.g., 5 to 10 membered heteroarylene, 5 to 9 membered heteroarylene, or 5 to 6 membered heteroarylene). In some embodiments, L² is an unsubstituted heteroarylene (e.g., 5 to 10 membered heteroarylene, 5 to 9 membered heteroarylene, or 5 to 6 membered heteroarylene).

R⁴⁴ is independently oxo, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R⁴⁴ is independently oxo. In some embodiments, R⁴⁴ is independently halogen. In some embodiments, R⁴⁴ is independently —CCl₃. In some embodiments, R⁴⁴ is independently —CBr₃. In some embodiments, R⁴⁴ is independently —CF₃. In some embodiments, R⁴⁴ is independently —CI₃. In some embodiments, R⁴⁴ is independently —CHCl₂. In some embodiments, R⁴⁴ is independently —CHBr₂. In some embodiments, R⁴⁴ is independently —CHF₂. In some embodiments, R⁴⁴ is independently —CHI₂. In some embodiments, R⁴⁴ is independently —CH₂Cl. In some embodiments, R⁴⁴ is independently —CH₂Br. In some embodiments, R⁴⁴ is independently —CH₂F. In some embodiments, R⁴⁴ is independently —CH₂I. In some embodiments, R⁴⁴ is independently —CN. In some embodiments, R⁴⁴ is independently —OH. In some embodiments, R⁴⁴ is independently —NH₂. In some embodiments, R⁴⁴ is independently —COOH. In some embodiments, R⁴⁴ is independently —CONH₂. In some embodiments, R⁴⁴ is independently —NO₂. In some embodiments, R⁴⁴ is independently —SH. In some embodiments, R⁴⁴ is independently —SO₃H. In some embodiments, R⁴⁴ is independently —SO₄H. In some embodiments, R⁴⁴ is independently —SO₂NH₂. In some embodiments, R⁴⁴ is independently —NHNH₂. In some embodiments, R⁴⁴ is independently —ONH₂. In some embodiments, R⁴⁴ is independently —NHC(O)NHNH₂. In some embodiments, R⁴⁴ is independently —NHC(O)NH₂. In some embodiments, R⁴⁴ is independently —NHSO₂H. In some embodiments, R⁴⁴ is independently —NHC(O)H. In some embodiments, R⁴⁴ is independently —NHC(O)OH. In some embodiments, R⁴⁴ is independently —NHOH. In some embodiments, R⁴⁴ is independently —OCCl₃. In some embodiments, R⁴⁴ is independently —OCF₃. In some embodiments, R⁴⁴ is independently —OCBr₃. In some embodiments, R⁴⁴ is independently —OCI₃. In some embodiments, R⁴⁴ is independently —OCHCl₂. In some embodiments, R⁴⁴ is independently —OCHBr₂. In some embodiments, R⁴⁴ is independently —OCHI₂. In some embodiments, R⁴⁴ is independently —OCHF₂. In some embodiments, R⁴⁴ is independently —OCH₂Cl. In some embodiments, R⁴⁴ is independently —OCH₂Br. In some embodiments, R⁴⁴ is independently —OCH₂I. In some embodiments, R⁴⁴ is independently —OCH₂F. In some embodiments, R⁴⁴ is independently —N₃. In some embodiments, R⁴⁴ is independently —OCH₃. In some embodiments, R⁴⁴ is independently —CH₃. In some embodiments, R⁴⁴ is independently —CH₂CH₃. In some embodiments, R⁴⁴ is independently unsubstituted propyl. In some embodiments, R⁴⁴ is independently unsubstituted isopropyl. In some embodiments, R⁴⁴ is independently unsubstituted butyl. In some embodiments, R⁴⁴ is independently unsubstituted tert-butyl. In some embodiments, R⁴⁴ is independently —F. In some embodiments, R⁴⁴ is independently —Cl. In some embodiments, R⁴⁴ is independently —Br. In some embodiments, R⁴⁴ is independently —I.

In some embodiments, L³ is independently a bond, —S(O)₂—, —S(O)—, —NH—, —O—, —S—, —C(O)—, —C(O)NH—, —NHC(O)—, —NHC(O)NH—, —C(O)O—, —OC(O)—, substituted or unsubstituted alkylene (e.g., C₁-C₈ alkylene, C₁-C₆ alkylene, or C₁-C₄ alkylene), substituted or unsubstituted heteroalkylene (e.g., 2 to 8 membered heteroalkylene, 2 to 6 membered heteroalkylene, or 2 to 4 membered heteroalkylene), substituted or unsubstituted cycloalkylene (e.g., C₃-C₈ cycloalkylene, C₃-C₆ cycloalkylene, or C₅-C₆ cycloalkylene), substituted or unsubstituted heterocycloalkylene (e.g., 3 to 8 membered heterocycloalkylene, 3 to 6 membered heterocycloalkylene, or 5 to 6 membered heterocycloalkylene), substituted or unsubstituted arylene (e.g., C₆-C₁₀ arylene, C₁₀ arylene, or phenylene), or substituted or unsubstituted heteroarylene (e.g., 5 to 10 membered heteroarylene, 5 to 9 membered heteroarylene, or 5 to 6 membered heteroarylene). In some embodiments, L³ is independently a —S(O)₂—. In some embodiments, L³ is independently a —S(O)—. In some embodiments, L³ is independently a —NH—. In some embodiments, L³ is independently a —O—. In some embodiments, L³ is independently a —S—. In some embodiments, L³ is independently a —C(O)—. In some embodiments, L³ is independently a —C(O)NH—. In some embodiments, L³ is independently a —NHC(O)—. In some embodiments, L³ is independently a —NHC(O)NH—. In some embodiments, L³ is independently a —C(O)O—. In some embodiments, L³ is independently —OC(O)—. In some embodiments, L³ is independently a bond.

In some embodiments, L³ is substituted or unsubstituted alkylene (e.g., C₁-C₈ alkylene, C₁-C₆ alkylene, or C₁-C₄ alkylene). In some embodiments, L³ is substituted alkylene (e.g., C₁-C₈ alkylene, C₁-C₆ alkylene, or C₁-C₄ alkylene). In some embodiments, L³ is an unsubstituted alkylene (e.g., C₁-C₈ alkylene, C₁-C₆ alkylene, or C₁-C₄ alkylene). In some embodiments, L³ is substituted or unsubstituted heteroalkylene (e.g., 2 to 8 membered heteroalkylene, 2 to 6 membered heteroalkylene, or 2 to 4 membered heteroalkylene). In some embodiments, L³ is substituted heteroalkylene (e.g., 2 to 8 membered heteroalkylene, 2 to 6 membered heteroalkylene, or 2 to 4 membered heteroalkylene). In some embodiments, L³ is an unsubstituted heteroalkylene (e.g., 2 to 8 membered heteroalkylene, 2 to 6 membered heteroalkylene, or 2 to 4 membered heteroalkylene). In some embodiments, L³ is substituted or unsubstituted cycloalkylene (e.g., C₃-C₈ cycloalkylene, C₃-C₆ cycloalkylene, or C₅-C₆ cycloalkylene). In some embodiments, L³ is substituted cycloalkylene (e.g., C₃-C₈ cycloalkylene, C₃-C₆ cycloalkylene, or C₅-C₆ cycloalkylene). In some embodiments, L³ is an unsubstituted cycloalkylene (e.g., C₃-C₈ cycloalkylene, C₃-C₆ cycloalkylene, or C₅-C₆ cycloalkylene). In some embodiments, L³ is substituted or unsubstituted heterocycloalkylene (e.g., 3 to 8 membered heterocycloalkylene, 3 to 6 membered heterocycloalkylene, or 5 to 6 membered heterocycloalkylene). In some embodiments, L³ is substituted heterocycloalkylene (e.g., 3 to 8 membered heterocycloalkylene, 3 to 6 membered heterocycloalkylene, or 5 to 6 membered heterocycloalkylene). In some embodiments, L³ is an unsubstituted heterocycloalkylene (e.g., 3 to 8 membered heterocycloalkylene, 3 to 6 membered heterocycloalkylene, or 5 to 6 membered heterocycloalkylene). In some embodiments, L³ is substituted or unsubstituted arylene (e.g., C₆-C₁₀ arylene, C₁₀ arylene, or phenylene). In some embodiments, L³ is substituted arylene (e.g., C₆-C₁₀ arylene, C₁₀ arylene, or phenylene). In some embodiments, L³ is an unsubstituted arylene (e.g., C₆-C₁₀ arylene, C₁₀ arylene, or phenylene). In some embodiments, L³ is substituted or unsubstituted heteroarylene (e.g., 5 to 10 membered heteroarylene, 5 to 9 membered heteroarylene, or 5 to 6 membered heteroarylene). In some embodiments, L³ is substituted heteroarylene (e.g., 5 to 10 membered heteroarylene, 5 to 9 membered heteroarylene, or 5 to 6 membered heteroarylene). In some embodiments, L³ is an unsubstituted heteroarylene (e.g., 5 to 10 membered heteroarylene, 5 to 9 membered heteroarylene, or 5 to 6 membered heteroarylene).

In some embodiments, L³ is independently a bond, —S(O)₂—, —S(O)—, —NH—, —O—, —S—, —C(O)—, —C(O)NH—, —NHC(O)—, —NHC(O)NH—, —C(O)O—, —OC(O)—, R⁴⁵-substituted or unsubstituted alkylene (e.g., C₁-C₈ alkylene, C₁-C₆ alkylene, or C₁-C₄ alkylene), R⁴⁵-substituted or unsubstituted heteroalkylene (e.g., 2 to 8 membered heteroalkylene, 2 to 6 membered heteroalkylene, or 2 to 4 membered heteroalkylene), R⁴⁵-substituted or unsubstituted cycloalkylene (e.g., C₃-C₈ cycloalkylene, C₃-C₆ cycloalkylene, or C₅-C₆ cycloalkylene), R⁴⁵-substituted or unsubstituted heterocycloalkylene (e.g., 3 to 8 membered heterocycloalkylene, 3 to 6 membered heterocycloalkylene, or 5 to 6 membered heterocycloalkylene), R⁴⁵-substituted or unsubstituted arylene (e.g., C₆-C₁₀ arylene, C₁₀ arylene, or phenylene), or R⁴⁵-substituted or unsubstituted heteroarylene (e.g., 5 to 10 membered heteroarylene, 5 to 9 membered heteroarylene, or 5 to 6 membered heteroarylene). In some embodiments, L³ is independently a bond, —S(O)₂—, —S(O)—, —NH—, —O—, —S—, —C(O)—, —C(O)NH—, —NHC(O)—, —NHC(O)NH—, —C(O)O—, —OC(O)—, unsubstituted alkylene (e.g., C₁-C₈ alkylene, C₁-C₆ alkylene, or C₁-C₄ alkylene), unsubstituted heteroalkylene (e.g., 2 to 8 membered heteroalkylene, 2 to 6 membered heteroalkylene, or 2 to 4 membered heteroalkylene), unsubstituted cycloalkylene (e.g., C₃-C₈ cycloalkylene, C₃-C₆ cycloalkylene, or C₅-C₆ cycloalkylene), unsubstituted heterocycloalkylene (e.g., 3 to 8 membered heterocycloalkylene, 3 to 6 membered heterocycloalkylene, or 5 to 6 membered heterocycloalkylene), unsubstituted arylene (e.g., C₆-C₁₀ arylene, C₁₀ arylene, or phenylene), or unsubstituted heteroarylene (e.g., 5 to 10 membered heteroarylene, 5 to 9 membered heteroarylene, or 5 to 6 membered heteroarylene).

In some embodiments, L³ is R⁴⁵-substituted or unsubstituted alkylene (e.g., C₁-C₈ alkylene, C₁-C₆ alkylene, or C₁-C₄ alkylene). In some embodiments, L³ is R⁴⁵-substituted alkylene (e.g., C₁-C₈ alkylene, C₁-C₆ alkylene, or C₁-C₄ alkylene). In some embodiments, L³ is an unsubstituted alkylene (e.g., C₁-C₈ alkylene, C₁-C₆ alkylene, or C₁-C₄ alkylene). In some embodiments, L³ is R⁴⁵-substituted or unsubstituted heteroalkylene (e.g., 2 to 8 membered heteroalkylene, 2 to 6 membered heteroalkylene, or 2 to 4 membered heteroalkylene). In some embodiments, L³ is R⁴⁵-substituted heteroalkylene (e.g., 2 to 8 membered heteroalkylene, 2 to 6 membered heteroalkylene, or 2 to 4 membered heteroalkylene). In some embodiments, L³ is an unsubstituted heteroalkylene (e.g., 2 to 8 membered heteroalkylene, 2 to 6 membered heteroalkylene, or 2 to 4 membered heteroalkylene). In some embodiments, L³ is R⁴⁵-substituted or unsubstituted cycloalkylene (e.g., C₃-C₈ cycloalkylene, C₃-C₆ cycloalkylene, or C₅-C₆ cycloalkylene). In some embodiments, L³ is R⁴⁵-substituted cycloalkylene (e.g., C₃-C₈ cycloalkylene, C₃-C₆ cycloalkylene, or C₅-C₆ cycloalkylene). In some embodiments, L³ is an unsubstituted cycloalkylene (e.g., C₃-C₈ cycloalkylene, C₃-C₆ cycloalkylene, or C₅-C₆ cycloalkylene). In some embodiments, L³ is R⁴⁵-substituted or unsubstituted heterocycloalkylene (e.g., 3 to 8 membered heterocycloalkylene, 3 to 6 membered heterocycloalkylene, or 5 to 6 membered heterocycloalkylene). In some embodiments, L³ is R⁴⁵-substituted heterocycloalkylene (e.g., 3 to 8 membered heterocycloalkylene, 3 to 6 membered heterocycloalkylene, or 5 to 6 membered heterocycloalkylene). In some embodiments, L³ is an unsubstituted heterocycloalkylene (e.g., 3 to 8 membered heterocycloalkylene, 3 to 6 membered heterocycloalkylene, or 5 to 6 membered heterocycloalkylene). In some embodiments, L³ is R⁴⁵-substituted or unsubstituted arylene (e.g., C₆-C₁₀ arylene, C₁₀ arylene, or phenylene). In some embodiments, L³ is R⁴⁵-substituted arylene (e.g., C₆-C₁₀ arylene, C₁₀ arylene, or phenylene). In some embodiments, L³ is an unsubstituted arylene (e.g., C₆-C₁₀ arylene, C₁₀ arylene, or phenylene). In some embodiments, L³ is R⁴⁵-substituted or unsubstituted heteroarylene (e.g., 5 to 10 membered heteroarylene, 5 to 9 membered heteroarylene, or 5 to 6 membered heteroarylene). In some embodiments, L³ is R⁴⁵-substituted heteroarylene (e.g., 5 to 10 membered heteroarylene, 5 to 9 membered heteroarylene, or 5 to 6 membered heteroarylene). In some embodiments, L³ is an unsubstituted heteroarylene (e.g., 5 to 10 membered heteroarylene, 5 to 9 membered heteroarylene, or 5 to 6 membered heteroarylene).

In some embodiments, R⁴⁵ is independently oxo, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R⁴⁵ is independently oxo. In some embodiments, R⁴⁵ is independently halogen. In some embodiments, R⁴⁵ is independently —CCl₃. In some embodiments, R⁴⁵ is independently —CBr₃. In some embodiments, R⁴⁵ is independently —CF₃. In some embodiments, R⁴⁵ is independently —CI₃. In some embodiments, R⁴⁵ is independently —CHCl₂. In some embodiments, R⁴⁵ is independently —CHBr₂. In some embodiments, R⁴⁵ is independently —CHF₂. In some embodiments, R⁴⁵ is independently —CHI₂. In some embodiments, R⁴⁵ is independently —CH₂Cl. In some embodiments, R⁴⁵ is independently —CH₂Br. In some embodiments, R⁴⁵ is independently —CH₂F. In some embodiments, R⁴⁵ is independently —CH₂I. In some embodiments, R⁴⁵ is independently —CN. In some embodiments, R⁴⁵ is independently —OH. In some embodiments, R⁴⁵ is independently —NH₂. In some embodiments, R⁴⁵ is independently —COOH. In some embodiments, R⁴⁵ is independently —CONH₂. In some embodiments, R⁴⁵ is independently —NO₂. In some embodiments, R⁴⁵ is independently —SH. In some embodiments, R⁴⁵ is independently —SO₃H. In some embodiments, R⁴⁵ is independently —SO₄H. In some embodiments, R⁴⁵ is independently —SO₂NH₂. In some embodiments, R⁴⁵ is independently —NHNH₂. In some embodiments, R⁴⁵ is independently —ONH₂. In some embodiments, R⁴⁵ is independently —NHC(O)NHNH₂. In some embodiments, R⁴⁵ is independently —NHC(O)NH₂. In some embodiments, R⁴⁵ is independently —NHSO₂H. In some embodiments, R⁴⁵ is independently —NHC(O)H. In some embodiments, R⁴⁵ is independently —NHC(O)OH. In some embodiments, R⁴⁵ is independently —NHOH. In some embodiments, R⁴⁵ is independently —OCCl₃. In some embodiments, R⁴⁵ is independently —OCF₃. In some embodiments, R⁴⁵ is independently —OCBr₃. In some embodiments, R⁴⁵ is independently —OCI₃. In some embodiments, R⁴⁵ is independently —OCHCl₂. In some embodiments, R⁴⁵ is independently —OCHBr₂. In some embodiments, R⁴⁵ is independently —OCHI₂. In some embodiments, R⁴⁵ is independently —OCHF₂. In some embodiments, R⁴⁵ is independently —OCH₂Cl. In some embodiments, R⁴⁵ is independently —OCH₂Br. In some embodiments, R⁴⁵ is independently —OCH₂I. In some embodiments, R⁴⁵ is independently —OCH₂F. In some embodiments, R⁴⁵ is independently —N₃. In some embodiments, R⁴⁵ is independently —OCH₃. In some embodiments, R⁴⁵ is independently —CH₃. In some embodiments, R⁴⁵ is independently —CH₂CH₃. In some embodiments, R⁴⁵ is independently unsubstituted propyl. In some embodiments, R⁴⁵ is independently unsubstituted isopropyl. In some embodiments, R⁴⁵ is independently unsubstituted butyl. In some embodiments, R⁴⁵ is independently unsubstituted tert-butyl. In some embodiments, R⁴⁵ is independently —F. In some embodiments, R⁴⁵ is independently —Cl. In some embodiments, R⁴⁵ is independently —Br. In some embodiments, R⁴⁵ is independently —I.

In some embodiments, L⁴ is independently a bond, —S(O)₂—, —S(O)—, —NH—, —O—, —S—, —C(O)—, —C(O)NH—, —NHC(O)—, —NHC(O)NH—, —C(O)O—, —OC(O)—, substituted or unsubstituted alkylene (e.g., C₁-C₈ alkylene, C₁-C₆ alkylene, or C₁-C₄ alkylene), substituted or unsubstituted heteroalkylene (e.g., 2 to 8 membered heteroalkylene, 2 to 6 membered heteroalkylene, or 2 to 4 membered heteroalkylene), substituted or unsubstituted cycloalkylene (e.g., C₃-C₈ cycloalkylene, C₃-C₆ cycloalkylene, or C₅-C₆ cycloalkylene), substituted or unsubstituted heterocycloalkylene (e.g., 3 to 8 membered heterocycloalkylene, 3 to 6 membered heterocycloalkylene, or 5 to 6 membered heterocycloalkylene), substituted or unsubstituted arylene (e.g., C₆-C₁₀ arylene, C₁₀ arylene, or phenylene), or substituted or unsubstituted heteroarylene (e.g., 5 to 10 membered heteroarylene, 5 to 9 membered heteroarylene, or 5 to 6 membered heteroarylene). In some embodiments, L⁴ is independently a —S(O)₂—. In some embodiments, L⁴ is independently a —S(O)—. In some embodiments, L⁴ is independently a —NH—. In some embodiments, L⁴ is independently a —O—. In some embodiments, L⁴ is independently a —S—. In some embodiments, L⁴ is independently a —C(O)—. In some embodiments, L⁴ is independently a —C(O)NH—. In some embodiments, L⁴ is independently a —NHC(O)—. In some embodiments, L⁴ is independently a —NHC(O)NH—. In some embodiments, L⁴ is independently a —C(O)O—. In some embodiments, L⁴ is independently —OC(O)—. In some embodiments, L⁴ is independently a bond.

In some embodiments, L⁴ is substituted or unsubstituted alkylene (e.g., C₁-C₈ alkylene, C₁-C₆ alkylene, or C₁-C₄ alkylene). In some embodiments, L⁴ is substituted alkylene (e.g., C₁-C₈ alkylene, C₁-C₆ alkylene, or C₁-C₄ alkylene). In some embodiments, L⁴ is an unsubstituted alkylene (e.g., C₁-C₈ alkylene, C₁-C₆ alkylene, or C₁-C₄ alkylene). In some embodiments, L⁴ is substituted or unsubstituted heteroalkylene (e.g., 2 to 8 membered heteroalkylene, 2 to 6 membered heteroalkylene, or 2 to 4 membered heteroalkylene). In some embodiments, L⁴ is substituted heteroalkylene (e.g., 2 to 8 membered heteroalkylene, 2 to 6 membered heteroalkylene, or 2 to 4 membered heteroalkylene). In some embodiments, L⁴ is an unsubstituted heteroalkylene (e.g., 2 to 8 membered heteroalkylene, 2 to 6 membered heteroalkylene, or 2 to 4 membered heteroalkylene). In some embodiments, L⁴ is substituted or unsubstituted cycloalkylene (e.g., C₃-C₈ cycloalkylene, C₃-C₆ cycloalkylene, or C₅-C₆ cycloalkylene). In some embodiments, L⁴ is substituted cycloalkylene (e.g., C₃-C₈ cycloalkylene, C₃-C₆ cycloalkylene, or C₅-C₆ cycloalkylene). In some embodiments, L⁴ is an unsubstituted cycloalkylene (e.g., C₃-C₈ cycloalkylene, C₃-C₆ cycloalkylene, or C₅-C₆ cycloalkylene). In some embodiments, L⁴ is substituted or unsubstituted heterocycloalkylene (e.g., 3 to 8 membered heterocycloalkylene, 3 to 6 membered heterocycloalkylene, or 5 to 6 membered heterocycloalkylene). In some embodiments, L⁴ is substituted heterocycloalkylene (e.g., 3 to 8 membered heterocycloalkylene, 3 to 6 membered heterocycloalkylene, or 5 to 6 membered heterocycloalkylene). In some embodiments, L⁴ is an unsubstituted heterocycloalkylene (e.g., 3 to 8 membered heterocycloalkylene, 3 to 6 membered heterocycloalkylene, or 5 to 6 membered heterocycloalkylene). In some embodiments, L⁴ is substituted or unsubstituted arylene (e.g., C₆-C₁₀ arylene, C₁₀ arylene, or phenylene). In some embodiments, L⁴ is substituted arylene (e.g., C₆-C₁₀ arylene, C₁₀ arylene, or phenylene). In some embodiments, L⁴ is an unsubstituted arylene (e.g., C₆-C₁₀ arylene, C₁₀ arylene, or phenylene). In some embodiments, L⁴ is substituted or unsubstituted heteroarylene (e.g., 5 to 10 membered heteroarylene, 5 to 9 membered heteroarylene, or 5 to 6 membered heteroarylene). In some embodiments, L⁴ is substituted heteroarylene (e.g., 5 to 10 membered heteroarylene, 5 to 9 membered heteroarylene, or 5 to 6 membered heteroarylene). In some embodiments, L⁴ is an unsubstituted heteroarylene (e.g., 5 to 10 membered heteroarylene, 5 to 9 membered heteroarylene, or 5 to 6 membered heteroarylene).

In some embodiments, L⁴ is independently a bond, —S(O)₂—, —S(O)—, —NH—, —O—, —S—, —C(O)—, —C(O)NH—, —NHC(O)—, —NHC(O)NH—, —C(O)O—, —OC(O)—, R⁴⁶-substituted or unsubstituted alkylene (e.g., C₁-C₈ alkylene, C₁-C₆ alkylene, or C₁-C₄ alkylene), R⁴⁶-substituted or unsubstituted heteroalkylene (e.g., 2 to 8 membered heteroalkylene, 2 to 6 membered heteroalkylene, or 2 to 4 membered heteroalkylene), R⁴⁶-substituted or unsubstituted cycloalkylene (e.g., C₃-C₈ cycloalkylene, C₃-C₆ cycloalkylene, or C₅-C₆ cycloalkylene), R⁴⁶-substituted or unsubstituted heterocycloalkylene (e.g., 3 to 8 membered heterocycloalkylene, 3 to 6 membered heterocycloalkylene, or 5 to 6 membered heterocycloalkylene), R⁴⁶-substituted or unsubstituted arylene (e.g., C₆-C₁₀ arylene, C₁₀ arylene, or phenylene), or R⁴⁶-substituted or unsubstituted heteroarylene (e.g., 5 to 10 membered heteroarylene, 5 to 9 membered heteroarylene, or 5 to 6 membered heteroarylene). In some embodiments, L⁴ is independently a bond, —S(O)₂—, —S(O)—, —NH—, —O—, —S—, —C(O)—, —C(O)NH—, —NHC(O)—, —NHC(O)NH—, —C(O)O—, —OC(O)—, unsubstituted alkylene (e.g., C₁-C₈ alkylene, C₁-C₆ alkylene, or C₁-C₄ alkylene), unsubstituted heteroalkylene (e.g., 2 to 8 membered heteroalkylene, 2 to 6 membered heteroalkylene, or 2 to 4 membered heteroalkylene), unsubstituted cycloalkylene (e.g., C₃-C₈ cycloalkylene, C₃-C₆ cycloalkylene, or C₅-C₆ cycloalkylene), unsubstituted heterocycloalkylene (e.g., 3 to 8 membered heterocycloalkylene, 3 to 6 membered heterocycloalkylene, or 5 to 6 membered heterocycloalkylene), unsubstituted arylene (e.g., C₆-C₁₀ arylene, C₁₀ arylene, or phenylene), or unsubstituted heteroarylene (e.g., 5 to 10 membered heteroarylene, 5 to 9 membered heteroarylene, or 5 to 6 membered heteroarylene).

In some embodiments, L⁴ is R⁴⁶-substituted or unsubstituted alkylene (e.g., C₁-C₈ alkylene, C₁-C₆ alkylene, or C₁-C₄ alkylene). In some embodiments, L⁴ is R⁴⁶-substituted alkylene (e.g., C₁-C₈ alkylene, C₁-C₆ alkylene, or C₁-C₄ alkylene). In some embodiments, L⁴ is an unsubstituted alkylene (e.g., C₁-C₈ alkylene, C₁-C₆ alkylene, or C₁-C₄ alkylene). In some embodiments, L⁴ is R⁴⁶-substituted or unsubstituted heteroalkylene (e.g., 2 to 8 membered heteroalkylene, 2 to 6 membered heteroalkylene, or 2 to 4 membered heteroalkylene). In some embodiments, L⁴ is R⁴⁶-substituted heteroalkylene (e.g., 2 to 8 membered heteroalkylene, 2 to 6 membered heteroalkylene, or 2 to 4 membered heteroalkylene). In some embodiments, L⁴ is an unsubstituted heteroalkylene (e.g., 2 to 8 membered heteroalkylene, 2 to 6 membered heteroalkylene, or 2 to 4 membered heteroalkylene). In some embodiments, L⁴ is R⁴⁶-substituted or unsubstituted cycloalkylene (e.g., C₃-C₈ cycloalkylene, C₃-C₆ cycloalkylene, or C₅-C₆ cycloalkylene). In some embodiments, L⁴ is R⁴⁶-substituted cycloalkylene (e.g., C₃-C₈ cycloalkylene, C₃-C₆ cycloalkylene, or C₅-C₆ cycloalkylene). In some embodiments, L⁴ is an unsubstituted cycloalkylene (e.g., C₃-C₈ cycloalkylene, C₃-C₆ cycloalkylene, or C₅-C₆ cycloalkylene). In some embodiments, L⁴ is R⁴⁶-substituted or unsubstituted heterocycloalkylene (e.g., 3 to 8 membered heterocycloalkylene, 3 to 6 membered heterocycloalkylene, or 5 to 6 membered heterocycloalkylene). In some embodiments, L⁴ is R⁴⁶-substituted heterocycloalkylene (e.g., 3 to 8 membered heterocycloalkylene, 3 to 6 membered heterocycloalkylene, or 5 to 6 membered heterocycloalkylene). In some embodiments, L⁴ is an unsubstituted heterocycloalkylene (e.g., 3 to 8 membered heterocycloalkylene, 3 to 6 membered heterocycloalkylene, or 5 to 6 membered heterocycloalkylene). In some embodiments, L⁴ is R⁴⁶-substituted or unsubstituted arylene (e.g., C₆-C₁₀ arylene, C₁₀ arylene, or phenylene). In some embodiments, L⁴ is R⁴⁶-substituted arylene (e.g., C₆-C₁₀ arylene, C₁₀ arylene, or phenylene). In some embodiments, L⁴ is an unsubstituted arylene (e.g., C₆-C₁₀ arylene, C₁₀ arylene, or phenylene). In some embodiments, L⁴ is R⁴⁶-substituted or unsubstituted heteroarylene (e.g., 5 to 10 membered heteroarylene, 5 to 9 membered heteroarylene, or 5 to 6 membered heteroarylene). In some embodiments, L⁴ is R⁴⁶-substituted heteroarylene (e.g., 5 to 10 membered heteroarylene, 5 to 9 membered heteroarylene, or 5 to 6 membered heteroarylene). In some embodiments, L⁴ is an unsubstituted heteroarylene (e.g., 5 to 10 membered heteroarylene, 5 to 9 membered heteroarylene, or 5 to 6 membered heteroarylene).

R⁴⁶ is independently oxo, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R⁴⁶ is independently oxo. In some embodiments, R⁴⁶ is independently halogen. In some embodiments, R⁴⁶ is independently —CCl₃. In some embodiments, R⁴⁶ is independently —CBr₃. In some embodiments, R⁴⁶ is independently —CF₃. In some embodiments, R⁴⁶ is independently —CI₃. In some embodiments, R⁴⁶ is independently —CHCl₂. In some embodiments, R⁴⁶ is independently —CHBr₂. In some embodiments, R⁴⁶ is independently —CHF₂. In some embodiments, R⁴⁶ is independently —CHI₂. In some embodiments, R⁴⁶ is independently —CH₂Cl. In some embodiments, R⁴⁶ is independently —CH₂Br. In some embodiments, R⁴⁶ is independently —CH₂F. In some embodiments, R⁴⁶ is independently —CH₂I. In some embodiments, R⁴⁶ is independently —CN. In some embodiments, R⁴⁶ is independently —OH. In some embodiments, R⁴⁶ is independently —NH₂. In some embodiments, R⁴⁶ is independently —COOH. In some embodiments, R⁴⁶ is independently —CONH₂. In some embodiments, R⁴⁶ is independently —NO₂. In some embodiments, R⁴⁶ is independently —SH. In some embodiments, R⁴⁶ is independently —SO₃H. In some embodiments, R⁴⁶ is independently —SO₄H. In some embodiments, R⁴⁶ is independently —SO₂NH₂. In some embodiments, R⁴⁶ is independently —NHNH₂. In some embodiments, R⁴⁶ is independently —ONH₂. In some embodiments, R⁴⁶ is independently —NHC(O)NHNH₂. In some embodiments, R⁴⁶ is independently —NHC(O)NH₂. In some embodiments, R⁴⁶ is independently —NHSO₂H. In some embodiments, R⁴⁶ is independently —NHC(O)H. In some embodiments, R⁴⁶ is independently —NHC(O)OH. In some embodiments, R⁴⁶ is independently —NHOH. In some embodiments, R⁴⁶ is independently —OCCl₃. In some embodiments, R⁴⁶ is independently —OCF₃. In some embodiments, R⁴⁶ is independently —OCBr₃. In some embodiments, R⁴⁶ is independently —OCI₃. In some embodiments, R⁴⁶ is independently —OCHCl₂. In some embodiments, R⁴⁶ is independently —OCHBr₂. In some embodiments, R⁴⁶ is independently —OCHI₂. In some embodiments, R⁴⁶ is independently —OCHF₂. In some embodiments, R⁴⁶ is independently —OCH₂Cl. In some embodiments, R⁴⁶ is independently —OCH₂Br. In some embodiments, R⁴⁶ is independently —OCH₂I. In some embodiments, R⁴⁶ is independently —OCH₂F. In some embodiments, R⁴⁶ is independently —N₃. In some embodiments, R⁴⁶ is independently —OCH₃. In some embodiments, R⁴⁶ is independently —CH₃. In some embodiments, R⁴⁶ is independently —CH₂CH₃. In some embodiments, R⁴⁶ is independently unsubstituted propyl. In some embodiments, R⁴⁶ is independently unsubstituted isopropyl. In some embodiments, R⁴⁶ is independently unsubstituted butyl. In some embodiments, R⁴⁶ is independently unsubstituted tert-butyl. In some embodiments, R⁴⁶ is independently —F. In some embodiments, R⁴⁶ is independently —Cl. In some embodiments, R⁴⁶ is independently —Br. In some embodiments, R⁴⁶ is independently —I.

### Cellular Component Binder

In some embodiments, the cellular component binder is a compound described herein. In some embodiments, the cellular component binder is an oligonucleotide (e.g., DNA, RNA, or siRNA), protein (e.g., antibody or antibody fragment), or compound (e.g., compound described herein).

In some embodiments, the cellular component is a protein, ion, lipid, nucleic acid, nucleotide, amino acid, protein, particle, organelle, cellular compartment, microorganism, virus, lipid droplet, vesicle, small molecule, protein complex, protein aggregate, or macromolecule. In some embodiments, the cellular component is a protein. In some embodiments, the cellular component is an ion. In some embodiments, the cellular component is a lipid. In some embodiments, the cellular component is a nucleic acid. In some embodiments, the cellular component is a nucleotide. In some embodiments, the cellular component is an amino acid. In some embodiments, the cellular component is a protein. In some embodiments, the cellular component is a particle. In some embodiments, the cellular component is an organelle. In some embodiments, the cellular component is a cellular compartment. In some embodiments, the cellular component is a microorganism. In some embodiments, the cellular component is a vesicle. In some embodiments, the cellular component is a small molecule. In some embodiments, the cellular component is a protein complex. In some embodiments, the cellular component is a protein aggregate. In some embodiments, the cellular component is a macromolecule. In some embodiments, the cellular component is a lipid droplet. In some embodiments, the cellular component is a virus.

In some embodiments, the compound including a monovalent cellular component binder covalently bound to a monovalent targeted autophagy protein binder, includes a plurality of optionally different monovalent targeted autophagy protein binders.

In some embodiments, the cellular component is a ion (e.g., Na⁺, Mg⁺, Cu⁺, Cu2+, Zn²⁺, Mn²⁺, Fe²⁺, and Co²⁺). In some embodiments, the cellular component is a polysaccharide. In some embodiments, the cellular component is a lipid (e.g., fats, waxes, sterols, fat-soluble vitamins such as vitamins A, D, E, and K, monoglycerides, diglycerides, triglycerides, or phospholipids). In some embodiments, the cellular component is a nucleic acid (e.g., DNA or RNA). In some embodiments, the cellular component is a nucleotide. In some embodiments, the cellular component is an amino acid. In some embodiments, the cellular component is a particle (e.g., nanoparticle). In some embodiments, the cellular component is a plurality of fiber (e.g., asbestos fibers). In some embodiments, the cellular component is an organelle (e.g., mitochondria, peroxisome, plastid, endoplasmic reticulum, flagellum, or Golgi apparatus). In some embodiments, the cellular component is a cellular compartment. In some embodiments, the cellular component is a microorganism (e.g., bacterium, virus, or fungus). In some embodiments, the cellular component is a virus. In some embodiments, the cellular component is a vesicle (e.g., lysosome, peroxisome). In some embodiments, the cellular component is a small molecule. In some embodiments, the cellular component is a protein complex. In some embodiments, the cellular component is a protein aggregate. In some embodiments, the cellular component is a macromolecule. In some embodiments, the cellular component is a biomolecule. In some embodiments, the cellular component is a protein aggregate, soluble protein, midbody ring, damaged mitochodria, peroxisomes, intracellular bacteria, phagocytic membrane remnants, or viral capsid proteins. In some embodiments, the cellular component is a misfolded protein.

In some embodiments, the monovalent cellular component binder is a substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In some embodiments, the monovalent cellular component binder is a substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In some embodiments, the monovalent cellular component binder is a substituted or unsubstituted alkyl (e.g., C₁-C₈ alkyl, C₁-C₆ alkyl, or C₁-C₄ alkyl). In some embodiments, the monovalent cellular component binder is a substituted alkyl (e.g., C₁-C₈ alkyl, C₁-C₆ alkyl, or C₁-C₄ alkyl). In some embodiments, the monovalent cellular component binder is an unsubstituted alkyl (e.g., C₁-C₈ alkyl, C₁-C₆ alkyl, or C₁-C₄ alkyl). In some embodiments, the monovalent cellular component binder is a substituted or unsubstituted heteroalkyl (e.g., 2 to 8 membered heteroalkyl, 2 to 6 membered heteroalkyl, or 2 to 4 membered heteroalkyl). In some embodiments, the monovalent cellular component binder is a substituted heteroalkyl (e.g., 2 to 8 membered heteroalkyl, 2 to 6 membered heteroalkyl, or 2 to 4 membered heteroalkyl). In some embodiments, the monovalent cellular component binder is an unsubstituted heteroalkyl (e.g., 2 to 8 membered heteroalkyl, 2 to 6 membered heteroalkyl, or 2 to 4 membered heteroalkyl). In some embodiments, the monovalent cellular component binder is a substituted or unsubstituted cycloalkyl (e.g., C₃-C₈ cycloalkyl, C₃-C₆ cycloalkyl, or C₅-C₆ cycloalkyl). In some embodiments, the monovalent cellular component binder is a substituted cycloalkyl (e.g., C₃-C₈ cycloalkyl, C₃-C₆ cycloalkyl, or C₅-C₆ cycloalkyl). In some embodiments, the monovalent cellular component binder is an unsubstituted cycloalkyl (e.g., C₃-C₈ cycloalkyl, C₃-C₆ cycloalkyl, or C₅-C₆ cycloalkyl). In some embodiments, the monovalent cellular component binder is a substituted or unsubstituted heterocycloalkyl (e.g., 3 to 8 membered heterocycloalkyl, 3 to 6 membered heterocycloalkyl, or 5 to 6 membered heterocycloalkyl). In some embodiments, the monovalent cellular component binder is a substituted heterocycloalkyl (e.g., 3 to 8 membered heterocycloalkyl, 3 to 6 membered heterocycloalkyl, or 5 to 6 membered heterocycloalkyl). In some embodiments, the monovalent cellular component binder is an unsubstituted heterocycloalkyl (e.g., 3 to 8 membered heterocycloalkyl, 3 to 6 membered heterocycloalkyl, or 5 to 6 membered heterocycloalkyl). In some embodiments, the monovalent cellular component binder is a substituted or unsubstituted aryl (e.g., C₆-C₁₀ aryl, C₁₀ aryl, or phenyl). In some embodiments, the monovalent cellular component binder is a substituted aryl (e.g., C₆-C₁₀ aryl, C₁₀ aryl, or phenyl). In some embodiments, the monovalent cellular component binder is an unsubstituted aryl (e.g., C₆-C₁₀ aryl, C₁₀ aryl, or phenyl). In some embodiments, the monovalent cellular component binder is a substituted or unsubstituted heteroaryl (e.g., 5 to 10 membered heteroaryl, 5 to 9 membered heteroaryl, or 5 to 6 membered heteroaryl). In some embodiments, the monovalent cellular component binder is a substituted heteroaryl (e.g., 5 to 10 membered heteroaryl, 5 to 9 membered heteroaryl, or 5 to 6 membered heteroaryl). In some embodiments, the monovalent cellular component binder is an unsubstituted heteroaryl (e.g., 5 to 10 membered heteroaryl, 5 to 9 membered heteroaryl, or 5 to 6 membered heteroaryl).

In some embodiments, the monovalent cellular component binder is a R⁴⁹-substituted or unsubstituted alkyl (e.g., C₁-C₈ alkyl, C₁-C₆ alkyl, or C₁-C₄ alkyl), R⁴⁹-substituted or unsubstituted heteroalkyl (e.g., 2 to 8 membered heteroalkyl, 2 to 6 membered heteroalkyl, or 2 to 4 membered heteroalkyl), R⁴⁹-substituted or unsubstituted cycloalkyl (e.g., C₃-C₈ cycloalkyl, C₃-C₆ cycloalkyl, or C₅-C₆ cycloalkyl), R⁴⁹-substituted or unsubstituted heterocycloalkyl (e.g., 3 to 8 membered heterocycloalkyl, 3 to 6 membered heterocycloalkyl, or 5 to 6 membered heterocycloalkyl), R⁴⁹-substituted or unsubstituted aryl (e.g., C₆-C₁₀ aryl, C₁₀ aryl, or phenyl), or R⁴⁹-substituted or unsubstituted heteroaryl (e.g., 5 to 10 membered heteroaryl, 5 to 9 membered heteroaryl, or 5 to 6 membered heteroaryl). In some embodiments, the monovalent cellular component binder is an unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, the monovalent cellular component binder is a R⁴⁹-substituted or unsubstituted alkyl (e.g., C₁-C₈ alkyl, C₁-C₆ alkyl, or C₁-C₄ alkyl). In some embodiments, the monovalent cellular component binder is a R⁴⁹-substituted alkyl (e.g., C₁-C₈ alkyl, C₁-C₆ alkyl, or C₁-C₄ alkyl). In some embodiments, the monovalent cellular component binder is an unsubstituted alkyl (e.g., C₁-C₈ alkyl, C₁-C₆ alkyl, or C₁-C₄ alkyl). In some embodiments, the monovalent cellular component binder is a R⁴⁹-substituted or unsubstituted heteroalkyl (e.g., 2 to 8 membered heteroalkyl, 2 to 6 membered heteroalkyl, or 2 to 4 membered heteroalkyl). In some embodiments, the monovalent cellular component binder is a R⁴⁹-substituted heteroalkyl (e.g., 2 to 8 membered heteroalkyl, 2 to 6 membered heteroalkyl, or 2 to 4 membered heteroalkyl). In some embodiments, the monovalent cellular component binder is an unsubstituted heteroalkyl (e.g., 2 to 8 membered heteroalkyl, 2 to 6 membered heteroalkyl, or 2 to 4 membered heteroalkyl). In some embodiments, the monovalent cellular component binder is a R⁴⁹-substituted or unsubstituted cycloalkyl (e.g., C₃-C₈ cycloalkyl, C₃-C₆ cycloalkyl, or C₅-C₆ cycloalkyl). In some embodiments, the monovalent cellular component binder is a R⁴⁹-substituted cycloalkyl (e.g., C₃-C₈ cycloalkyl, C₃-C₆ cycloalkyl, or C₅-C₆ cycloalkyl). In some embodiments, the monovalent cellular component binder is an unsubstituted cycloalkyl (e.g., C₃-C₈ cycloalkyl, C₃-C₆ cycloalkyl, or C₅-C₆ cycloalkyl). In some embodiments, the monovalent cellular component binder is a R⁴⁹-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, the monovalent cellular component binder is a R⁴⁹-substituted heterocycloalkyl (e.g., 3 to 8 membered heterocycloalkyl, 3 to 6 membered heterocycloalkyl, or 5 to 6 membered heterocycloalkyl). In some embodiments, the monovalent cellular component binder is an unsubstituted heterocycloalkyl (e.g., 3 to 8 membered heterocycloalkyl, 3 to 6 membered heterocycloalkyl, or 5 to 6 membered heterocycloalkyl). In some embodiments, the monovalent cellular component binder is a R⁴⁹-substituted or unsubstituted aryl (e.g., C₆-C₁₀ aryl, C₁₀ aryl, or phenyl). In some embodiments, the monovalent cellular component binder is a R⁴⁹-substituted aryl (e.g., C₆-C₁₀ aryl, C₁₀ aryl, or phenyl). In some embodiments, the monovalent cellular component binder is an unsubstituted aryl (e.g., C₆-C₁₀ aryl, C₁₀ aryl, or phenyl). In some embodiments, the monovalent cellular component binder is a R⁴⁹-substituted or unsubstituted heteroaryl (e.g., 5 to 10 membered heteroaryl, 5 to 9 membered heteroaryl, or 5 to 6 membered heteroaryl). In some embodiments, the monovalent cellular component binder is a R⁴⁹-substituted heteroaryl (e.g., 5 to 10 membered heteroaryl, 5 to 9 membered heteroaryl, or 5 to 6 membered heteroaryl). In some embodiments, the monovalent cellular component binder is an unsubstituted heteroaryl (e.g., 5 to 10 membered heteroaryl, 5 to 9 membered heteroaryl, or 5 to 6 membered heteroaryl).

In some embodiments, R⁴⁹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R⁵⁰-substituted or unsubstituted alkyl (e.g., C₁-C₈ alkyl, C₁-C₆ alkyl, or C₁-C₄ alkyl), R⁵⁰-substituted or unsubstituted heteroalkyl (e.g., 2 to 8 membered heteroalkyl, 2 to 6 membered heteroalkyl, or 2 to 4 membered heteroalkyl), R⁵⁰-substituted or unsubstituted cycloalkyl (e.g., C₃-C₈ cycloalkyl, C₃-C₆ cycloalkyl, or C₅-C₆ cycloalkyl), R⁵⁰-substituted or unsubstituted heterocycloalkyl (e.g., 3 to 8 membered heterocycloalkyl, 3 to 6 membered heterocycloalkyl, or 5 to 6 membered heterocycloalkyl), R⁵⁰-substituted or unsubstituted aryl (e.g., C₆-C₁₀ aryl, C₁₀ aryl, or phenyl), or R⁵⁰-substituted or unsubstituted heteroaryl (e.g., 5 to 10 membered heteroaryl, 5 to 9 membered heteroaryl, or 5 to 6 membered heteroaryl). In some embodiments, R⁴⁹ is independently oxo, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R⁴⁹ is independently oxo. In some embodiments, R⁴⁹ is independently halogen. In some embodiments, R⁴⁹ is independently —CCl₃. In some embodiments, R⁴⁹ is independently —CBr₃. In some embodiments, R⁴⁹ is independently —CF₃. In some embodiments, R⁴⁹ is independently —CI₃. In some embodiments, R⁴⁹ is independently —CHCl₂. In some embodiments, R⁴⁹ is independently —CHBr₂. In some embodiments, R⁴⁹ is independently —CHF₂. In some embodiments, R⁴⁹ is independently —CHI₂. In some embodiments, R⁴⁹ is independently —CH₂Cl. In some embodiments, R⁴⁹ is independently —CH₂Br. In some embodiments, R⁴⁹ is independently —CH₂F. In some embodiments, R⁴⁹ is independently —CH₂I. In some embodiments, R⁴⁹ is independently —CN. In some embodiments, R⁴⁹ is independently —OH. In some embodiments, R⁴⁹ is independently —NH₂. In some embodiments, R⁴⁹ is independently —COOH. In some embodiments, R⁴⁹ is independently —CONH₂. In some embodiments, R⁴⁹ is independently —NO₂. In some embodiments, R⁴⁹ is independently —SH. In some embodiments, R⁴⁹ is independently —SO₃H. In some embodiments, R⁴⁹ is independently —SO₄H. In some embodiments, R⁴⁹ is independently —SO₂NH₂. In some embodiments, R⁴⁹ is independently —NHNH₂. In some embodiments, R⁴⁹ is independently —ONH₂. In some embodiments, R⁴⁹ is independently —NHC(O)NHNH₂. In some embodiments, R⁴⁹ is independently —NHC(O)NH₂. In some embodiments, R⁴⁹ is independently —NHSO₂H. In some embodiments, R⁴⁹ is independently —NHC(O)H. In some embodiments, R⁴⁹ is independently —NHC(O)OH. In some embodiments, R⁴⁹ is independently —NHOH. In some embodiments, R⁴⁹ is independently —OCCl₃. In some embodiments, R⁴⁹ is independently —OCF₃. In some embodiments, R⁴⁹ is independently —OCBr₃. In some embodiments, R⁴⁹ is independently —OCI₃. In some embodiments, R⁴⁹ is independently —OCHCl₂. In some embodiments, R⁴⁹ is independently —OCHBr₂. In some embodiments, R⁴⁹ is independently —OCHI₂. In some embodiments, R⁴⁹ is independently —OCHF₂. In some embodiments, R⁴⁹ is independently —OCH₂Cl. In some embodiments, R⁴⁹ is independently —OCH₂Br. In some embodiments, R⁴⁹ is independently —OCH₂I. In some embodiments, R⁴⁹ is independently —OCH₂F. In some embodiments, R⁴⁹ is independently —N₃. In some embodiments, R⁴⁹ is independently —OCH₃. In some embodiments, R⁴⁹ is independently —CH₃. In some embodiments, R⁴⁹ is independently —CH₂CH₃. In some embodiments, R⁴⁹ is independently unsubstituted propyl. In some embodiments, R⁴⁹ is independently unsubstituted isopropyl. In some embodiments, R⁴⁹ is independently unsubstituted butyl. In some embodiments, R⁴⁹ is independently unsubstituted tert-butyl. In some embodiments, R⁴⁹ is independently —F. In some embodiments, R⁴⁹ is independently —Cl. In some embodiments, R⁴⁹ is independently —Br. In some embodiments, R⁴⁹ is independently —I.

In some embodiments, R⁴⁹ is independently R⁵⁰-substituted or unsubstituted alkyl (e.g., C₁-C₈ alkyl, C₁-C₆ alkyl, or C₁-C₄ alkyl). In some embodiments, R⁴⁹ is independently R⁵⁰-substituted alkyl (e.g., C₁-C₈ alkyl, C₁-C₆ alkyl, or C₁-C₄ alkyl). In some embodiments, R⁴⁹ is independently an unsubstituted alkyl (e.g., C₁-C₈ alkyl, C₁-C₆ alkyl, or C₁-C₄ alkyl). In some embodiments, R⁴⁹ is independently R⁵⁰-substituted or unsubstituted heteroalkyl (e.g., 2 to 8 membered heteroalkyl, 2 to 6 membered heteroalkyl, or 2 to 4 membered heteroalkyl). In some embodiments, R⁴⁹ is independently R⁵⁰-substituted heteroalkyl (e.g., 2 to 8 membered heteroalkyl, 2 to 6 membered heteroalkyl, or 2 to 4 membered heteroalkyl). In some embodiments, R⁴⁹ is independently an unsubstituted heteroalkyl (e.g., 2 to 8 membered heteroalkyl, 2 to 6 membered heteroalkyl, or 2 to 4 membered heteroalkyl). In some embodiments, R⁴⁹ is independently R⁵⁰-substituted or unsubstituted cycloalkyl (e.g., C₃-C₈ cycloalkyl, C₃-C₆ cycloalkyl, or C₅-C₆ cycloalkyl). In some embodiments, R⁴⁹ is independently R⁵⁰-substituted cycloalkyl (e.g., C₃-C₈ cycloalkyl, C₃-C₆ cycloalkyl, or C₅-C₆ cycloalkyl). In some embodiments, R⁴⁹ is independently an unsubstituted cycloalkyl (e.g., C₃-C₈ cycloalkyl, C₃-C₆ cycloalkyl, or C₅-C₆ cycloalkyl). In some embodiments, R⁴⁹ is independently R⁵⁰-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R⁴⁹ is independently R⁵⁰-substituted heterocycloalkyl (e.g., 3 to 8 membered heterocycloalkyl, 3 to 6 membered heterocycloalkyl, or 5 to 6 membered heterocycloalkyl). In some embodiments, R⁴⁹ is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R⁴⁹ is independently R⁰-substituted or unsubstituted aryl (e.g., C₆-C₁₀ aryl, C₁₀ aryl, or phenyl). In some embodiments, R⁴⁹ is independently R⁵⁰-substituted aryl (e.g., C₆-C₁₀ aryl, C₁₀ aryl, or phenyl). In some embodiments, R⁴⁹ is independently an unsubstituted aryl (e.g., C₆-C₁₀ aryl, C₁₀ aryl, or phenyl). In some embodiments, R⁴⁹ is independently R⁵⁰-substituted or unsubstituted heteroaryl (e.g., 5 to 10 membered heteroaryl, 5 to 9 membered heteroaryl, or 5 to 6 membered heteroaryl). In some embodiments, R⁴⁹ is independently R⁵⁰-substituted heteroaryl (e.g., 5 to 10 membered heteroaryl, 5 to 9 membered heteroaryl, or 5 to 6 membered heteroaryl). In some embodiments, R⁴⁹ is independently an unsubstituted heteroaryl (e.g., 5 to 10 membered heteroaryl, 5 to 9 membered heteroaryl, or 5 to 6 membered heteroaryl).

In some embodiments, R⁵⁰ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R⁵¹-substituted or unsubstituted alkyl (e.g., C₁-C₈ alkyl, C₁-C₆ alkyl, or C₁-C₄ alkyl), R⁵¹-substituted or unsubstituted heteroalkyl (e.g., 2 to 8 membered heteroalkyl, 2 to 6 membered heteroalkyl, or 2 to 4 membered heteroalkyl), R⁵¹-substituted or unsubstituted cycloalkyl (e.g., C₃-C₈ cycloalkyl, C₃-C₆ cycloalkyl, or C₅-C₆ cycloalkyl), R⁵¹-substituted or unsubstituted heterocycloalkyl (e.g., 3 to 8 membered heterocycloalkyl, 3 to 6 membered heterocycloalkyl, or 5 to 6 membered heterocycloalkyl), R⁵¹-substituted or unsubstituted aryl (e.g., C₆-C₁₀ aryl, C₁₀ aryl, or phenyl), or R⁵¹-substituted or unsubstituted heteroaryl (e.g., 5 to 10 membered heteroaryl, 5 to 9 membered heteroaryl, or 5 to 6 membered heteroaryl). In some embodiments, R⁵⁰ is independently oxo, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R⁵⁰ is independently R⁵¹-substituted or unsubstituted alkyl (e.g., C₁-C₈ alkyl, C₁-C₆ alkyl, or C₁-C₄ alkyl). In some embodiments, R⁵⁰ is independently R⁵¹-substituted alkyl (e.g., C₁-C₈ alkyl, C₁-C₆ alkyl, or C₁-C₄ alkyl). In some embodiments, R⁵⁰ is independently an unsubstituted alkyl (e.g., C₁-C₈ alkyl, C₁-C₆ alkyl, or C₁-C₄ alkyl). In some embodiments, R⁵⁰ is independently R⁵¹-substituted or unsubstituted heteroalkyl (e.g., 2 to 8 membered heteroalkyl, 2 to 6 membered heteroalkyl, or 2 to 4 membered heteroalkyl). In some embodiments, R⁵⁰ is independently R⁵¹-substituted heteroalkyl (e.g., 2 to 8 membered heteroalkyl, 2 to 6 membered heteroalkyl, or 2 to 4 membered heteroalkyl). In some embodiments, R⁵⁰ is independently an unsubstituted heteroalkyl (e.g., 2 to 8 membered heteroalkyl, 2 to 6 membered heteroalkyl, or 2 to 4 membered heteroalkyl). In some embodiments, R⁵⁰ is independently R⁵¹-substituted or unsubstituted cycloalkyl (e.g., C₃-C₈ cycloalkyl, C₃-C₆ cycloalkyl, or C₅-C₆ cycloalkyl). In some embodiments, R⁵⁰ is independently R⁵¹-substituted cycloalkyl (e.g., C₃-C₈ cycloalkyl, C₃-C₆ cycloalkyl, or C₅-C₆ cycloalkyl). In some embodiments, R⁵⁰ is independently an unsubstituted cycloalkyl (e.g., C₃-C₈ cycloalkyl, C₃-C₆ cycloalkyl, or C₅-C₆ cycloalkyl). In some embodiments, R⁵⁰ is independently R⁵¹-substituted or unsubstituted heterocycloalkyl (e.g., 3 to 8 membered heterocycloalkyl, 3 to 6 membered heterocycloalkyl, or 5 to 6 membered heterocycloalkyl). In some embodiments, R⁵⁰ is independently R⁵¹-substituted heterocycloalkyl (e.g., 3 to 8 membered heterocycloalkyl, 3 to 6 membered heterocycloalkyl, or 5 to 6 membered heterocycloalkyl). In some embodiments, R⁵⁰ is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R⁵⁰ is independently R⁵¹-substituted or unsubstituted aryl (e.g., C₆-C₁₀ aryl, C₁₀ aryl, or phenyl). In some embodiments, R⁵⁰ is independently R⁵¹-substituted aryl (e.g., C₆-C₁₀ aryl, C₁₀ aryl, or phenyl). In some embodiments, R⁵⁰ is independently an unsubstituted aryl (e.g., C₆-C₁₀ aryl, C₁₀ aryl, or phenyl). In some embodiments, R⁵⁰ is independently R⁵¹-substituted or unsubstituted heteroaryl (e.g., 5 to 10 membered heteroaryl, 5 to 9 membered heteroaryl, or 5 to 6 membered heteroaryl). In some embodiments, R⁵⁰ is independently R⁵¹-substituted heteroaryl (e.g., 5 to 10 membered heteroaryl, 5 to 9 membered heteroaryl, or 5 to 6 membered heteroaryl). In some embodiments, R⁵⁰ is independently an unsubstituted heteroaryl (e.g., 5 to 10 membered heteroaryl, 5 to 9 membered heteroaryl, or 5 to 6 membered heteroaryl).

In some embodiments, R⁵⁰ is independently oxo. In some embodiments, R⁵⁰ is independently halogen. In some embodiments, R⁵⁰ is independently —CCl₃. In some embodiments, R⁵⁰ is independently —CBr₃. In some embodiments, R⁵⁰ is independently —CF₃. In some embodiments, R⁵⁰ is independently —CI₃. In some embodiments, R⁵⁰ is independently —CHCl₂. In some embodiments, R⁵⁰ is independently —CHBr₂. In some embodiments, R⁵⁰ is independently —CHF₂. In some embodiments, R⁵⁰ is independently —CHI₂. In some embodiments, R⁵⁰ is independently —CH₂Cl. In some embodiments, R⁵⁰ is independently —CH₂Br. In some embodiments, R⁵⁰ is independently —CH₂F. In some embodiments, R⁵⁰ is independently —CH₂I. In some embodiments, R⁵⁰ is independently —CN. In some embodiments, R⁵⁰ is independently —OH. In some embodiments, R⁵⁰ is independently —NH₂. In some embodiments, R⁵⁰ is independently —COOH. In some embodiments, R⁵⁰ is independently —CONH₂. In some embodiments, R⁵⁰ is independently —NO₂. In some embodiments, R⁵⁰ is independently —SH. In some embodiments, R⁵⁰ is independently —SO₃H. In some embodiments, R⁵⁰ is independently —SO₄H. In some embodiments, R⁵⁰ is independently —SO₂NH₂. In some embodiments, R⁵⁰ is independently —NHNH₂. In some embodiments, R⁵⁰ is independently —ONH₂. In some embodiments, R⁵⁰ is independently —NHC(O)NHNH₂. In some embodiments, R⁵⁰ is independently —NHC(O)NH₂. In some embodiments, R⁵⁰ is independently —NHSO₂H. In some embodiments, R⁵⁰ is independently —NHC(O)H. In some embodiments, R⁵⁰ is independently —NHC(O)OH. In some embodiments, R⁵⁰ is independently —NHOH. In some embodiments, R⁵⁰ is independently —OCCl₃. In some embodiments, R⁵⁰ is independently —OCF₃. In some embodiments, R⁵⁰ is independently —OCBr₃. In some embodiments, R⁵⁰ is independently —OCI₃. In some embodiments, R⁵⁰ is independently —OCHCl₂. In some embodiments, R⁵⁰ is independently —OCHBr₂. In some embodiments, R⁵⁰ is independently —OCHI₂. In some embodiments, R⁵⁰ is independently —OCHF₂. In some embodiments, R⁵⁰ is independently —OCH₂Cl. In some embodiments, R⁵⁰ is independently —OCH₂Br. In some embodiments, R⁵⁰ is independently —OCH₂I. In some embodiments, R⁵⁰ is independently —OCH₂F. In some embodiments, R⁵⁰ is independently —N₃. In some embodiments, R⁵⁰ is independently —OCH₃. In some embodiments, R⁵⁰ is independently —CH₃. In some embodiments, R⁵⁰ is independently —CH₂CH₃. In some embodiments, R⁵⁰ is independently unsubstituted propyl.

In some embodiments, R⁵⁰ is independently unsubstituted isopropyl. In some embodiments, R⁵⁰ is independently unsubstituted butyl. In some embodiments, R⁵⁰ is independently unsubstituted tert-butyl. In some embodiments, R⁵⁰ is independently —F. In some embodiments, R⁵⁰ is independently —Cl. In some embodiments, R⁵⁰ is independently —Br. In some embodiments, R⁵⁰ is independently —I.

In some embodiments, R⁵¹ is independently oxo, halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In some embodiments, R⁵¹ is independently an unsubstituted alkyl (e.g., C₁-C₈ alkyl, C₁-C₆ alkyl, or C₁-C₄ alkyl). In some embodiments, R⁵¹ is independently an unsubstituted heteroalkyl (e.g., 2 to 8 membered heteroalkyl, 2 to 6 membered heteroalkyl, or 2 to 4 membered heteroalkyl). In some embodiments, R⁵¹ is independently an unsubstituted cycloalkyl (e.g., C₃-C₈ cycloalkyl, C₃-C₆ cycloalkyl, or C₅-C₆ cycloalkyl). In some embodiments, R⁵¹ is independently an unsubstituted heterocycloalkyl (e.g., 3 to 8 membered heterocycloalkyl, 3 to 6 membered heterocycloalkyl, or 5 to 6 membered heterocycloalkyl). In some embodiments, R⁵¹ is independently an unsubstituted aryl (e.g., C₆-C₁₀ aryl, C₁₀ aryl, or phenyl). In some embodiments, R⁵¹ is independently an unsubstituted heteroaryl (e.g., 5 to 10 membered heteroaryl, 5 to 9 membered heteroaryl, or 5 to 6 membered heteroaryl).

In some embodiments, R⁵¹ is independently oxo. In some embodiments, R⁵¹ is independently halogen. In some embodiments, R⁵¹ is independently —CCl₃. In some embodiments, R⁵¹ is independently —CBr₃. In some embodiments, R⁵¹ is independently —CF₃. In some embodiments, R⁵¹ is independently —CI₃. In some embodiments, R⁵¹ is independently —CHCl₂. In some embodiments, R⁵¹ is independently —CHBr₂. In some embodiments, R⁵¹ is independently —CHF₂. In some embodiments, R⁵¹ is independently —CHI₂. In some embodiments, R⁵¹ is independently —CH₂Cl. In some embodiments, R⁵¹ is independently —CH₂Br. In some embodiments, R⁵¹ is independently —CH₂F. In some embodiments, R⁵¹ is independently —CH₂I. In some embodiments, R⁵¹ is independently —CN. In some embodiments, R⁵¹ is independently —OH. In some embodiments, R⁵¹ is independently —NH₂. In some embodiments, R⁵¹ is independently —COOH. In some embodiments, R⁵¹ is independently —CONH₂. In some embodiments, R⁵¹ is independently —NO₂. In some embodiments, R⁵¹ is independently —SH. In some embodiments, R⁵¹ is independently —SO₃H. In some embodiments, R⁵¹ is independently —SO₄H. In some embodiments, R⁵¹ is independently —SO₂NH₂. In some embodiments, R⁵¹ is independently —NHNH₂. In some embodiments, R⁵¹ is independently —ONH₂. In some embodiments, R⁵¹ is independently —NHC(O)NHNH₂. In some embodiments, R⁵¹ is independently —NHC(O)NH₂. In some embodiments, R⁵¹ is independently —NHSO₂H. In some embodiments, R⁵¹ is independently —NHC(O)H. In some embodiments, R⁵¹ is independently —NHC(O)OH. In some embodiments, R⁵¹ is independently —NHOH. In some embodiments, R⁵¹ is independently —OCCl₃. In some embodiments, R⁵¹ is independently —OCF₃. In some embodiments, R⁵¹ is independently —OCBr₃. In some embodiments, R⁵¹ is independently —OCI₃. In some embodiments, R⁵¹ is independently —OCHCl₂. In some embodiments, R⁵¹ is independently —OCHBr₂. In some embodiments, R⁵¹ is independently —OCHI₂. In some embodiments, R⁵¹ is independently —OCHF₂. In some embodiments, R⁵¹ is independently —OCH₂Cl. In some embodiments, R⁵¹ is independently —OCH₂Br. In some embodiments, R⁵¹ is independently —OCH₂I. In some embodiments, R⁵¹ is independently —OCH₂F. In some embodiments, R⁵¹ is independently —N₃. In some embodiments, R⁵¹ is independently —OCH₃. In some embodiments, R⁵¹ is independently —CH₃. In some embodiments, R⁵¹ is independently —CH₂CH₃. In some embodiments, R⁵¹ is independently unsubstituted propyl. In some embodiments, R⁵¹ is independently unsubstituted isopropyl. In some embodiments, R⁵¹ is independently unsubstituted butyl. In some embodiments, R⁵¹ is independently unsubstituted tert-butyl. In some embodiments, R⁵¹ is independently —F. In some embodiments, R⁵¹ is independently —Cl. In some embodiments, R⁵¹ is independently —Br. In some embodiments, R⁵¹ is independently —I.

In some embodiments, the monovalent cellular component binder is capable of binding the protein BRD4. In some embodiments, the monovalent cellular component binder is capable of binding the protein thioflavin T. In some embodiments, the monovalent cellular component binder is capable of binding the protein amyloid beta plaques. In some embodiments, the monovalent cellular component binder is capable of binding Bromodomain-containing protein 4 (BRD4), KRAS, Myc proto-oncogene protein (MYC), yes-associated protein 1 (YAP), tafazzin (TAZ), Catenin beta-1 (CTNNB1), Amyloid precursor protein (APP), huntingtin protein (HTT), Alpha-synuclein (SNCA), Nuclear factor (erythroid-derived 2)-like 2 (NRF2), or microtubule-associated protein tau (MAPT). In some embodiments, the monovalent cellular component binder is capable of binding a protein aggregate (e.g., HTT, APP, SNCA, or MAPT). In some embodiments, the monovalent cellular component binder is capable of binding PTEN-induced putative kinase 1 (PINK1), Autophagy-related protein 32 (ATG32); Extended synaptotagmin-1 (ESYT1), Extended synaptotagmin-2 (ESYT2), Phosphatidylinositol 3-kinase catalytic subunit type 3 (PI3KC3), Ras-related protein Rab-10 (RAB10), or Adipose triglyceride lipase (ATGL). In some embodiments, the monovalent cellular component binder is capable of binding a microorganism. In some embodiments, the monovalent cellular component binder is capable of binding a virus. In some embodiments, the monovalent cellular component binder is capable of binding a lipid droplet. In some embodiments, the monovalent cellular component binder is capable of binding a bacterial cell-surface glycan or bacterial cell surface protein.

In some embodiments, the protein aggregate is Beta amyloid, Amyloid precursor protein, IAPP (Amylin), Alpha-synuclein, PrPSc, PrPSc, Huntingtin, Calcitonin, Atrial natriuretic factor, Apolipoprotein AI, Serum amyloid A, Medin, Prolactin, Transthyretin, Lysozyme, Beta-2 microglobulin, Gelsolin, Keratoepithelin, Beta amyloid, Cystatin, Immunoglobulin light chain AL, or S-IBM.

In some embodiments, the protein aggregate includes Beta amyloid, Amyloid precursor protein, IAPP (Amylin), Alpha-synuclein, PrPSc, PrPSc, Huntingtin, Calcitonin, Atrial natriuretic factor, Apolipoprotein AI, Serum amyloid A, Medin, Prolactin, Transthyretin, Lysozyme, Beta-2 microglobulin, Gelsolin, Keratoepithelin, Beta amyloid, Cystatin, Immunoglobulin light chain AL, or S-IBM.

In some embodiments, the protein aggregate includes Beta amyloid. In some embodiments, the protein aggregate includes Amyloid precursor protein. In some embodiments, the protein aggregate includes IAPP (Amylin). In some embodiments, the protein aggregate includes Alpha-synuclein. In some embodiments, the protein aggregate includes PrPSc. In some embodiments, the protein aggregate includes PrPSc. In some embodiments, the protein aggregate includes Huntingtin. In some embodiments, the protein aggregate includes Calcitonin. In some embodiments, the protein aggregate includes Atrial natriuretic factor. In some embodiments, the protein aggregate includes Apolipoprotein AI. In some embodiments, the protein aggregate includes Serum amyloid A. In some embodiments, the protein aggregate includes Medin. In some embodiments, the protein aggregate includes Prolactin. In some embodiments, the protein aggregate includes Transthyretin. In some embodiments, the protein aggregate includes Lysozyme. In some embodiments, the protein aggregate includes Beta-2 microglobulin. In some embodiments, the protein aggregate includes Gelsolin. In some embodiments, the protein aggregate includes Keratoepithelin. In some embodiments, the protein aggregate includes Beta amyloid. In some embodiments, the protein aggregate includes Cystatin. In some embodiments, the protein aggregate includes Immunoglobulin light chain AL. In some embodiments, the protein aggregate includes S-IBM.

In some embodiments, the protein aggregate is Beta amyloid. In some embodiments, the protein aggregate is Amyloid precursor protein. In some embodiments, the protein aggregate is IAPP (Amylin). In some embodiments, the protein aggregate is Alpha-synuclein. In some embodiments, the protein aggregate is PrPSc. In some embodiments, the protein aggregate is PrPSc. In some embodiments, the protein aggregate is Huntingtin. In some embodiments, the protein aggregate is Calcitonin. In some embodiments, the protein aggregate is Atrial natriuretic factor. In some embodiments, the protein aggregate is Apolipoprotein AI. In some embodiments, the protein aggregate is Serum amyloid A. In some embodiments, the protein aggregate is Medin. In some embodiments, the protein aggregate is Prolactin. In some embodiments, the protein aggregate is Transthyretin. In some embodiments, the protein aggregate is Lysozyme. In some embodiments, the protein aggregate is Beta-2 microglobulin. In some embodiments, the protein aggregate is Gelsolin. In some embodiments, the protein aggregate is Keratoepithelin. In some embodiments, the protein aggregate is Beta amyloid. In some embodiments, the protein aggregate is Cystatin. In some embodiments, the protein aggregate is Immunoglobulin light chain AL. In some embodiments, the protein aggregate is S-IBM.

In some embodiments, the protein aggregate is a huntingtin aggregate. In some embodiments, the protein aggregate is a polyQ huntingtin aggregate.

In some embodiments, the monovalent cellular component binder is capable of binding a protein aggregate. In some embodiments, the monovalent cellular component binder is capable of binding a huntingtin aggregate. In some embodiments, the monovalent cellular component binder is capable of binding a polyQ huntingtin aggregate.

In some embodiments, the monovalent cellular component binder is capable of binding BRD4. In some embodiments, the monovalent cellular component binder is capable of binding KRAS. In some embodiments, the monovalent cellular component binder is capable of binding MYC. In some embodiments, the monovalent cellular component binder is capable of binding YAP. In some embodiments, the monovalent cellular component binder is capable of binding TAZ. In some embodiments, the monovalent cellular component binder is capable of binding CTNNB1. In some embodiments, the monovalent cellular component binder is capable of binding APP. In some embodiments, the monovalent cellular component binder is capable of binding HTT. In some embodiments, the monovalent cellular component binder is capable of binding SNCA. In some embodiments, the monovalent cellular component binder is capable of binding NRF2. In some embodiments, the monovalent cellular component binder is capable of binding or MAPT.

In some embodiments, the monovalent cellular component binder is capable of binding HTT. In some embodiments, the monovalent cellular component binder is capable of binding APP. In some embodiments, the monovalent cellular component binder is capable of binding SNCA. In some embodiments, the monovalent cellular component binder is capable of binding MAPT. In some embodiments, the monovalent cellular component binder is capable of binding PINK1. In some embodiments, the monovalent cellular component binder is capable of binding ATG32. In some embodiments, the monovalent cellular component binder is capable of binding ESYT. In some embodiments, the monovalent cellular component binder is capable of binding PI3KC3. In some embodiments, the monovalent cellular component binder is capable of binding RAB10. In some embodiments, the monovalent cellular component binder is capable of binding or ATGL.

In some embodiments, the monovalent cellular component binder has the formula:

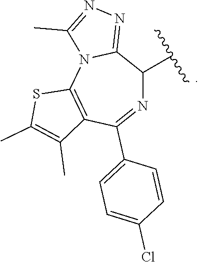 [SMILES: *C1N=C(c2ccc(Cl)cc2)c2c(sc(C)c2C)-n2c(C)nnc21]

In some embodiments, the monovalent cellular component binder is capable of binding a protein aggregate. In some embodiments, the monovalent cellular component binder has the formula:

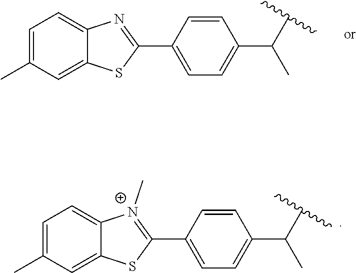

In some embodiments, the monovalent cellular component binder is capable of binding a protein aggregate. In some embodiments, the monovalent cellular component binder is a monovalent form of thioflavin or a derivative thereof. In some embodiments the monovalent cellular component binder has the formula:

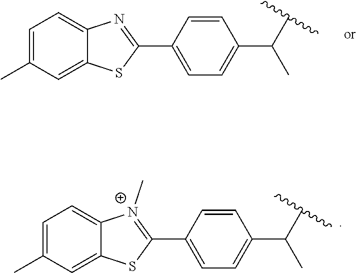

In some embodiments, the monovalent cellular component binder is capable of binding a protein aggregate. In some embodiments, the monovalent cellular component binder is a monovalent form of thioflavin or a derivative thereof. In some embodiments, the monovalent cellular component binder is a monovalent form of the formula:

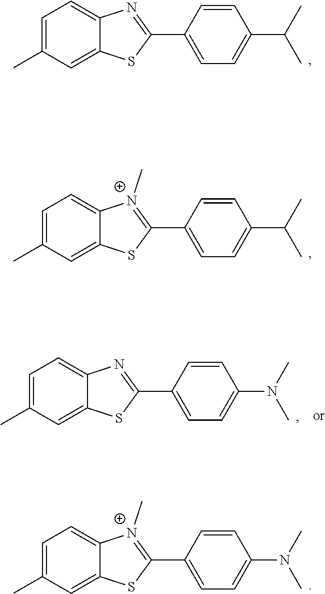

In some embodiments, the monovalent cellular component binder is capable of binding a protein aggregate. In some embodiments, the monovalent cellular component binder has the formula:

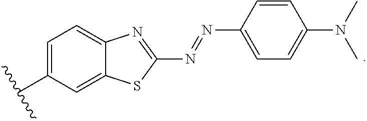 [SMILES: *c1ccc2nc(/N=N/c3ccc(N(C)C)cc3)sc2c1]

### Autophagy Adapter Protein

In another aspect, provided herein an autophagy adapter protein (e.g., p62) covalently bonded to a compound described herein. In some embodiments, the compound is covalently bonded to a cysteine residue of the protein. In some embodiments, the compound is irreversibly covalently bonded to a cysteine residue of the protein. In some embodiments, the compound is a targeted autophagy degrader (e.g., as described herein), for example a compound including a monovalent cellular component binder (e.g., as described herein) and a monovalent autophagy adapter protein binder (e.g., as described herein).

In some embodiments, the compound is covalently (e.g., irreversibly) bonded to an amino acid corresponding to C26 of human p62/SQSTM1 protein. In some embodiments, the compound is covalently (e.g., irreversibly) bonded to an amino acid corresponding to C27 of human p62/SQSTM1protein. In some embodiments, the compound is covalently (e.g., irreversibly) bonded to an amino acid corresponding to C113 of human p62/SQSTM1protein.

In another aspect, provided herein is an autophagy adapter protein (e.g., p62) covalently bonded to a fragment (e.g., moiety, moiety of a fragment) of a compound described herein.

In some embodiments, the compound fragment is covalently (e.g., irreversibly) bonded to an amino acid corresponding to C26 of human p62/SQSTM1 protein. In some embodiments, the compound fragment is covalently (e.g., irreversibly) bonded to an amino acid corresponding to C27 of human p62/SQSTM1protein. In some embodiments, the compound fragment is covalently (e.g., irreversibly) bonded to an amino acid corresponding to C113 of human p62/SQSTM1protein.

In some embodiments, the autophagy adapter protein covalently bonded to a autophagy adapter protein binder or compound described herein is the product of a reaction between the autophagy adapter protein and a autophagy adapter protein binder or compound described herein. It will be understood that the covalently bonded autophagy adapter protein and autophagy adapter protein binder (e.g., compound described herein) are the remnants of the reactant autophagy adapter protein and autophagy adapter protein binder or compound, wherein each reactant now participates in the covalent bond between the autophagy adapter protein and autophagy adapter protein binder or compound. In some embodiments, of the covalently bonded autophagy adapter protein and compound described herein, the remnant of the E substituent is a linker including a covalent bond between the autophagy adapter protein and the remainder of the compound described herein. It will be understood by a person of ordinary skill in the art that when a autophagy adapter protein is covalently bonded to a autophagy adapter protein binder (e.g., compound described herein), the autophagy adapter protein binder (e.g., compound described herein) forms a remnant of the pre-reacted autophagy adapter protein binder (e.g., compound described herein) wherein a bond connects the remnant of the autophagy adapter protein binder (e.g., compound described herein) to the remnant of the autophagy adapter protein (e.g., cysteine sulfur, sulfur of amino acid corresponding to C26 of human p62/SQSTM1 protein, sulfur of amino acid corresponding to C27 of human p62/SQSTM1protein, sulfur of C26 of human p62/SQSTM1 protein, sulfur of C27 of human p62/SQSTM1protein, sulfur of C113 of human p62/SQSTM1protein). The remnant of the autophagy adapter protein binder (e.g., a compound described herein) may also be called a portion of the autophagy adapter protein binder.

### III. Pharmaceutical Compositions

In a further aspect, provided herein is a pharmaceutical composition including a compound described herein (e.g., a targeted autophagy degrader) and a pharmaceutically acceptable excipient.

In some embodiments, the pharmaceutical composition includes an effective amount of the compound. In some embodiments, the pharmaceutical composition includes a therapeutically effective amount of the compound. In some embodiments, the pharmaceutical composition includes a second agent. In some embodiments, of the pharmaceutical compositions, the pharmaceutical composition includes a second agent in a therapeutically effective amount.

The pharmaceutical compositions may include optical isomers, diastereomers, or pharmaceutically acceptable salts of the modulators disclosed herein. The compound included in the pharmaceutical composition may be covalently attached to a carrier moiety. Alternatively, the compound included in the pharmaceutical composition is not covalently linked to a carrier moiety.

In another aspect, provided herein is a pharmaceutical composition including a targeted autophagy degrader (e.g., as described herein or a compound described herein) and a pharmaceutically acceptable excipient.

In some embodiments, the pharmaceutical composition includes an effective amount of the targeted autophagy degrader. In some embodiments, the pharmaceutical composition includes a therapeutically effective amount of the targeted autophagy degrader. In some embodiments, the pharmaceutical composition includes a second agent. In some embodiments, of the pharmaceutical compositions, the pharmaceutical composition includes a second agent in a therapeutically effective amount. In some embodiments, the second agent is an agent for treating cancer. In some embodiments, the second agent is an agent for treating a neurodegenerative disease (e.g., Huntington's Disease, Alzheimer Disease, or Parkinson's Disease). In some embodiments, the second agent is an agent for treating a disease associated with a protein aggregate. In some embodiments, the second agent is an agent for treating a metabolic disease. In some embodiments, the second agent is an agent for treating an autoimmune disease. In some embodiments, the second agent is an agent for treating an infectious disease. In some embodiments, the second agent is an agent for treating an inflammatory disease. In some embodiments, the second agent is an agent for treating Huntington's disease.

### IV. Methods for Treating Diseases

In a further aspect, provided herein is a method for treating a disease associated with a cellular component (e.g., aberrant level of a cellular component), the method including contacting the cellular component with a targeted autophagy degrader (e.g., as described herein). A targeted autophagy degrader includes any compound described herein wherein the monovalent targeted autophay protein binder is a monovalent form of formula (A), (B), (C), (I), (I-a), (II), (II-a), (II-b), (III), (III-a), (III-b), (III-c), (III-d), (III-e), (III-f), (III-g), (III-h), (III-i), (III-j) (IV), (IV-a), (IV-b), (IV-c), (IV-d), (IV-e), (IV-f), (IV-g), (IV-h), (IV-i), (V), (V-a), (V-b), (V-c), (V-d), (V-e), (V-f), (V-g), (V-h), (VI), (VI-a), (VI-b), (VI-c), (VI-d), (VI-g), (VI-h), (VII), or (VII-a), or any variation or embodiment thereof. In another aspect, provided herein is a method for treating a disease associated with a cellular component (e.g., aberrant level of a cellular component), the method including administering to a subject in need thereof a therapeutically effective amount of a compound described herein.

In another aspect, provided herein is a method for treating cancer, the method including contacting a cellular component associated with cancer with a targeted autophagy degrader (e.g., as described herein).

In another aspect, provided herein is a method for treating cancer, the method including administering to a subject in need thereof a therapeutically effective amount of a compound described herein. In another aspect, provided herein is a method for treating cancer, the method including administering to a subject in need thereof a therapeutically effective amount of a targeted autophagy degrader (e.g., as described herein).

In another aspect, provided herein is a method for treating neurodegenerative disease, the method including contacting a cellular component associated with the neurodegenerative disease with a targeted autophagy degrader (e.g., as described herein).

In another aspect, provided herein is a method for treating a neurodegenerative disease, the method including administering to a subject in need thereof a therapeutically effective amount of a compound described herein. In another aspect, provided herein is a method for treating a neurodegenerative disease, the method including administering to a subject in need thereof a therapeutically effective amount of a targeted autophagy degrader (e.g., as described herein). In some embodiments, the neurodegenerative disease is Huntington Disease, Alzheimer Disease, or Parkinson's Disease.

In another aspect, provided herein is a method for treating a metabolic disease, the method including contacting a cellular component associated with the metabolic disease with a targeted autophagy degrader (e.g., as described herein).

In another aspect, provided herein is a method for treating a metabolic disease, the method including administering to a subject in need thereof a therapeutically effective amount of a compound described herein. In another aspect, provided herein is a method for treating a metabolic disease, the method including administering to a subject in need thereof a therapeutically effective amount of a targeted autophagy degrader (e.g., as described herein).

In another aspect, provided herein is a method for treating an infectious disease, the method including contacting a cellular component associated with the infectious disease with a targeted autophagy degrader (e.g., as described herein).

In another aspect, provided herein is a method for treating an infectious disease, the method including administering to a subject in need thereof a therapeutically effective amount of a compound described herein. In another aspect, provided herein is a method for treating an infectious disease, the method including administering to a subject in need thereof a therapeutically effective amount of a targeted autophagy degrader (e.g., as described herein).

In another aspect, provided herein is a method for treating an autoimmune disease, the method including contacting a cellular component associated with the autoimmune disease with a targeted autophagy degrader (e.g., as described herein).

In another aspect, provided herein is a method for treating an autoimmune disease, the method including administering to a subject in need thereof a therapeutically effective amount of a compound described herein. In another aspect, provided herein is a method for treating an autoimmune disease, the method including administering to a subject in need thereof a therapeutically effective amount of a targeted autophagy degrader (e.g., as described herein).

In another aspect, provided herein is a method for treating an inflammatory disease, the method including contacting a cellular component associated with the inflammatory disease with a targeted autophagy degrader (e.g., as described herein).

In another aspect, provided herein is a method for treating an inflammatory disease, the method including administering to a subject in need thereof a therapeutically effective amount of a compound described herein. In another aspect, provided herein is a method for treating an inflammatory disease, the method including administering to a subject in need thereof a therapeutically effective amount of a targeted autophagy degrader (e.g., as described herein).

In some embodiments, the method includes modulating (e.g., inhibiting relative to a control) the level or activity of a protein (e.g., BRD4, KRAS, MYC, YAP, TAZ, CTNNB1, APP, HTT, SNCA, NRF2, or MAPT).

In some embodiments, the method includes modulating (e.g., inhibiting relative to a control) the level or activity of a protein aggregate (e.g., HTT, APP, SNCA, or MAPT).

In some embodiments, the method includes modulating (e.g., inhibiting relative to a control) the level or activity of a protein associated with an organelle (e.g., PINK1, ATG32, ESYT, PI3KC3, RAB10, or ATGL). In some embodiments, the method includes modulating (e.g., inhibiting relative to a control) the level or activity of a protein associated with the mitochondria (e.g., ATG32). In some embodiments, the method includes modulating (e.g., inhibiting relative to a control) the level or activity of a protein associated with the endoplasmic reticuluum (e.g., ESYT or PI3KC3). In some embodiments, the method includes modulating (e.g., inhibiting relative to a control) the level or activity of an organelle.

In some embodiments, the method includes modulating (e.g., inhibiting relative to a control) the level or activity of a mitochondria. In some embodiments, the method includes modulating (e.g., inhibiting relative to a control) the level or activity of an endoplasmic reticuluum.

The compounds described herein can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation or anti-cancer agents).

In another aspect, provided herein is a method for treating a disease associated with a protein aggregate, the method including administering to a subject in need thereof a therapeutically effective amount of a compound described herein. In another aspect, provided herein is a method for treating a disease associated with a protein aggregate, the method including administering to a subject in need thereof a therapeutically effective amount of a targeted autophagy degrader (e.g., as described herein). In some embodiments, the disease associated with a protein aggregate is a neurodegenerative disease (e.g., Huntington's Disease, Alzheimer Disease, or Parkinson's Disease). In some embodiments, the disease associated with a protein aggregate is Alzheimer's disease and the protein aggregate is an aggregate including beta amyloid. In some embodiments, the disease associated with a protein aggregate is diabetes mellitus type 2 and the protein aggregate is an aggregate including IAPP. In some embodiments, the disease associated with a protein aggregate is Parkinson's disease and the protein aggregate is an aggregate including alpha-synuclein. In some embodiments, the disease associated with a protein aggregate is transmissible spongiform encephalopathy and the protein aggregate is an aggregate including PrP (e.g., PrP(Sc)). In some embodiments, the disease associated with a protein aggregate is fatal familial insomnia and the protein aggregate is an aggregate including PrP (e.g., PrP(Sc)). In some embodiments, the disease associated with a protein aggregate is Huntington's disease and the protein aggregate is an aggregate including Huntingtin. In some embodiments, the disease associated with a protein aggregate is medullary carcinoma of the thyroid and the protein aggregate is an aggregate including calcitonin. In some embodiments, the disease associated with a protein aggregate is cardiac arrhythmia (e.g., isolated atrial amyloidosis) and the protein aggregate is an aggregate including atrial natriuretic factor. In some embodiments, the disease associated with a protein aggregate is atherosclerosis and the protein aggregate is an aggregate including apolipoprotein A1. In some embodiments, the disease associated with a protein aggregate is rheumatoid arthritis and the protein aggregate is an aggregate including serum amyloid A. In some embodiments, the disease associated with a protein aggregate is aortic medial amyloid and the protein aggregate is an aggregate including medin. In some embodiments, the disease associated with a protein aggregate is prolactinomas and the protein aggregate is an aggregate including prolactin. In some embodiments, the disease associated with a protein aggregate is familial amyloid polyneuropathy and the protein aggregate is an aggregate including transthyretin. In some embodiments, the disease associated with a protein aggregate is hereditary non-neuropathic systemic amyloidosis and the protein aggregate is an aggregate including lysozyme. In some embodiments, the disease associated with a protein aggregate is dialysis related amyloidosis and the protein aggregate is an aggregate including beta-2 microglobulin. In some embodiments, the disease associated with a protein aggregate is Finnish amyloidosis and the protein aggregate is an aggregate including gelsolin. In some embodiments, the disease associated with a protein aggregate is lattice corneal dystrophy and the protein aggregate is an aggregate including keratoepithelin. In some embodiments, the disease associated with a protein aggregate is cerebral amyloid angiopathy and the protein aggregate is an aggregate including beta amyloid. In some embodiments, the disease associated with a protein aggregate is cerebral amyloid angiopathy (Icelandic type) and the protein aggregate is an aggregate including cystatin. In some embodiments, the disease associated with a protein aggregate is systemic AL amyloidosis and the protein aggregate is an aggregate including immunoglobulin light chain AL. In some embodiments, the disease associated with a protein aggregate is sporadic inclusion body myositis and the protein aggregate is an aggregate including S-IBM. In some embodiments, the disease associated with a protein aggregate is a tauopathy and the protein aggregate is an aggregate including tau protein. In some embodiments, the tauopathy is primary age-related tauopathy, CTE, progressive supranuclear palsy, corticobasal degeneration, frontotemporal demential and parkinsonism linked to chromosome 17, Lytico-Bodig disease, ganglioglioma, gangliocytoma, meningioangiomatosis, postencephalitic parkinsonism, subacute sclerosing panencephalitis, lead encephalopathy, tuberous sclerosis, pantothenate kinase-associated neurodegeneration, lipofuscinosis, or Pick's disease. In some embodiments, the disease associated with a protein aggregate is amyloidosis. In some embodiments, the disease associated with a protein aggregate is a proteinopathy. In some embodiments, the disease associated with a protein aggregate is amyotrophic lateral sclerosis and the protein aggregate is an aggregate including superoxide dismutase, TDP043, FUS, C90RF72, and/or ubiquilin-2 (UBQLN2). In some embodiments, the disease associated with a protein aggregate is a trinucleotide repeat disorder. In some embodiments, the disease associated with a protein aggregate is a synucleinopathy. In some embodiments, the disease associated with a protein aggregate is prion disease and the protein aggregate is an aggregate including prion protein. In some embodiments, the method includes reducing the protein aggregate (e.g., reducing aggregate size, number of aggregates, or occurrence of aggregates).

### V. Methods of Modulating Activity

In a further aspect, provided herein is a method of reducing the level of a cellular component, the method including contacting the cellular component with a targeted autophagy degrader (e.g., as described herein).

In some embodiments, the targeted autophagy degrader (e.g., as described herein) includes a monovalent cellular component binder (e.g., as described herein) and a monovalent autophagy adapter protein binder (e.g., as described herein). In some embodiments, the monovalent cellular component binder and monovalent autophagy adapter protein binder are covalently bonded by a linker (e.g., as described herein).

In some embodiments, the cellular component is a protein. In some embodiments, the cellular component is an organelle. In some embodiments, the cellular component is a complex of a plurality of optionally different proteins. In some embodiments, the cellular component is a protein aggregate. In some embodiments, the cellular component is a macromolecule. In some embodiments, the cellular component is an ion, lipid, nucleic acid, nucleotide, amino acid, protein, particle, organelle, cellular compartment, microorganism, vesicle, or small molecule.

In some embodiments, the method includes modulating (e.g., inhibiting relative to a control) the level or activity of a protein associated with an organelle (e.g., PINK1ATG32, ESYT, PI3KC3, RAB10, or ATGL). In some embodiments, the method includes modulating (e.g., inhibiting relative to a control) the level or activity of a protein associated with the mitochondria (e.g., ATG32). In some embodiments, the method includes modulating (e.g., inhibiting relative to a control) the level or activity of a protein associated with the endoplasmic reticuluum (e.g., ESYT or PI3KC3). In some embodiments, the method includes modulating (e.g., inhibiting relative to a control) the level or activity of an organelle.

In some embodiments, the method includes modulating (e.g., inhibiting relative to a control) the level or activity of a mitochondria. In some embodiments, the method includes modulating (e.g., inhibiting relative to a control) the level or activity of an endoplasmic reticuluum.

In another aspect, provided herein is a method of reducing the level of a cellular component, the method including contacting the cellular component with a targeted autophagy degrader, wherein the targeted autophagy degrader is a compound described herein.

In some embodiments, the method further including the steps: A) allowing formation of an autophagosome including the cellular component-targeted autophagy degrader-autophagy adapter protein complex; B) allowing the autophagosome to acidify; and C) allowing degradation of the cellular component.

### VI. Process

In a further aspect, provided herein is a method of reducing the level of a cellular component, the method comprising contacting a cellular component with a targeted autophagy degrader; wherein the targeted autophagy degrader comprises: i) a monovalent autophagy associated protein binder; ii) a monovalent cellular component binder; and iii) a covalent linker directly bonded to the monovalent autophagy associated protein binder and the monovalent cellular component binder.

In some embodiments, the autophagy associated protein is an autophagy adapter protein. In some embodiments, the cellular component is a protein, ion, lipid, nucleic acid, nucleotide, amino acid, particle, organelle, cellular compartment, microorganism, virus, vesicle, small molecule, protein complex, protein aggregate, or macromolecule.

In some embodiments, prior to the contacting, the targeted autophagy degrader is synthesized by reacting a cellular component binder, a linker, and an autophagy associated protein binder to produce the targeted autophagy degrader (e.g., a compound or composition described herein).

In some embodiments, prior to the contacting, the targeted autophagy degrader is synthesized by covalently reacting a cellular component binder, a linker, and an autophagy associated protein binder to produce the targeted autophagy degrader.

In some embodiments, prior to the synthesizing, the autophagy associated protein binder is identified. In some embodiments, prior to the synthesizing, the autophagy associated protein binder is selected and ranked according to a quantifiable property (e.g., binding ability, Lipinski's rule, or level of inhibition).

In some embodiments, the autophagy associated protein binder is identified by a method comprising the steps: i) mixing an autophagy associated protein with a library of candidate autophagy associated protein binders (e.g., in a reaction vessel); and ii) identifying the candidate autophagy associated protein binders that bind to the autophagy associated protein. In some embodiments, the candidate autophagy associated protein binders comprise a covalent cysteine modifier moiety and a candidate autophagy associated protein binder is identified as an autophagy associated protein binder by detection of covalent binding of the autophagy associated protein binder to the autophagy associated protein. In some embodiments, the detection of covalent binding of the candidate autophagy associated protein binder to the autophagy associated protein includes use of a detectable label or mass spectroscopic detection of the covalent binding. In some embodiments, prior to the synthesizing, the cellular component binder is identified.

In some embodiments, the detection of covalent binding of the candidate autophagy associated protein binder to the autophagy associated protein comprises competing candidate autophagy associated protein binders against reactivity-based probes (e.g., probes described herein) in the autophagy associated protein. In some embodiments, the detection includes comparing isotopically light to heavy ratios of probe-modified autophagy associated proteins.

In some embodiments, the cellular component binder is identified by a method comprising the steps: i) mixing a cellular component protein with a library of candidate cellular component binders; and ii) identifying the candidate cellular component binders that bind to the cellular component. In some embodiments, the candidate cellular component binders comprise a covalent cysteine modifier moiety and a candidate cellular component binder is identified as a cellular component binder by detection of covalent binding of the cellular component binder to the cellular component. In some embodiments, the detection of covalent binding of the candidate cellular component binder to the cellular component includes use of a detectable label or mass spectroscopic detection of the covalent binding.

In some embodiments, prior to synthesizing, the autophagy associated protein binder is modified to remove a covalent cysteine modifier moiety.

In some embodiments, the targeted autophagy degrader is a compound as described herein.

### EXAMPLES

It is understood that the present disclosure has been made only by way of example, and that numerous changes in the combination and arrangement of parts can be resorted to by those skilled in the art without departing from the spirit and scope of present disclosure.

### Synthetic Examples

The chemical reactions in the Examples described can be readily adapted to prepare a number of other compounds disclosed herein, and alternative methods for preparing the compounds of this disclosure are deemed to be within the scope of this disclosure. For example, the synthesis of non-exemplified compounds according to the present disclosure can be successfully performed by modifications apparent to those skilled in the art, e.g., by appropriately protecting interfering groups, by utilizing other suitable reagents known in the art other than those described, or by making routine modifications of reaction conditions, reagents, and starting materials. Alternatively, other reactions disclosed herein or known in the art will be recognized as having applicability for preparing other compounds of the present disclosure.

Abbreviations used in the Examples include the following:

ACN: acetonitrile
DCM: dichloromethane

### DMAP: 4-Dimethylaminopyridine

DMSO: dimethyl sulfoxide
¹H NMR: proton nuclear magnetic resonance
HPLC: high-performance liquid chromatography
TFA: trifluoroacetic acid

### Example S1: Synthesis of Compound 1

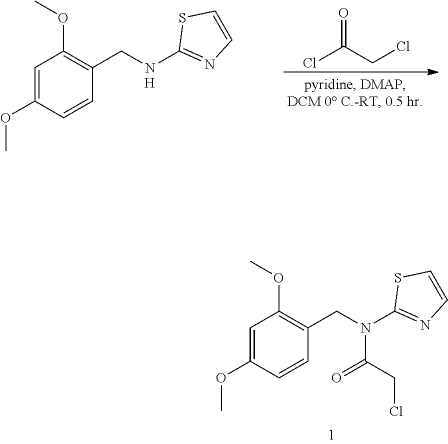

To a solution of N-(2,4-dimethoxybenzyl)thiazol-2-amine (70.0 mg, 279.7 umol, 1 eq.) in DCM (1.0 mL) was added pyridine (88.5 mg, 1.1 mmol, 4.0 eq.) and DMAP (3.4 mg, 28.0 umol, 0.1 eq.) followed by addition of 2-chloroacetyl chloride (47.4 mg, 419.5 umol, 1.5 eq.) at 0° C. The mixture was stirred at 25° C. for 0.5 hr and then concentrated. The residue was purified by prep-HPLC (column: Phenomenex Luna C18 100*30 mm*5 um; mobile phase: [water (0.1% TFA)-ACN]; B %: 40%-70%, 12 min) to give 2-chloro-N-(2,4-dimethoxybenzyl)-N-(thiazol-2-yl)acetamide (1) (2.1 mg, 6.1 umol, 2.2%) as a yellow solid. ¹H NMR (400 MHz, DMSO-d6) δ=7.48 (d, J=3.3 Hz, 1H), 7.33 (d, J=3.7 Hz, 1H), 6.77 (br d, J=8.4 Hz, 1H), 6.57 (d, J=2.0 Hz, 1H), 6.42 (dd, J=2.2, 8.4 Hz, 1H), 5.28 (br s, 2H), 4.70 (s, 2H), 3.80 (s, 3H), 3.70 (s, 3H). ESI [M+Na]=349.0.

### Example S2: Synthesis of Compound 2

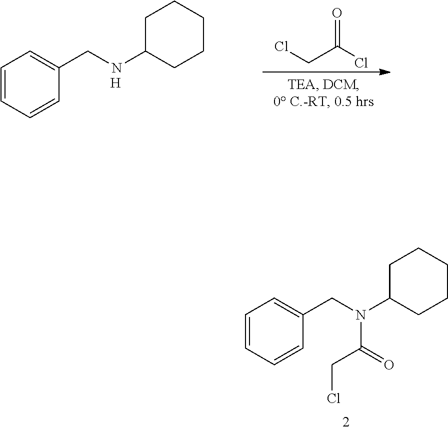

To a solution of N-benzylcyclohexanamine (90.0 mg, 475.4 umol, 1.0 eq.) and TEA (192.4 mg, 1.9 mmol, 4.0 eq.) in DCM (1.0 mL) was added 2-chloroacetyl chloride (80.6 mg, 713.2 umol, 1.5 eq.) at 0° C. The mixture was stirred at 25° C. for 1 hr and then concentrated. The residue was purified by prep-HPLC (column: Nano-micro Kromasil C18 80*25 mm 3 um; mobile phase: [water (0.10% TFA)-ACN]; B %: 44%-64%, 7 min) to give N-benzyl-2-chloro-N-cyclohexylacetamide (2) (35.7 mg, 120.2 umol, 25.3%) as light yellow oil. ¹H NMR (400 MHz, DMSO-d6) δ=7.46-7.12 (m, 5H), 4.67-4.49 (m, 3H), 4.18 (s, 1H), 3.83-3.45 (m, 1H), 1.76-1.49 (m, 5H), 1.47-1.15 (m, 4H), 1.10-0.89 (m, 1H). ESI [M+H]=266.0.

### Example S3: Synthesis of Compound 3

Compound 3 was prepared following a similar procedure as described for Compound 2.

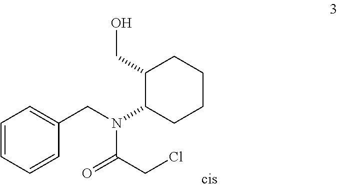

N-benzyl-2-chloro-N-(cis-2-(hydroxymethyl)cyclohexyl)acetamide (3) (12.8 mg, 40.9 umol, 10.5%) was obtained as light yellow oil after prep-HPLC purification. Purification condition: column: Nano-micro Kromasil C18 80*25 mm 3 um; mobile phase: [water (0.1% TFA)-ACN]; B %: 29%-59%, 7 min. ¹H NMR (400 MHz, DMSO-d6) δ ppm 7.55-7.42 (m, 1H), 7.41-7.32 (m, 1H), 7.26 (br d, J=6.39 Hz, 1H), 7.16 (br s, 2H), 4.99-4.64 (m, 1H), 4.58-4.46 (m, 1H), 4.44-4.18 (m, 3H), 4.16-3.97 (m, 1H), 3.90 (br d, J=13.89 Hz, 1H), 3.64 (br t, J=9.26 Hz, 1H), 2.25-2.00 (m, 1H), 1.97-1.55 (m, 3H), 1.52-1.17 (m, 5H). ESI [M+H]=296.1.

### Example S4: Synthesis of Compound 4

Compound 4 was prepared following a similar procedure as described for Compound 2.

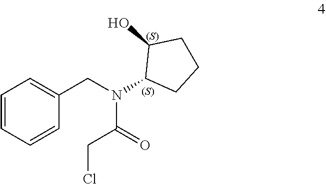

N-benzyl-2-chloro-N-((1S,2S)-2-hydroxycyclopentyl)acetamide (4) (49.0 mg, 181.1 umol, 49.5%) was obtained as light yellow oil after prep-HPLC purification. Purification condition: column: Phenomenex Luna C18 100*30 mm*5 um; mobile phase: [water (0.1% TFA)-ACN]; B %: 20%-50%, 12 min. ¹H NMR (400 MHz, DMSO-d6) δ=7.54-7.19 (m, 3H), 7.17 (br d, J=7.3 Hz, 2H), 4.83-4.48 (m, 2H), 4.40 (d, J=13.2 Hz, 1H), 4.28-4.04 (m, 2H), 4.02-3.86 (m, 2H), 1.88-1.63 (m, 2H), 1.61-1.33 (m, 4H). ESI [M+H]=268.1.

### Example S5: Synthesis of Compound 5

Compound 5 was prepared following a similar procedure as described for Compound 2.

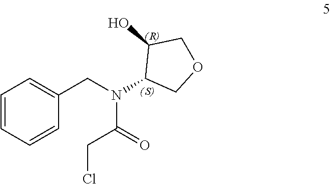

N-benzyl-2-chloro-N-((3S,4R)-4-hydroxytetrahydrofuran-3-yl)acetamide (5) (36.5 mg, 133.4 umol, 25.8%) was obtained as light yellow oil after prep-HPLC purification. Purification condition: column: Phenomenex Gemini-NX C18 75*30 mm*3 um; mobile phase: [water (0.1% TFA)-ACN]; B %: 20%-50%, 10 min. ¹H NMR (400 MHz, DMSO-d6) δ=7.49-7.05 (m, 5H), 4.84-4.48 (m, 3H), 4.44-4.17 (m, 3H), 3.96-3.77 (m, 2H), 3.56 (br dd, J=5.3, 9.5 Hz, 2H). ESI [M+H]=270.0.

### Example S6: Synthesis of Compound 6

Compound 6 was prepared following a similar procedure as described for Compound 2.

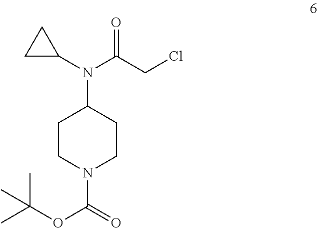 [SMILES: CC(C)(C)OC(=O)N1CCC(N(C(=O)CCl)C2CC2)CC1]

Tert-butyl 4-(2-chloro-N-cyclopropylacetamido)piperidine-1-carboxylate (6) (24.5 mg, 74.9 umol, 20.0%) was obtained as a yellow solid after prep-HPLC purification. Purification condition: column: Waters Xbridge BEH C18 100*30 mm*10 um; mobile phase: [water (10 mM NH₄HCO₃)-ACN]; B %: 30%-60%, 10 min. ¹H NMR (400 MHz, DMSO-d6) δ=4.54 (s, 2H), 4.08-3.81 (m, 3H), 2.68 (br s, 3H), 1.94-1.77 (m, 2H), 1.62 (br d, J=10.9 Hz, 2H), 1.41 (s, 9H), 0.92-0.78 (m, 4H). ESI [M−tBu+H]=261.1.

### Example S7: Synthesis of Compound 7

Compound 7 was prepared following a similar procedure as described for Compound 2.

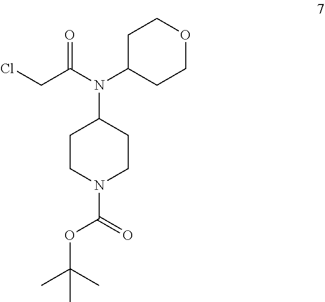 [SMILES: CC(C)(C)OC(=O)N1CCC(N(C(=O)CCl)C2CCOCC2)CC1]

Tert-butyl 4-(2-chloro-N-(tetrahydro-2H-pyran-4-yl)acetamido)piperidine-1-carboxylate (7) (20.0 mg, 55.4 umol, 22.5%) was obtained as a pale yellow solid after prep-HPLC purification. Purification condition: column: Waters Xbridge BEH C18 100*25 mm*5 um; mobile phase: [water (10 mM NH₄HCO₃)-ACN]; B %: 25%-55%, 8 min. ¹H NMR (400 MHz, DMSO-d6) δ=4.41 (s, 2H), 4.05-3.88 (m, 3H), 3.81 (br d, J=8.6 Hz, 2H), 3.37 (br s, 1H), 3.25 (br s, 1H), 2.77 (br s, 2H), 2.38 (br d, J=10.6 Hz, 1H), 1.82 (br d, J=7.2 Hz, 1H), 1.71-1.56 (m, 3H), 1.41 (s, 11H), 1.36-1.23 (m, 2H). ESI [M−tBu+H]=305.1.

### Example S8: Synthesis of Compound 8

Compound 8 was prepared following a similar procedure as described for Compound 2.

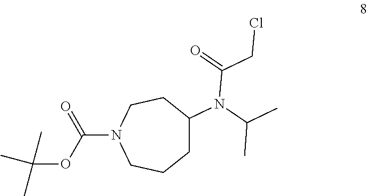 [SMILES: CC(C)N(C(=O)CCl)C1CCCN(C(=O)OC(C)(C)C)CC1]

Tert-butyl 4-(2-chloro-N-isopropylacetamido)azepane-1-carboxylate (8) (26.8 mg, 77.4 umol, 22.0%) was obtained as yellow oil after prep-HPLC purification. Purification condition: column: Waters Xbridge Prep OBD C18 150*40 mm*10 um; mobile phase: [water (10 mM NH₄HCO₃)-ACN]; B %: 35%-65%, 8 min. ¹H NMR (400 MHz, DMSO-d6) δ=4.27 (s, 2H), 4.04-3.91 (m, 1H), 3.54 (br d, J=10.5 Hz, 1H), 3.29-2.91 (m, 3H), 2.35 (br d, J=5.3 Hz, 1H), 1.98-1.61 (m, 3H), 1.50 (br s, 2H), 1.41 (s, 10H), 1.31 (br d, J=5.0 Hz, 2H), 1.14 (br d, J=4.4 Hz, 4H). ESI [M−tBu+H]=277.1.

### Example S9: Synthesis of Compound 9

Compound 9 was prepared following a similar procedure as described for Compound 2.

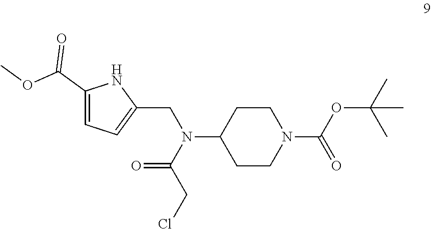 [SMILES: COC(=O)c1ccc(CN(C(=O)CCl)C2CCN(C(=O)OC(C)(C)C)CC2)[nH]1]

Tert-butyl 4-(2-chloro-N-((5-(methoxycarbonyl)-1H-pyrrol-2-yl)methyl)acetamido) piperidine-1-carboxylate (9) (30.0 mg, 66.7 umol, 22.5%) was obtained as a white solid after prep-HPLC purification. Purification condition: column: Phenomenex Luna C18 100*30 mm*5 um; mobile phase: [water (0.1% TFA)-ACN]; B %: 35%-65%, 12 min. ¹H NMR (400 MHz, DMSO-d6) δ=12.07-11.41 (m, 1H), 6.81-6.64 (m, 1H), 6.20-5.89 (m, 1H), 4.66-4.32 (m, 4H), 3.96 (br s, 3H), 3.75 (br d, J=4.5 Hz, 3H), 2.86-2.57 (m, 2H), 1.64-1.43 (m, 4H), 1.42-1.32 (m, 9H). ESI [M−Boc+H]=314.1.

### Example S10: Synthesis of Compound 2

Compound 10 was prepared following a similar procedure as described for Compound 2.

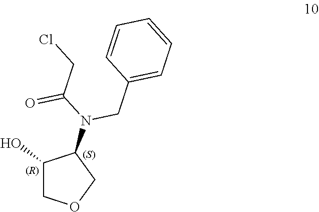

N-benzyl-2-chloro-N-[(3S,4R)-4-hydroxytetrahydrofuran-3-yl]acetamide (10) (44.6 mg, 165.2 umol, 53.2%) was obtained as light yellow oil after prep-HPLC purification. Purification condition: column: Phenomenex Luna C18 100*30 mm*5 um; mobile phase: [water (0.1% TFA)-ACN]; B %: 10%-45%, 12 min. ¹H NMR (400 MHz, DMSO-d6) δ=7.39-7.16 (m, 5H), 4.86-4.45 (m, 3H), 4.40-4.19 (m, 3H), 3.94-3.73 (m, 2H), 3.62-3.32 (m, 3H). ESI [M+H]=270.1.

### Example S11: Synthesis of Compound 11

Compound 11 was prepared following a similar procedure as described for Compound 2.

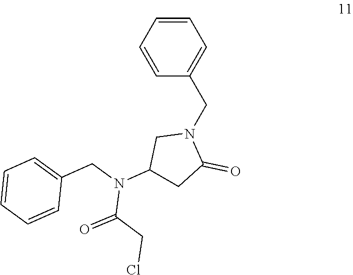 [SMILES: O=C1CC(N(Cc2ccccc2)C(=O)CCl)CN1Cc1ccccc1]

N-benzyl-N-(1-benzyl-5-oxo-pyrrolidin-3-yl)-2-chloro-acetamide (11) (15.5 mg, 43.3 umol, 20.2%) was got as light yellow oil after prep-HPLC purification. Purification condition: column: Phenomenex Luna C18 100*30 mm*5 um; mobile phase: [water (0.1% TFA)-ACN]; B %: 35%-55%, 12 min. ¹H NMR (400 MHz, DMSO-d6) δ=7.40-7.04 (m, 10H), 4.85 (br d, J=6.4 Hz, 0.5H), 4.66-4.18 (m, 7H), 3.50-3.49 (m, 0.5H), 3.32-3.25 (m, 0.5H), 3.24-3.23 (m, 0.5H), 2.64-2.61 (m, 1H), 2.53-2.43 (m, 1H). ESI [M+H]=357.1.

### Example S12: Synthesis of Compound 12

Compound 12 was prepared following a similar procedure as described for Compound 2.

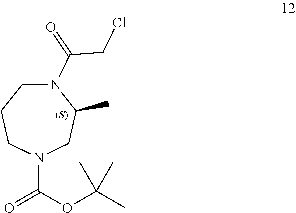

(S)-tert-butyl 4-(2-chloroacetyl)-3-methyl-1,4-diazepane-1-carboxylate (12) (45.0 mg, 143.9 umol, 30.8%) was obtained as light yellow oil after prep-HPLC purification. Purification condition: column: Waters Xbridge BEH C18 100*30 mm*10 um; mobile phase: [water (10 mM NH₄HCO₃)-ACN]; B %: 25%-55%, 10 min. ¹H NMR (400 MHz, DMSO-d6) δ=4.71-4.09 (m, 3H), 3.95-3.58 (m, 3H), 3.03-2.69 (m, 3H), 1.70-1.43 (m, 2H), 1.42-1.26 (m, 9H), 1.14-0.88 (m, 3H). ESI [M−tBu+H]=235.0.

### Example S13: Synthesis of Compound 13

Compound 13 was prepared following a similar procedure as described for Compound 2.

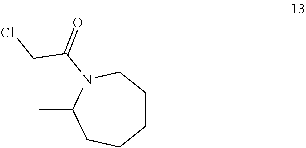

2-chloro-1-(2-methylazepan-1-yl)ethan-1-one (13) (1.1 mg, 5.9 umol, 1.5%) was obtained as light yellow oil after prep-HPLC purification. Purification condition: column: Phenomenex Luna C18 100*30 mm*5 um; mobile phase: [water (0.1% TFA)-ACN]; B %: 30%-45%, 12 min. ¹H NMR (400 MHz, DMSO-d6) δ=4.48-4.39 (m, 1H), 4.30-4.17 (m, 1.5H), 3.88-3.74 (m, 1H), 3.52 (br d, J=16.1 Hz, 0.5H), 3.17-3.00 (m, 0.5H), 2.79-2.63 (m, 0.5H), 2.08-1.87 (m, 1H), 1.82-1.54 (m, 3H), 1.46-0.91 (m, 7H). ESI [M+H]=190.1.

### Example S14: Synthesis of Compound 14

Compound 14 was prepared following a similar procedure as described for

Compound 2.

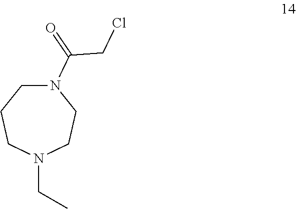

2-chloro-1-(4-ethyl-1,4-diazepan-1-yl)ethan-1-one (14) (22.6 mg, 61.7 umol, 3.7%) was obtained as colorless oil after prep-HPLC purification. Purification condition: column: Phenomenex Luna C18 150*30 mm*5 um; mobile phase: [water (0.1% TFA)-ACN]; B %: 1%-10%, 10 min. ¹H NMR (400 MHz, DMSO-d6) δ=4.43 (s, 2H), 4.06-3.95 (m, 1H), 3.91-3.65 (m, 1H), 3.63-3.51 (m, 4H), 3.30-2.93 (m, 4H), 2.24-1.99 (m, 2H), 1.22 (dt, J=3.2, 7.1 Hz, 3H). ESI [M+H]=205.1.

### Example S15: Synthesis of Compound 15

Compound 15 was prepared following a similar procedure as described for Compound 2.

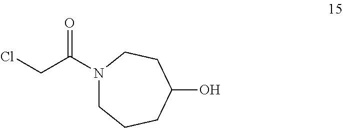 [SMILES: O=C(CCl)N1CCCC(O)CC1]

2-chloro-1-(4-hydroxyazepan-1-yl)ethan-1-one (15) (14.1 mg, 71.4 umol, 18.1%) was obtained as light yellow oil after prep-HPLC purification. Purification condition: (column: Waters Xbridge BEH C18 100*30 mm*10 um; mobile phase: [water (10 mM NH₄HCO₃)-ACN]; B %: 1%-20%, 10 min. ¹H NMR (400 MHz, DMSO-d₆) δ=4.58 (br s, 1H), 4.38-4.27 (m, 2H), 3.72-3.61 (m, 1H), 3.52-3.44 (m, 2H), 3.34-3.24 (m, 2H), 1.92-1.69 (m, 2H), 1.69-1.40 (m, 4H). ESI [M+H]=192.0.

### Example S16: Synthesis of Compound 16

Compound 16 was prepared following a similar procedure as described for Compound 2.

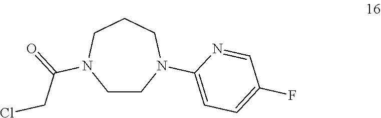 [SMILES: O=C(CCl)N1CCCN(c2ccc(F)cn2)CC1]

2-chloro-1-(4-(5-fluoropyridin-2-yl)-1,4-diazepan-1-yl)ethanone (16) (29.0 mg, 105.9 umol, 49.1%) was obtained as light yellow oil after prep-HPLC purification. Purification condition: column: Phenomenex Gemini-NX C18 75*30 mm*3 um; mobile phase: [water (10 mM NH₄HCO₃)-ACN]; B %: 20%-50%, 10 min. ¹H NMR (400 MHz, DMSO-d6) δ=8.04 (t, J=3.7 Hz, 1H), 7.50-7.41 (m, 1H), 6.72 (br s, 1H), 4.36 (s, 1H), 4.25 (s, 1H), 3.86-3.78 (m, 1H), 3.69-3.57 (m, 5H), 3.46-3.42 (m, 1H), 3.34 (br d, J=6.2 Hz, 1H), 1.92-1.72 (m, 2H). ESI [M+H]=272.1.

### Example S17: Synthesis of Compound 17

Compound 17 was prepared following a similar procedure as described for Compound 2.

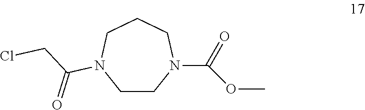

Methyl 4-(2-chloroacetyl)-1,4-diazepane-1-carboxylate (17) (11.7 mg, 50.1 umol, 13.9%) was obtained as colorless oil after prep-HPLC purification after prep-HPLC purification. Purification condition: (column: Phenomenex Luna C18 100*30 mm*5 um; mobile phase: [water (0.1% TFA)-ACN]; B %: 1%-30%, 12 min. ¹H NMR (400 MHz, DMSO-d6) δ=4.39-4.30 (m, 2H), 3.60-3.55 (m, 3H), 3.55-3.47 (m, 4H), 3.43 (br d, J=4.9 Hz, 3H), 3.35 (br s, 1H), 1.80-1.59 (m, 2H). ESI [M+H]=235.1.

### Example S18: Synthesis of Compound 18

Compound 18 was prepared following a similar procedure as described for Compound 2.

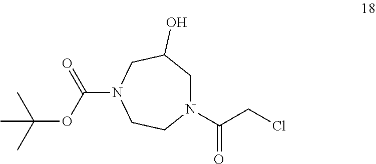 [SMILES: CC(C)(C)OC(=O)N1CCN(C(=O)CCl)CC(O)C1]

Tert-butyl 4-(2-chloroacetyl)-6-hydroxy-1,4-diazepane-1-carboxylate (18) (42.1 mg, 142.0 umol, 34.1%) was obtained as a yellow solid after prep-HPLC purification. Purification condition: column: Waters Xbridge BEH C18 100*30 mm*10 um; mobile phase: [water (10 mM NH₄HCO₃)-ACN]; B %: 15%-45%, 10 min. ¹H NMR (400 MHz, DMSO-d6) δ=5.33 (br s, 1H), 4.70-4.13 (m, 2H), 4.09-3.64 (m, 4H), 3.61-3.44 (m, 2H), 3.30-3.11 (m, 1H), 3.07-2.69 (m, 2H), 1.38 (br s, 9H). ESI [M−tBu+H]=237.0.

### Example S19: Synthesis of Compound 19

Compound 19 was prepared following a similar procedure as described for Compound 2.

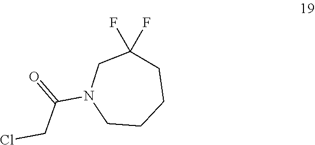 [SMILES: O=C(CCl)N1CCCCC(F)(F)C1]

2-chloro-1-(3,3-difluoroazepan-1-yl)ethanone (19) (1.3 mg, 6.5 umol, 1.6%) was obtained as light yellow oil after prep-HPLC purification. Purification condition: column: Phenomenex Luna C18 100*30 mm*5 um; mobile phase: [water (0.1% TFA)-ACN]; B %: 20%-45%, 12 min. ¹H NMR (400 MHz, DMSO-d6) δ=4.50-4.37 (m, 2H), 4.03-3.86 (m, 2H), 3.59-3.45 (m, 2H), 2.13-1.91 (m, 2H), 1.79-1.49 (m, 4H). ESI [M+H]=212.1.

### Example S20: Synthesis of Compound 20

Compound 20 was prepared following a similar procedure as described for Compound 2.

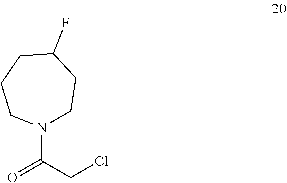 [SMILES: O=C(CCl)N1CCCC(F)CC1]

2-chloro-1-(4-fluoroazepan-1-yl)ethanone (20) (1.4 mg, 6.7 umol, 1.1%) was obtained as colorless oil after prep-HPLC purification. Purification condition: column: Phenomenex Luna C18 100*30 mm*5 um; mobile phase: [water (0.1% TFA)-ACN]; B %: 10%-40%, 12 min. ¹H NMR (400 MHz, DMSO-d6) δ=4.89-4.67 (m, 1H), 4.50-4.30 (m, 2H), 3.55-3.41 (m, 3H), 3.42-3.41 (m, 1H), 2.13-1.52 (m, 6H). ESI [M+H]=194.1.

### Example S21: Synthesis of Compound 21

Compound 21 was prepared following a similar procedure as described for Compound 2.

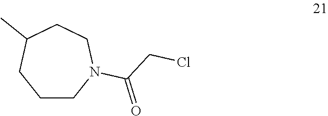

2-chloro-1-(4-methylazepan-1-yl)ethanone (21) (1.1 mg, 5.9 umol, 1.0%) was obtained as colorless oil after prep-HPLC purification. Purification condition: column: Phenomenex Luna C18 100*30 mm*5 um; mobile phase: [water (0.1% TFA)-ACN]; B %: 35%-45%, 12 min. ¹H NMR (400 MHz, DMSO-d6) δ=4.38-4.24 (m, 2H), 3.68-3.59 (m, 0.5H), 3.56-3.40 (m, 2H), 3.32-3.25 (m, 1H), 3.13 (ddd, J=3.2, 10.1, 13.6 Hz, 0.5H), 1.87-1.66 (m, 2H), 1.64-1.39 (m, 3H), 1.38-1.00 (m, 2H), 0.93-0.83 (m, 3H). ESI [M+H]=190.1.

### Example S22: Synthesis of Compound 22

Compound 22 was prepared following a similar procedure as described for Compound 2.

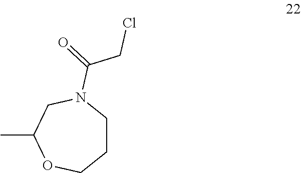

2-chloro-1-(2-methyl-1,4-oxazepan-4-yl)ethanone (22) (1.3 mg, 6.5 umol, 1.0%) was obtained as colorless oil after prep-HPLC purification. Purification condition: column: Phenomenex Luna C18 100*30 mm*5 um; mobile phase: [water (0.1% TFA)-ACN]; B %: 1%-27%, 12 min. ¹H NMR (400 MHz, DMSO-d6) δ=4.46-4.32 (m, 2H), 3.98-3.80 (m, 2H), 3.80-3.64 (m, 2H), 3.61-3.50 (m, 0.5H), 3.45-3.41 (m, 0.5H), 3.33 (br d, J=5.1 Hz, 0.5H), 3.22 (ddd, J=5.7, 7.9, 13.7 Hz, 0.5H), 3.06 (dd, J=9.8, 14.4 Hz, 0.5H), 2.88 (dd, J=9.7, 13.9 Hz, 0.5H), 1.92-1.70 (m, 2H), 1.11-1.01 (m, 3H). ESI [M+H]=192.0.

### Example S23: Synthesis of Compound 23

Compound 23 was prepared following a similar procedure as described for Compound 2.

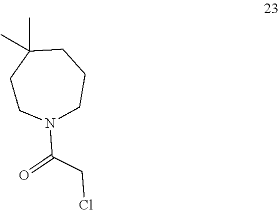

2-chloro-1-(4,4-dimethylazepan-1-yl)ethanone (23) (1.1 mg, 5.7 umol, 1.0%) was obtained as colorless oil after prep-HPLC purification. Purification condition: column: Phenomenex Luna C18 100*30 mm*5 um; mobile phase: [water (0.1% TFA)-ACN]; B %: 30%-55%, 12 min. ¹H NMR (400 MHz, DMSO-d6) δ=4.77 (s, 2H), 3.90-3.81 (m, 4H), 2.12 (td, J=6.1, 11.9 Hz, 1H), 2.04 (td, J=6.1, 11.9 Hz, 1H), 1.97-1.92 (m, 1H), 1.84 (td, J=2.8, 5.6 Hz, 1H), 1.80-1.72 (m, 2H), 1.32 (d, J=7.5 Hz, 6H). ESI [M+H]=204.0.

### Example S24: Synthesis of Compound 24

Compound 24 was prepared following a similar procedure as described for Compound 2.

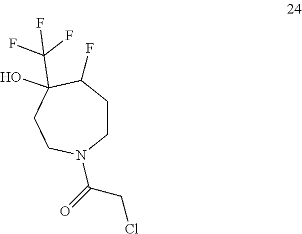 [SMILES: O=C(CCl)N1CCC(F)C(O)(C(F)(F)F)CC1]

2-chloro-1-(5-fluoro-4-hydroxy-4-(trifluoromethyl)azepan-1-yl)ethanone (24) (12.9 mg, 46.1 umol, 13.2%) was obtained as colorless oil after prep-HPLC purification. Purification condition: column: Phenomenex Luna C18 100*30 mm*5 um; mobile phase: [water(0.1% TFA)-ACN]; B %: 10%-45%, 12 min. ¹H NMR (400 MHz, DMSO-d6) δ=6.58 (br s, 1H), 4.99-4.69 (m, 1H), 4.45-4.31 (m, 2H), 3.83 (br dd, J=2.5, 14.4 Hz, 0.5H), 3.65-3.52 (m, 1H), 3.50-3.43 (m, 2H), 3.18-3.04 (m, 0.5H), 2.49-2.22 (m, 1H), 2.04-1.76 (m, 2H), 1.69-1.53 (m, 1H). ESI [M+H]=278.1.

### Example S25: Synthesis of Compound 25

Compound 25 was prepared following a similar procedure as described for Compound 2.

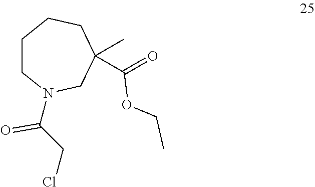 [SMILES: CCOC(=O)C1(C)CCCCN(C(=O)CCl)C1]

Ethyl 1-(2-chloroacetyl)-3-methylazepane-3-carboxylate (25) (1.1 mg, 4.4 umol, 1.1%) was obtained as colorless oil after prep-HPLC purification. Purification condition: column: Phenomenex Luna C18 100*30 mm*5 um; mobile phase: [water (0.1% TFA)-ACN]; B %: 30%-55%, 12 min. ¹H NMR (400 MHz, DMSO-d6) δ=4.59-4.30 (m, 2H), 4.14-3.90 (m, 2H), 3.70-3.50 (m, 2H), 3.44 (t, J=6.0 Hz, 1H), 3.30-3.19 (m, 1H), 1.96-1.79 (m, 1H), 1.77-1.49 (m, 4H), 1.49-1.35 (m, 1H), 1.21-1.13 (m, 3H), 1.08 (s, 3H). ESI [M+H]=262.1.

### Example S26: Synthesis of Compound 26

Compound 26 was prepared following a similar procedure as described for Compound 2.

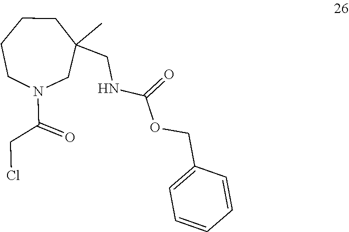 [SMILES: CC1(CNC(=O)OCc2ccccc2)CCCCN(C(=O)CCl)C1]

Benzyl ((1-(2-chloroacetyl)-3-methylazepan-3-yl)methyl)carbamate (26) (20.0 mg, 56.7 umol, 31.3%) was obtained as light yellow oil after prep-HPLC purification. Purification condition: column: Phenomenex Luna C18 100*30 mm*5 um; mobile phase: [water (0.1% TFA)-ACN]; B %: 35%-65%, 12 min. ¹H NMR (400 MHz, DMSO-d6) δ=7.45-7.23 (m, 5H), 7.07 (br t, J=6.3 Hz, 1H), 5.08-5.00 (m, 2H), 4.43-4.26 (m, 2H), 3.54-3.46 (m, 1H), 3.41-3.16 (m, 3H), 3.03-2.92 (m, 1H), 2.83 (dd, J=5.7, 13.7 Hz, 1H), 1.81-1.33 (m, 5H), 1.30-1.17 (m, 1H), 0.86-0.72 (m, 3H). ESI [M+H]=353.1.

### Example S27: Synthesis of Compound 27

Compound 27 was prepared following a similar procedure as described for Compound 2.

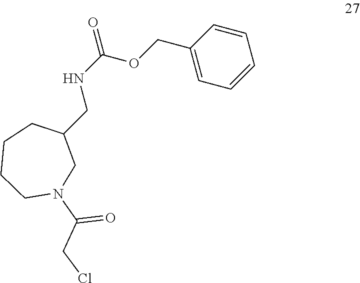 [SMILES: O=C(NCC1CCCCN(C(=O)CCl)C1)OCc1ccccc1]

Benzyl ((1-(2-chloroacetyl)azepan-3-yl)methyl)carbamate (27) (24.4 mg, 71.9 umol, 22.2%) was obtained as a white solid after prep-HPLC purification. Purification condition: column: Phenomenex Luna C18 100*30 mm*5 um; mobile phase: [water (0.1% TFA)-ACN]; B %: 30%-55%, 12 min. ¹H NMR (400 MHz, DMSO-d6) δ=7.45-7.25 (m, 6H), 5.02 (d, J=4.4 Hz, 2H), 4.39-4.18 (m, 2H), 3.81-3.49 (m, 2H), 3.35-3.11 (m, 1H), 3.05-2.81 (m, 3H), 1.86-1.49 (m, 5H), 1.40-1.01 (m, 2H). ESI [M+H]=339.1.

### Example S28: Synthesis of Compound 28

Compound 28 was prepared following a similar procedure as described for Compound 2.

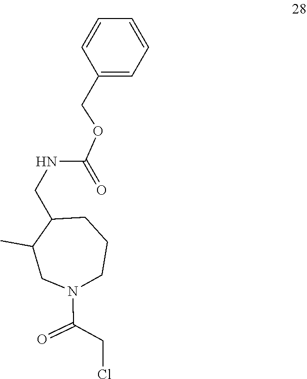 [SMILES: CC1CN(C(=O)CCl)CCCC1CNC(=O)OCc1ccccc1]

Benzyl ((1-(2-chloroacetyl)-3-methylazepan-4-yl)methyl)carbamate (28) (9.3 mg, 26.3 umol, 11.7%) was obtained as yellow oil after prep-HPLC purification. Purification condition: column: Phenomenex Luna C18 100*30 mm*5 um; mobile phase: [water (0.1% TFA)-ACN]; B %: 30%-55%, 12 min. ¹H NMR (400 MHz, DMSO-d6) δ=7.41-7.24 (m, 5H), 5.01 (s, 2H), 4.48-4.23 (m, 2H), 3.75-3.56 (m, 1H), 3.41-3.22 (m, 2H), 3.17-3.05 (m, 1H), 3.02-2.76 (m, 2H), 2.21-2.03 (m, 1H), 1.91-1.11 (m, 5H), 0.73 (dd, J=6.9, 18.3 Hz, 3H). ESI [M+H]=353.1.

### Example S29: Synthesis of Compound 29

Compound 29 was prepared following a similar procedure as described for Compound 2.

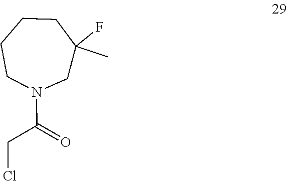

2-chloro-1-(3-fluoro-3-methylazepan-1-yl)ethanone (29) (1.9 mg, 9.0 umol, 3.0%) was obtained as light yellow oil after prep-HPLC purification. Purification condition: column: Phenomenex Luna C18 100*30 mm*5 um; mobile phase: [water (0.1% TFA)-ACN]; B %: 20%-45%, 12 min. ¹H NMR (400 MHz, DMSO-d6) δ=4.54 (d, J=13.6 Hz, 0.5H), 4.40 (d, J=1.1 Hz, 1H), 4.20 (d, J=13.7 Hz, 0.5H), 4.05-3.87 (m, 1H), 3.83-3.71 (m, 0.5H), 3.66-3.41 (m, 2H), 2.94 (ddd, J=5.0, 9.0, 13.7 Hz, 0.5H), 1.87-1.73 (m, 6H), 1.72-1.45 (m, 3H). ESI [M+H]=208.0.

### Example S30: Synthesis of Compound 30

Compound 30 was prepared following a similar procedure as described for Compound 2.

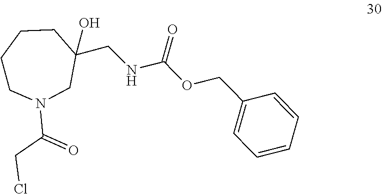 [SMILES: O=C(NCC1(O)CCCCN(C(=O)CCl)C1)OCc1ccccc1]

Benzyl ((1-(2-chloroacetyl)-3-hydroxyazepan-3-yl)methyl)carbamate (30) (5.2 mg, 14.6 umol, 6.6%) was obtained as light yellow oil after prep-HPLC purification. Purification condition: column: Phenomenex Luna C18 100*30 mm*5 um; mobile phase: [water (0.1% TFA)-ACN]; B %: 20%-50%, 12 min. ¹H NMR (400 MHz, DMSO-d6) δ=7.46-7.21 (m, 5H), 5.16-4.96 (m, 2H), 4.60 (d, J=13.2 Hz, 1H), 4.44-4.23 (m, 1H), 4.02-3.93 (m, 1H), 3.70-3.50 (m, 1H), 3.32-3.24 (m, 1H), 3.19-2.93 (m, 3H), 2.82 (br t, J=8.7 Hz, 1H), 1.79-1.33 (m, 6H). ESI [M+H]=355.1.

### Example S31: Synthesis of Compound 31

Compound 31 was prepared following a similar procedure as described for Compound 2.

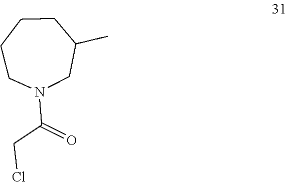

2-chloro-1-(3-methylazepan-1-yl)ethanone (31) (1.2 mg, 6.5 umol, 1.2%) was obtained as light yellow oil after prep-HPLC purification. Purification condition: column: Phenomenex Luna C18 100*30 mm*5 um; mobile phase: [water (0.1% TFA)-ACN]; B %: 25%-50%, 12 min. ¹H NMR (400 MHz, DMSO-d6) δ=4.46-4.26 (m, 2H), 3.78-3.50 (m, 2H), 3.33-3.11 (m, 1H), 2.92-2.66 (m, 1H), 1.91-1.45 (m, 5H), 1.41-1.07 (m, 2H), 0.87 (dd, J=6.7, 12.9 Hz, 3H). ESI [M+H]=190.1.

### Example S32: Synthesis of Compound 32

Compound 32 was prepared following a similar procedure as described for Compound 2.

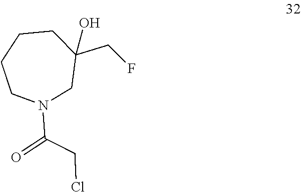 [SMILES: O=C(CCl)N1CCCCC(O)(CF)C1]

2-chloro-1-(3-(fluoromethyl)-3-hydroxyazepan-1-yl)ethanone (32) (14.0 mg, 61.6 umol, 22.6%) was obtained as light yellow oil after prep-HPLC purification. Purification condition: column: Waters Xbridge Prep OBD C18 150*40 mm*10 um; mobile phase: [water(10 mM NH₄HCO₃)-ACN]; B %: 1%-25%, 8 min. ¹H NMR (400 MHz, CHLOROFORM-d) δ=4.45-4.29 (m, 1H), 4.22-4.03 (m, 2H), 3.98-3.82 (m, 1H), 3.78-3.44 (m, 2H), 3.42-3.02 (m, 2H), 2.10-1.48 (m, 6H). ESI [M+H]=224.0.

### Example S33: Synthesis of Compound 33

Compound 33 was prepared following a similar procedure as described for Compound 2.

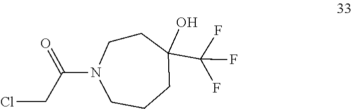 [SMILES: O=C(CCl)N1CCCC(O)(C(F)(F)F)CC1]

2-chloro-1-(4-hydroxy-4-(trifluoromethyl)azepan-1-yl)ethanone (33) (2.6 mg, 9.8 umol, 4.3%) was obtained as light yellow oil after prep-HPLC purification. Purification condition: column: Phenomenex Luna C18 100*30 mm*5 um; mobile phase: [water (0.1% TFA)-ACN]; B %: 5%-40%, 12 min. ¹H NMR (400 MHz, DMSO-d6) δ=5.98-5.86 (m, 1H), 4.46-4.30 (m, 2H), 3.85 (br d, J=16.5 Hz, 1H), 3.65-3.42 (m, 2H), 3.12-3.03 (m, 1H), 2.11-1.44 (m, 6H). ESI [M+H]=260.0.

### Example S34: Synthesis of Compound 34

Compound 34 was prepared following a similar procedure as described for Compound 2.

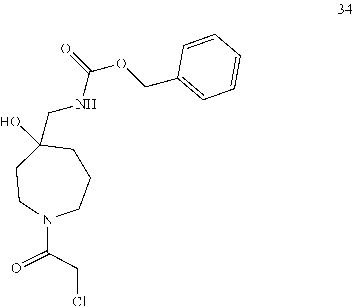 [SMILES: O=C(NCC1(O)CCCN(C(=O)CCl)CC1)OCc1ccccc1]

Benzyl ((1-(2-chloroacetyl)-4-hydroxyazepan-4-yl)methyl)carbamate (34) (30.0 mg, 83.6 umol, 13.4%) was obtained as light yellow oil after prep-HPLC purification. Purification condition: column: Phenomenex Luna C18 100*30 mm*5 um; mobile phase: [water (0.1% TFA)-ACN]; B %: 15%-45%, 12 min. ¹H NMR (400 MHz, DMSO-d6) δ=7.43-7.27 (m, 5H), 7.18-7.06 (m, 1H), 5.03 (s, 2H), 4.35 (br s, 1H), 4.32 (br s, 1H), 3.77-3.67 (m, 1H), 3.55-3.26 (m, 3H), 3.16-2.91 (m, 2H), 2.08-1.80 (m, 1H), 1.69-1.28 (m, 5H). ESI [M+H]=355.1.

### Example S35: Synthesis of Compound 35

Compound 35 was prepared following a similar procedure as described for Compound 2.

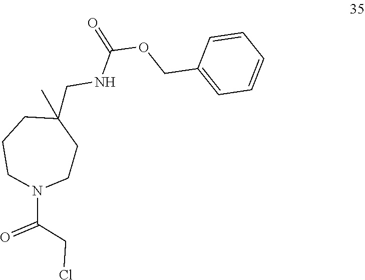 [SMILES: CC1(CNC(=O)OCc2ccccc2)CCCN(C(=O)CCl)CC1]

Benzyl ((1-(2-chloroacetyl)-4-methylazepan-4-yl)methyl)carbamate (35) (24.4 mg, 69.0 umol, 14.4%) was obtained as colorless oil after prep-HPLC purification. Purification condition: (column: Waters Xbridge BEH C18 100*30 mm*10 um; mobile phase: [water (0.1% TFA)-ACN]; B %: 35%-45%, 12 min. ¹H NMR (400 MHz, DMSO-d6) δ=7.40-7.27 (m, 5H), 5.03 (s, 2H), 4.40-4.27 (m, 2H), 3.47-3.22 (m, 4H), 2.97-2.80 (m, 2H), 1.80-1.60 (m, 2H), 1.57 (br dd, J=5.4, 9.0 Hz, 1H), 1.50-1.41 (m, 1H), 1.33 (br d, J=7.7 Hz, 2H), 0.81 (br d, J=7.2 Hz, 3H). ESI [M+H]=353.1.

### Example S36: Synthesis of Compound 36

Compound 36 was prepared following a similar procedure as described for Compound 2.

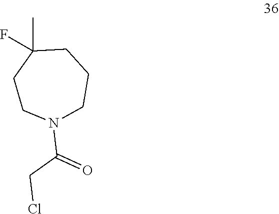

2-chloro-1-(4-fluoro-4-methylazepan-1-yl)ethanone (36) (1.1 mg, 5.3 umol, 1.8%) was obtained as light yellow oil after prep-HPLC purification. Purification condition: column: Waters Xbridge BEH C18 100*30 mm*10 um; mobile phase: [water (0.1% TFA)-ACN]; B %: 15%-35%, 12 min. ¹H NMR (400 MHz, DMSO-d6) δ=4.35-4.31 (m, 2H), 3.49-3.43 (m, 2H), 3.39-3.09 (m, 2H), 2.07-1.53 (m, 6H), 1.38-1.24 (m, 3H). ESI [M+H]=208.1.

### Example S37: Synthesis of Compound 37

Compound 37 was prepared following a similar procedure as described for Compound 2.

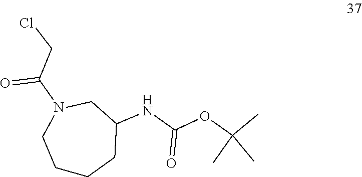 [SMILES: CC(C)(C)OC(=O)NC1CCCCN(C(=O)CCl)C1]

Tert-butyl (1-(2-chloroacetyl)azepan-3-yl)carbamate (37) (11.8 mg, 40.2 umol, 9.6%) was obtained as a yellow solid after prep-HPLC purification. Purification condition: column: Waters Xbridge Prep OBD C18 150*40 mm*10 um; mobile phase: [water (10 mM NH₄HCO₃)-ACN]; B %: 20%-50%, 8 min. ¹H NMR (400 MHz, DMSO-d6) δ=7.07-6.66 (m, 1H), 4.61-4.30 (m, 2H), 3.86-3.39 (m, 4H), 3.22-2.97 (m, 1H), 1.85-1.50 (m, 4H), 1.39 (br d, J=7.1 Hz, 11H). ESI [M−Boc+H]=191.1.

### Example S38: Synthesis of Compound 38

Compound 38 was prepared following a similar procedure as described for Compound 2.

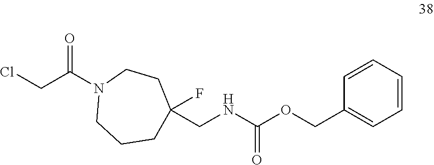 [SMILES: O=C(NCC1(F)CCCN(C(=O)CCl)CC1)OCc1ccccc1]

Benzyl ((1-(2-chloroacetyl)-4-fluoroazepan-4-yl)methyl)carbamate (38) (11.5 mg, 31.4 umol, 11.1%) was obtained as yellow oil after prep-HPLC purification. Purification condition: column: Welch Ultimate AQ-C18 150*30 mm*5 um; mobile phase: [water (0.1% TFA)-ACN]; B %: 25%-55%, 12 min. ¹H NMR (400 MHz, DMSO-d6) δ=7.67-7.49 (m, 1H), 7.43-7.23 (m, 5H), 5.17-4.93 (m, 2H), 4.49-4.30 (m, 2H), 3.81-3.56 (m, 1H), 3.46-3.31 (m, 2H), 3.28-3.03 (m, 3H), 2.08-1.45 (m, 6H). ESI [M+H]=357.1.

### Example S39: Synthesis of Compound 39

Compound 39 was prepared following a similar procedure as described for Compound 2.

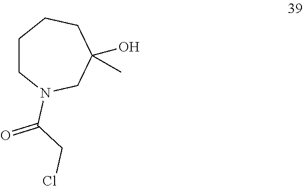

2-chloro-1-(3-hydroxy-3-methylazepan-1-yl)ethanone (39) (1.1 mg, 5.4 umol, 1.0%) was obtained as light yellow oil after prep-HPLC purification. Purification condition: column: Welch Ultimate AQ-C18 150*30 mm*5 um; mobile phase: [water (0.1% TFA)-ACN]; B %: 2%-32%, 12 min. ¹H NMR (400 MHz, DMSO-d6) δ=4.72-4.56 (m, 1H), 4.44-4.32 (m, 1H), 4.07-3.94 (m, 1H), 3.69-3.45 (m, 1H), 3.25-3.05 (m, 1H), 2.85 (ddd, J=5.0, 9.0, 13.6 Hz, 1H), 1.79-1.46 (m, 5H), 1.37 (br d, J=5.9 Hz, 1H), 1.20-1.04 (m, 3H). ESI [M+H]=206.1.

### Example S40: Synthesis of Compound 40

Compound 40 was prepared following a similar procedure as described for Compound 2.

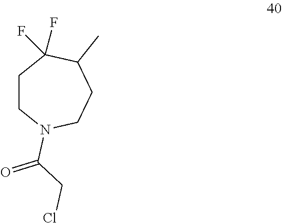

2-chloro-1-(4,4-difluoro-5-methylazepan-1-yl)ethanone (40) (1.2 mg, 5.2 umol, 1.1%) was obtained as light yellow oil after prep-HPLC purification. Purification condition: column: Welch Ultimate AQ-C18 150*30 mm*5 um; mobile phase: [water (0.1% TFA)-ACN]; B %: 17%-47%, 12 min. ¹H NMR (400 MHz, DMSO-d6) δ=4.45-4.35 (m, 2H), 3.67-3.49 (m, 3H), 3.42-3.35 (m, 1H), 2.40-2.01 (m, 3H), 1.87-1.73 (m, 1H), 1.62 (br d, J=1.6 Hz, 1H), 0.99 (dd, J=7.0, 9.4 Hz, 3H). ESI [M+H]=226.0.

### Example S41: Synthesis of Compound 41

Compound 41 was prepared following a similar procedure as described for Compound 2.

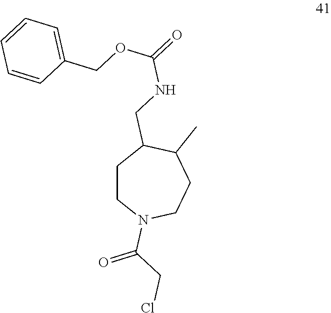 [SMILES: CC1CCN(C(=O)CCl)CCC1CNC(=O)OCc1ccccc1]

Benzyl ((1-(2-chloroacetyl)-5-methylazepan-4-yl)methyl)carbamate (41) (9.2 mg, 23.9 umol, 7.34%) was obtained as yellow oil after prep-HPLC purification. Purification condition: column: Welch Ultimate AQ-C18 150*30 mm*5 um; mobile phase: [water (0.1% TFA)-ACN]; B %: 28%-58%, 12 min. ¹H NMR (400 MHz, DMSO-d6) δ=7.43-7.24 (m, 5H), 5.01 (d, J=1.7 Hz, 2H), 4.50-4.26 (m, 2H), 3.56-3.45 (m, 2H), 3.44-3.26 (m, 2H), 3.01-2.77 (m, 2H), 2.04-1.85 (m, 1H), 1.80-1.47 (m, 4H), 1.43-1.24 (m, 1H), 0.82 (t, J=7.2 Hz, 3H). ESI [M+H]=353.1.

### Example S42: Synthesis of Compound 42

Compound 42 was prepared following a similar procedure as described for Compound 2.

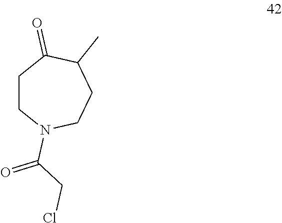

1-(2-chloroacetyl)-5-methylazepan-4-one (42) (8.7 mg, 42.0 umol, 7.6%) was obtained as light yellow oil after prep-HPLC purification. Purification condition: column: Welch Ultimate AQ-C18 150*30 mm*5 um; mobile phase: [water (0.1% TFA)-ACN]; B %: 1%-30%, 12 min. ¹H NMR (400 MHz, DMSO-d6) δ=4.56-4.25 (m, 2H), 4.22-3.98 (m, 1H), 3.89-3.59 (m, 1H), 3.44-3.37 (m, 1H), 3.33-3.28 (m, 1H), 3.15-2.93 (m, 1H), 2.71-2.53 (m, 2H), 1.76-1.65 (m, 1H), 1.59-1.16 (m, 1H), 0.96 (d, J=6.6 Hz, 3H). ESI [M+H]=204.1.

### Example S43: Synthesis of Compound 43

Compound 43 was prepared following a similar procedure as described for Compound 2.

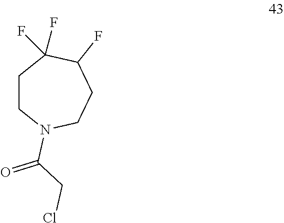 [SMILES: O=C(CCl)N1CCC(F)C(F)(F)CC1]

2-chloro-1-(4,4,5-trifluoroazepan-1-yl)ethanone (43) (1.1 mg, 4.5 umol, 1.0%) was obtained as light yellow oil after prep-HPLC purification. Purification condition: column: Welch Ultimate AQ-C18 150*30 mm*5 um; mobile phase: [water (0.1% TFA)-ACN]; B %: 10%-40%, 12 min. ¹H NMR (400 MHz, DMSO-d6) δ=5.16-4.85 (m, 1H), 4.49-4.33 (m, 2H), 3.62-3.51 (m, 3H), 3.47-3.40 (m, 1H), 2.43-1.97 (m, 4H). ESI [M+H]=230.0.

### Example S44: Synthesis of Compound 44

Compound 44 was prepared following a similar procedure as described for Compound 2.

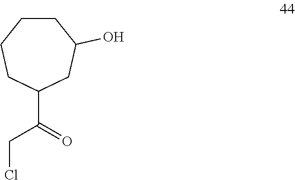 [SMILES: O=C(CCl)C1CCCCC(O)C1]

2-chloro-1-(3-hydroxyazepan-1-yl)ethanone (44) (14.6 mg, 76.2 umol, 12.8%) was obtained as light yellow oil after prep-HPLC purification. Purification condition: column: Welch Ultimate AQ-C18 150*30 mm*5 um; mobile phase: [water (0.1% TFA)-ACN]; B %: 1%-27%, 12 min. ¹H NMR (400 MHz, DMSO-d6) δ=4.99-4.72 (m, 1H), 4.56-4.24 (m, 2H), 4.00-3.76 (m, 1H), 3.71-3.40 (m, 2H), 3.31-3.20 (m, 1H), 2.82 (br dd, J=9.0, 13.1 Hz, 1H), 1.86-1.58 (m, 4H), 1.53-1.17 (m, 2H). ESI [M+H]=192.1.

### Example S45: Synthesis of Compound 45

Compound 45 was prepared following a similar procedure as described for Compound 2.

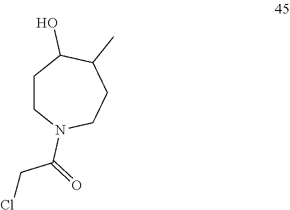

2-chloro-1-(4-hydroxy-5-methylazepan-1-yl)ethanone (45) (41.3 mg, 200.0 umol, 66.3%) was obtained as light yellow oil after prep-HPLC purification. Purification condition: column: Waters Xbridge BEH C18 100*30 mm*10 um; mobile phase: [water (0.1% TFA)-ACN]; B %: 5%-30%, 12 min. ¹H NMR (400 MHz, DMSO-d6) δ=4.58-4.47 (m, 1H), 4.36-4.30 (m, 2H), 3.71-3.55 (m, 2H), 3.46-3.40 (m, 2H), 3.28-3.18 (m, 1H), 1.90-1.76 (m, 1H), 1.75-1.54 (m, 3H), 1.51-1.41 (m, 1H), 0.99-0.87 (m, 3H). ESI [M+H]=206.1.

### Example S46: Synthesis of Compound 46

Compound 46 was prepared following a similar procedure as described for Compound 2.

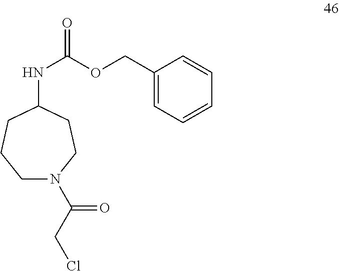 [SMILES: O=C(NC1CCCN(C(=O)CCl)CC1)OCc1ccccc1]

Benzyl (1-(2-chloroacetyl)azepan-4-yl)carbamate (46) (83.5 mg, 256.6 umol, 48.7%) was obtained as yellow oil after prep-HPLC purification. Purification condition: column: Waters Xbridge BEH C18 100*30 mm*10 um; mobile phase: [water (0.1% TFA)-ACN]; B %: 25%-40%, 12 min. ¹H NMR (400 MHz, DMSO-d6) δ=7.41-7.27 (m, 5H), 5.01 (s, 2H), 4.44-4.27 (m, 2H), 3.67-3.57 (m, 1H), 3.49 (br dd, J=4.2, 8.6 Hz, 2H), 3.36-3.14 (m, 2H), 2.05-1.36 (m, 6H). ESI [M+H]=325.1.

### Example S47: Synthesis of Compound 47

Compound 47 was prepared following a similar procedure as described for Compound 2.

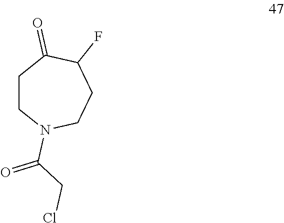 [SMILES: O=C1CCN(C(=O)CCl)CCC1F]

1-(2-chloroacetyl)-5-fluoroazepan-4-one (47) (14.1 mg, 59.5 umol, 19.9%) was obtained as light yellow oil after prep-HPLC purification. Purification condition: column: Phenomenex Luna C18 100*30 mm*5 um; mobile phase: [water (0.1% TFA)-ACN]; B %: 1%-22%, 12 min. ¹H NMR (400 MHz, DMSO-d6) δ=5.71-5.32 (m, 1H), 4.50-4.31 (m, 2H), 4.06-3.74 (m, 2H), 3.68-3.49 (m, 1H), 3.40 (br d, J=3.9 Hz, 1H), 2.85-2.68 (m, 1H), 2.66-2.57 (m, 1H), 2.24-1.71 (m, 2H). ESI [M+H]=208.1.

### Example S48: Synthesis of Compound 48

Compound 48 was prepared following a similar procedure as described for Compound 2.

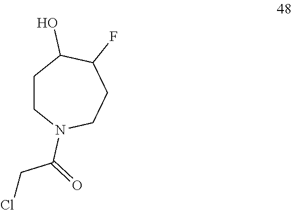 [SMILES: O=C(CCl)N1CCC(O)C(F)CC1]

2-chloro-1-(4-fluoro-5-hydroxyazepan-1-yl)ethanone (48) (5.7 mg, 25.9 umol, 8.8%) was obtained as light yellow oil after prep-HPLC purification. Purification condition: column: Phenomenex Luna C18 100*30 mm*5 um; mobile phase: [water(0.1% TFA)-ACN]; B %: 1%-17%, 12 min. ¹H NMR (400 MHz, DMSO-d6) δ=5.20 (br dd, J=4.2, 9.9 Hz, 1H), 4.59-4.45 (m, 0.5H), 4.42-4.30 (m, 2H), 3.84-3.68 (m, 1H), 3.64-3.41 (m, 4H), 3.26 (ddd, J=2.6, 9.3, 14.1 Hz, 0.5H), 2.22-1.48 (m, 4H). ESI [M+H]=210.0.

### Example S49: Synthesis of Compound 49

Compound 49 was prepared following a similar procedure as described for Compound 2.

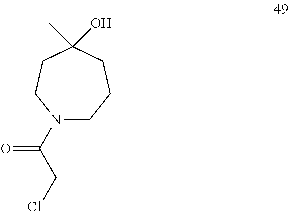

2-chloro-1-(4-hydroxy-4-methylazepan-1-yl)ethanone (49) (10.3 mg, 49.7 umol, 16.5%) was obtained as light yellow oil after prep-HPLC purification. Purification condition: column: Phenomenex Luna C18 100*30 mm*5 um; mobile phase: [water (0.1% TFA)-ACN]; B %: 1%-30%, 12 min. ¹H NMR (400 MHz, DMSO-d6) δ=4.39-4.29 (m, 2H), 3.66-3.42 (m, 3H), 3.30 (br s, 1H), 3.15 (ddd, J=1.9, 10.3, 13.7 Hz, 1H), 2.19-1.82 (m, 1H), 1.74-1.34 (m, 5H), 1.12 (d, J=8.4 Hz, 3H). ESI [M+H]=206.1.

### Example S50: Synthesis of Compound 50

Compound 50 was prepared following a similar procedure as described for Compound 2.

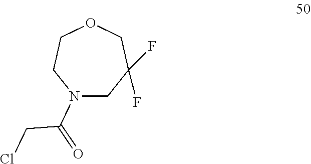 [SMILES: O=C(CCl)N1CCOCC(F)(F)C1]

2-chloro-1-(6,6-difluoro-1,4-oxazepan-4-yl)ethanone (50) (5 mg, 23.3 umol, 4.2%) was obtained as light yellow oil after prep-HPLC purification. Purification condition: column: Phenomenex Luna C18 100*30 mm*5 um; mobile phase: [water (0.1% TFA)-ACN]; B %: 10%-30%, 12 min. ¹H NMR (400 MHz, DMSO-d6) δ=4.53-4.43 (m, 2H), 4.17-3.99 (m, 2H), 3.97-3.83 (m, 3H), 3.80-3.63 (m, 3H). ESI [M+H]=214.0.

### Example S51: Synthesis of Compound 51

Compound 51 was prepared following a similar procedure as described for Compound 2.

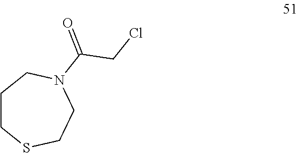 [SMILES: O=C(CCl)N1CCCSCC1]

2-chloro-1-(1,4-thiazepan-4-yl)ethanone (51) (6.2 mg, 31.6 umol, 9.7%) was obtained as light yellow oil after prep-HPLC purification. Purification condition: column: Phenomenex Luna C18 100*30 mm*5 um; mobile phase: [water (0.1% TFA)-ACN]; B %: 5%-35%, 12 min. ¹H NMR (400 MHz, DMSO-d6) δ=4.38 (d, J=12.5 Hz, 2H), 3.74-3.58 (m, 3H), 3.57-3.48 (m, 1H), 2.87-2.78 (m, 1H), 2.74-2.68 (m, 1H), 2.63 (td, J=6.2, 10.7 Hz, 2H), 2.01-1.83 (m, 2H). ESI [M+H]=194.0.

### Example S52: Synthesis of Compound 52

Compound 52 was prepared following a similar procedure as described for Compound 2.

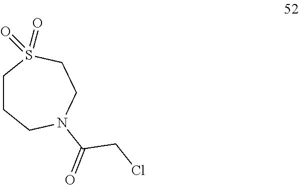 [SMILES: O=C(CCl)N1CCCS(=O)(=O)CC1]

2-chloro-1-(1,1-dioxido-1,4-thiazepan-4-yl)ethanone (52) (8.8 mg, 38.8 umol, 14.4%) was obtained as light yellow oil after prep-HPLC purification. Purification condition: column: Phenomenex Luna C18 100*30 mm*5 um; mobile phase: [water (0.1% TFA)-ACN]; B %: 1%-15%, 12 min. ¹H NMR (400 MHz, DMSO-d6) δ=4.43 (d, J=11.2 Hz, 2H), 3.80-3.49 (m, 5H), 3.34-3.17 (m, 3H), 2.12-2.04 (m, 1H), 1.89 (quin, J=6.2 Hz, 1H). ESI [M+H]=226.0.

### Example S53: Synthesis of Compound 53

Compound 53 was prepared following a similar procedure as described for Compound 2.

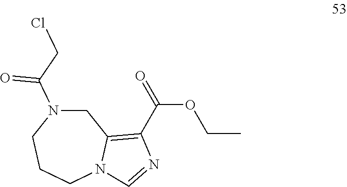 [SMILES: CCOC(=O)c1ncn2c1CN(C(=O)CCl)CCC2]

Ethyl 8-(2-chloroacetyl)-6,7,8,9-tetrahydro-5H-imidazo[1,5-a][1,4]diazepine-1-carboxylate (53) (15.7 mg, 39.2 umol, 19.3%) was obtained as light yellow oil after prep-HPLC purification. Purification condition: column: Phenomenex Luna C18 100*30 mm*5 um; mobile phase: [water (0.1% TFA)-ACN]; B %: 1%-15%, 12 min. ¹H NMR (400 MHz, DMSO-d6) δ=8.38-8.08 (m, 1H), 5.17-4.98 (m, 2H), 4.41 (s, 1H), 4.36 (s, 1H), 4.32 (d, J=1.8 Hz, 1H), 4.30 (br d, J=1.8 Hz, 2H), 4.29-4.28 (m, 1H), 3.79 (br d, J=4.5 Hz, 2H), 2.05-1.75 (m, 2H), 1.32 (dt, J=5.1, 7.1 Hz, 3H). ESI [M+H]=286.1.

### Example S54: Synthesis of Compound 54

Compound 54 was prepared following a similar procedure as described for Compound 2.

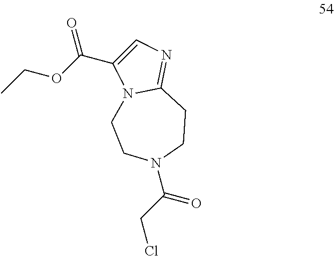 [SMILES: CCOC(=O)c1cnc2n1CCN(C(=O)CCl)CC2]

Ethyl 7-(2-chloroacetyl)-6,7,8,9-tetrahydro-5H-imidazo[1,2-d][1,4]diazepine-3-carboxylate (54) (11.0 mg, 26.2 umol, 12.9%) was obtained as light yellow oil after prep-HPLC purification. Purification condition: column: Waters Xbridge BEH C18 100*30 mm*10 um; mobile phase: [water (0.1% TFA)-ACN]; B %: 5%-16%, 12 min. ¹H NMR (400 MHz, DMSO-d6) δ=7.94 (br d, J=4.5 Hz, 1H), 4.83-4.65 (m, 2H), 4.52 (s, 2H), 4.30 (q, J=7.1 Hz, 2H), 3.86 (br d, J=7.5 Hz, 4H), 3.33-3.12 (m, 2H), 1.30 (t, J=7.1 Hz, 3H). ESI [M+H]=286.0.

### Example S55: Synthesis of Compound 55

Compound 55 was prepared following a similar procedure as described for Compound 2.

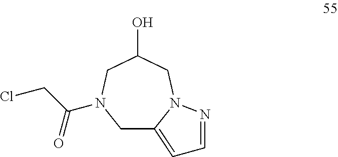 [SMILES: O=C(CCl)N1Cc2ccnn2CC(O)C1]

2-chloro-1-(7-hydroxy-7,8-dihydro-4H-pyrazolo[1,5-a][1,4]diazepin-5(6H)-yl)ethanone (55) (5.8 mg, 24.7 umol, 9.4%) was obtained as a pale yellow solid after prep-HPLC purification. Purification condition: column: Waters Xbridge BEH C18 100*30 mm*10 um; mobile phase: [water (0.1% TFA)-ACN]; B %: 5%-15%, 12 min. ¹H NMR (400 MHz, DMSO-d6) δ=7.31-7.19 (m, 1H), 6.40-6.11 (m, 1H), 5.42 (br s, 1H), 4.87-4.72 (m, 1H), 4.45-4.25 (m, 4H), 4.17-3.88 (m, 1H), 3.86-3.57 (m, 2H), 3.46 (br d, J=7.7 Hz, 1H). ESI [M+H]=230.1.

### Example S56: Synthesis of Compound 56

Compound 56 was prepared following a similar procedure as described for Compound 2.

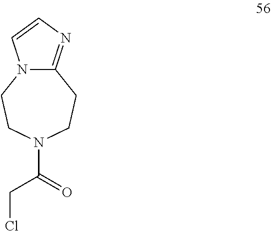 [SMILES: O=C(CCl)N1CCc2nccn2CC1]

2-chloro-1-(8,9-dihydro-5H-imidazo[1,2-d][1,4]diazepin-7(6H)-yl)ethanone (56) (15.3 mg, 43.1 umol, 15.0%) was obtained as a pale yellow solid after prep-HPLC purification. Purification condition: column: Waters Xbridge BEH C18 100*30 mm*10 um; mobile phase: [water (0.1% TFA)-ACN]; B %: 1%-8%, 12 min. ¹H NMR (400 MHz, DMSO-d6) δ=7.61 (br d, J=13.8 Hz, 1H), 7.50 (br s, 1H), 4.54 (s, 2H), 4.47-4.40 (m, 1H), 4.38-4.30 (m, 1H), 3.89-3.84 (m, 2H), 3.79 (br s, 2H), 3.35 (br d, J=4.5 Hz, 1H), 3.23-3.16 (m, 1H). ESI [M+H]=214.1.

### Example S57: Synthesis of Compound 57

Compound 57 was prepared following a similar procedure as described for Compound 2.

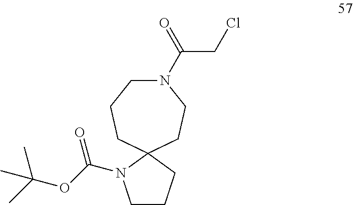 [SMILES: CC(C)(C)OC(=O)N1CCCC12CCCN(C(=O)CCl)CC2]

Tert-butyl 8-(2-chloroacetyl)-1,8-diazaspiro[4.6]undecane-1-carboxylate (57) (7.6 mg, 21.5 umol, 6.1%) was obtained as light yellow oil after prep-HPLC purification. Purification condition: column: Waters Xbridge BEH C18 100*25 mm*5 um; mobile phase: [water (10 mM NH₄HCO₃)-ACN]; B %: 25%-55%, 8 min. ¹H NMR (400 MHz, DMSO-d6) δ=4.45-4.25 (m, 2H), 3.74-3.44 (m, 3H), 3.26-2.90 (m, 3H), 2.69-2.51 (m, 1H), 2.41-2.23 (m, 2H), 1.93 (br s, 1H), 1.81-1.59 (m, 5H), 1.45 (br d, J=8.8 Hz, 1H), 1.35 (d, J=2.9 Hz, 9H). ESI [M−Boc+H]=231.2.

### Example S58: Synthesis of Compound 58

Compound 58 was prepared following a similar procedure as described for Compound 2.

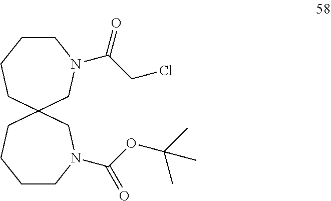 [SMILES: CC(C)(C)OC(=O)N1CCCCC2(CCCCN(C(=O)CCl)C2)C1]

Tert-butyl 9-(2-chloroacetyl)-2,9-diazaspiro[6.6]tridecane-2-carboxylate (58) (20.0 mg, 54.0 umol, 17.0%) was obtained as a pale yellow solid after prep-HPLC purification. Purification condition: column: Waters Xbridge BEH C18 100*25 mm*5 um; mobile phase: [water (10 mM NH₄HCO₃)-ACN]; B %: 35%-65%, 8 min. ¹H NMR (400 MHz, DMSO-d6) δ=4.38 (s, 2H), 3.79-3.39 (m, 4H), 3.30-2.79 (m, 4H), 1.80-1.42 (m, 10H), 1.39 (s, 9H), 1.27-1.03 (m, 2H). ESI [M−Boc+H]=259.1.

### Example S59: Synthesis of Compound 59

Compound 59 was prepared following a similar procedure as described for Compound 2.

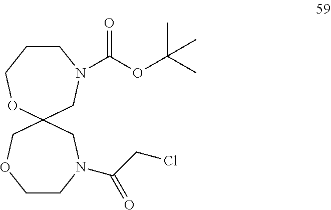 [SMILES: CC(C)(C)OC(=O)N1CCCOC2(COCCN(C(=O)CCl)C2)C1]

Tert-butyl 11-(2-chloroacetyl)-1,8-dioxa-4,11-diazaspiro[5.6]dodecane-4-carboxylate (59) (20.0 mg, 57.2 umol, 31.2%) was obtained as light yellow oil after prep-HPLC purification. Purification condition: column: Waters Xbridge Prep OBD C18 150*40 mm*10 um; mobile phase: [water (10 mM NH₄HCO₃)-ACN]; B %: 15%-45%, 8 min. ¹H NMR (400 MHz, DMSO-d6) δ=4.66-4.32 (m, 2H), 4.02-3.88 (m, 1H), 3.83-3.42 (m, 10H), 3.31-3.23 (m, 1H), 3.18-2.93 (m, 2H), 1.41 (s, 9H). ESI [M−Boc+H]=249.1.

### Example S60: Synthesis of Compound 60

Compound 60 was prepared following a similar procedure as described for Compound 2.

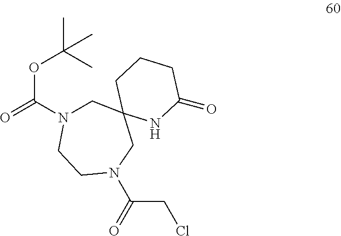 [SMILES: CC(C)(C)OC(=O)N1CCN(C(=O)CCl)CC2(CCCC(=O)N2)C1]

Tert-butyl 11-(2-chloroacetyl)-2-oxo-1,8,11-triazaspiro[5.6]dodecane-8-carboxylate (60) (4.3 mg, 11.4 umol, 3.6%) was obtained as a white solid after prep-HPLC purification. Purification condition: column: Waters Xbridge Prep OBD C18 150*40 mm*10 um; mobile phase: [water (10 mM NH₄HCO₃)-ACN]; B %: 10%-40%, 8 min. ¹H NMR (400 MHz, DMSO-d6) δ=7.44-7.04 (m, 1H), 4.53-4.40 (m, 1H), 4.36-4.25 (m, 1H), 3.87-3.39 (m, 7H), 3.11 (d, J=14.5 Hz, 1H), 2.20-2.02 (m, 2H), 1.77-1.50 (m, 4H), 1.45-1.32 (m, 9H). ESI [M+H]=360.1.

### Example S61: Synthesis of Compound 61

Compound 61 was prepared following a similar procedure as described for Compound 2.

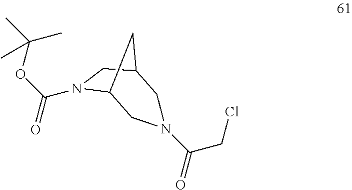 [SMILES: CC(C)(C)OC(=O)N1CC2CC1CN(C(=O)CCl)C2]

Tert-butyl 3-(2-chloroacetyl)-3,6-diazabicyclo[3.2.1]octane-6-carboxylate (61) (21.6 mg, 74.6 umol, 17.6%) was obtained as yellow oil after prep-HPLC purification. Purification condition: column: Welch Xtimate C18 150*30 mm*5 um; mobile phase: [water (10 mM NH₄HCO₃)-ACN]; B %: 20%-50%, 3 min. ¹H NMR (400 MHz, DMSO-d6) δ=4.48-4.29 (m, 1H), 4.27-4.06 (m, 2H), 4.03-3.89 (m, 1H), 3.85-3.63 (m, 1H), 3.29-3.12 (m, 2H), 3.07-2.79 (m, 1H), 2.67 (br dd, J=8.3, 12.8 Hz, 1H), 2.44 (br s, 1H), 1.94-1.67 (m, 2H), 1.44-1.34 (m, 9H). ESI [M−tBu+H]=233.0.

### Example S62: Synthesis of Compound 62

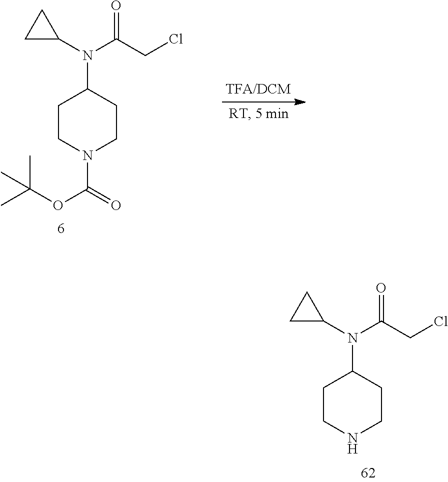 [SMILES: CC(C)(C)OC(=O)N1CCC(N(C(=O)CCl)C2CC2)CC1 ; O=C(CCl)N(C1CCNCC1)C1CC1]

A mixture of tert-butyl 4-[(2-chloroacetyl)-cyclopropyl-amino]piperidine-1-carboxylate (6) (20.8 mg, 65.9 umol, 1 eq.) in TFA (0.2 mL)/DCM (0.8 mL) was stirred at 25° C. for 5 min and then mixture was concentrated. The residue was diluted with deionized water (10 mL) and then lyophilized to give the product 2-chloro-N-cyclopropyl-N-(4-piperidyl)acetamide (62) (16.8 mg, 47.0 umol, 71.3%) as light yellow oil. ¹H NMR (400 MHz, methanol-d4) δ=4.41 (s, 2H), 3.91 (tt, J=3.8, 12.2 Hz, 1H), 3.41-3.32 (m, 2H), 3.04-2.92 (m, 2H), 2.75 (br d, J=3.9 Hz, 1H), 2.37 (dq, J=4.0, 13.2 Hz, 2H), 1.87 (br d, J=13.6 Hz, 2H), 0.98-0.79 (m, 4H). ESI [M+H]=217.1.

### Example S63: Synthesis of Compound 63

Compound 63 was prepared following a similar procedure as described for Compound 62.

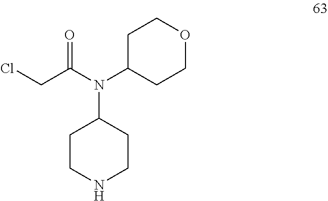 [SMILES: O=C(CCl)N(C1CCNCC1)C1CCOCC1]

2-chloro-N-(piperidin-4-yl)-N-(tetrahydro-2H-pyran-4-yl)acetamide (63) (12.3 mg, 32.8 umol, 78.9%) was obtained as light yellow oil. ¹H NMR (400 MHz, methanol-d4) δ=4.33 (s, 2H), 4.07-3.88 (m, 3H), 3.58-3.38 (m, 5H), 3.22-2.84 (m, 4H), 2.27-1.88 (m, 2H), 1.75 (br d, J=12.3 Hz, 3H), 1.42 (brs, 1H). ESI [M+H]=261.1.

### Example S64: Synthesis of Compound 64

Compound 64 was prepared following a similar procedure as described for Compound 62.

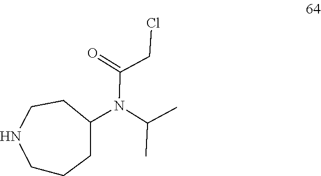

N-(azepan-4-yl)-2-chloro-N-isopropyl-acetamide (64) (16.8 mg, 46.5 umol, 67.2%, 84.9% purity) was obtained as colorless oil. ¹H NMR (400 MHz, methanol-d4) δ=4.24 (s, 2H), 4.17-4.06 (m, 1H), 3.57-3.41 (m, 2H), 3.30-3.11 (m, 3H), 2.72-2.56 (m, 1H), 2.55-2.40 (m, 1H), 2.14-1.97 (m, 2H), 1.92-1.76 (m, 2H), 1.44-1.25 (m, 6H). ESI [M+H]=233.1.

### Example S65: Synthesis of Compound 65

Compound 65 was prepared following a similar procedure as described for Compound 62.

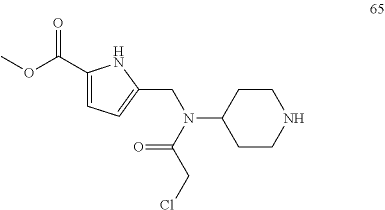 [SMILES: COC(=O)c1ccc(CN(C(=O)CCl)C2CCNCC2)[nH]1]

Methyl 5-[[(2-chloroacetyl)-(4-piperidyl)amino]methyl]-1H-pyrrole-2-carboxylate (65) (16.3 mg, 30.8 umol, 51.1%, 81.7% purity) was obtained as light yellow oil. ¹H NMR (400 MHz, methanol-d4) δ=6.83-6.61 (m, 1H), 6.18-5.91 (m, 1H), 4.58-4.33 (m, 3H), 4.31-4.19 (m, 1H), 4.07 (br d, J=12.8 Hz, 1H), 3.71 (br s, 3H), 3.46-3.27 (m, 2H), 3.13-2.82 (m, 2H), 2.19-1.68 (m, 4H). ESI [M+H]=314.1.

### Example S66: Synthesis of Compound 66

Compound 66 was prepared following a similar procedure as described for Compound 62.

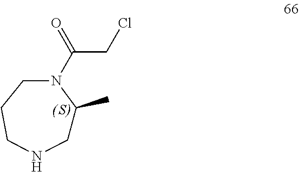

(S)-2-chloro-1-(2-methyl-1,4-diazepan-1-yl)ethanone (66) (41.7 mg, 117.1 umol, 85.2%) was obtained as brown oil. ¹H NMR (400 MHz, methanol-d4) δ=4.82-4.62 (m, 1H), 4.59-4.12 (m, 3H), 3.99-3.41 (m, 3H), 3.25-2.98 (m, 2H), 2.17-1.89 (m, 2H), 1.39-1.08 (m, 3H). ESI [M+H]=191.1.

### Example S67: Synthesis of Compound 67

Compound 67 was prepared following a similar procedure as described for Compound 62.

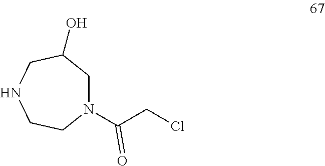 [SMILES: O=C(CCl)N1CCNCC(O)C1]

2-chloro-1-(6-hydroxy-1,4-diazepan-1-yl)ethanone (67) (17.5 mg, 50.7 umol, 87.3%) was obtained as colorless oil. ¹H NMR (400 MHz, methanol-d4) δ=4.45 (d, J=13.4 Hz, 1H), 4.30-4.16 (m, 2H), 4.12-4.03 (m, 1H), 3.90-3.79 (m, 1H), 3.50-3.29 (m, 4H), 3.22 (td, J=1.7, 3.2 Hz, 1H), 3.16-3.07 (m, 1H). ESI [M+H]=193.1.

### Example S68: Synthesis of Compound 68

Compound 68 was prepared following a similar procedure as described for Compound 62.

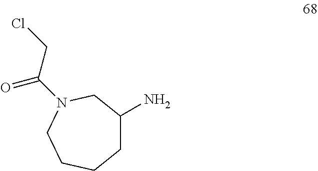 [SMILES: NC1CCCCN(C(=O)CCl)C1]

1-(3-aminoazepan-1-yl)-2-chloro-ethanone (68) (7.3 mg, 19.9 umol, 82.7%, 82.6% purity) was obtained as colorless oil. ¹H NMR (400 MHz, methanol-d4) δ=4.33-4.14 (m, 2H), 3.78-3.58 (m, 3H), 3.45-3.28 (m, 2H), 2.10-1.94 (m, 1H), 1.91-1.58 (m, 3H), 1.53-1.37 (m, 2H). ESI [M+H]=191.1.

### Example S69: Synthesis of Compound 69

Compound 69 was prepared following a similar procedure as described for Compound 62.

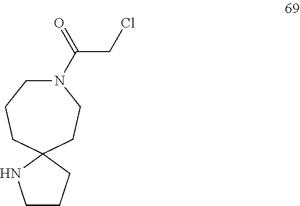 [SMILES: O=C(CCl)N1CCCC2(CCCN2)CC1]

2-chloro-1-(1,8-diazaspiro[4.6]undecan-8-yl)ethanone (69) (17.2 mg, 45.4 umol, 37.6%) was obtained as light yellow oil. ¹H NMR (400 MHz, methanol-d4) δ=4.25-4.13 (m, 2H), 3.70-3.57 (m, 1H), 3.57-3.39 (m, 2H), 3.39-3.18 (m, 3H), 2.15-1.62 (m, 10H). ESI [M+H]=231.1.

### Example S70: Synthesis of Compound 70

Compound 70 was prepared following a similar procedure as described for Compound 62.

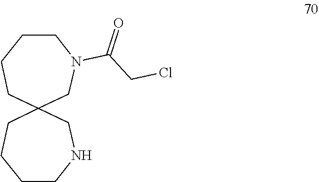 [SMILES: O=C(CCl)N1CCCCC2(CCCCNC2)C1]

2-chloro-1-(2,9-diazaspiro[6.6]tridecan-2-yl)ethanone (70) (13.4 mg, 32.9 umol, 71.1%) was obtained as light yellow oil. ¹H NMR (400 MHz, methanol-d4) δ=4.28-4.13 (m, 2H), 4.06 (br d, J=14.5 Hz, 1H), 3.78 (td, J=4.8, 14.2 Hz, 1H), 3.25 (br d, J=5.1 Hz, 1H), 3.19-3.15 (m, 1H), 3.15-3.10 (m, 1H), 2.99-2.87 (m, 1H), 2.76-2.60 (m, 2H), 1.90-1.45 (m, 10H), 1.34-1.17 (m, 2H). ESI [M+H]=259.1.

### Example S71: Synthesis of Compound 71

Compound 71 was prepared following a similar procedure as described for Compound 62.

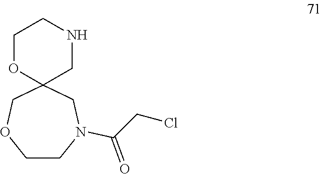 [SMILES: O=C(CCl)N1CCOCC2(CNCCO2)C1]

2-chloro-1-(1,8-dioxa-4,11-diazaspiro[5.6]dodecan-11-yl)ethanone (71) (19.4 mg, 63.7 umol, 57.7%, 71.3% purity) was obtained as colorless oil. ¹H NMR (400 MHz, methanol-d4) δ=4.48 (br d, J=14.3 Hz, 1H), 4.40-4.21 (m, 2H), 4.13-3.74 (m, 6H), 3.67-3.37 (m, 3H), 3.29-2.96 (m, 4H). ESI [M+H]=249.1.

### Example S72: Synthesis of Compound 72

Compound 72 was prepared following a similar procedure as described for Compound 62.

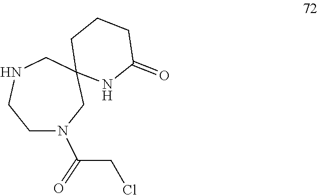 [SMILES: O=C1CCCC2(CNCCN(C(=O)CCl)C2)N1]

8-(2-chloroacetyl)-1,8,11-triazaspiro[5.6]dodecan-2-one (72) (10 mg, 22.4 umol, 80.6%, 83.8% purity) was obtained as light yellow oil. ¹H NMR (400 MHz, methanol-d4) δ=4.52-4.27 (m, 2H), 4.20-3.75 (m, 3H), 3.74-3.35 (m, 4H), 3.30-3.16 (m, 1H), 2.50-2.26 (m, 2H), 2.11-1.64 (m, 4H). ESI [M+H]=260.1.

### Example S73: Synthesis of Compound 73

Compound 73 was prepared following a similar procedure as described for Compound 62.

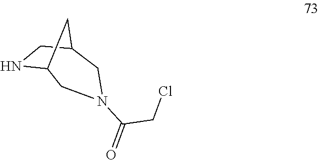 [SMILES: O=C(CCl)N1CC2CNC(C2)C1]

2-chloro-1-(3,6-diazabicyclo[3.2.1]octan-3-yl)ethanone (73) (16.5 mg, 54.5 umol, 92.6%, 71.8% purity) was obtained as colorless oil. ¹H NMR (400 MHz, methanol-d4) δ=4.50-4.24 (m, 3H), 4.14 (br s, 1H), 4.07-3.83 (m, 1H), 3.57-3.35 (m, 2H), 3.22 (br d, J=11.8 Hz, 1H), 3.10-2.95 (m, 1H), 2.80 (br s, 1H), 2.19-2.01 (m, 2H). ESI [M+H]=189.1.

### BIOLOGICAL EXAMPLES

### Example B1. Inhibition Studies of the Targeted Autophagy Protein Binder

Recombinant pure human SQSTM1 protein was purchased from Origene. SQSTM1 protein (0.21 μg) was diluted into 50 μL of PBS and 1 μL of either DMSO (vehicle) or covalently acting small molecule (test compound) to achieve the desired concentration. After 30 min. at 37° C., the samples were treated with 100 nM IA-Rhodamine (tetramethylrhodamine-5-iodoacetamide dihydroiodide; Setareh Biotech, 6222, prepared in anhydrous DMSO) for 1 hour at room temperature. Samples were then diluted with 20 μL of 4× reducing Laemmli SDS sample loading buffer (Alfa Aesar) and heated at 90° C. for 5 min. The samples were separated on precast 4-20% Criterion TGX gels (Bio-Rad Laboratories, Inc.). Fluorescent imaging was performed on a ChemiDoc MP instrument (Bio-Rad Laboratories, Inc.), and inhibition of target labeling was assessed by densitometry using ImageJ software. The initial screen was performed using 50 μM of test compound. Inhibition data for the tested compounds is provided in Table 2.

TABLE 2

| Compound | % inhibition |
|---|---|
| 1 | 79 |
| 2 | 50 |
| 3 | 58 |
| 4 | 18 |
| 5 | 25 |
| 6 | 56 |
| 7 | 61 |
| 8 | 50 |
| 9 | 64 |
| 10 | 17 |
| 11 | 57 |
| 12 | not active |
| 13 | 49 |
| 14 | 38 |
| 15 | 29 |
| 16 | 20 |
| 17 | 9 |
| 18 | 21 |
| 19 | 83 |
| 20 | 9 |
| 21 | 11 |
| 22 | not active |
| 23 | 21 |
| 24 | not active |
| 25 | 31 |
| 26 | 70 |
| 27 | 58 |
| 28 | 59 |
| 29 | 38 |
| 30 | 50 |
| 31 | 39 |
| 32 | 17 |
| 33 | 32 |
| 34 | 14 |
| 35 | 17 |
| 36 | 34 |
| 37 | 30 |
| 38 | 40 |
| 39 | 20 |
| 40 | not active |
| 41 | 54 |
| 42 | 32 |
| 43 | 25 |
| 44 | 13 |
| 45 | not active |
| 46 | 31 |
| 47 | 36 |
| 48 | 7 |
| 49 | 9 |
| 50 | 17 |
| 51 | 33 |
| 52 | 40 |
| 53 | 52 |
| 54 | 46 |
| 55 | 35 |
| 56 | 50 |
| 57 | 58 |
| 58 | 82 |
| 59 | 22 |
| 60 | 21 |
| 61 | 18 |
| 62 | 27 |
| 63 | not active |
| 64 | 22 |
| 65 | 7 |
| 66 | not active |
| 67 | 35 |
| 68 | 44 |
| 69 | 22 |
| 70 | 22 |
| 71 | not active |
| 72 | not active |
| 73 | 38 |

### Example B2. Dose Response Studies Using Compound 1 and Compound 19

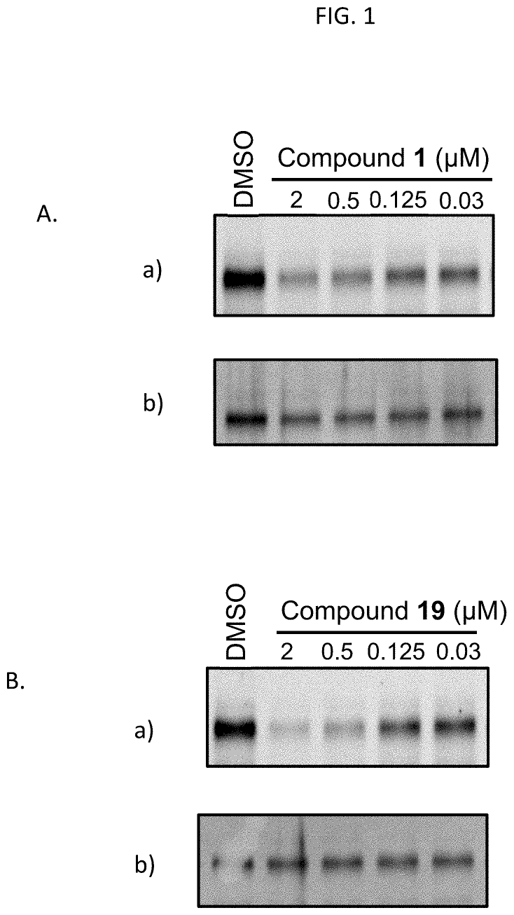

Protein SQSTM1 (p62) was pre-incubated with either Compound 1 or Compound 19 at concentrations of 2, 0.5, 0.125, and 0.03 μM, followed by addition of the reactive probe 5-carboxytetramethylrhodamine (TAMRA). Measurements were made using TAMRA fluorescence. Silver staining of the protein was used as a protein loading control. Decreasing fluorescence of TAMRA indicates binding of the test compound. The dose-response of Compound 1 and Compound 19 against SQSTM1 is shown in FIG. 1. The data demonstrate that both Compound 1 and Compound 19 covalently bind to SQSTM1.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A compound comprising a monovalent cellular component binder covalently bound to a monovalent targeted autophagy protein binder, wherein the monovalent targeted autophagy protein binder is a monovalent form of the formula: 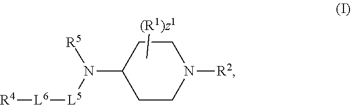
wherein z1 is an integer from 0-9; 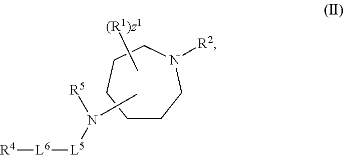
wherein z1 is an integer from 0-11; 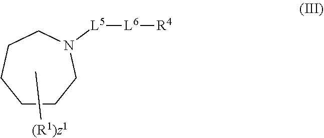
wherein z1 is an integer from 0-12; 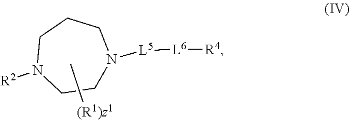
wherein z1 is an integer from 0-10; 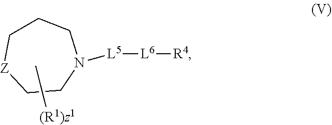
wherein Z is O, S, or SO₂, and z1 is an integer from 0-10; 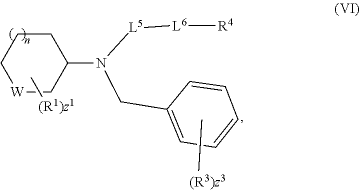
wherein W is O, NH, NR¹, or CH₂; n is 0 or 1; z1 is an integer from 0-11; and z3 is an integer from 0-5; 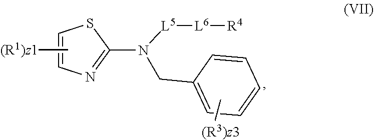
wherein z1 is an integer from 0-2, and z3 is an integer from 0-5;
R¹ is independently oxo, halogen, —CX¹₃, —CHX¹₂, —CH₂X¹, —OCX¹₃, —OCH₂X¹, —OCHX¹₂, —CN, —SOₙ₁R^{1D}, SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR_{1D}, —NR^{1A}SO₂R^{1D}, —NR^{1A}C(O)R^{1C}, —NR^{1A}C(O)OR^{1C}, —NR^{1A}OR^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or two R¹ substituents are taken together to form a substituted or unsubstituted alkylene, substituted or unsubstituted cycloalkyl, substituted or unsubstituted heterocycloalkyl, substituted or unsubstituted aryl, or substituted or unsubstituted heteroaryl; or one R¹ substituent is taken together with R² to form a substituted or unsubstituted heterocycloalkyl, or substituted or unsubstituted heteroaryl;
R² is independently H, oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)m2, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R² is taken together with one R¹ substituent to form a substituted or unsubstituted heterocycloalkyl, or substituted or unsubstituted heteroaryl;
R³ is independently oxo, halogen, —CX³₃, —CHX³₂, —CH₂X³, —OCX³₃, —OCH₂X³, —OCHX³₂, —CN, —SOₙ₃R^{3D}, SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)O R^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or two R³ substituents are taken together to form a substituted or unsubstituted cycloalkyl, substituted or unsubstituted heterocycloalkyl, substituted or unsubstituted aryl, or substituted or unsubstituted heteroaryl;
R⁴ is 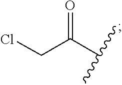 [SMILES: *C(=O)CCl]
L⁵ is a bond, —S(O)₂—, —S(O)—, —NR⁵—, ═N—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁵ is hydrogen, —CX⁵₃, —CHX⁵₂, —CH₂X⁵, —OCX⁵₃, —OCH₂X⁵, —OCHX⁵₂, —CN, —C(O)R^{5C}, —C(O)—OR^{5C}, —C(O)NR^{5A}R^{5B}, —OR^{5D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L⁶ is a bond, —S(O)₂—, —S(O)—, —NR⁶—, ═N—, —O—, —S—, —C(O)—, —C(O)NR⁶—, —NR⁶C(O)—, —NR⁶C(O)NH—, —NHC(O)NR⁶—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁶ is hydrogen, —CX⁶₃, —CHX⁶₂, —CH₂X⁶, —OCX⁶₃, —OCH₂X⁶, —OCHX⁶₂, —CN, —C(O)R^{6C}, —C(O)—OR^{6C}, —C(O)NR^{6A}R^{6B}, —OR^{6D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}, R^{2D}, R^{3A}, R^{3B}, R^{3C}, R^{3D}, R^{5A}, R^{5B}, R^{5C}, R^{5D}, R^{6A}, R^{6B}, R^{6C}, and R^{6D} are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom can be taken together to form a substituted or unsubstituted heterocycloalkyl or substituted or unsubstituted heteroaryl; R^{2A} and R^{2B} substituents bonded to the same nitrogen atom can be taken together to form a substituted or unsubstituted heterocycloalkyl or substituted or unsubstituted heteroaryl; R^{3A} and R^{3B} substituents bonded to the same nitrogen atom can be taken together to form a substituted or unsubstituted heterocycloalkyl or substituted or unsubstituted heteroaryl; R^{5A} and R^{5B} substituents bonded to the same nitrogen atom can be taken together to form a substituted or unsubstituted heterocycloalkyl or substituted or unsubstituted heteroaryl; R^{6A} and R^{6B} substituents bonded to the same nitrogen atom can be taken together to form a substituted or unsubstituted heterocycloalkyl or substituted or unsubstituted heteroaryl;
X, X¹, X², X³, X⁵, and X⁶ are independently —F, —Cl, —Br, or —I;
n1, n2, and n3 are independently an integer from 0 to 4; and
m1, m2, m3, v1, v2, and v3 are independently 1 or 2.

2. The compound of claim 1, wherein a divalent linker binds said monovalent cellular component binder to said monovalent targeted autophagy protein binder.

3. The compound of claim 1 or 2, wherein the cellular component is a protein, ion, lipid, nucleic acid, nucleotide, amino acid, particle, organelle, cellular compartment, microorganism, virus, lipid droplet, vesicle, small molecule, protein complex, protein aggregate, or macromolecule.

4. The compound of claim 3, wherein the cellular component is associated with a disease.

5. The compound of claim 4, wherein the disease is cancer, a neurodegenerative disease, a metabolic disease, an infectious disease, an autoimmune disease, or an inflammatory disease.

6. The compound of claim 2, wherein the divalent linker has the formula -L¹-L²-L³-L⁴-, wherein:
L¹ is connected directly to said monovalent targeted autophagy protein binder;
L¹ is —S(O)₂—, —S(O)—, —NH—, —O—, —S—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L² is a bond, —S(O)₂—, —S(O)—, —NH—, —O—, —S—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L³ is a bond, —S(O)₂—, —S(O)—, —NH—, —O—, —S—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
L⁴ is a bond, —S(O)₂—, —S(O)—, —NH—, —O—, —S—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

7. The compound of any one of claims 1-6, wherein the targeted autophagy protein binder is capable of contacting an amino acid corresponding to C26 of human p62/SQSTM1 protein.

8. The compound of any one of claims 1-6, wherein the targeted autophagy protein binder is capable of contacting an amino acid corresponding to C27 of human p62/SQSTM1protein.

9. The compound of any one of claims 1-6, wherein the targeted autophagy protein binder is capable of contacting an amino acid corresponding to C113 of human p62/SQSTM1protein.

10. The compound of any one of claims 7-9, wherein the targeted autophagy protein binder is capable of forming a covalent bond to the cysteine.

11. The compound of any one of claims 1-10, wherein the monovalent targeted autophagy protein binder is a monovalent form of the formula: 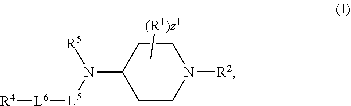
wherein z1 is an integer from 0-9.

12. The compound of claim 11, wherein z1 is 0, 1, or 2.

13. The compound of claim 11 or 12, wherein the monovalent targeted autophagy protein binder is a monovalent form of the formula:

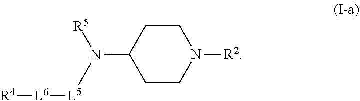

14. The compound of any one of claims 11-13, wherein:
R² is H, —C(O)—OR^{2C}, or substituted or unsubstituted alkyl; and
R^{2C} is substituted or unsubstituted alkyl.

15. The compound of claim 14, wherein:
R² is H or —C(O)OC(CH₃)₃.

16. The compound of any one of claims 11, 12, 14, and 15, wherein:
each R¹ is independently halogen, or substituted or unsubstituted alkyl.

17. The compound of claim 16, wherein:
each R¹ is independently —F, —Cl, or —CH₃.

18. The compound of any one of claims 11-17, wherein:
R⁵ is hydrogen, substituted or unsubstituted alkyl, substituted or unsubstituted heteroalkyl, substituted or unsubstituted cycloalkyl, or substituted or unsubstituted heterocycloalkyl.

19. The compound of claim 18, wherein:
R is

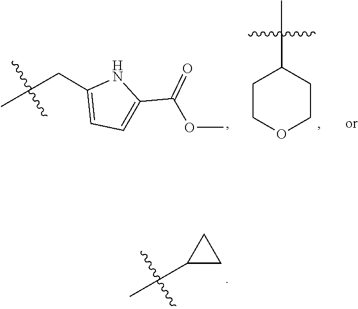

20. The compound of any one of claims 11-19, wherein:
L⁵ is a bond, substituted or unsubstituted alkylene, or substituted or unsubstituted heteroalkylene; and
L⁶ is a bond, substituted or unsubstituted alkylene, or substituted or unsubstituted heteroalkylene.

21. The compound of claim 20, wherein:
L⁵ and L⁶ are each a bond.

22. The compound of any one of claims 11-21, wherein the monovalent targeted autophagy protein binder is a monovalent form of the formula:

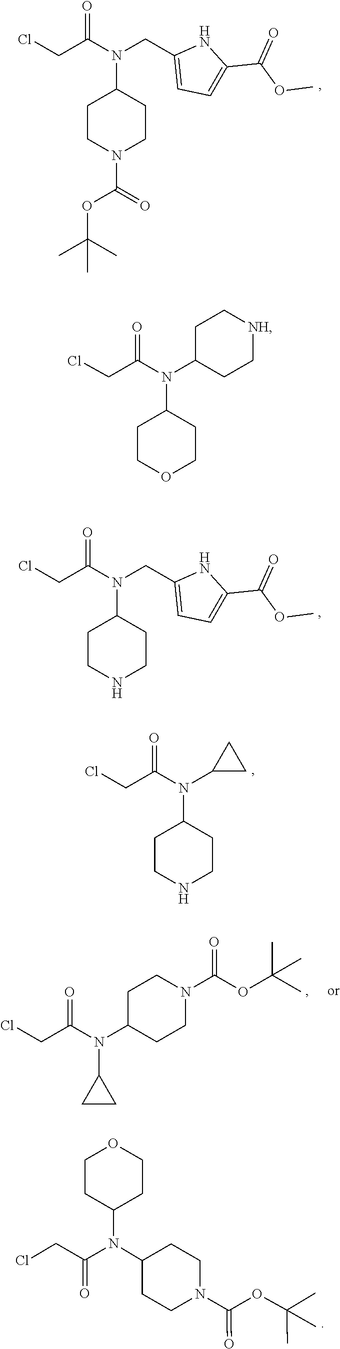

23. The compound of any one of claims 1-10, wherein the monovalent targeted autophagy protein binder is a monovalent form of the formula:

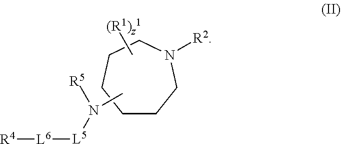

24. The compound of claim 23, wherein z1 is 0, 1, or 2.

25. The compound of claim 23 or 24, wherein the monovalent targeted autophagy protein binder is a monovalent form of the formula:

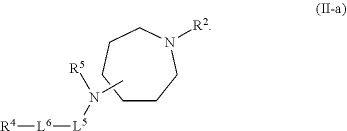

26. The compound of any one of claims 23-25, wherein the monovalent targeted autophagy protein binder is a monovalent form of the formula:

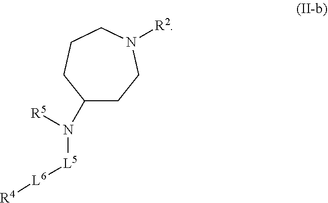

27. The compound of any one of claims 23-26, wherein:
R² is H, —C(O)—OR^{2C}, or substituted or unsubstituted alkyl; and
R^{2C} is substituted or unsubstituted alkyl.

28. The compound of claim 27, wherein:
R² is H or —C(O)OC(CH₃)₃.

29. The compound of any one of claims 23-28, wherein:
R⁵ is hydrogen, substituted or unsubstituted alkyl, substituted or unsubstituted heteroalkyl, substituted or unsubstituted cycloalkyl, or substituted or unsubstituted heterocycloalkyl.

30. The compound of claim 29, wherein:
R⁵ is isopropyl.

31. The compound of any one of claims 23-30, wherein:
L⁵ is a bond, substituted or unsubstituted alkylene, or substituted or unsubstituted heteroalkylene; and
L⁶ is a bond, substituted or unsubstituted alkylene, or substituted or unsubstituted heteroalkylene.

32. The compound of claim 31, wherein:
L⁵ and L⁶ are each a bond.

33. The compound of any one of claims 23-32, wherein the monovalent targeted autophagy protein binder is a monovalent form of the formula:

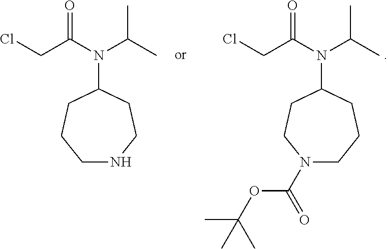 [SMILES: CC(C)N(C(=O)CCl)C1CCCNCC1 ; CC(C)N(C(=O)CCl)C1CCCN(C(=O)OC(C)(C)C)CC1]

34. The compound of any one of claims 1-10, wherein the monovalent targeted autophagy protein binder is a monovalent form of the formula:

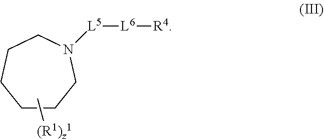

35. The compound of claim 34, wherein z1 is 1, 2, or 3.

36. The compound of claim 34 or 35, wherein the monovalent targeted autophagy protein binder is a monovalent form of the formula: 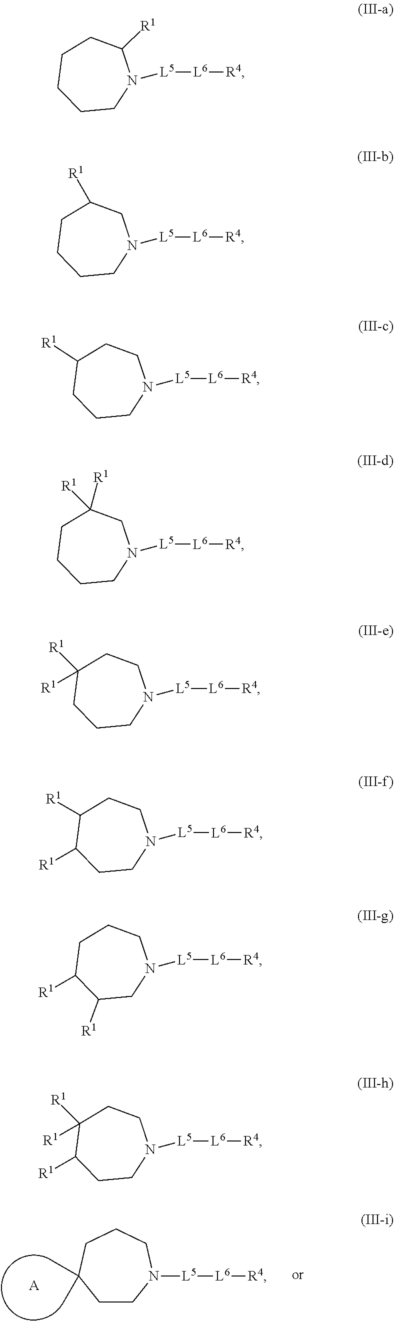 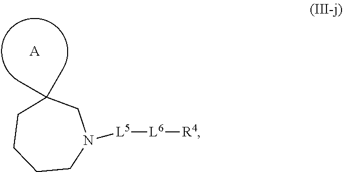
wherein the Ring A moiety is a substituted or unsubstituted cycloalkyl, substituted or unsubstituted heterocycloalkyl, substituted or unsubstituted aryl, or substituted or unsubstituted heteroaryl.

37. The compound of any one of claims 34-36, wherein:
each R¹ is independently oxo, halogen, —CX¹₃, —CHX¹₂, —CH₂X¹, —NR^{1A}R^{1B}, —C(O)—OR^{1C}, —NR^{1A}C(O)OR^{1C}, substituted or unsubstituted alkyl, or substituted or unsubstituted heteroalkyl; or two R¹ substituents are taken together to form a substituted or unsubstituted cycloalkyl, or substituted or unsubstituted heterocycloalkyl;
each R^{1A}, R^{1B}, and R^{1C} is independently hydrogen, or substituted or unsubstituted alkyl; and
each X¹ is independently —F or —Cl.

38. The compound of claim 37, wherein:
each R¹ is independently F, —CH₃, —OH, —CF₃, —CH₂F, —C(O)OCH₂CH₃, —NH₂, oxo, —CH₂N(H)C(O)OCH₂(C₆H₅), or —N(H)C(O)OC(CH₃)₃;
or two R¹ groups are taken together to form

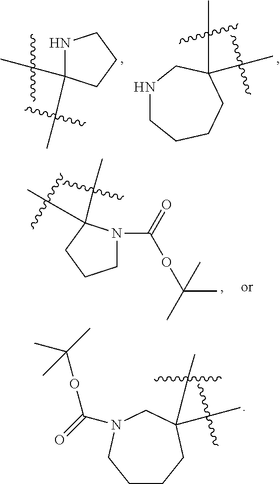

39. The compound of any one of claims 34-38, wherein:
L⁵ is a bond, substituted or unsubstituted alkylene, or substituted or unsubstituted heteroalkylene; and
L⁶ is a bond, substituted or unsubstituted alkylene, or substituted or unsubstituted heteroalkylene.

40. The compound of claim 39, wherein:
L⁵ and L⁶ are each a bond.

41. The compound of any one of claims 34-40, wherein the monovalent targeted autophagy protein binder is a monovalent form of the formula:

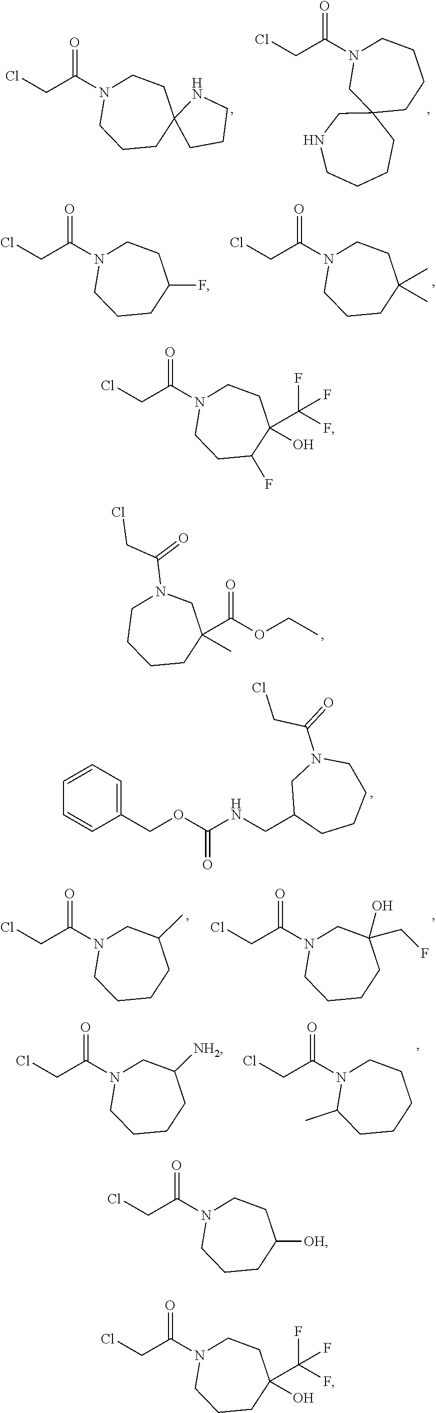

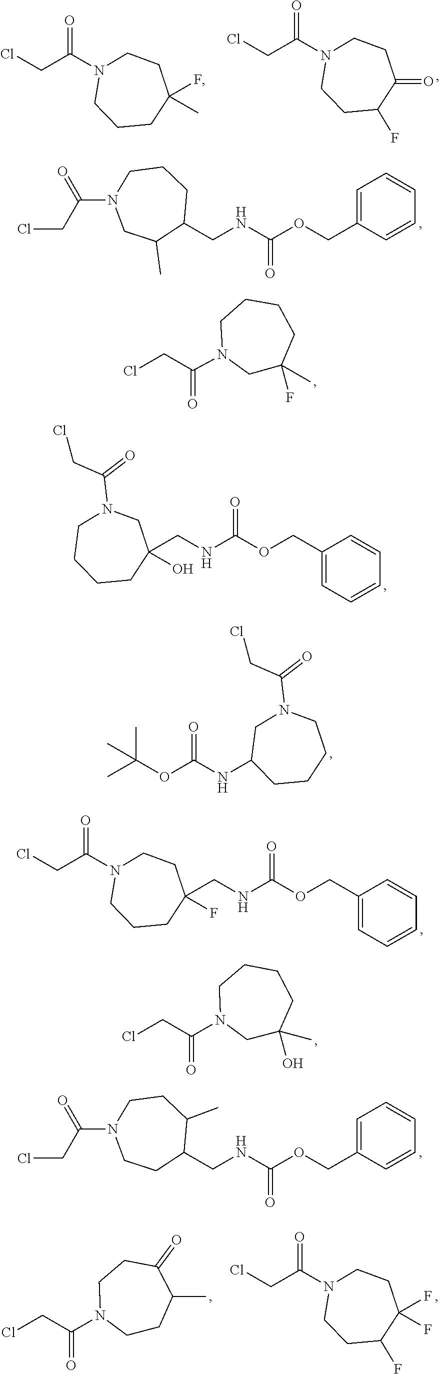

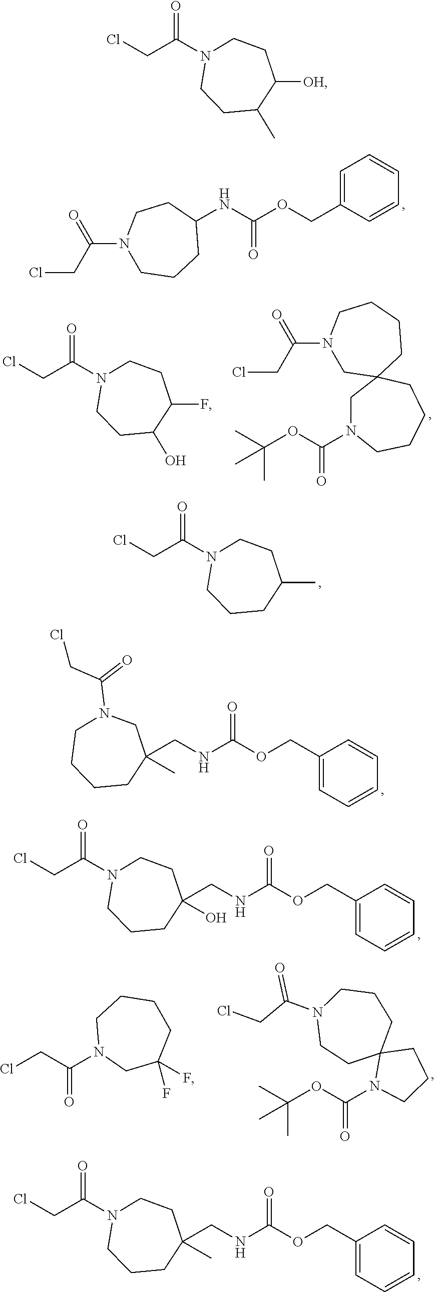

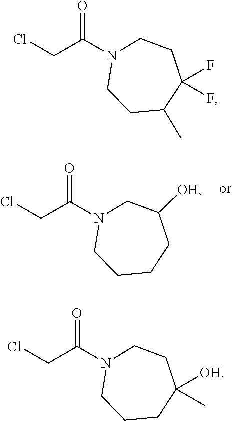

42. The compound of any one of claims 1-10, wherein the monovalent targeted autophagy protein binder is a monovalent form of the formula:

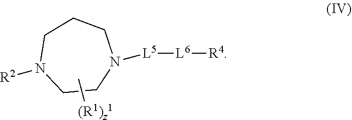

43. The compound of claim 42, wherein z1 is 0, 1, or 2.

44. The compound of claim 42 or 43, wherein the monovalent targeted autophagy protein binder is a monovalent form of the formula: 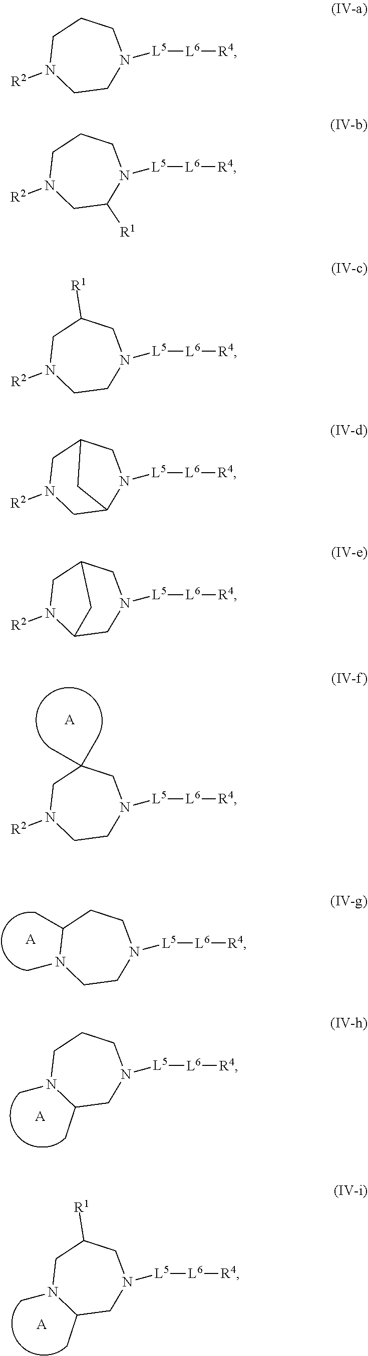
wherein the Ring A moiety is a substituted or unsubstituted cycloalkyl, substituted or unsubstituted heterocycloalkyl, substituted or unsubstituted aryl, or substituted or unsubstituted heteroaryl.

45. The compound of any one of claims 42-44, wherein:
each R¹ is independently halogen, —CX¹₃, —OR^{1D}, substituted or unsubstituted alkyl, or substituted or unsubstituted heteroalkyl; or two R¹ substituents are taken together to form a substituted or unsubstituted alkylene, substituted or unsubstituted cycloalkyl, or substituted or unsubstituted heterocycloalkyl; or one R¹ substituent is taken together with R² to form a substituted or unsubstituted heteroaryl;
each R^{1D} is independently hydrogen, or substituted or unsubstituted alkyl; and
each X¹ is independently —F or —Cl.

46. The compound of claim 45, wherein:
each R¹ is independently —CH₃ or —OH;
or two R¹ groups are taken together to form —CH₂—, 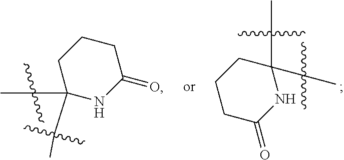 [SMILES: *C1(*)CCCC(=O)N1 ; *C1(*)CCCC(=O)N1]
or R¹ and R² are taken together to form

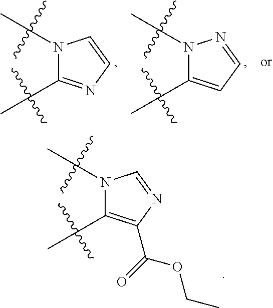 [SMILES: *c1nccn1* ; *c1ccnn1* ; *c1c(C(=O)OCC)ncn1*]

47. The compound of any one of claims 42-46, wherein:
each R² is independently hydrogen, substituted or unsubstituted alkyl, —C(O)OR^{2C}, or substituted or unsubstituted heteroaryl; or R² is taken together with one R¹ substituent to form a substituted or unsubstituted heteroaryl; and
each R^{2C} is independently substituted or unsubstituted alkyl.

48. The compound of claim 47, wherein:
R² is H, —CH₂CH₃, —C(O)OCH₃, —C(O)OC(CH₃)₃, or 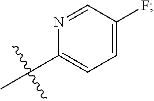 [SMILES: *c1ccc(F)cn1]
or R² and R¹ are taken together to form

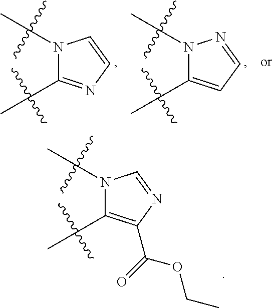 [SMILES: *c1nccn1* ; *c1ccnn1* ; *c1c(C(=O)OCC)ncn1*]

49. The compound of any one of claims 42-48, wherein:
L⁵ is a bond, substituted or unsubstituted alkylene, or substituted or unsubstituted heteroalkylene; and
L⁶ is a bond, substituted or unsubstituted alkylene, or substituted or unsubstituted heteroalkylene.

50. The compound of claim 49, wherein:
L⁵ and L⁶ are each a bond.

51. The compound of any one of claims 42-50, wherein the monovalent targeted autophagy protein binder is a monovalent form of the formula:

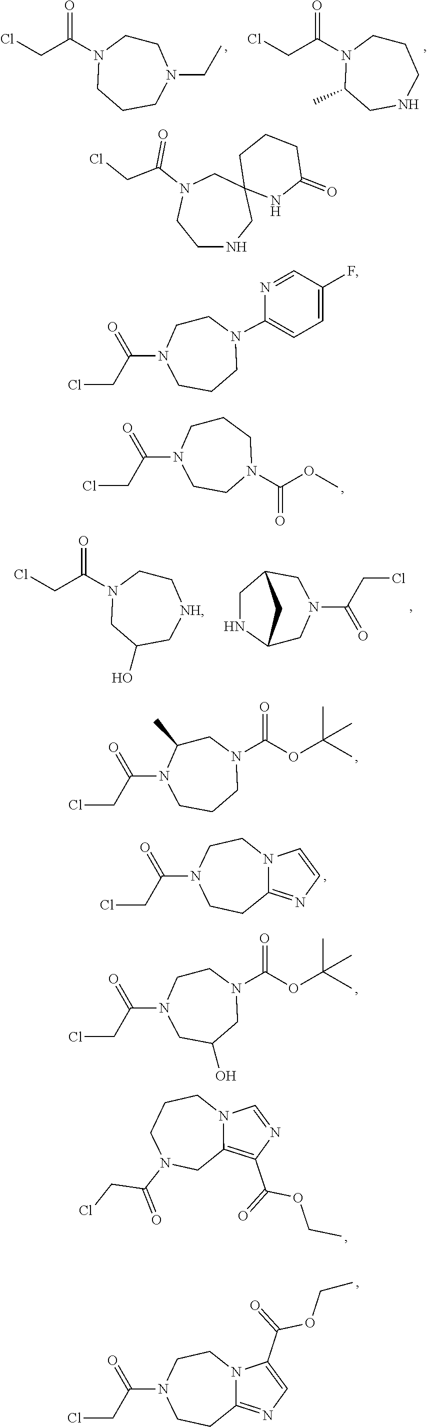

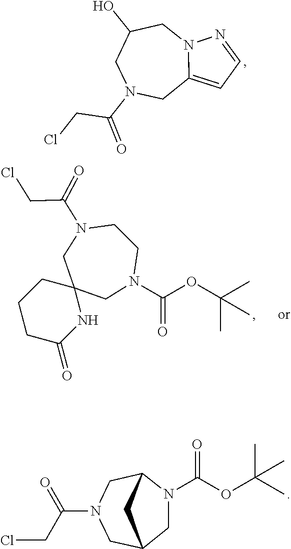

52. The compound of any one of claims 1-10, wherein the monovalent targeted autophagy protein binder is a monovalent form of the formula:

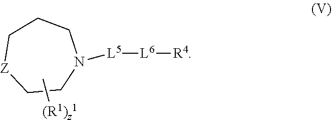

53. The compound of claim 51, wherein z1 is 0-3.

54. The compound of claim 52 or 53, wherein Z is O.

55. The compound of claim 54, wherein the monovalent targeted autophagy protein binder is a monovalent form of the formula:

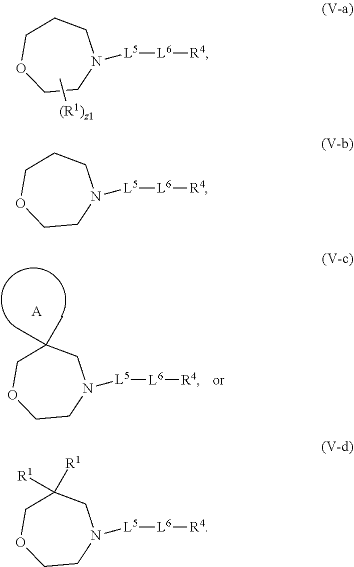

56. The compound of claim 52 or 53, wherein Z is S.

57. The compound of claim 56, wherein the monovalent targeted autophagy protein binder is a monovalent form of the formula:

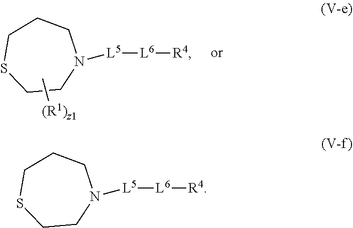

58. The compound of claim 52 or 53, wherein Z is SO₂.

59. The compound of claim 58, wherein the monovalent targeted autophagy protein binder is a monovalent form of the formula:

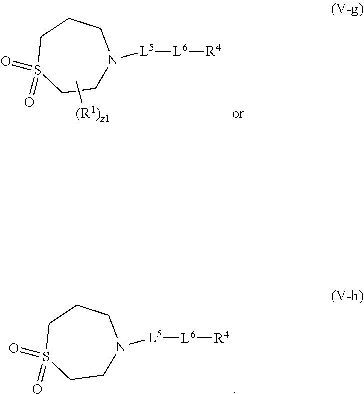

60. The compound of any one of claims 52-59, wherein:
each R¹ is independently halogen, —CX¹₃, substituted or unsubstituted alkyl, or substituted or unsubstituted heteroalkyl; or two R¹ substituents are taken together to form a substituted or unsubstituted cycloalkyl, or substituted or unsubstituted heterocycloalkyl; and
each X¹ is independently —F or —Cl.

61. The compound of claim 60, wherein:
each R¹ is independently —CH₃ or F;
or two R¹ substituents are taken together to form

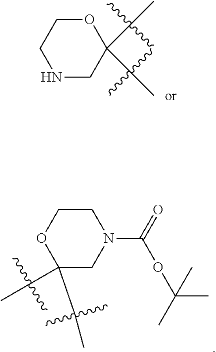

62. The compound of any one of claims 52-61, wherein:
L⁵ is a bond, substituted or unsubstituted alkylene, or substituted or unsubstituted heteroalkylene; and
L⁶ is a bond, substituted or unsubstituted alkylene, or substituted or unsubstituted heteroalkylene.

63. The compound of claim 62, wherein:
L⁵ and L⁶ are each a bond.

64. The compound of any one of claims 52-63, wherein the monovalent targeted autophagy protein binder is a monovalent form of the formula:

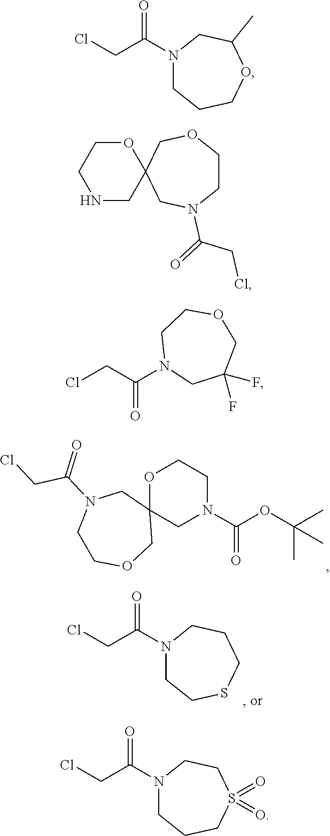

65. The compound of any one of claims 1-10, wherein the monovalent targeted autophagy protein binder is a monovalent form of the formula:

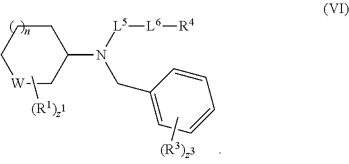

66. The compound of claim 65, wherein z1 is 0-3.

67. The compound of claim 65 or 66, wherein z3 is 0-2.

68. The compound of any one of claims 65-67, wherein n is 0.

69. The compound of any one of claims 65-67, wherein n is 1.

70. The compound of any one of claims 65-69, wherein W is CH₂.

71. The compound of claim 70, wherein the monovalent targeted autophagy protein binder is a monovalent form of the formula:

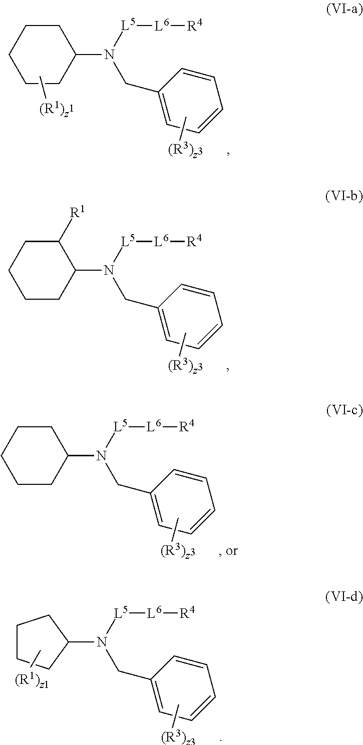

72. The compound of any one of claims 65-69, wherein W is O.

73. The compound of claim 72, wherein the monovalent targeted autophagy protein binder is a monovalent form of the formula:

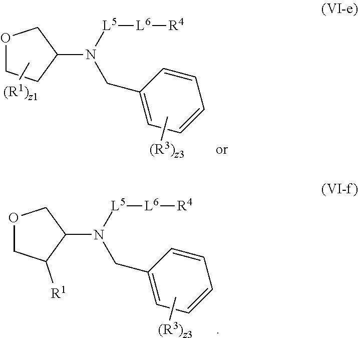

74. The compound of any one of claims 65-69, wherein W is NH or NR¹.

75. The compound of claim 74, wherein the monovalent targeted autophagy protein binder is a monovalent form of the formula:

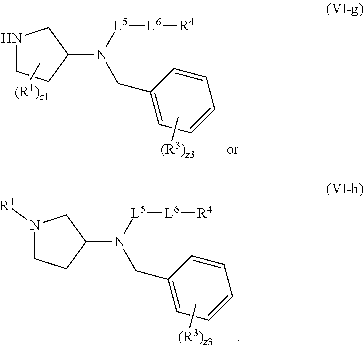

76. The compound of claim 75, wherein:
each R¹ is independently oxo, halogen, —OR^{1D}, or substituted or unsubstituted alkyl; and
each R^{1D} is independently hydrogen, or substituted or unsubstituted alkyl.

77. The compound of claim 76, wherein:
each R¹ is independently —CH₂OH, —OH, oxo, or —CH₂(C₆H₅).

78. The compound of any one of claims 65-77, wherein:
L⁵ is a bond, substituted or unsubstituted alkylene, or substituted or unsubstituted heteroalkylene; and
L⁶ is a bond, substituted or unsubstituted alkylene, or substituted or unsubstituted heteroalkylene.

79. The compound of claim 78, wherein:
L⁵ and L⁶ are each a bond.

80. The compound of any one of claims 65-79, wherein the monovalent targeted autophagy protein binder is a monovalent form of the formula:

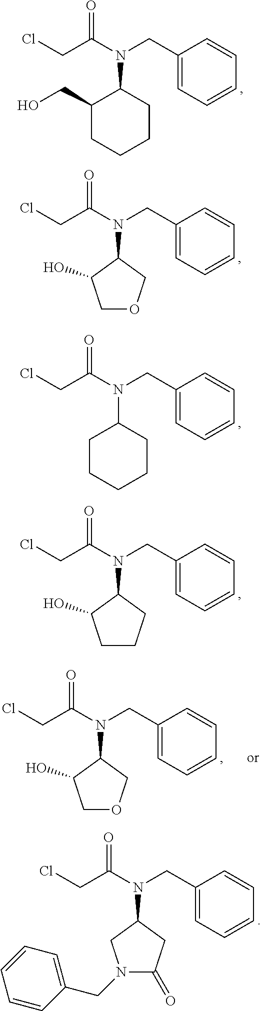

81. The compound of any one of claims 1-10, wherein the monovalent targeted autophagy protein binder is a monovalent form of the formula:

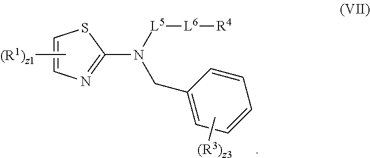

82. The compound of claim 81, wherein z1 is an integer from 0 or 1.

83. The compound of claim 81 or 82, where z3 is 0, 1, or 2.

84. The compound of any one of claims 81-83, wherein the monovalent targeted autophagy protein binder is a monovalent form of the formula:

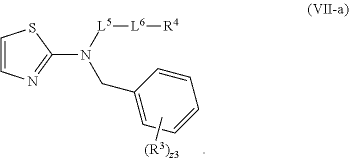

85. The compound of any one of claims 81-83, wherein:
each R¹ is independently halogen, —CX¹₃, substituted or unsubstituted alkyl, or substituted or unsubstituted heteroalkyl; and
each X¹ is independently —F or —Cl.

86. The compound of claim 85, wherein:
each R¹ is independently —F or —CH₃.

87. The compound of any one of claims 81-86, wherein:
each R³ is independently halogen, —OR^{3D}, or substituted or unsubstituted alkyl; and
each R^{3D} is independently hydrogen, or substituted or unsubstituted alkyl.

88. The compound of claim 87, wherein:
each R³ is —OCH₃.

89. The compound of any one of claims 81-88, wherein:
L⁵ is a bond, substituted or unsubstituted alkylene, or substituted or unsubstituted heteroalkylene; and
L⁶ is a bond, substituted or unsubstituted alkylene, or substituted or unsubstituted heteroalkylene.

90. The compound of claim 89, wherein:
L⁵ and L⁶ are each a bond.

91. The compound of any one of claims 81-90, wherein the monovalent targeted autophagy protein binder is a monovalent form of the formula:

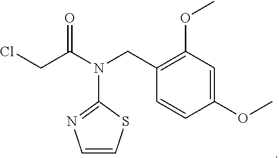 [SMILES: COc1ccc(CN(C(=O)CCl)c2nccs2)c(OC)c1 ; "."]

92. The compound of any one of claims 1-91, wherein the monovalent cellular component binder is a substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

93. The compound of any one of claims 1-92, wherein the monovalent cellular component binder is capable of binding BRD4.

94. The compound of claim 93, wherein the monovalent cellular component binder has the formula:

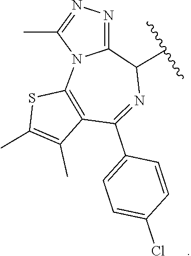 [SMILES: *C1N=C(c2ccc(Cl)cc2)c2c(sc(C)c2C)-n2c(C)nnc21 ; "."]

95. The compound of any one of claims 1-91, wherein the monovalent cellular component binder is capable of binding a protein aggregate.

96. The compound of claim 95, wherein the monovalent cellular component binder is capable of binding a huntingtin aggregate.

97. The compound of claim 96, wherein the monovalent cellular component binder is capable of binding a PolyQ huntingtin aggregate.

98. The compound of claim 95, wherein the monovalent cellular component binder is capable of binding an amyloid protein aggregate.

99. The compound of claim 95, wherein the monovalent cellular component binder is capable of binding a protein aggregate comprising a protein selected from the group consisting of amyloid precursor protein, beta amyloid, IAPP, alpha-synuclein, PrP, prion protein Sc, Huntingtin, calcitonin, atrial natriuretic factor, apolipoprotein A1, Serum amyloid A, medin, prolactin, transthyretin, lysozyme, beta-2 microglobulin, gelsolin, keratoepithelin, cystatin, immunoglobulin light chain AL, and S-IBM.

100. The compound of claim 95, wherein the monovalent cellular component binder is a monovalent form of thioflavin or a derivative thereof.

101. The compound of claim 95, wherein the monovalent cellular component binder is a monovalent form of the formula:

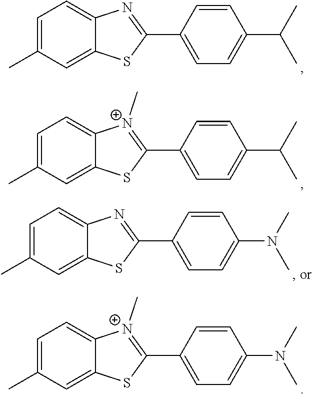

102. The compound of claim 95, wherein the monovalent cellular component binder is a monovalent form of the formula:

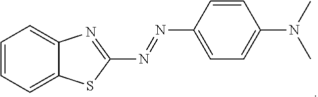 [SMILES: CN(C)c1ccc(/N=N/c2nc3ccccc3s2)cc1 ; "."]

103. The compound of claim 95, wherein the monovalent cellular component binder has the formula:

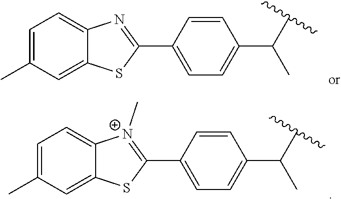

104. The compound of claim 95, wherein the monovalent cellular component binder has the formula:

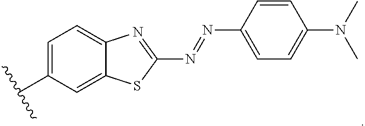 [SMILES: *c1ccc2nc(/N=N/c3ccc(N(C)C)cc3)sc2c1 ; "."]

105. An autophagy adapter protein covalently bonded to a fragment of the compound of any one of claims 1-104.

106. The autophagy adapter protein of claim 105, wherein the autophagy adapter protein is p62 or a derivative, fragment, or homolog thereof.

107. A pharmaceutical composition comprising the compound of any one of claims 1-104 and a pharmaceutically acceptable excipient.

108. A method of reducing the level of a cellular component, said method comprising contacting the cellular component with a targeted autophagy degrader, wherein the targeted autophagy degrader is the compound of any one of claims 1-104.

109. The method of claim 108, further comprising:
A) allowing formation of an autophagosome comprising the compound of claim 1;
B) allowing the autophagosome to acidify; and
C) allowing degradation of the cellular component.

110. A method of treating cancer, said method comprising contacting a cellular component associated with cancer with the compound of any one of claims 1-104.

111. A method of treating cancer, said method comprising administering to a subject in need thereof a therapeutically effective amount of the compound of any one of claims 1-104.

112. A method of treating neurodegenerative disease, said method comprising contacting a cellular component associated with the neurodegenerative disease with the compound of any one of claims 1-104.

113. A method of treating a neurodegenerative disease, said method comprising administering to a subject in need thereof a therapeutically effective amount of the compound of any one of claims 1-104.

114. The method of claim 113, wherein said neurodegenerative disease is Huntington Disease, Alzheimer Disease, or Parkinson's Disease.

115. A method of treating a metabolic disease, said method comprising contacting a cellular component associated with the metabolic disease with the compound of any one of claims 1-104.

116. A method of treating a metabolic disease, said method comprising administering to a subject in need thereof a therapeutically effective amount of the compound of any one of claims 1-104.

117. A method of treating an infectious disease, said method comprising contacting a cellular component associated with the infectious disease with the compound of any one of claims 1-104.

118. A method of treating an infectious disease, said method comprising administering to a subject in need thereof a therapeutically effective amount of the compound of any one of claims 1-104.

119. A method of treating an autoimmune disease, said method comprising contacting a cellular component associated with the autoimmune disease with the compound of any one of claims 1-104.

120. A method of treating an autoimmune disease, said method comprising administering to a subject in need thereof a therapeutically effective amount of the compound of any one of claims 1-104.

121. A method of treating an inflammatory disease, said method comprising contacting a cellular component associated with the inflammatory disease with the compound of any one of claims 1-104.

122. A method of treating an inflammatory disease, said method comprising administering to a subject in need thereof a therapeutically effective amount of the compound of any one of claims 1-104.

123. The method of claim 108, wherein the cellular component binder is associated with a disease.

124. The method of claim 123, wherein the disease is cancer, a neurodegenerative disease, a metabolic disease, an infectious disease, an autoimmune disease, or an inflammatory disease.

125. The method of claim 108, wherein the cellular component is a protein, ion, lipid, nucleic acid, nucleotide, amino acid, particle, organelle, cellular compartment, microorganism, virus, vesicle, small molecule, protein complex, protein aggregate, or macromolecule.
